(19) United States
(12) Patent Application Publication
(10) Pub. No.: US 2007/0037752 A1
(43) Pub. Date: Feb. 15, 2007

(54) Novel alanyl-amino peptidase inhibitors for functionally influencing different cells and treating immunological, inflammatory, neuronal, and other diseases

(76) Inventors: Siegfried Ansorge, Hohenwarthe (DE); Ute Bank, Stassfurt (DE); Karsten Nordhoff, Magdeburg (DE); Michael Taeger, Heinrichsberg (DE); Frank Striggow, Gerwisch (DE)

Correspondence Address: GREENBLUM & BERNSTEIN, P.L.C., 1950 ROLAND CLARKE PLACE, RESTON, VA, 20191, US

(21) Appl. No.: 10/575,882

(22) PCT Filed: Oct. 15, 2004
(86) PCT No.: PCT/EP04/11643

§ 371 Date: Sep. 15, 2006

(30) Foreign Application Priority Data
Oct. 15, 2003 (DE) 103 48 023.4

(51) Int. Cl. A61K 38/06; A61K 31/655; A61K 31/55; A61K 31/5415; A61K 31/5377; A61K 31/53; A61K 31/522; A61K 31/407; A61K 31/4439; A61K 31/4152

(52) U.S. Cl. 514/18; 514/242; 514/575; 514/422; 514/296; 514/379; 514/450; 514/359; 514/249; 514/364; 514/433; 514/447; 514/224.2; 514/366; 514/408; 514/396; 514/150; 514/337; 514/326; 514/471; 514/323; 514/410; 514/301; 514/217.03; 514/365; 514/291; 514/231.5; 514/262.1; 514/229.8; 514/614; 514/252.16; 514/404; 514/406; 514/369; 514/263.33; 514/394; 514/218; 514/259.31; 514/303

(57) ABSTRACT

The invention relates to substances which specifically inhibit peptidases splitting ala-p-nitroanilide for use in medicine. The invention further relates to the use of at least one such substance or of a pharmaceutical or cosmetic composition containing at least one such substance for the prophylaxis and therapy of diseases, in particular the prophylaxis and therapy of diseases with an overshooting immune response (autoimmune diseases, allergies and transplant rejections), of other chronic inflammatory diseases, neuronal diseases and brain damages, skin diseases (acne and psoriasis, among others), tumor diseases and special virus infections (including SARS).

Aminopeptidase N (APN, CD13, EC 3.4.11.2) belongs to the (ubiquitously present) group of alanyl aminopeptidases predominantly occurring as a membrane protein of type II, as does the cytosolic soluble alanyl aminopeptidase (EC 3.4.11.14; Puromycine-sensitive aminopeptidase, aminopeptidase PS, ence-phaline-degrading aminopeptidase). Both peptidases act metal-dependant and catalyse the hydrolysis of peptide bonds after N-terminal amino acids of oligopeptides, in the case of APN with preference for alanine at the N-terminal end (A. J. Barrett et al.: Handbook of Proteolytic Enzymes, Academic Press, 1998). All inhibitors of aminopeptidase N also inhibit the cytosolic alanyl amino-peptidase, while specific inhibitors exist for the cytosolic aminopeptidase (M. Komodo et al.; Bioorg. and Med. Chem. 9, 121(2001).

For both enzymes, important biological functions were demonstrated in different cell systems. This is true for the immune system (U. Lendeckel et al.: Intern. J. Mol. Med. 4, 17, 1999; T. Osada et al.: J. Neurosciences 19, 6068 (1999); published International Patent Application WO 01/89569 A1; published International Patent Application No. WO 02/053170 A3; International Patent Application No. PCT/EP 03/07199), the neuronal system (published International Patent Application No. WO 02/053169 A2 and German Patent Application No. 103 37 074.9), the fibroblasts (German Patent Application No. 103 30 842.3), the keratinozytes (published International Patent Application No. WO 02/053170 A3), die sebaceous gland cells/Sebocytes (International Patent Application No. PCT/EP 03/02356), for tumors and for virus infections. The receptor for corona viruses is suppressed by inhibitors of this peptidase (D. P. Kontoyiannis et al.: Lancet 361, 1558, 2003).

For both alanyl aminopeptidases, distinguishable inhibitors are known (M.-C. Fournie-Zaluski and B. P. Roques, in: J. Langner and S. Ansorge, Ectopeptidases, Kluwer Academic/Plenum Publishers, p 51 (2002); M. Komodo et al.: Bioorg. and Med. Chem. 9, 121, 2001; Y. Hashimoto: Bioorg. and Med. Chem. 10, 461, 2002).

The isolated inhibition of the alanyl aminopeptidases and of analogous peptidases, but particularly the combined inhibition of these peptidases and of dipeptidyl peptidase IV and of analogous enzymes results into a strong inhibition of the DNA synthesis and, thereby, of the cell proliferation in immune cells as well as into a change of the cytokine production, particularly into an induction of the immunoregulatory effective TGF-β1 (published International Patent Application No. WO 01/89569 A1; published International Patent Application No. WO 02/053170 A3). For regulatory T-cells, alanyl aminopeptidase inhibitors effect a strong induction of TGF-β1 (International Patent Application No. PCT/EP 03/07199). In the neuronal system, a reduction or deceleration, respectively, of acute and chronic cerebral deterioration processes by an inhibition of alanyl aminopeptidases, but particularly by a combined inhibition of the alanyl amino-peptidases and of dipeptidyl peptidase IV or of analogous enzymes was demonstrated (published International Patent Application WO 02/053169 A3 and German laid-open Patent Application No. 103 37 074.9). It could be shown, too, for fibroblasts (German laid-open Patent Application No. 103 30 842.3), keratinocytes (published International Patent Application No. WO 02/053 170 A3) and Sebatocytes (International Patent Application No. PCT/EP 03/02356) that an inhibition of alanyl aminopeptidases, but particularly a combined inhibition of the two peptidase systems effects an inhibition of the growth and a change of the cytokine production.

Thus, there results the surprising fact that the alanyl aminopeptidases as well as analogously working enzymes perform fundamental central biological functions in several organs and cell systems, and that an inhibition of these peptidases alone, but particularly a combined inhibition of these enzymes together with an inhibition of the dipeptidyl peptidase IV and of analogous peptidases, represents a novel effective therapeutic principle for the treatment of different diseases which are chronic in most of the cases.

By using accepted animal models, the Inventors could demonstrate that, particularly, the combined administration of inhibitors of both peptidases effects, in fact, also in vivo an inhibition of the growth of different cell systems and a suppression of an excessive immune response, of chronic-inflammatory events as well as of cerebral damage (published International Patent Application WO 01/89569 A1).

The results achieved up to now were, predominantly, obtained by using known inhibitors of alanyl aminopeptidases, which are described in the literature and are, in part, commercially available, alone and, particularly, in combination.

It was an object of the present invention to find further effective inhibitors of alanyl aminopeptidases. In particular, lower molecular weight compounds and easily accessible compounds were to be found which allow an effective inhibition of alanyl aminopeptidases and of analogous enzymes.

Surprisingly, in the course of a high-throughput screening of substance data bases, there were now found novel, predominantly non-peptidic low-molecular inhibitors for the alanyl aminopeptidases.

The invention relates to novel substances specifically inhibiting peptidases cleaving Ala-p-nitroanilide.

Specifically, the present invention relates to substances of the general formulae A1 to A14 according to claims 1, 3, 5, 7, 9, 11, 13, 15, 17, 19, 21, 23, 25 and 27 as well as tautomers and stereoisomers of said compounds of the general formulae A1 to A14, as well as pharmaceutically acceptable salts, salt derivatives, tautomers and stereoisomers thereof, for a use in the medical field.

In a specific embodiment, the present invention relates to specific compounds having the specific formulae A1.001 to D14.003 which are covered by the above general formulae A1 to A14, which compounds, as examples and without restricting them to those, are listed in claims 2, 4, 6, 8, 10, 12, 14, 16, 18, 20, 22, 24, 26 and 28 in the form of tables, as well as tautomers and stereoisomers of said compounds of the general formulae A1.001 to A14.007, and pharmaceutically acceptable salts, salt derivatives, tautomers and stereoisomers thereof, for a use in the medical field.

Moreover, the invention relates to pharmaceutical compositions comprising at least one compound having one of the general formulae A1 to A14, optionally in combination with per se known and usual carriers and adjuvants.

Moreover, the invention relates to cosmetic compositions comprising at least one compound having one of the general formulae A1 to A14, optionally in combination with per se known and usual carriers and adjuvants.

Furthermore, the invention relates to the use of at least one compound of one of the general formulae A1 to A14 or of at least one of the above-mentioned pharmaceutical or cosmetic compositions for inhibiting the activity of alanyl aminopeptidases or of analogous enzymes, in a manner alone or in combination with inhibitors of dipeptidyl peptidase IV (DP IV) or of analogous enzymes.

Furthermore, the invention relates to the use of at least one compound of one of the general formulae A1 to A14 or of at least one of the above-mentioned pharmaceutical or cosmetic compositions for topically influencing the activity of alanyl aminopeptidases or of analogous enzymes, in a manner alone or in combination with inhibitors of DP IV or of analogous enzymes.

Moreover, the invention relates to the use of at least one compound of one of the general formulae A1 to A14 or of at least one of the above-mentioned pharmaceutical or optionally also cosmetic compositions for a prophylaxis and therapy of a number of diseases which, as a matter of an exemplary description, are claimed in claims 33 to 45. In particular embodiments, without that this should be interpreted as restricting the invention, compounds of the general formulae A1 to A14 in accordance with the invention, particularly any of the particularly preferred compounds A1.001 to A14.003 summarized in Tables 1 to 14, may be used as such, or may be used as starting compounds for further compounds or may be used in combination with inhibitors of DP IV and with inhibitors of analogous enzymes for a therapy of diseases accompanied by an excessive immune response (autoimmune diseases, allergies and transplant rejections), of other chronic-inflammatory diseases, of neuronal diseases and of cerebral damage, of diseases of the skin (inter alia acne and psoriasis), of tumor diseases and of specific virus infections (inter alia SARS).

Furthermore, the invention relates to the use of at least one compound of one of the general formulae A1 to A14 or of at least one of the above-mentioned pharmaceutical or cosmetic compositions for manufacturing a medicament for inhibiting the activity of alanyl aminopeptidases or of analogous enzymes, alone or in combination with inhibitors of DP IV or of analogous enzymes.

Furthermore, the invention relates to the use of at least one compound of one of the general formulae A1 to A14 or of at least one of the above-mentioned pharmaceutical or cosmetic compositions for manufacturing a medicament for topically influencing the activity of alanyl aminopeptidases or of analogous enzymes, alone or in combination with inhibitors of DP IV or of analogous enzymes.

Furthermore, the invention relates to the use of at least one compound of one of the general formulae A1 to A14 or of at least one of the above-mentioned pharmaceutical or optionally also cosmetic compositions for manufacturing a medicament for a prophylactic and therapeutic treatment of a number of diseases claimed, in an exemplifying way, in claims 48 to 60. In particular embodiments, without restricting the invention, the compounds of the general formulae A1 to A14, especially the particularly preferred single compounds A1.001 to A14.003 shown in Tables 1 to 14, may be used, as such or as starting substances for further substances and in combination with inhibitors of DP IV or of analogous enzymes, for manufacturing a medicament for a therapy of diseases associated with an excessive immune response (autoimmune diseases, allergies or transplant rejections), of other chronic-inflammatory diseases, of neuronal diseases and cerebral damage, of skin diseases (inter alia acne and psoriasis), of tumor diseases and of specific virus infections (inter alia SARS).

Moreover, the invention relates to a process for inhibiting the activity of alanyl aminopeptidases and of analogous enzymes, alone or in combination with inhibitors of DP IV and of analogous enzymes, by an administration of at least one compound of the general formulae A1 to A14 or of at least one of the above pharmaceutical or cosmetic compositions in an amount required for an inhibition of the enzymatic activity.

Moreover, the invention relates to a process for topically influencing the activity of alanyl aminopeptidases and of analogous enzymes, alone or in combination with inhibitors of DP IV and of analogous enzymes, by an administration of at least one compound of the general formulae A1 to A14 or of at least one of the above pharmaceutical or cosmetic compositions in an amount required for influencing the enzymatic activity.

Moreover, the invention relates to a process for a prophylaxis and/or therapy of one of the diseases or conditions claimed in the claims 63 to 76 by inhibiting the activity of alanyl aminopeptidases and of analogous enzymes, alone or in combination with inhibitors of DP IV or of analogous enzymes, by an administration of at least one compound of the general formulae A1 to A14 or of at least one of the above pharmaceutical or cosmetic compositions in an amount required for a prophylactic or therapeutic treatment.

The term “analogous enzymes” as used in the present specification and in the claims relates to enzymes having an enzymatic activity analogous to the one shown by the membrane-located alanyl aminopeptidase. This is applicable, for example, for the cytosolic alanyl aminopeptidase. The above term is also explained, in this sense, in the above-referenced textbook “A. J. Barrett et al.; Handbook of Proteolytic Enzymes, Academic Press, 1998”.

In the general formulae A1 to A14, as can be seen from claims 1, 3, 5, 7, 9, 11, 13, 15, 17, 19, 21, 23, 25 and 27 in a general form, the residues Rn, i.e. the residues R1, R2, R3, R4, R5, R6, R7, R8, R9, R10, R11, R12 and R13, independent of each other represent a residue selected from the group consisting of hydrogen, unsubstituted or substituted, straight chain or branched C₁- to C₁₂ alkyl, C₂- to C₁₂ alkenyl and C₂- to C₁₂ alkynyl, hydroxy, thiol, C₁- to C₁₂ alkoxy, C₁- to C₁₂ alkylthio, unsubstituted or substituted, uncondensed or condensed aryl and cycloalkyl optionally containing one or several hetero atoms from the group of N, O, P and S, unsubstituted or substituted amino, unsubstituted or substituted carbonyl, unsubstituted or substituted thiocarbonyl and unsubstituted or substituted imino.

In detail, the residues Rn, in embodiments of the invention where they represent unsubstituted straight chain or branched alkyl groups having 1 to 12 carbon atoms, represent in preferred embodiments methyl, ethyl, n-propyl, i-propyl, n-butyl, i-butyl, sec-butyl, tert-butyl, n-pentyl, i-pentyl, sec-pentyl, tert-pentyl, n-hexyl, i-hexyl, 3-methylpentyl, 2-ethylbutyl, 2,2-dimethylbutyl as well as all straight chain and branched isomers for the residues heptyl, octyl, nonyl, decyl, undecyl and dodecyl. In accordance with the invention, particularly preferred from the above-mentioned group are alkyl groups having 1 to 6 carbon atoms; among those, the residues methyl, ethyl, n-propyl, i-propyl, n-butyl, i-butyl, sec-butyl and tert-butyl are even more preferred.

In other embodiments according to the invention, the residues Rn, in cases where they represent unsubstituted straight chain or branched alkenyl groups having 2 to 12 carbon atoms, represent in preferred embodiments vinyl, allyl, 1-butenyl, 2-butenyl and all straight chain and branched residues for the radicals pentenyl, hexenyl, heptenyl, octenyl, nonenyl, decenyl, undecenyl and dodecenyl, also with respect to the position of the C═C double bond. In further embodiments of the invention, the residues Rn may also represent straight chain or branched alkenyl groups having several double bonds. Preferred residues of this group are the butadienyl group and the isoprenyl group. Among the above-mentioned groups, particularly preferred in accordance with the invention are the alkenyl groups having 2 to 6 carbon atoms; of those, the groups vinyl, allyl, 1-butenyl and 2-butenyl are even more preferred.

In other embodiments according to the invention, the residues Rn, in cases where they represent unsubstituted straight chain or branched alkynyl groups having 2 to 12 carbon atoms, represent in preferred embodiments ethynyl, propynyl, 1-butynyl, 2-butynyl and all straight chain and branched residues for the radicals pentynyl, hexynyl, heptynyl, octynyl, nonynyl, decynyl, undecynyl and dodecynyl, also with respect to the position of the C═C triple bond. Among the above-mentioned groups, particularly preferred in accordance with the invention are the alkynyl groups having 2 to 6 carbon atoms; of those, the groups ethynyl, propynyl, 1-butynyl and 2-butynyl are even more preferred.

In accordance with the invention, straight chain and branched alkyl, alkenyl and alkynyl residues may be substituted in a further embodiment of the invention. The substituent(s) may be positioned at any desired position of the backbone made of carbon atoms and may be selected from the group consisting of halogen atoms as fluorine, chlorine, bromine and iodine, alkyl groups having 1 to 6 carbon atoms, alkoxy groups having 1 to 6 carbon atoms in the alkyl residue and amino groups which may be unsubstituted or substituted with one or two alkyl residues independently of each other and having 1 to 6 carbon atoms.

In further embodiments of the invention, the residues Rn in the general formulae A1 to A14 represent C₁- to C₁₂ alkoxy residues or C₁- to C₁₂ alkylthio residues. Also for the C₁- to C₁₂ alkyl residues of these alkoxy and alkylthio groups, the above definitions of the straight chain and branched alkyl residues are applicable. Particularly preferred are straight chain C₁- to C₆ alkoxy groups and straight chain C₁- to C₆ alkylthio groups, and particularly preferred are the residues methoxy, ethoxy, n-propoxy, methylthio, ethylthio and n-propylthio.

In further embodiments of the invention, the residues Rn in the general formulae A1 to A14 may also represent unsubstituted or substituted cycloalkyl residues. In accordance with the invention, the cycloalkyl residues may preferably contain three to eight atoms in the ring and may consist exclusively of carbon atoms or may contain one or several hetero atom(s). Among the purely carbocyclic rings, the residues cyclopentyl, cyclopentenyl, cyclopentadienyl, cyclohexyl, cyclohexenyl, cyclohexadienyl, cycloheptyl, cycloheptenyl, cycloheptadienyl and cycloheptatrienyl are particularly preferred. Examples for hetero atom-containing cycloalkyl residues are, in further embodiments of the invention, the residues tetrahydrofuranyl, pyrrolidinyl, imidazolinidyl, piperidinyl, piperazinyl and morpholinyl. Substituents to these carbocyclic and heterocyclic cycloalkyl residues may be selected from the above group of substituents of linear alkyl groups.

In further embodiments of the invention, the residues Rn in the compounds of the general formulae A1 to A14 may represent uncondensed or condensed aryl residues optionally containing one or several hetero atoms from the group of N, O, P and S. The aryl residues may have one ring or may have several rings and, if having several rings, two rings are preferred. Moreover, one ring may preferably have five, six or seven ring members. In systems consisting of several rings condensed to each other, benzo-condensed rings are particularly preferred, i.e. ring systems wherein at least one of the rings is an aromatic six-membered ring. Particularly preferred are aryl residues purely consisting of carbon atoms, selected from phenyl, cyclopentadienyl, cycloheptatrienyl and naphthyl. Particularly preferred aryl residues containing hetero atoms are, for example, selected from the group consisting of indolyl, cumaronyl, thionaphthenyl, quinolinyl(benzopyridyl), quinazolinyl(benzopyrimidinyl) and quinoxylinyl(benzopyrazinyl).

In another embodiment of the invention, cyclic residues either consisting of one ring or consisting of several rings, either containing carbon atoms exclusively or also containing hetero atoms, either aromatic systems or non-aromatic systems, may be substituted. The substituents may be bound to any position of the ring system, either to a carbon atom or to a hetero atom. They may be selected from the group consisting of halogen atoms as, for example, fluorine, chlorine, bromine and iodine, alkyl groups having 1 to 6 carbon atoms, alkoxy groups having 1 to 6 carbon atoms in the alkyl group, and unsubstituted amino groups or amino groups substituted with one or two alkyl groups having—independent of each other—1 to 6 alkyl groups.

Moreover, in accordance with the invention, the residues Rn(=R1 to R13) may also represent unsubstituted amino residues (—NH₂) or unsubstituted imino residues (—NH—) or substituted amino residues (—NHR1 or —NR1Rm) or substituted imino residues (>NRm). Herein, the residues R1 and Rm may have the meanings defined above in detail for the residues Rn, and they may be identical or different.

In accordance with the invention, the residues Rn(=R1 to R13) may also represent unsubstituted carbonyl residues (H—(C═O)—) or unsubstituted thiocarbonyl residues (H—(C═S)—) or for substituted carbonyl residues (Rm-(C═O)—) or substituted thiocarbonyl residues (Rm-(C═S)—). In these residues, the substituents Rm of substituted carbonyl residues or substituted thiocarbonyl residues have the meanings defined above for the possible substituents of the residues Rn.

In accordance with the invention, the above-mentioned residues Rn(=R1, R2, R3, R4, R5, R6, R7, R8, R9, R10, R11, R12 and/or R13) may be bound to the respective basic structures of the general formulae A1 to A14 via one of their carbon atoms. In an alternative embodiment, it is also possible that the residues Rn are bound to the respective basic structures of the general formulae A1 to A14 via the hetero atom or via one of their hetero atoms.

In several of the general formulae A1 to A14 (for example in the general formulae A1, A6 (i.e. A6a, A6b and A6c), A8 and A14), Y, Y1 and Y2 represent residues bound to the basic structure of the respective formula via a C═Y double bond (or a C═Y1 double bond and/or a C═Y2 double bond). In the formulae where they appear, the groups Y represent—independent of each other—one of the residues O, S or NRn, for example NR3, NR4 or NR5, bound to a carbon atom via a double bond. In the latter residues, the radicals Rn (for example R3, R4, R5) may have the meanings mentioned above, including the meaning “hydrogen”. Particularly preferably, Y represents O bound to a carbon atom via a double bond.

In several of the general formulae A1 to A14 (for example in the formulae A3, A9, A12, A14), X, X1, X2 and Z represent residues bound to two different carbon atoms via a C—X single bond each (or via a C—X1 single bond or via a C—X2 single bond) or via a C-Z single bond each. In the general formulae where they appear, the residues X and Z represent—independent of each other—the residues >NH, >NRn (for example >NR5 or >NR10), —O—, —S— —CH₂—, —CHRn- or —CRn₂-, bound to two different carbon atoms by a single bond each, wherein the residues Rn have the meaning given above, or they represent the residues >N—, >CH— or >CRn- (for example >CR8— or >CR9—) bound to three different carbon atoms via a single bond each, wherein Rn (for example R8, R9) have the meanings given above.

In the compounds of the general formula A6, Z represents P or S.

In the compounds having the general formulae A8, X and Z independent of each other represent residues from the group consisting of hydroxy, thiol, C₁- to C₁₂ alkoxy, C₁- to C₁₂ alkylthio, unsubstituted or substituted, uncondensed or condensed aryl or cycloalkyl optionally containing one or several hetero atoms from the group of N, O, P and S, and amino (NH₂, NHR1, NR1R2), wherein all above-mentioned meanings of X and Z correspond to the meanings for alkoxy, alkylthio, aryl, cycloalkyl and amino which were defined above in detail for the residues Rn of the general formulae A1 to A14.

In the compounds of the general formula A12, X1 and X2 may be identical or different and—independent of each other—are selected from the group consisting of hydroxy, thiol, C₁- to C₁₂ alkoxy, C₁- to C₁₂ alkylthio, unsubstituted or substituted, uncondensed or condensed aryl or cycloalkyl optionally containing one or several hetero atoms from the group of N, O, P and S, hydroxyl, thiol and amino (NH₂, NHR1, NR1R2). Therein, R1 and R2 have the meanings given above.

In the compounds of the general formula A14, X represents N or CH or CR8, P, P═O, P(OH)₂, P(OH)(OR8) or P(OR8)(OR9), and Z represents NH, NR10, O or S. In these residues, R8, R9 and R10 have the meanings defined above.

The compounds of the general formulae A1 to A14 (in general) as defined in claims 1, 3, 5, 7, 9, 11, 13, 15, 17, 19, 21, 23, 25 and 27 and the compounds A10.001 to A14.007 in Tables 1 to 14 in the claims 2, 4, 6, 8, 10, 12, 14, 16, 18, 20, 22, 24, 26 and 28 (specifically) may be prepared in accordance with processes known from the literature or are commercially available.

The compounds corresponding to the general formulae A1 to A14 (in general) and the specific compounds A1.001 to A14.003 indicated in Tables 1 to 14 (in preferred embodiments of the invention) are claimed for a use in the medical field. The term “for a use in the medical field” is understood here, and in the claims as well, in its broadest sense and relates to all conceivable fields of application, where the compounds of the general formulae A1 to A14 defined by the present invention, and the compounds A1.001 to A14.003 as mentioned in Tables 1 to 14, in preferred embodiments, may exert an effect in connection to medically relevant conditions of the body of a mammal, in particular of the body of a human.

In connection to such medically relevant conditions, the compounds of the general formulae A1 to A14 (in general) and the preferred compounds A1.001 to A14.003 according to Tables 1 to 14 are used either in the form of a single compound or are used in the form of more than one compound, or several compounds, of the general formulae A1 to A14 (in particular of the compounds A1.001 to A14.003 according to Tables 1 to 14). Also covered by the scope of the present invention is a use of one or more than one compound of the general formulae A1 to A14, preferably of one or more than one compound selected from the group consisting of the compounds A1.001 to A14.003 according to Tables 1 to 14, in combination with other effective agents, for example one or more than one compound having an effect in the inhibition of alanyl aminopeptidases or of analogous enzymes (i.e. of enzymes having an equal substrate specificity) and/or having an effect in the inhibition of other enzymes, e.g. of dopeptidyl peptidase IV (DP IV) or of analogous enzymes (i.e. of enzymes having an equal substrate specificity). Examples of such compounds having an effect as enzyme inhibitor(s) are mentioned in parallel patent applications filed by the Applicants of the present application on the same filing date as the present application as well as in the Applicants' patent applications referred to in the introduction to the present description, the whole disclosed content of which applications is incorporated into the present specification by this reference.

Specific examples of inhibitors effective as inhibitors of dipeptidyl peptidase IV or of analogous enzymes, which are known from the prior art and may optionally be used together with the compounds of the present invention particularly with one or several of the compounds A1.001 to A14.003 according to Tables 1 to 14, include, for example: Xaa-Pro dipeptides, corresponding derivatives, preferably dipeptide phosphonic acid diaryl esters, dipeptide boronic acids (e.g. Pro-bobo-Pro) and their salts, Xaa-Xaa-(Trp)-Pro-(Xaa)n peptides (n=0 to 10), corresponding derivatives and their salts, and amino acid (Xaa) amides, corresponding derivatives and their salts, wherein Xaa is an α-amino acid/imino acid or an α-amino acid derivative/imino acid derivative, preferably N′-4-nitrobenzyl-oxycarbonyl-L-lysine, L-proline, L-tryptophane, L-isoleucine, L-valine, and cyclic amines as, for example pyrrolidine, piperidine, thiazolidine and their derivatives act as the amide structure. Such compounds and their preparation were described in an earlier patent (K. Neubert et al.; DD 29 60 75 A5). Furthermore, tryptophane-1,2,3,4-tetrahydroisoquinoline-3-carboxylic acid derivatives (TSL) and (2S,2S′,2S″)-2-[2′-[2″-amino-3″-(indol-3′″-yl)-1″-oxoprolyl]-1′,2′,3′,4′-tetrahydro-6′8′-dihydroxy-7-methoxyisoquinol-3-yl-carbonyl-amino]-4-hydrome-thyl-5-hydropentanoic acid (TMC-2A) may advantageously be used as the effectors for the DP IV together with the compounds of the general formulae A1 to A14. One example of an inhibitor of DP IV preferably useable together with the compounds of the general formulae A1 to A14 is Lys[Z(NO₂)]thiazolidide, wherein Lys represents an L-lysine residue an Z(NO₂) represents 4-nitrobenzyl-oxycarbonyl (see also DD 29 60 75 A5).

Specific examples of inhibitors effective as inhibitors of alalyl aminopeptidase, which are known from the prior art and may optionally be used together with the compounds of the present invention particularly with one or several of the compounds A1.001 to A14.003 according to Tables 1 to 14, include, for example: actinonine, leuhistine, phebestine, amastatine, bestatine, probestine, β-amino thiols, α-amino phosphinic acids, α-amino phosphinic acid derivatives, preferably D-Phe-ψ-[PO(OH)—CH₂]-Phe-Phe. Known alanyl aminopeptidase inhibitors particularly preferred and useable together with the compounds of the present invention are bestatine (Ubenimex), actinonine, probestine, phebestine, RB3014 or leuhistine.

Another embodiment of the present invention relates to pharmaceutical compositions, which comprise at least one, optionally two or even more, compound(s) of the general formulae A1 to A14, particularly preferably selected from the compounds A1.001 to A14.003 according to Tables 1 to 14. Such pharmaceutical compositions comprise one or several of said compounds in such amounts required for exerting a pharmaceutical effect. Such amounts may in detail be determined by a skilled person by a few routine tests and without adding an inventive activity. In general, these amounts are in ranges of from 0.01 to 1000 mg of each of the compounds of the general formulae A1 to A14, particularly preferred of the compounds A1.001 to A14.003 according to Tables 1 to 14, per administration unit, even more preferred in ranges of from 0.1 to 100 mg of each of said compounds per administration unit. Moreover, amounts adjusted to the respective single mammalian organism or human organism may easily be determined by a skilled person, and it may also be provided that a sufficient concentration of the compound(s) to be used may be achieved by an administration of divided or of several administration units.

Another embodiment of the present invention relates to cosmetic compositions, which comprise at least one, optionally two or even more, compound(s) of the general formulae A1 to A14, particularly preferably selected from the com-pounds A1.001 to A14.003 according to Tables 1 to 14. Such cosmetic compo-sitions comprise one or several of said compounds in such amounts required for exerting a desired effect, for example a cosmetic effect. Such amounts may in detail be determined by a skilled person by a few routine tests and without adding an inventive activity. In general, these amounts are in ranges of from 0.01 to 1000 mg of each of the compounds of the general formulae A1 to A14, particularly preferred of the compounds A1.001 to A14.003 according to Tables 1 to 14, per administration unit, even more preferred in ranges of from 0.1 to 100 mg of each of said compounds per administration unit. Moreover, amounts adjusted to the respective single mammalian organism or human organism may easily be determined by a skilled person, and it may also be provided that a sufficient concentration of the compound(s) to be used may be achieved by an administration of divided or of several administration units.

The one compound or the several compounds according to the present invention or pharmaceutical or cosmetic compositions containing it/them is/are administered simultaneously with known carrier substances and/or auxiliary substances (adjuvants). Such carrier and auxiliary substances are known to a skilled person as such and also with respect to their function and way of application and need no detailed explanation here.

The invention also comprises pharmaceutical compositions which comprise: one or several of the inhibitors of the DP IV or of the inhibitors of enzymes having a DP IV-analogous enzymatic activity and/or of the inhibitors of the APN or of the inhibitors of enzymes having an APN-analogous enzymatic activity in accordance with the prior art, together with one or with several compound(s) of the general formulae A1 to A14, particularly preferably together with one or several compound(s) which are selected from the compounds A1.001 to A14.003 of the Tables 1 to 14, in a spaced apart formulation in combination with known carrier substances, auxiliary substances and/or additives for a simulta-neous or, with respect to the time, immediately successive administration with the aim of a joint effect.

The administration of the compounds of the general formulae A1 to A14 in general and, preferably, of the compounds A1.001 to A14.007 according to Tables 1 to 14 or the administration of pharmaceutical or cosmetic compositions comprising one or several of the above compounds together with usual carrier substances, auxiliary substances and/or additives, is effected, on the one hand, as a topical application in the form of, for example, creams, ointments, pastes, gels, solutions, sprays, liposomes and nanosomes, lotion, “pegylated” formul-ations, degradable (i.e. decomposable under physiological conditions) depot matrices, hydrocolloid dressings, plasters, micro-sponges, prepolymers and similar novel carrier substrates, jet injections and other dermatological bases/vehicles including instillative application, and on the other hand, as a systemic application for an oral, transdermal, intravenous, subcutaneous, intracutaneous, intramuscular or intrathecal application in suitable recipes or in suitable galenic forms.

In accordance with the invention, the compounds of the general formulae A1 to A14 in general, and preferably the compounds A1.001 to A14.003 according to Tables 1 to 14, alone or in combination, or pharmaceutical or cosmetic compositions comprising one or several of said compounds are used for an inhibition of the activity of the alanyl aminopeptidases or of analogous enzymes, alone or in combination with inhibitors of the dipeptidyl peptidase IV or with inhibitors of analogous enzymes.

In another embodiment, the compounds of the general formulae A1 to A14 in general, and preferably the compounds A1.001 to A14.003 according to Tables 1 to 14, alone or in combination, or pharmaceutical or cosmetic compositions comprising one or several of said compounds are used for topically influencing the activity of the alanyl aminopeptidases or of analogous enzymes, alone or in combination with inhibitors of the dipeptidyl peptidase IV or with inhibitors of analogous enzymes.

In preferred embodiments of the invention, the compounds of the general formulae A1 to A14 in general, and preferably the compounds A1.001 to A14.003 according to Tables 1 to 14, alone or in combination, or pharma-ceutical or cosmetic compositions comprising one or several of said compounds are used for a prophylaxis and a therapy of diseases as, for example: multiple sclerosis, Morbus Crohn, Colitis ulcerosa and of other autoimmune diseases as well as of inflammatory diseases, of Asthma bronchiale and of other allergic diseases, of skin and mucosa diseases, for example psoriasis, acne, and of dermatologic diseases being accompanied by a hyperproliferation and by changed differentiation states of fibroblasts, of benign fibrosing and sclerosing skin diseases and of malign fibroblastar hyperproliferation states, of acute neuronal diseases as, for example, ischemia-caused cerebral damage after an ischemic or hemorrhagic stroke, craniocerebral trauma, heart arrest, myocardial infarct or as a consequence of heart surgery, of chronic neuronal diseases, for example Morbus Alzheimer, Pick's disease, of the progressive supranuclear palsy, of a corticobasal degeneration, of a frontotemporal dementia, of Morbus Parkinson, particularly of Morbus Parkinson coupled to the chromosome 17, of Morbus Huntington, of disease states caused by prions, and od amyotrophic lateral sclerosis, of artherosclerosis, of arterial inflammations, of a stent restenosis, of chronic obstructive pulmonal diseases (Chronisch Obstruktive Lungenerkrankungen; COPD), of tumors, of metastases, of prostata tumors, of the Heavy Acute Respiratory Syndrome (SARS) and of sepsis and sepsis-like conditions.

In a further preferred embodiment of the invention, the compounds of the general formulae A1 to A14 in general, and preferably the compounds A1.001 to A14.003 according to Tables 1 to 14, alone or in combination, or pharmaceutical or cosmetic compositions comprising one or several of said compounds are used for a prophylaxis and a therapy of a rejection of transplanted tissues and cells. As an example of such an application, there may be mentioned the use of one or of several of the above-mentioned compounds or of a pharmaceutical composition containing one or several of the said compounds in connection with allogenic kidney transplants or stem cell trans-plants.

In a further preferred embodiment of the invention, the compounds of the general formulae A1 to A14 in general, and preferably the compounds A1.001 to A14.003 according to Tables 1 to 14, alone or in combination, or pharmaceutical or cosmetic compositions comprising one or several of said compounds are used for a prophylaxis and a therapy of rejection and inflammation reactions at, or by, medical devices implanted into an organism (“medical devices”). These may comprise, for example, stents, articulation implants (knee joint implants, hip joint implants), bone implants, heart pacemakers, or other implants. In a further preferred embodiment of the invention, the compounds of the general formulae A1 to A14 in general, and preferably the compounds A1.001 to A14.003 according to Tables 1 to 14, alone or in combination, or pharmaceutical or cosmetic compositions comprising one or several of said compounds are used in such a way that the compound(s) or composition(s) is/are applied onto the article or articles in the form of a coating or layer, or at least one of the compounds or compositions is admixed, as a substance, to the material of the article or articles. Also in this case, it is possible—of course—that at least one of the compounds or compositions is administered locally or systemically, optionally successively or parallel in time.

In a similar way as described above, and for similar purposes or for the prophylaxis and therapy of the above diseases and conditions mentioned as examples, however without any restriction, the compounds of the general formulae A1 to A14 in general, and preferably the compounds A1.001 to A14.003 according to Tables 1 to 14, alone or in combination, or the above-mentioned pharmaceutical or cosmetic compositions comprising one or several of said above-mentioned compounds may be used for the preparation of a medicament for a prophylaxis and a therapy of the above-mentioned diseases or conditions. These medicaments may comprise said compounds in the amounts specified above, optionally together with known carrier substances, auxiliary substances and/or additives.

Finally, the invention also relates to a process for inhibiting the activity of alanyl aminopeptidases and of analogous enzymes, alone or in combination with inhibitors of the DP IV and of analogous enzymes by an administration of at least one compound or pharmaceutical or cosmetic composition according to the above detailed description in an amount required for an inhibition of the enzyme activity. The amounts of one of the compounds of the general formulae A1 to A14 in general and of the compounds A1.001 to A14.003 according to Tables 1 to 14 are—as indicated above—in the range of from 0.01 to 1000 mg of one compound per administration unit, preferably in the range of from 0.1 to 100 mg of one compound per administration unit.

The invention also relates to a process for topically influencing the activity of alanyl aminopeptidases and of analogous enzymes, alone or in combination with inhibitors of the DP IV or of analogous enzymes by an administration of at least one compound or pharmaceutical or cosmetic composition according to the above detailed description in an amount required for topically influencing the enzyme activity. Also in these cases, the amounts of said compound(s) are in the above-indicated range.

Furthermore, the invention also relates to a process for the prophylaxis and therapy of a plurality of diseases, for example diseases accompanied by an excessive immune response (autoimmune diseases, allergies, transplant rejections), of other chronically inflammatory diseases, of neuronal diseases and cerebral damage, of skin diseases (inter alia acne and psoriasis), of tumor diseases and of specific virus diseases (inter alia SARS), and particularly of the diseases mentioned above in detail, by an administration of at least one compound or of a pharmaceutical or cosmetic composition in accordance with the above detailed description in an amount required for the prophylaxis and therapy of the respective disease. Also in these cases, the amounts of the above compound(s) are in the above-mentioned range of from 0.01 to 1000 mg of one compound per administration unit, preferably in the range of from 0.1 to 100 mg of one compound per administration unit.

In the following, the invention is in more detail explained by specific preferred exemplary embodiments. Those exemplary embodiments, however, do not serve a limitation of the invention, but only an exemplifying explanation.

### EXAMPLES

### Example 1

### Inhibition Characteristics of the Novel Inhibitors of the Alanyl Aminopeptidases

In the following Tables 1 to 14, novel inhibitors are summarized, for which the inventors could show that these substances are capable of inhibiting alanyl aminopeptidases and enzymes having an analog effect in their enzymatic activity. The inhibition characteristics measured are referred to as IC-50 values or ID50 values (the latter marked with “*”) for said enzyme. The enzymatic activity was determined by means of the fluorogenic substrate/product (Ala)₂-rhodamine 110.

TABLE 1

| Compound ID. | Structure | IC50_{APN} [μM] |
|---|---|---|
| A1.001 | 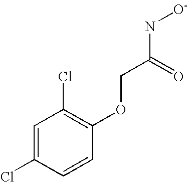 | 0.8* |
| A1.002 | 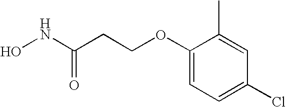 | 5.6 |
| A1.003 | 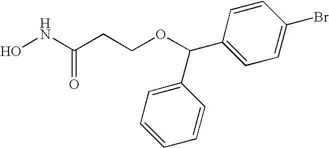 | 6.3 |
| A1.004 | 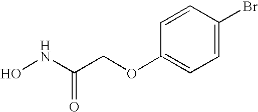 | 6.4 |
| A1.005 | 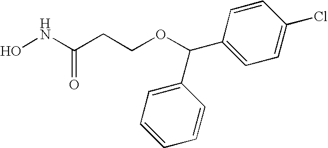 | 7.3 |
| A1.006 | 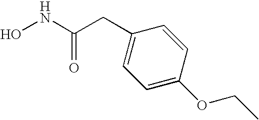 | 7.5 |
| A1.007 | 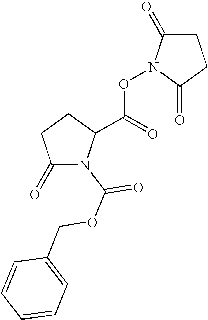 | 8.4 |
| A1.008 | 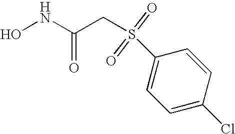 | 11.5 |
| A1.009 | 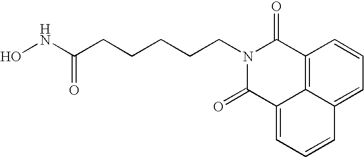 | 14.1 |
| A1.010 | 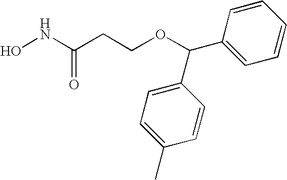 | 14.2 |
| A1.011 | 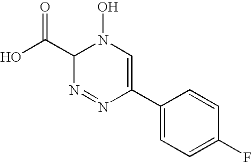 | 17.1 |
| A1.012 | 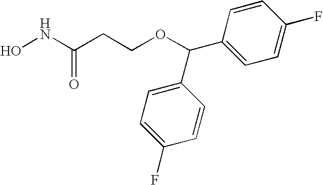 | 21.8 |
| A1.013 | 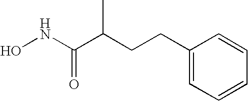 | 25.2 |
| A1.014 | 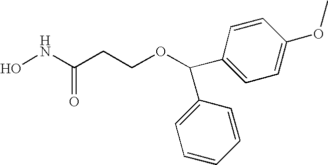 | 33.0 |
| A1.015 | 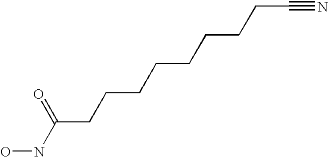 | 80.6 |

TABLE 2

| Compound ID. | Structure | IC50_{APN} [μM] |
|---|---|---|
| A2.001 | 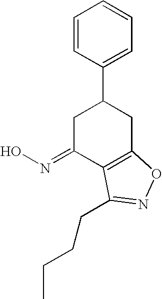 | 2.2 |
| A2.002 | 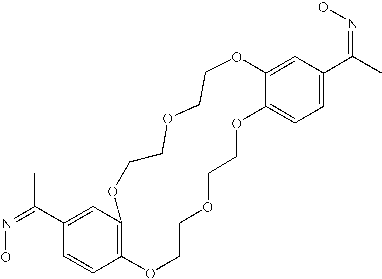 | 8.6 |
| A2.003 | 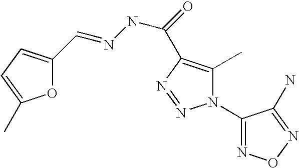 | 10.9 |
| A2.004 | 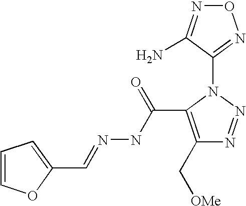 | 12.5 |
| A2.005 | 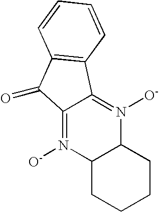 | 13.7 |
| A2.006 | 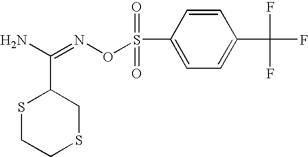 | 13.4 |
| A2.007 | 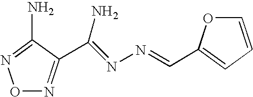 | 14.6 |
| A2.008 | 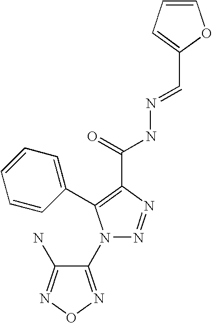 | 46.6 |
| A2.010 | 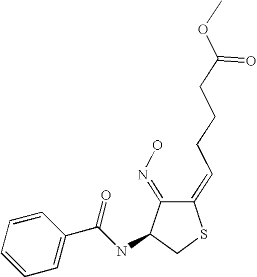 | 93.6 |

TABLE 3

| Compound ID. | Structure | IC50_{APN} [μM] |
|---|---|---|
| A3.001 | 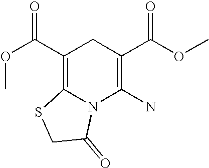 | “very high |
| | | active” |
| A3.002 | 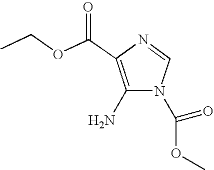 | 0.9* |
| A3.003 | 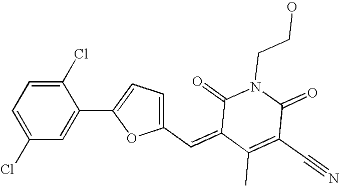 | 1.2* |
| A3.006 | 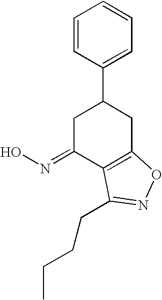 | 2.2* |
| A3.007 | 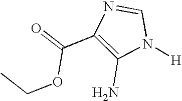 | 2.6* |
| A3.008 | 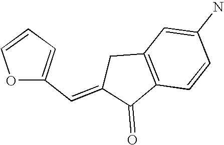 | 2.6* |
| A3.009 | 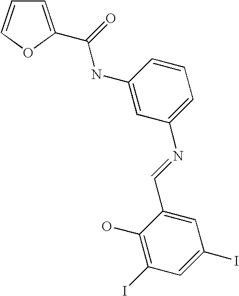 | 3.0 |
| A3.010 | 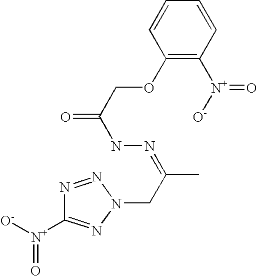 | 3.4 |
| A3.011 | 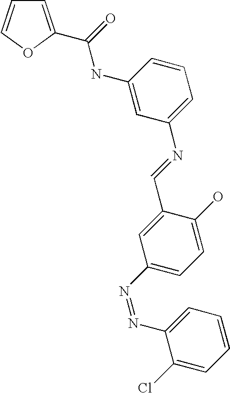 | 3.6 |
| A3.012 | 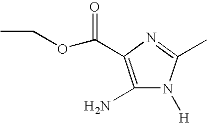 | 4.3 |
| A3.014 | 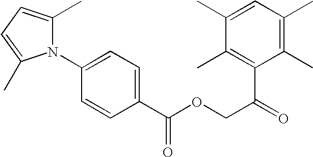 | 4.5 |
| A3.015 | 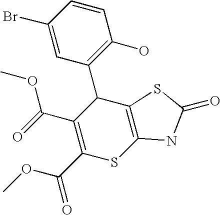 | 4.7 |
| A3.016 | 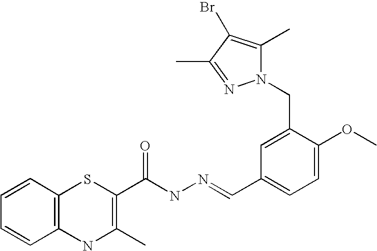 | 4.8 |
| A3.017 | 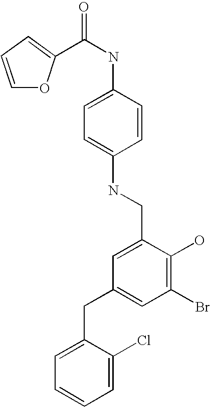 | 4.9 |
| A3.018 | 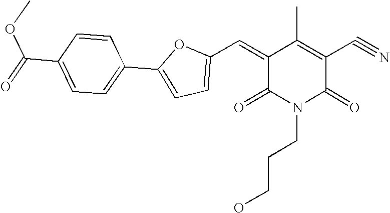 | 5.0 |
| A3.019 | 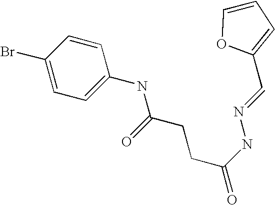 | 5.2 |
| A3.020 | 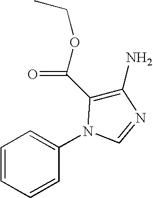 | 5.4 |
| A3.022 | 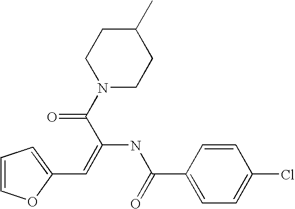 | 6.2 |
| A3.023 | 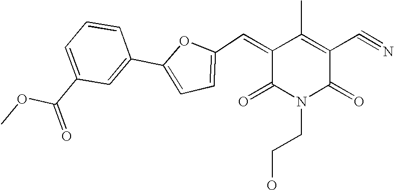 | 6.3 |
| A3.024 | 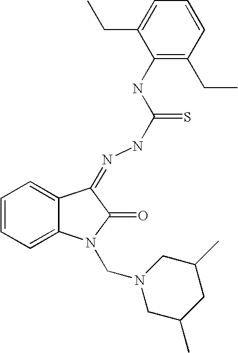 | 6.6 |
| A3.025 | 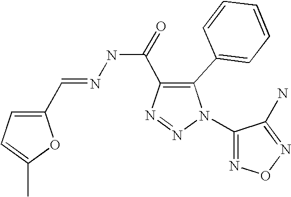 | 6.9 |
| A3.026 | 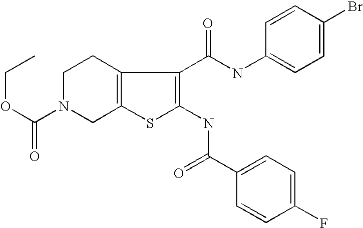 | 7.3 |
| A3.027 | 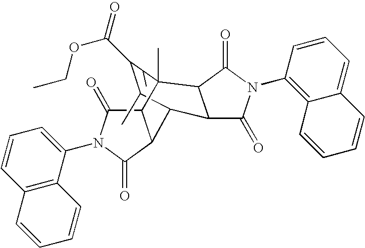 | 7.4 |
| A3.029 | 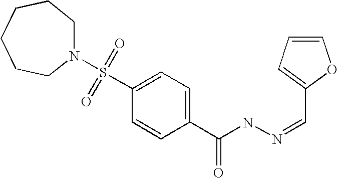 | 8.0 |
| A3.030 | 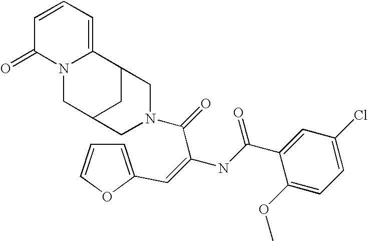 | 8.1 |
| A3.031 | 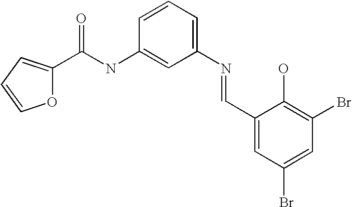 | 8.2 |
| A3.032 | 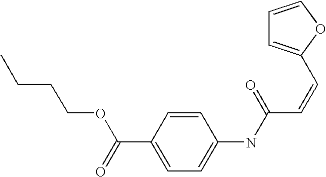 | 8.3 |
| A3.033 | 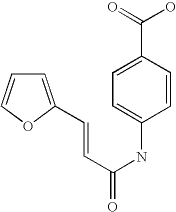 | 8.4 |
| A3.035 | 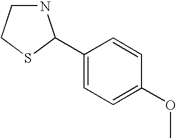 | 9.5 |
| A3.037 | 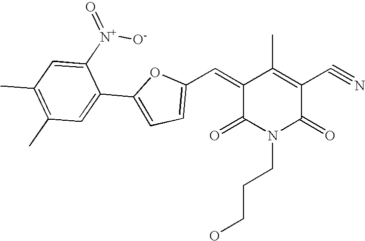 | 9.7 |
| A3.038 | 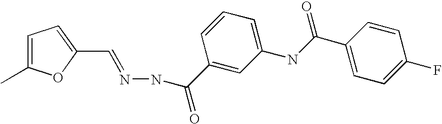 | 9.8 |
| A3.039 | 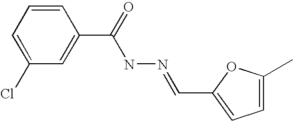 | 10.5 |
| A3.040 | 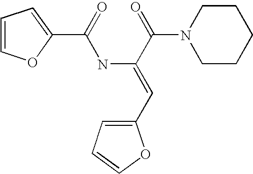 | 10.6 |
| A3.041 | 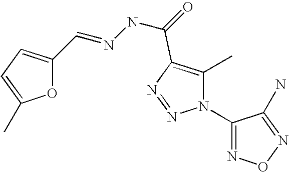 | 10.9 |
| A3.042 | 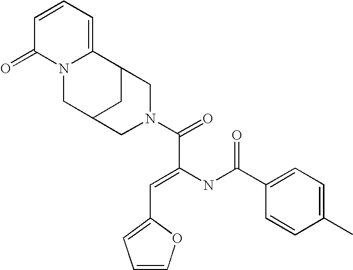 | 11.0 |
| A3.043 | 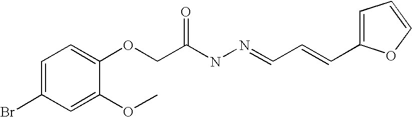 | 11.0 |
| A3.045 | 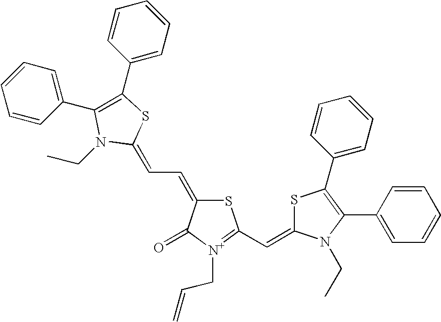 | 11.1 |
| A3.046 | 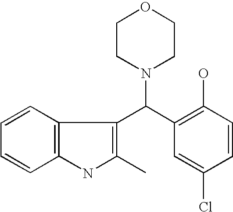 | 11.7 |
| A3.047 | 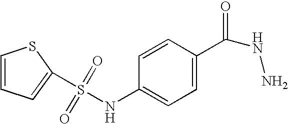 | 11.8 |
| A3.048 | 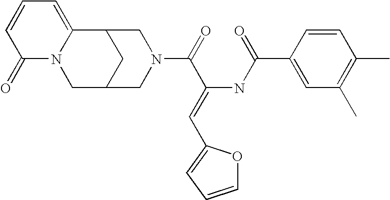 | 11.8 |
| A3.050 | 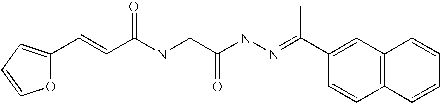 | 12.0 |
| A3.051 | 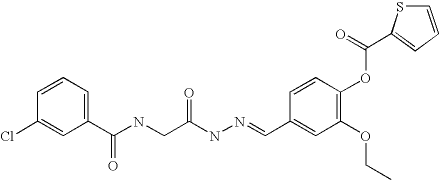 | 12.2 |
| A3.052 | 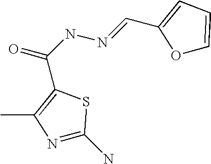 | 12.3 |
| A3.053 | 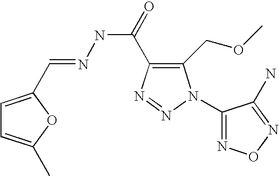 | 12.5 |
| A3.054 | 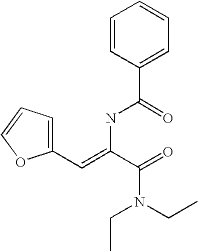 | 12.8 |
| A3.055 | 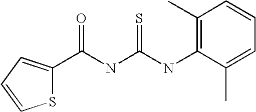 | 12.9 |
| A3.056 | 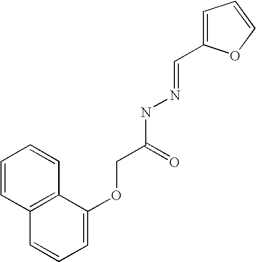 | 12.9 |
| A3.057 | 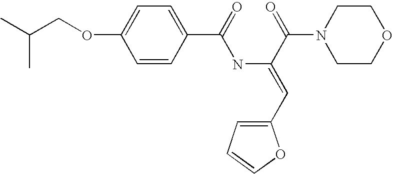 | 13.1 |
| A3.058 | 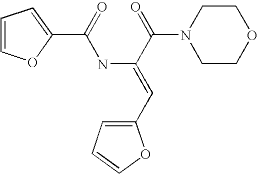 | 13.5 |
| A3.059 | 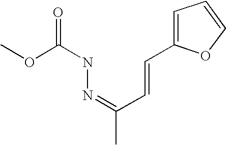 | 13.5 |
| A3.060 | 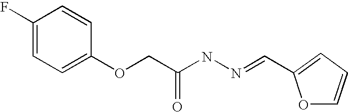 | 13.6 |
| A3.061 | 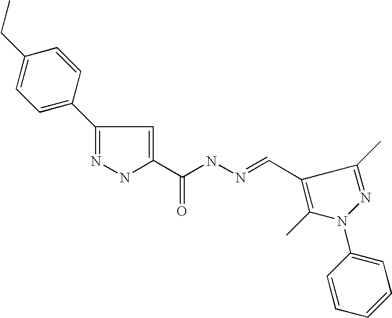 | 13.6 |
| A3.062 | 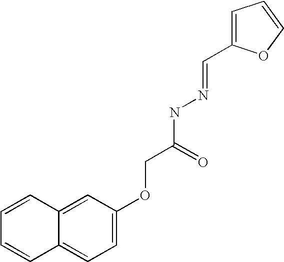 | 13.9 |
| A3.063 | 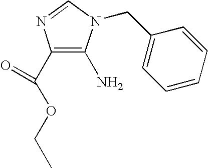 | 14.0 |
| A3.064 | 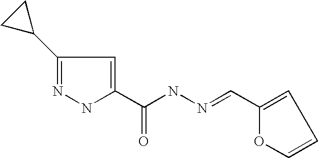 | 14.3 |
| A3.065 | 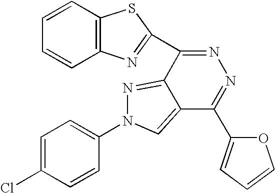 | 14.3 |
| A3.066 | 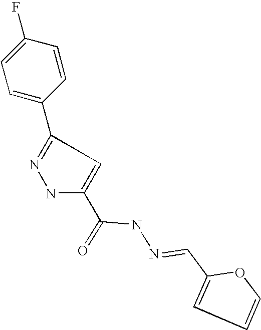 | 14.4 |
| A3.067 | 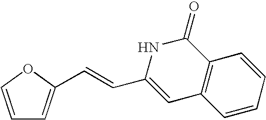 | 14.6 |
| A3.068 | 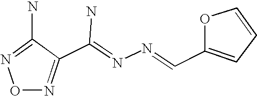 | 14.6 |
| A3.069 | 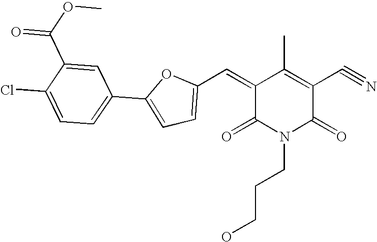 | 15.2 |
| A3.070 | 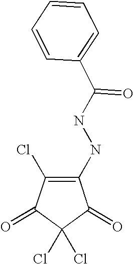 | 15.2 |
| A3.071 | 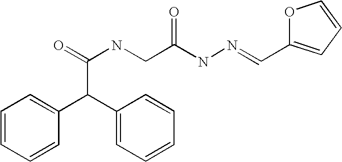 | 15.6 |
| A3.072 | 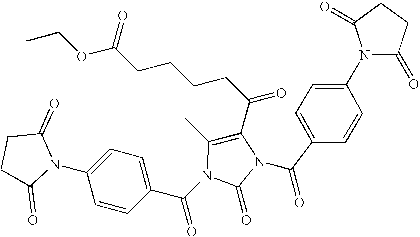 | 15.6 |
| A3.073 | 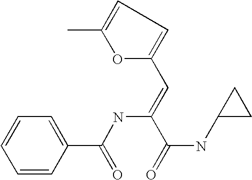 | 15.6 |
| A3.074 | 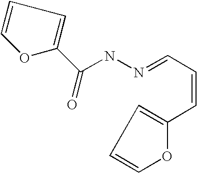 | 16.0 |
| A3.075 | 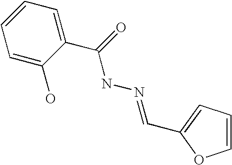 | 16.0 |
| A3.076 | 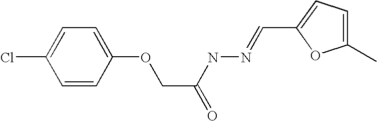 | 16.0 |
| A3.077 | 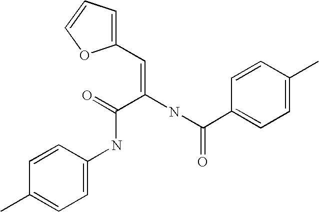 | 16.4 |
| A3.078 | 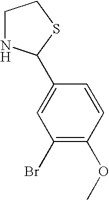 | 16.4 |
| A3.080 | 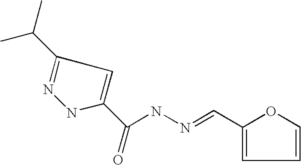 | 16.6 |
| A3.081 | 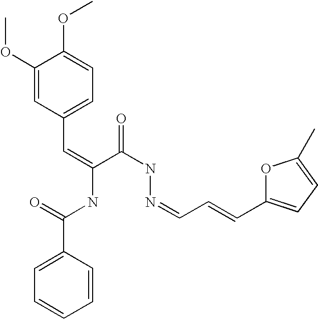 | 16.7 |
| A3.082 | 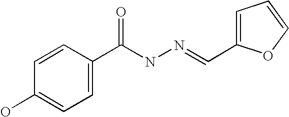 | 16.8 |
| A3.083 | 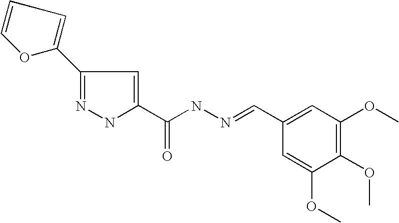 | 17.0 |
| A3.084 | 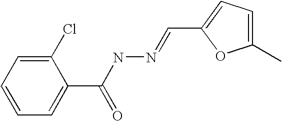 | 17.7 |
| A3.086 | 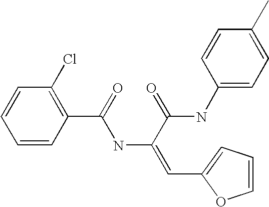 | 17.9 |
| A3.087 | 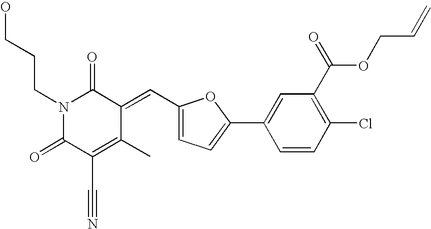 | 18.1 |
| A3.088 | 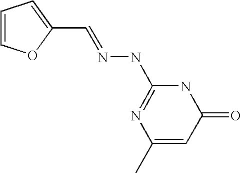 | 18.2 |
| A3.089 | 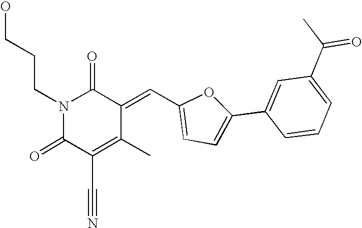 | 18.2 |
| A3.090 | 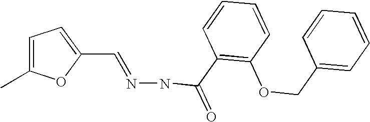 | 18.4 |
| A3.091 | 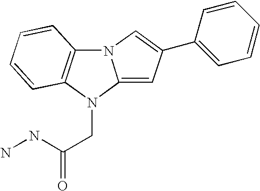 | 18.7 |
| A3.092 | 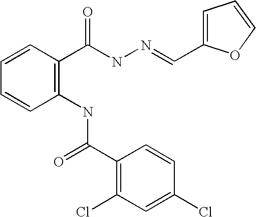 | 18.7 |
| A3.093 | 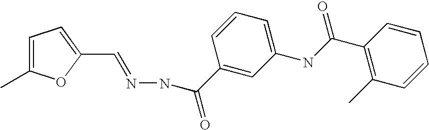 | 18.9 |
| A3.094 | 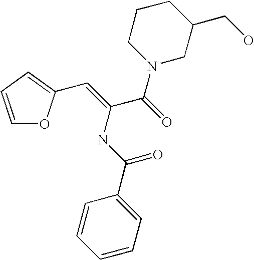 | 19.0 |
| A3.095 | 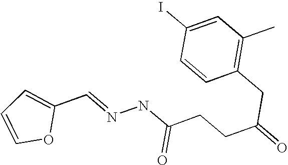 | 9.5 |
| A3.097 | 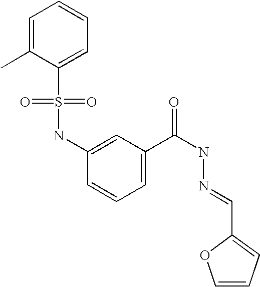 | 19.2 |
| A3.098 | 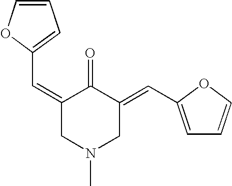 | 19.2 |
| A3.099 | 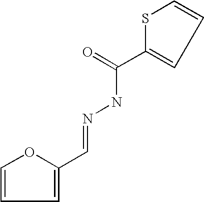 | 19.4 |
| A3.100 | 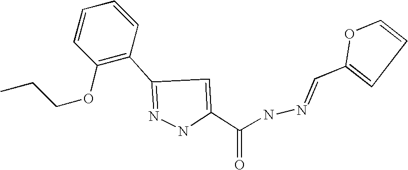 | 19.5 |
| A3.101 | 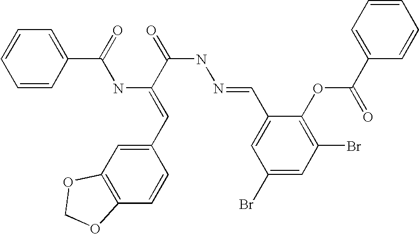 | 19.8 |
| A3.102 | 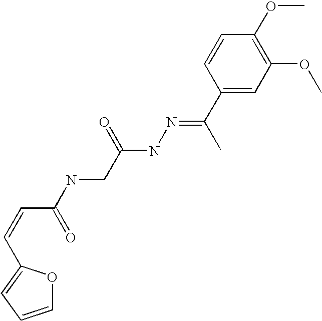 | 19.9 |
| A3.103 | 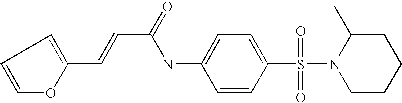 | 19.9 |
| A3.104 | 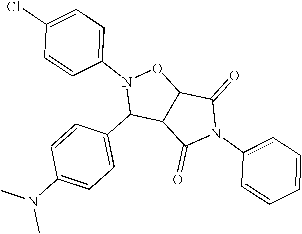 | 20.0 |
| A3.105 | 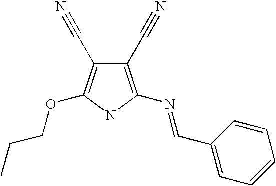 | 20.0 |
| A3.106 | 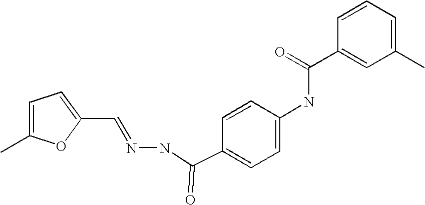 | 20.2 |
| A3.107 | 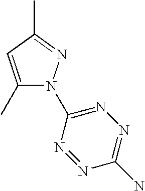 | 20.3 |
| A3.109 | 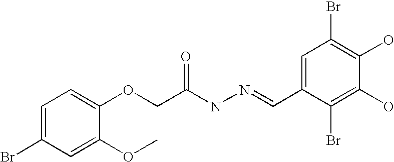 | 20.5 |
| A3.110 | 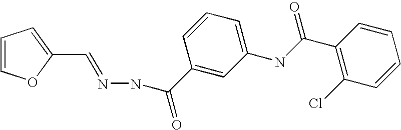 | 20.6 |
| A3.111 | 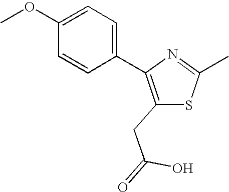 | 20.8 |
| A3.112 | 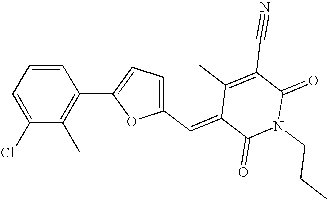 | 20.8 |
| A3.113 | 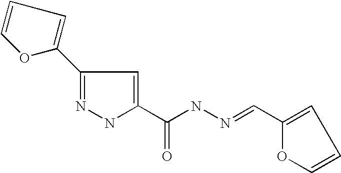 | 20.9 |
| A3.114 | 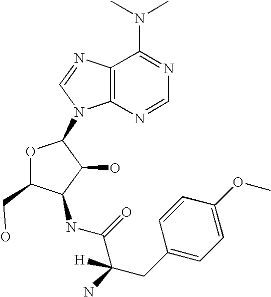 | 20.9 |
| A3.115 | 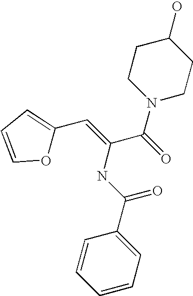 | 21.1 |
| A3.116 | 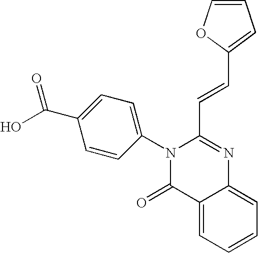 | 21.2 |
| A3.117 | 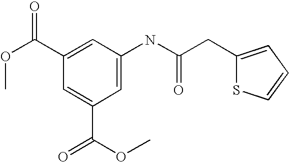 | 21.2 |
| A3.118 | 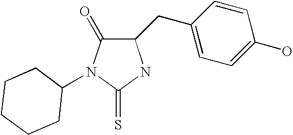 | 21.3 |
| A3.120 | 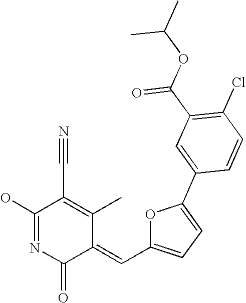 | 21.4 |
| A3.121 | 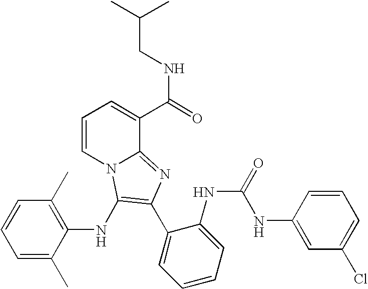 | 21.6 |
| A3.122 | 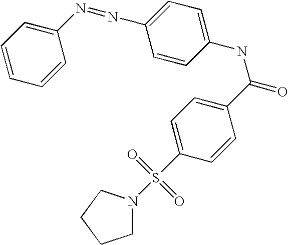 | 21.6 |
| A3.124 | 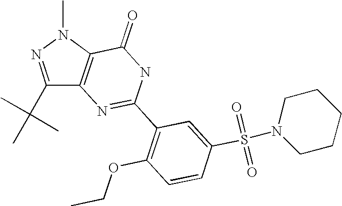 | 21.8 |
| A3.125 | 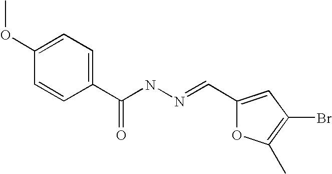 | 21.8 |
| A3.128 | 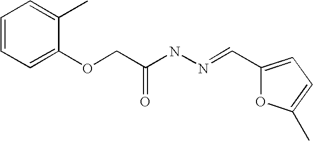 | 22.2 |
| A3.129 | 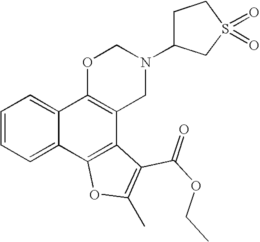 | 22.3 |
| A3.130 | 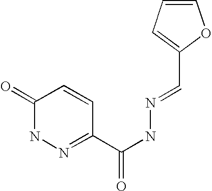 | 22.4 |
| A3.131 | 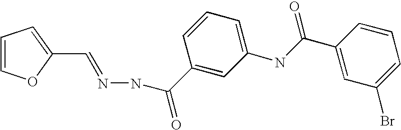 | 22.4 |
| A3.132 | 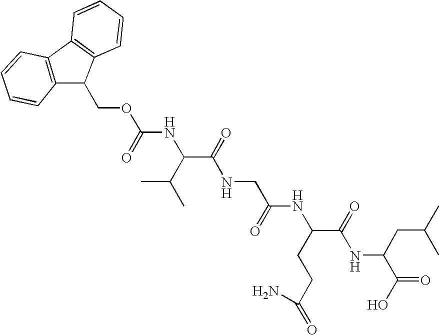 | 22.5 |
| A3.133 | 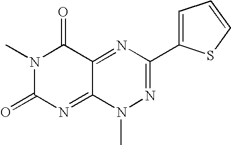 | 25.0 |
| A3.134 | 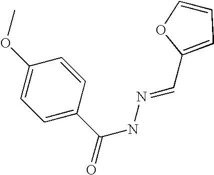 | 22.6 |
| A3.135 | 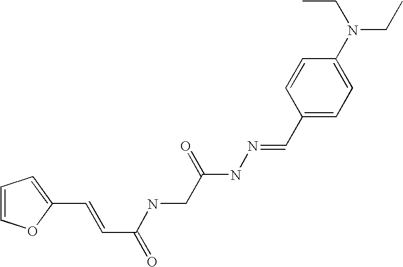 | 22.8 |
| A3.136 | 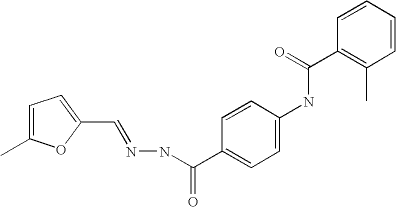 | 22.8 |
| A3.137 | 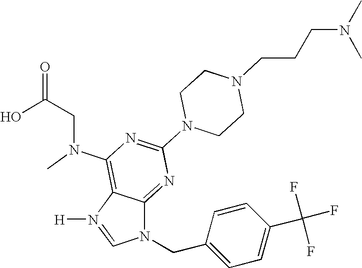 | 23.0 |
| A3.138 | 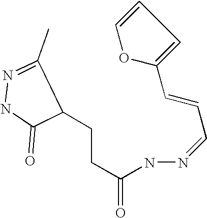 | 23.0 |
| A3.139 | 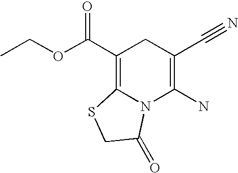 | 23.1 |
| A3.140 | 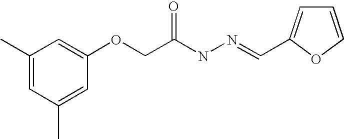 | 23.4 |
| A3.141 | 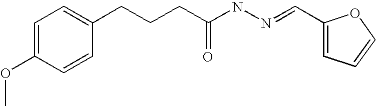 | 23.4 |
| A3.142 | 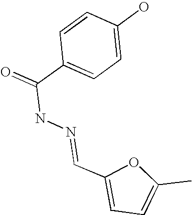 | 23.4 |
| A3.143 | 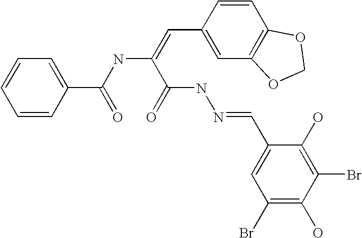 | 23.6 |
| A3.144 | 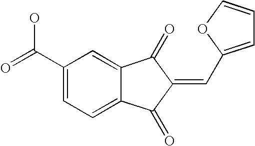 | 23.7 |
| A3.145 | 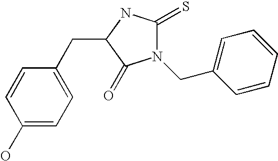 | 23.7* |
| A3.146 | 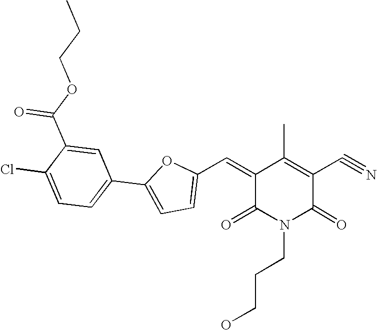 | 23.8 |
| A3.147 | 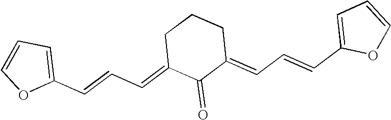 | 24.4 |
| A3.148 | 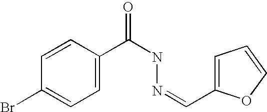 | 24.5 |
| A3.149 | 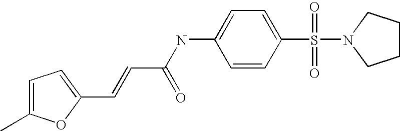 | 24.8 |
| A3.150 | 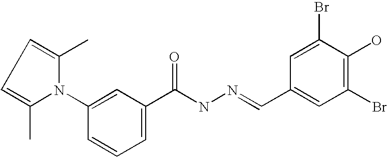 | 24.9 |
| A3.151 | 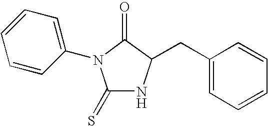 | 26.1 |
| A3.152 | 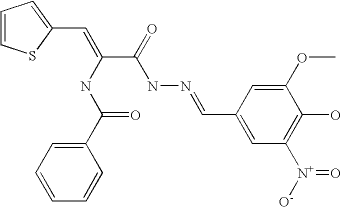 | 25.1 |
| A3.153 | 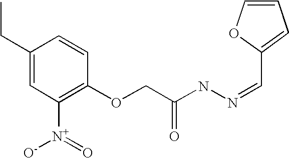 | 25.1 |
| A3.154 | 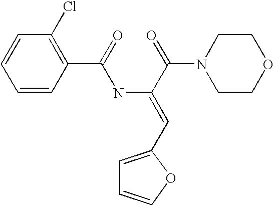 | 25.5 |
| A3.155 | 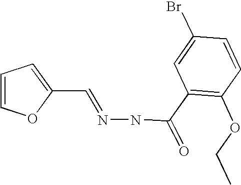 | 25.8 |
| A3.156 | 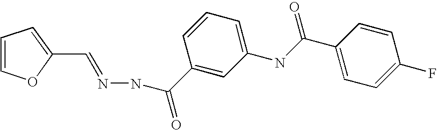 | 25.9 |
| A3.157 | 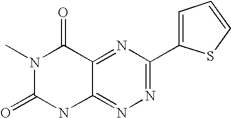 | 26.1 |
| A3.158 | 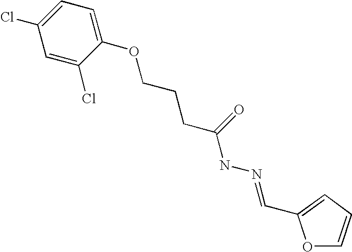 | 26.3 |
| A3.159 | 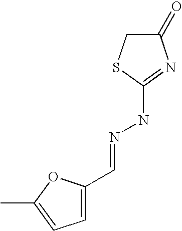 | 22.6 |
| A3.160 | 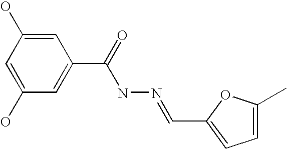 | 26.4 |
| A3.161 | 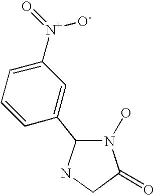 | 26.4 |
| A3.162 | 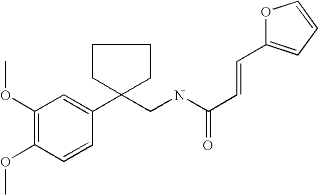 | 26.6 |
| A3.163 | 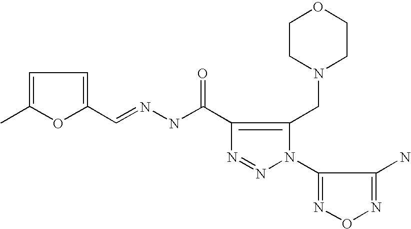 | 26.7 |
| A3.164 | 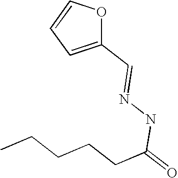 | 26.7 |
| A3.165 | 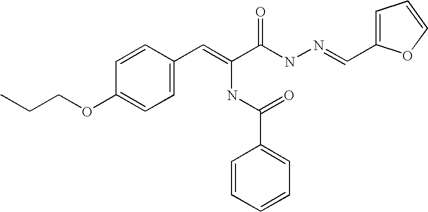 | 27.3 |
| A3.166 | 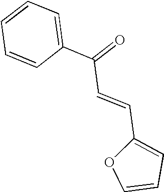 | 27.3 |
| A3.167 | 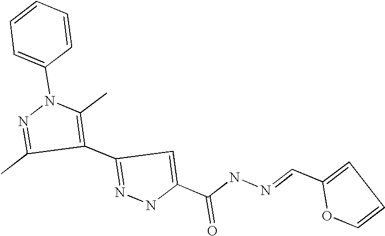 | 27.4 |
| A3.168 | 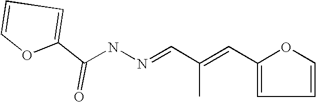 | 27.5 |
| A3.169 | 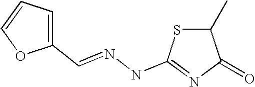 | 27.7 |
| A3.170 | 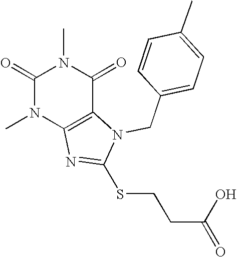 | 28.0 |
| A3.171 | 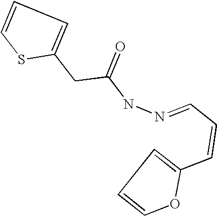 | 28.0 |
| A3.172 | 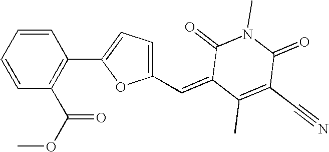 | 28.1 |
| A3.173 | 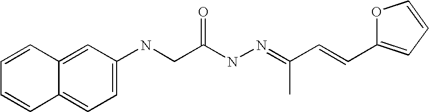 | 28.5 |
| A3.174 | 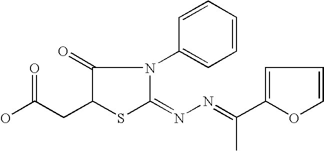 | 28.6 |
| A3.175 | 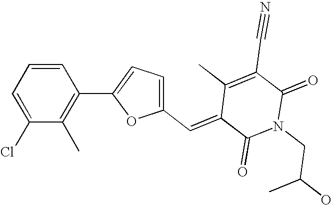 | 28.6 |
| A3.176 | 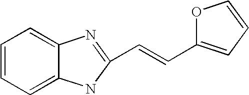 | 28.7 |
| A3.177 | 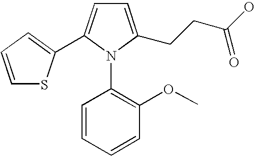 | 28.7 |
| A3.178 | 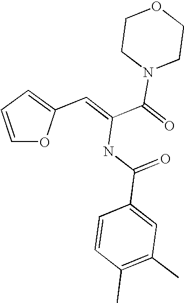 | 28.8 |
| A3.179 | 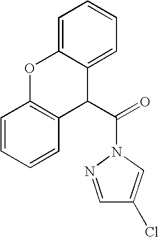 | 29.1 |
| A3.180 | 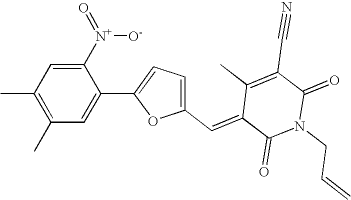 | 29.3 |
| A3.181 | 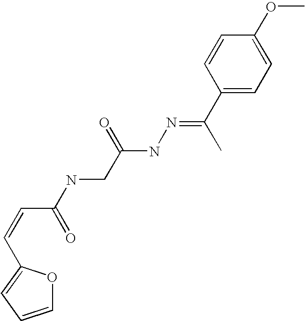 | 29.4 |
| A3.182 | 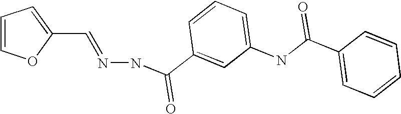 | 29.5 |
| A3.183 | 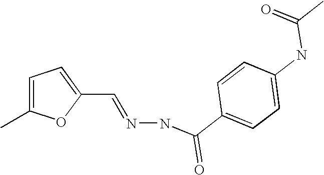 | 29.7 |
| A3.184 | 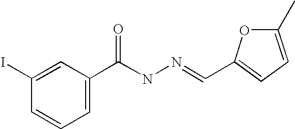 | 30.0 |
| A3.185 | 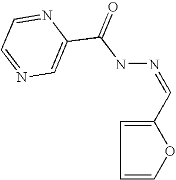 | 30.1 |
| A3.186 | 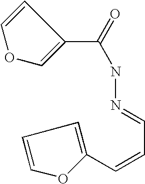 | 30.1 |
| A3.187 | 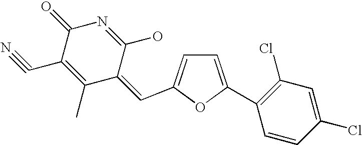 | 30.3 |
| A3.188 | 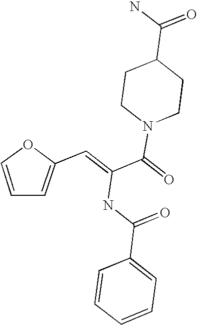 | 30.5 |
| A3.189 | 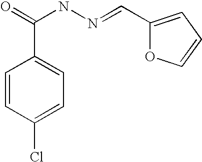 | 30.8 |
| A3.190 | 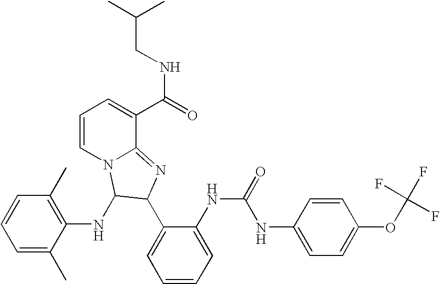 | 31.1 |
| A3.191 | 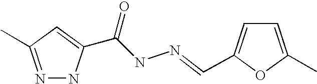 | 31.8 |
| A3.192 | 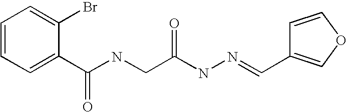 | 31.9 |
| A3.193 | 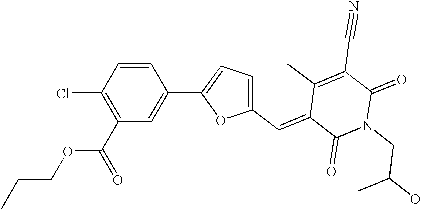 | 32.0 |
| A3.194 | 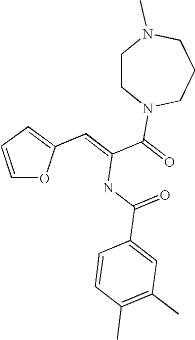 | 32.1 |
| A3.195 | 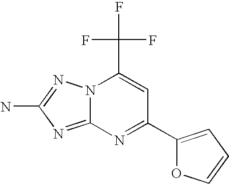 | 32.1 |
| A3.196 | 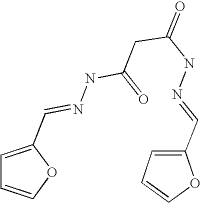 | 32.4 |
| A3.197 | 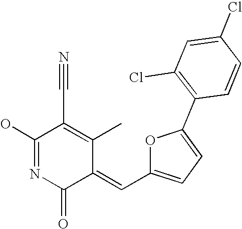 | 32.4 |
| A3.198 | 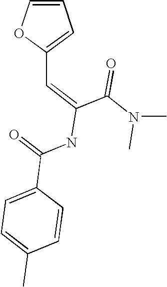 | 32.5 |
| A3.199 | 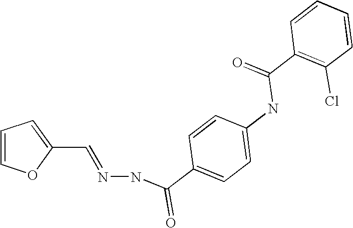 | 32.5 |
| A3.200 | 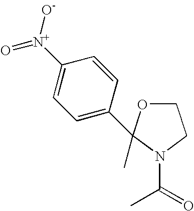 | 32.8 |
| A3.201 | 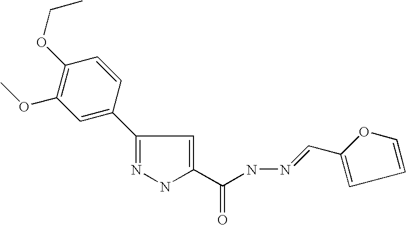 | 33.4 |
| A3.202 | 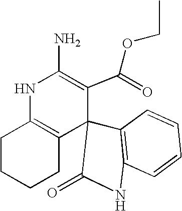 | 33.6 |
| A3.203 | 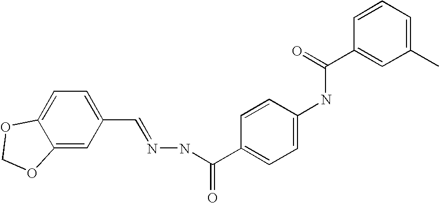 | 33.8* |
| A3.204 | 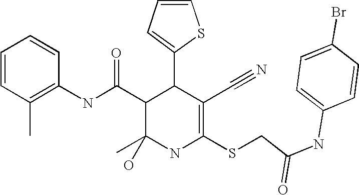 | 33.9 |
| A3.205 | 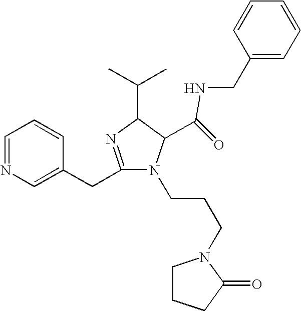 | 34.0 |
| A3.206 | 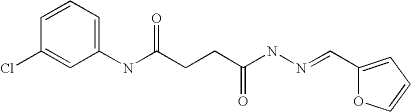 | 34.0 |
| A3.207 | 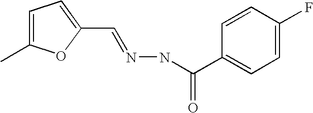 | 34.4 |
| A3.208 | 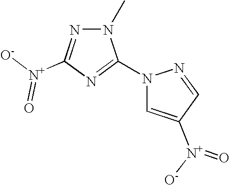 | 34.5 |
| A3.209 | 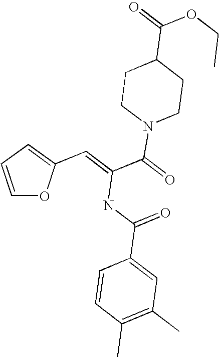 | 34.5 |
| A3.210 | 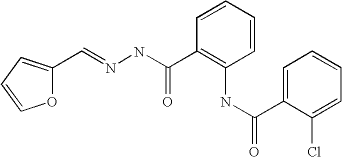 | 34.7 |
| A3.211 | 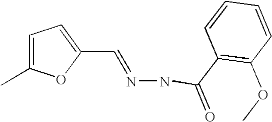 | 34.7 |
| A3.212 | 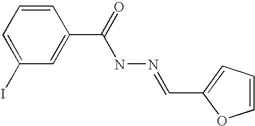 | 34.7 |
| A3.213 | 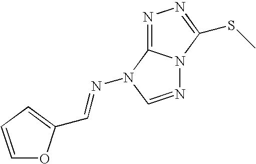 | 34.7 |
| A3.214 | 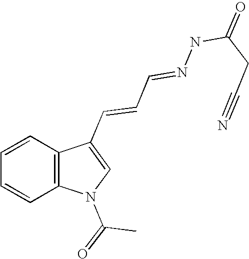 | 35.5 |
| A3.215 | 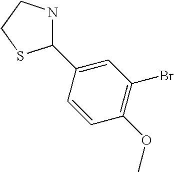 | 35.7 |
| A3.216a | 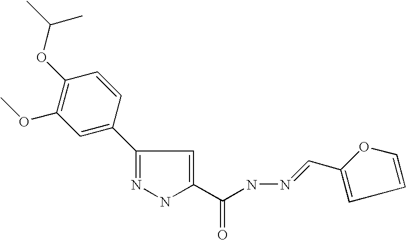 | 36.1 |
| A3.216b | 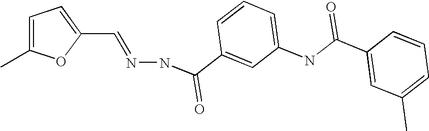 | 36.2 |
| A3.217 | 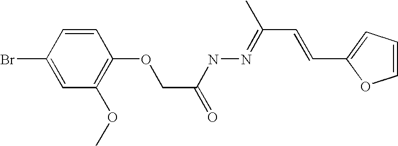 | 36.3 |
| A3.218 | 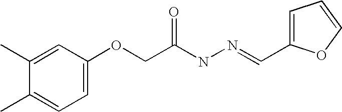 | 36.3 |
| A3.219 | 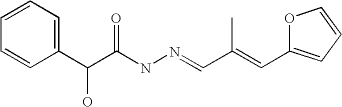 | 36.3 |
| A3.220 | 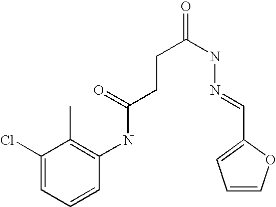 | 36.6 |
| A3.221 | 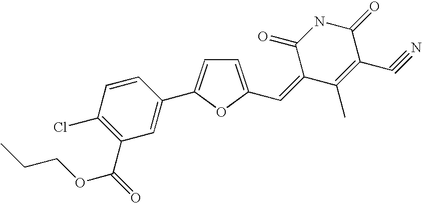 | 36.8 |
| A3.222 | 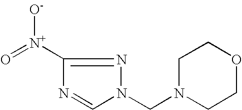 | 36.6 |
| A3.223 | 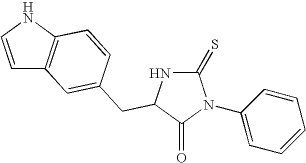 | 36.9 |
| A3.224 | 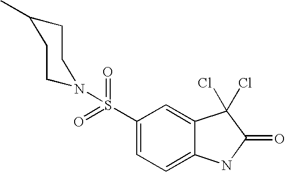 | 37.2 |
| A3.225 | 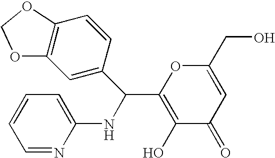 | 37.3 |
| A3.226 | 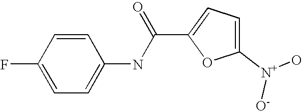 | 37.4 |
| A3.227 | 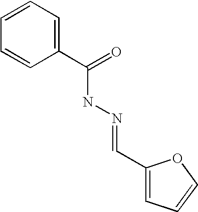 | 37.7 |
| A3.228 | 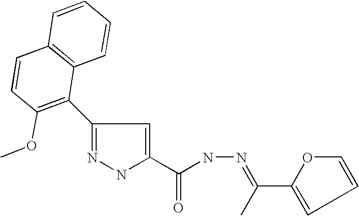 | 38.2 |
| A3.229 | 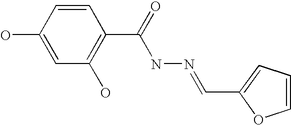 | 38.3 |
| A3.230 | 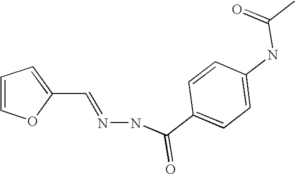 | 38.4 |
| A3.231 | 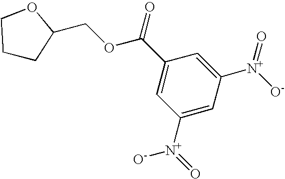 | 39.3 |
| A3.232 | 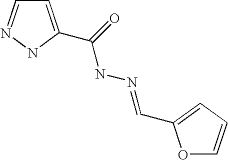 | 40.3 |
| A3.233 | 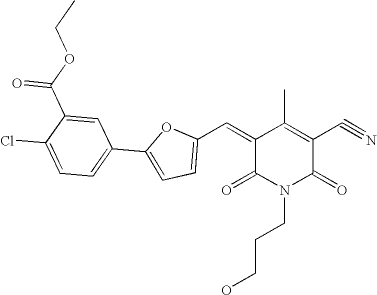 | 41.0 |
| A3.234 | 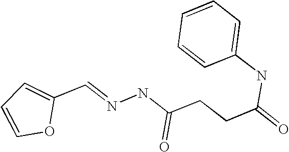 | 41.1 |
| A3.235 | 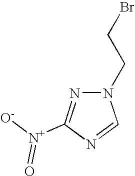 | 41.1 |
| A3.236 | 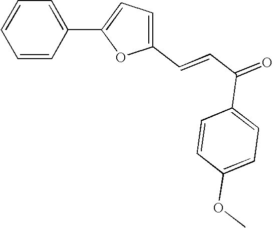 | 41.2 |
| A3.237 | 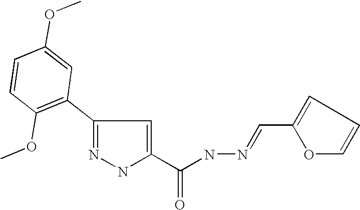 | 43.2 |
| A3.238 | 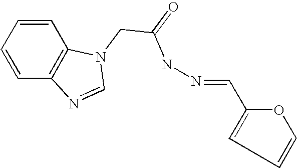 | 43.3 |
| A3.239 | 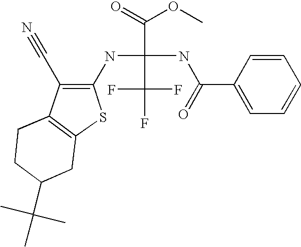 | 43.6 |
| A3.240 | 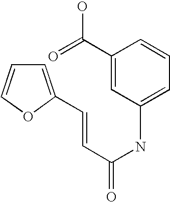 | 44.2 |
| A3.241 | 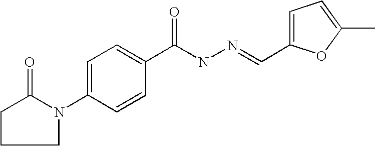 | 44.2 |
| A3.242 | 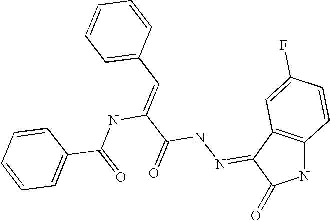 | 44.2 |
| A3.243 | 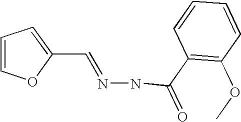 | 44.7 |
| A3.244 | 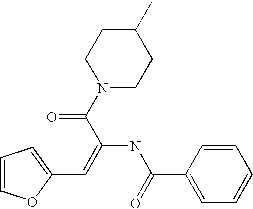 | 45.0 |
| A3.245 | 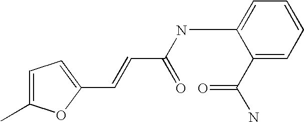 | 45.0 |
| A3.246 | 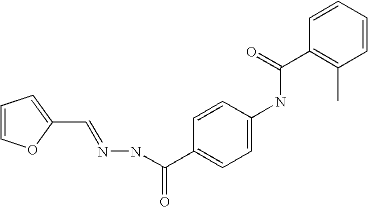 | 45.4 |
| A3.247 | 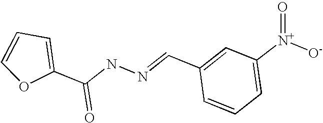 | 45.4 |
| A3.248 | 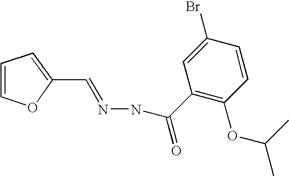 | 46.6 |
| A3.249 | 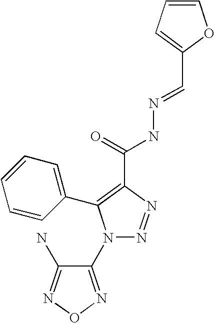 | 46.6 |
| A3.250 | 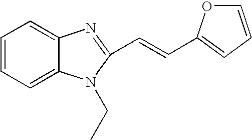 | 46.6 |
| A3.251 | 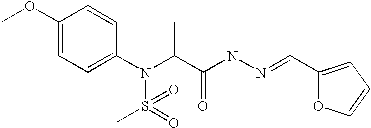 | 47.0 |
| A3.252 | 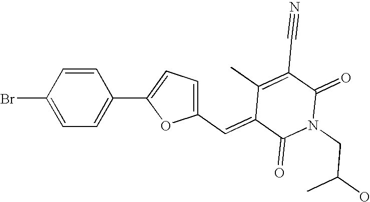 | 47.2* |
| A3.253 | 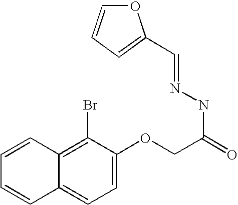 | 47.3 |
| A3.254 | 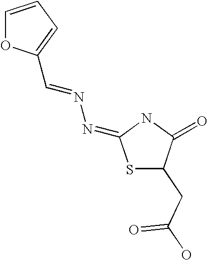 | 48.1* |
| A3.255 | 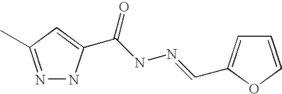 | 48.2 |
| A3.256 | 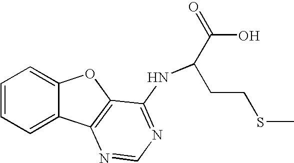 | 48.4 |
| A3.257 | 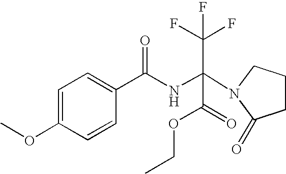 | 48.7 |
| A3.258 | 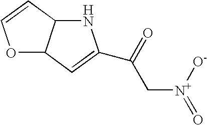 | 49.5 |
| A3.259 | 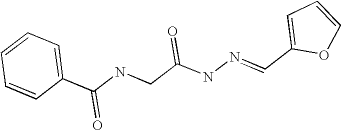 | 49.4* |
| A3.260 | 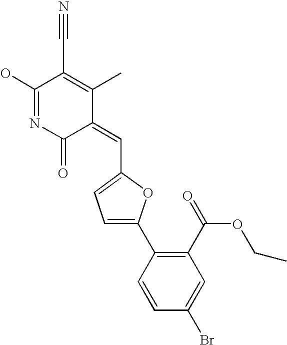 | 49.9 |
| A3.261 | 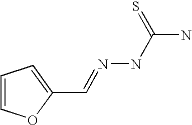 | 50.0 |
| A3.262 | 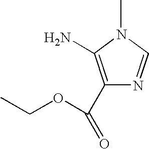 | 50.1 |
| A3.263 | 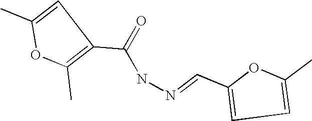 | 50.2 |
| A3.264 | 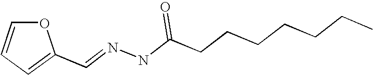 | 50.3 |
| A3.265 | 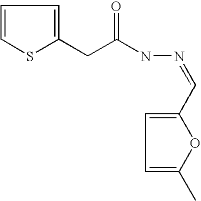 | 51.1 |
| A3.266 | 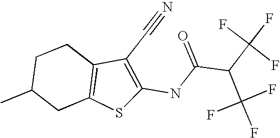 | 51.3 |
| A3.267 | 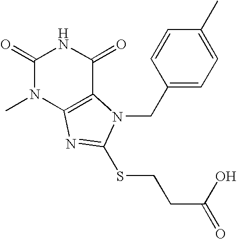 | 53.2* |
| A3.268 | 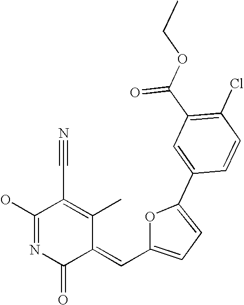 | 52.1 |
| A3.269 | 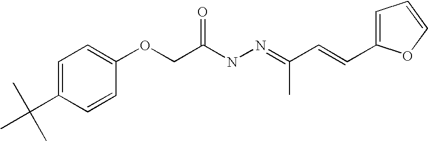 | 50.0 |
| A3.270 | 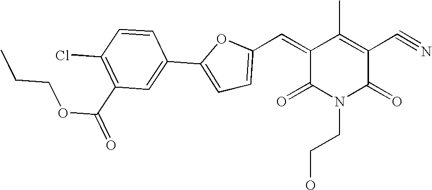 | 54.0 |
| A3.271 | 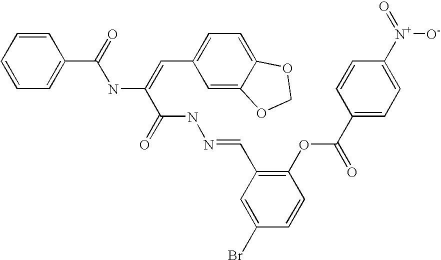 | 54.2 |
| A3.272 | 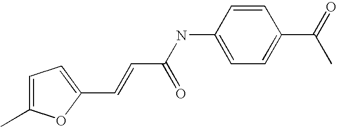 | 54.4 |
| A3.273 | 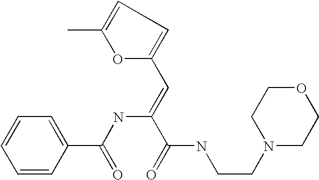 | 55.0* |
| A3.274 | 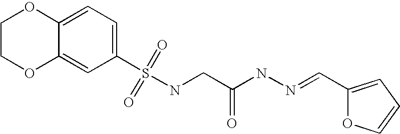 | 55.6 |
| A3.275 | 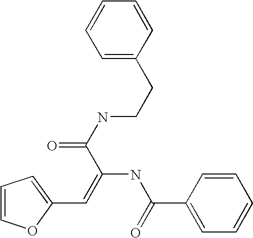 | 55.8 |
| A3.276 | 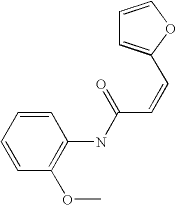 | 56.4 |
| A3.277 | 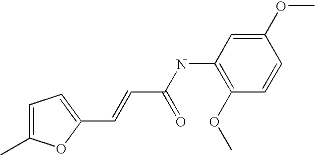 | 56.8 |
| A3.278 | 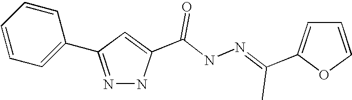 | 58.3 |
| A3.279 | 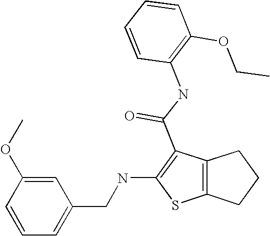 | 58.4 |
| A3.280 | 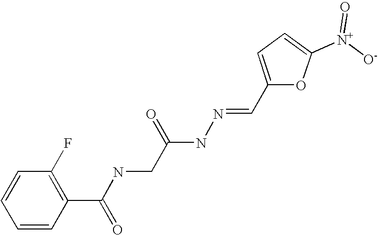 | 60.0 |
| A3.281 | 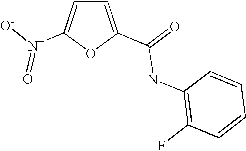 | 62.4 |
| A3.282 | 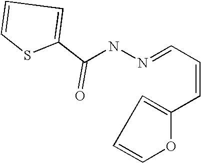 | 62.7 |
| A3.283 | 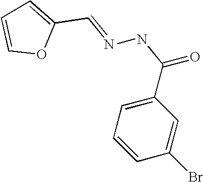 | 62.7 |
| A3.284 | 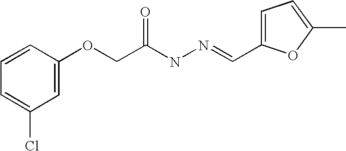 | 62.7 |
| A3.285 | 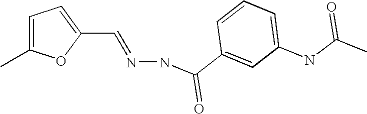 | 63.1* |
| A3.286 | 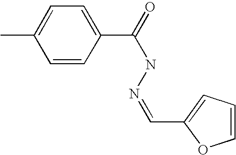 | 64.2 |
| A3.287 | 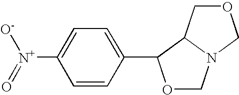 | 64.5 |
| A3.288 | 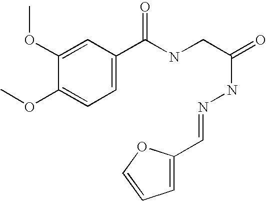 | 64.9* |
| A3.289 | 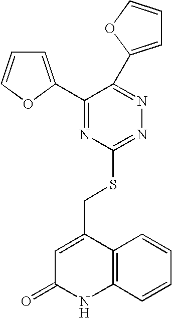 | 65.1* |
| A3.290 | 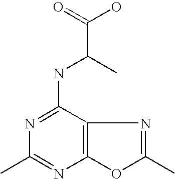 | 65.8 |
| A3.291 | 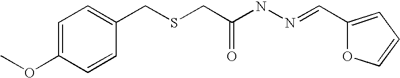 | 66.0* |
| A3.292 | 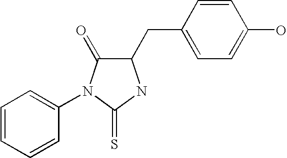 | 66.6 |
| A3.293 | 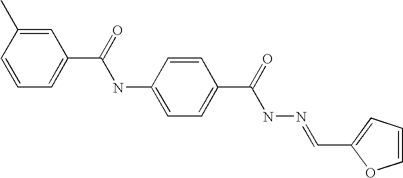 | 67.1* |
| A3.294 | 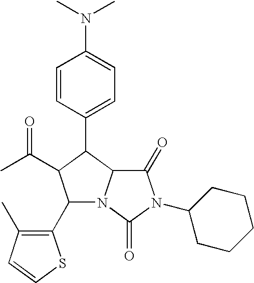 | 68.0 |
| A3.295 | 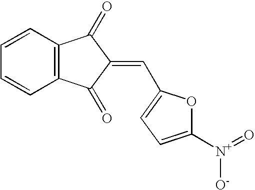 | 68.0 |
| A3.296 | 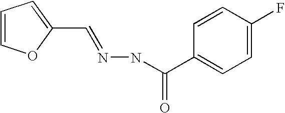 | 68.9 |
| A3.297 | 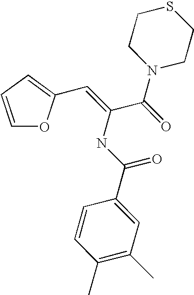 | 68.9 |
| A3.298 | 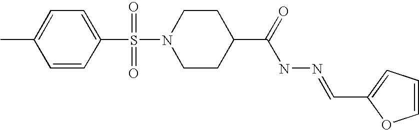 | 69.3 |
| A3.299 | 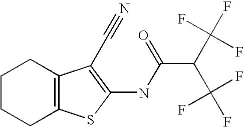 | 71.4 |
| A3.300 | 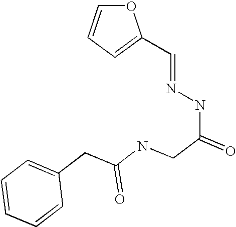 | 73.1* |
| A3.302 | 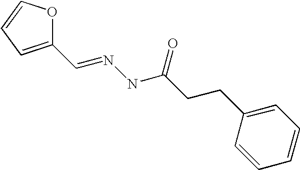 | 74.7 |
| A3.303 | 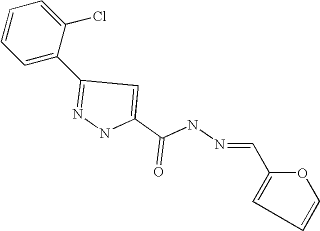 | 75.8* |
| A3.305 | 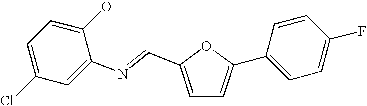 | 77.3 |
| A3.307 | 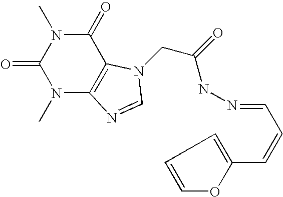 | 79.8* |
| A3.309 | 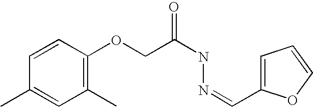 | 81.8* |
| A3.310 | 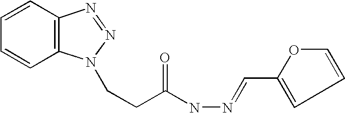 | 82.9 |
| A3.311 | 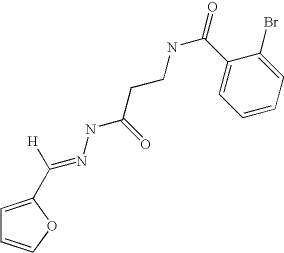 | 85.8* |
| A3.312 | 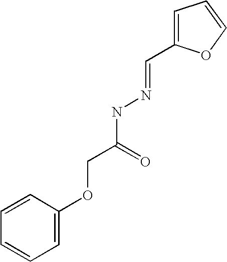 | 87.5 |
| A3.313 | 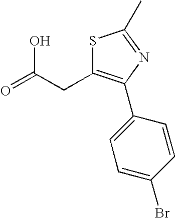 | 88.3 |
| A3.314 | 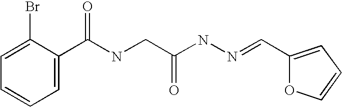 | 91.2* |
| A3.315 | 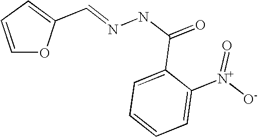 | 92.2 |
| A3.316 | 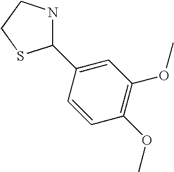 | 92.1 |
| A3.317 | 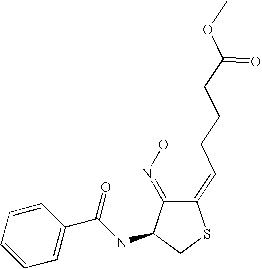 | 93.6 |
| A3.318 | 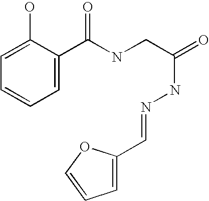 | 98.8* |
| A3.319 | 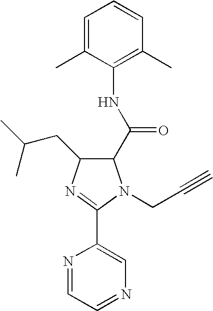 | 102.6* |
| A3.320 | 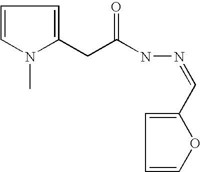 | 112.8* |
| A3.321 | 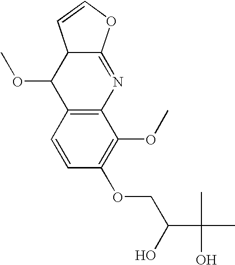 | 125.4 |
| A3.322 | 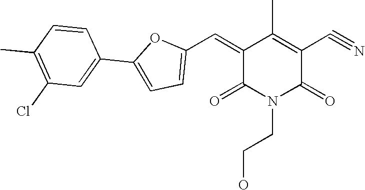 | 125.4 |
| A3.323 | 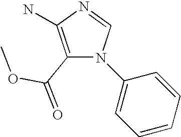 | 126.2* |
| A3.324 | 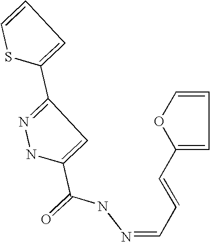 | 131.9* |
| A3.325 | 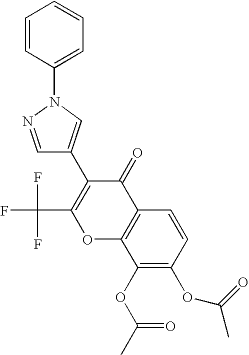 | 133.7* |
| A3.326 | 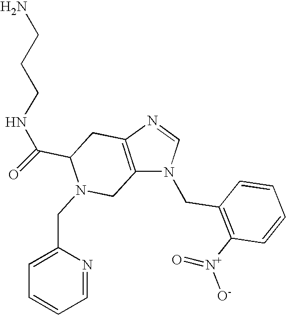 | 138.4 |
| A3.327 | 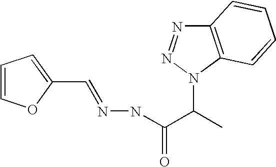 | 146.5* |
| A3.328 | 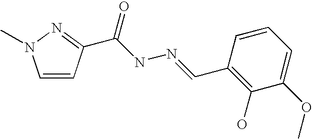 | 142.2 |
| A3.329 | 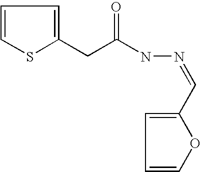 | 143.1 |
| A3.330 | 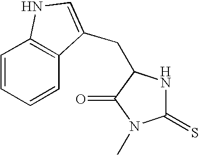 | 146.5* |
| A3.331 | 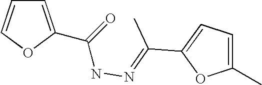 | 152.4 |
| A3.332 | 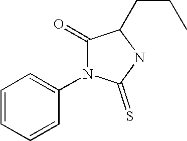 | 154.4* |
| A3.333 | 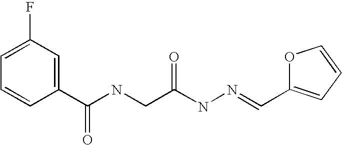 | 155.0* |
| A3.334 | 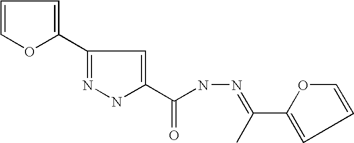 | 160.7* |
| A3.335 | 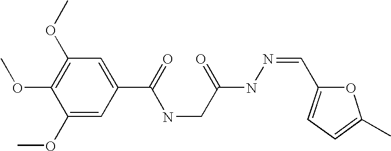 | 161.2* |
| A3.336 | 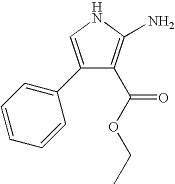 | 168.7* |
| A3.337 | 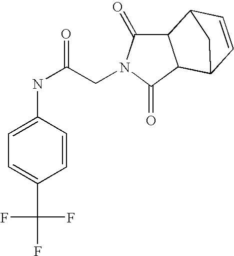 | 223.0* |
| A3.338 | 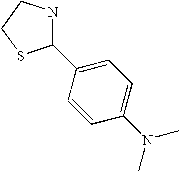 | 229.8* |
| A3.339 | 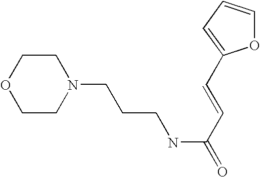 | 208.5* |
| A3.340 | 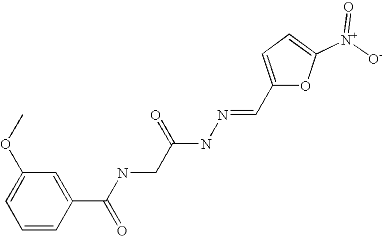 | 221.8* |
| A3.341 | 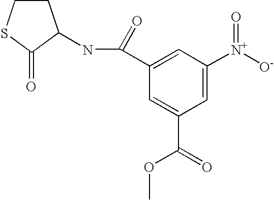 | 238.4* |
| A3.342 | 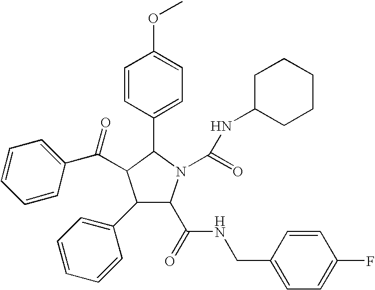 | 231.1* |
| A3.345 | 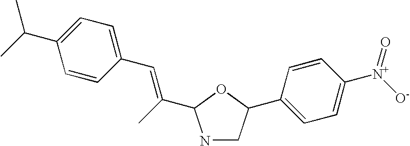 | 293.0* |
| A3.346 | 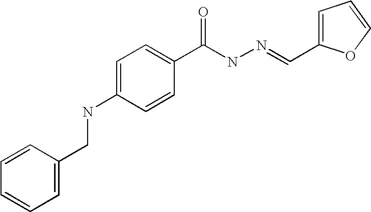 | 293.8* |
| A3.347 | 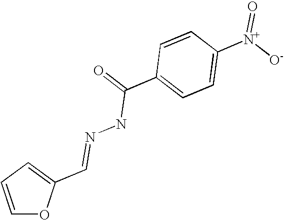 | 305.2* |
| A3.348 | 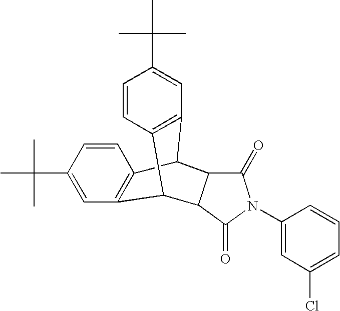 | 321.1* |
| A3.349 | 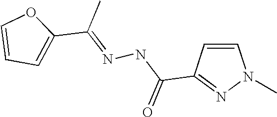 | 322.8* |
| A3.350 | 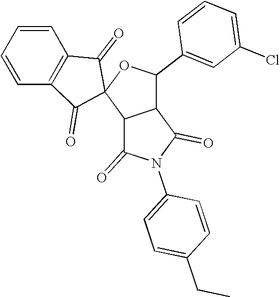 | 335.9* |
| A3.351 | 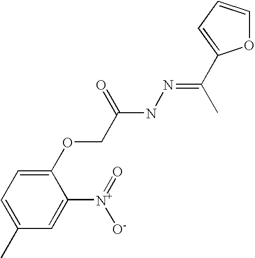 | 422.4* |
| A3.352 | 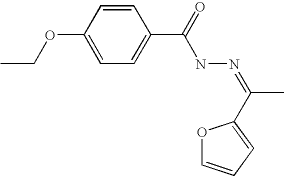 | 451.4* |

TABLE 4

| Compound ID. | Structure | IC50_{APN}[μM] |
|---|---|---|
| A4.001 | 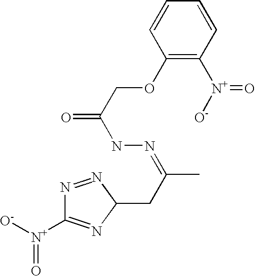 | 3.4 |
| A4.002 | 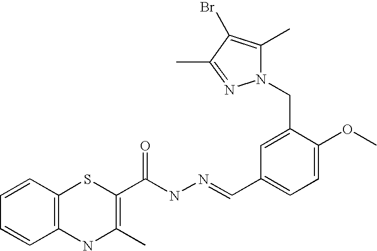 | 4.8 |
| A4.003 | 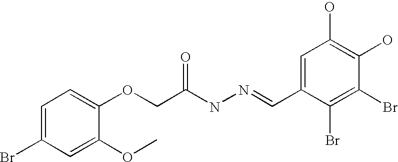 | 5.0 |
| A4.004 | 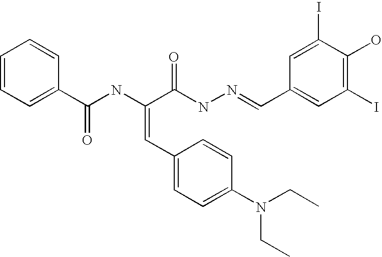 | 5.1 |
| A4.005 | 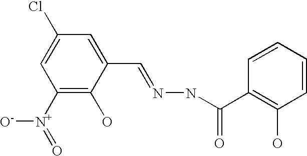 | 5.8 |
| A4.006 | 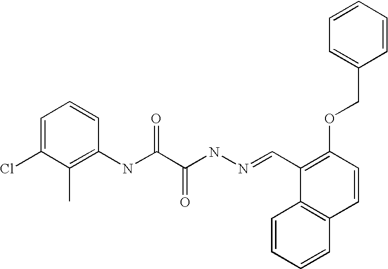 | 6.6 |
| A4.007 | 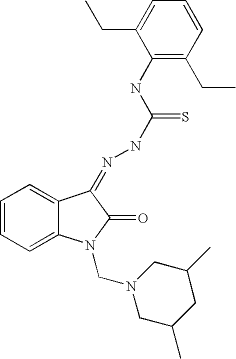 | 6.7 |
| A4.008 | 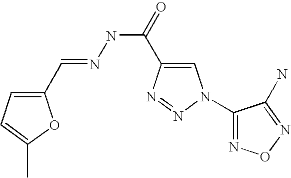 | 6.9 |
| A4.009 | 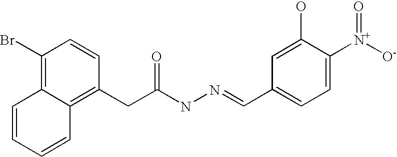 | 7.0 |
| A4.010 | 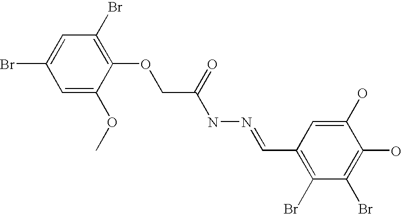 | 7.2 |
| A4.011 | 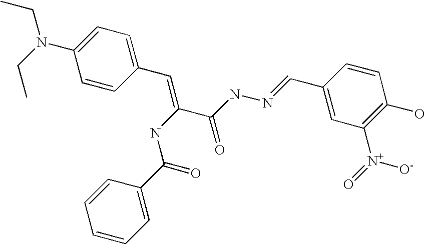 | 7.6 |
| A4.012 | 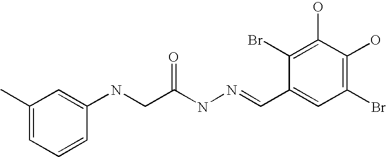 | 7.8 |
| A4.013 | 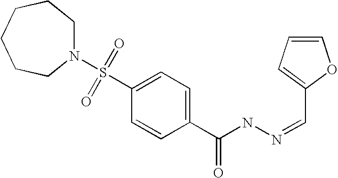 | 8.0 |
| A4.014 | 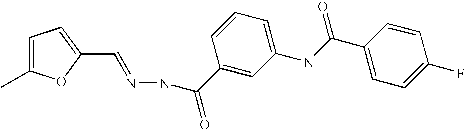 | 9.8 |
| A4.015 | 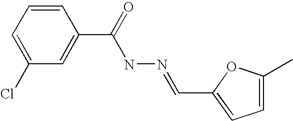 | 10.5 |
| A4.016 | 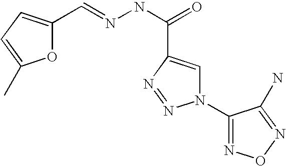 | 10.9 |
| A4.017 | 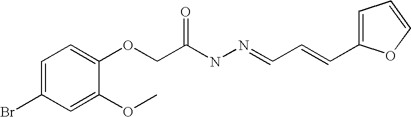 | 11.0 |
| A4.018 | 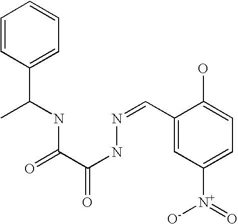 | 11.4 |
| A4.019 | 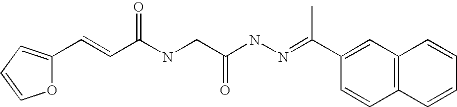 | 12.0 |
| A4.020 | 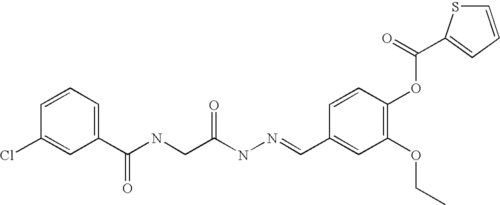 | 12.2 |
| A4.021 | 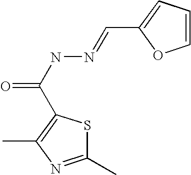 | 12.3 |
| A4.022 | 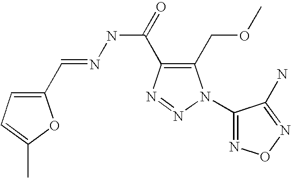 | 12.5 |
| A4.023 | 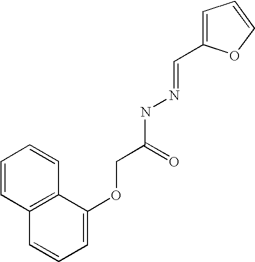 | 12.9 |
| A4.024 | 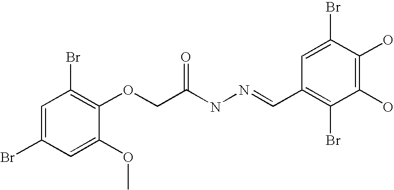 | 13.2 |
| A4.025 | 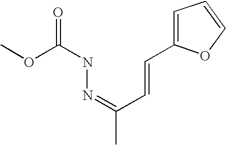 | 13.5 |
| A4.026 | 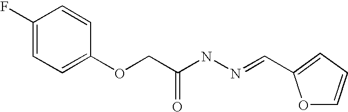 | 13.6 |
| A4.027 | 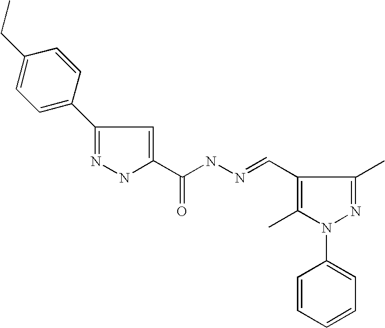 | 13.6 |
| A4.028 | 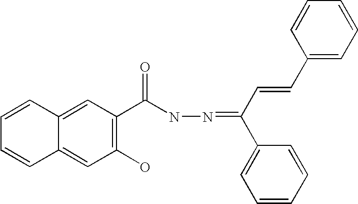 | 13.7 |
| A4.029 | 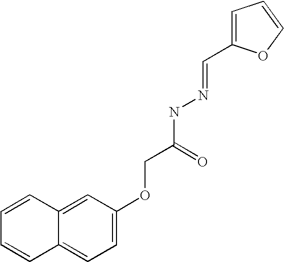 | 13.9 |
| A4.030 | 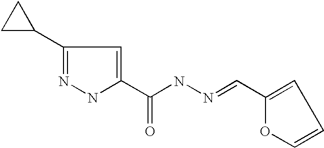 | 14.3 |
| A4.031 | 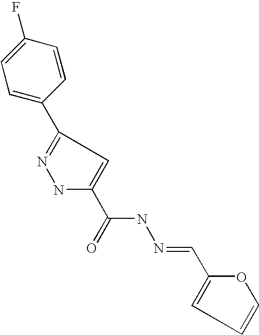 | 14.4 |
| A4.032 | 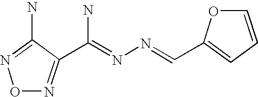 | 14.6 |
| A4.033 | 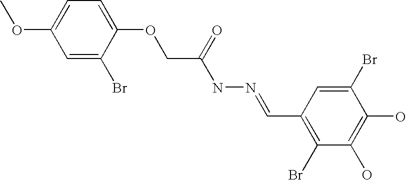 | 15.0 |
| A4.034 | 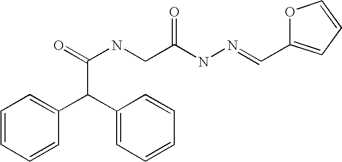 | 15.6 |
| A4.035 | 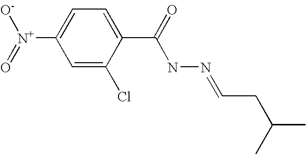 | 15.6 |
| A4.036 | 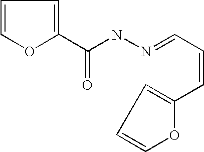 | 16.0 |
| A4.037 | 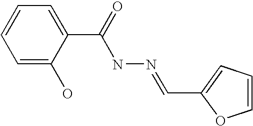 | 16.0 |
| A4.038 | 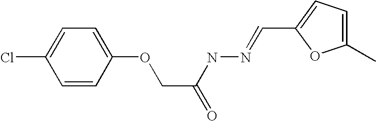 | 16.0 |
| A4.040 | 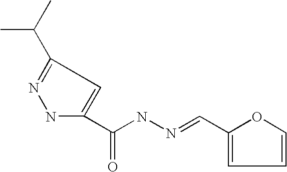 | 16.6 |
| A4.041 | 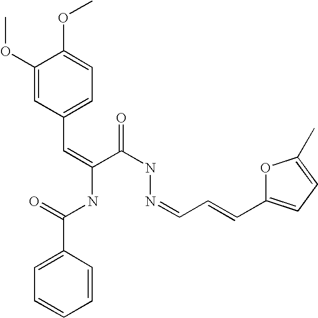 | 16.7 |
| A4.042 | 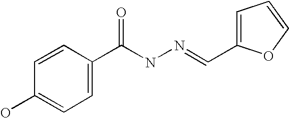 | 16.8 |
| A4.043 | 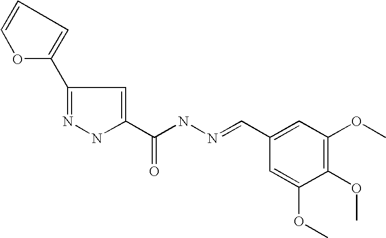 | 17.0 |
| A4.044 | 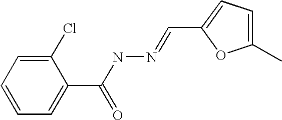 | 17.7 |
| A4.045 | 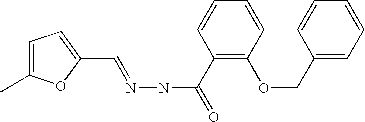 | 18.4 |
| A4.046 | 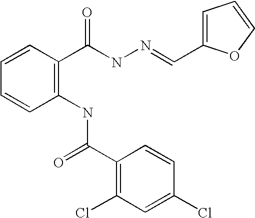 | 18.7 |
| A4.047 | 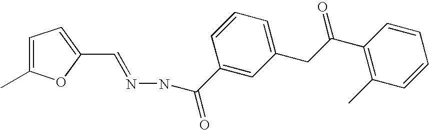 | 18.9 |
| A4.048 | 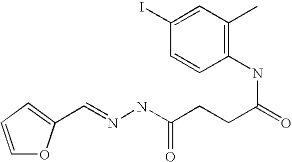 | 9.5 |
| A4.049 | 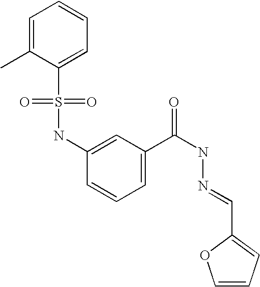 | 19.2 |
| A4.050 | 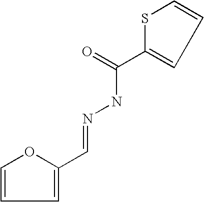 | 19.4 |
| A4.051 | 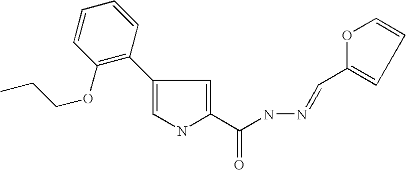 | 19.5 |
| A4.052 | 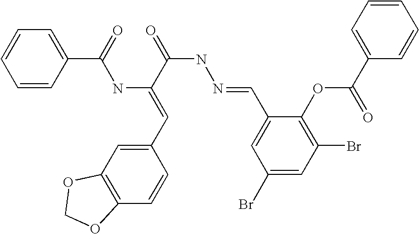 | 19.8 |
| A4.053 | 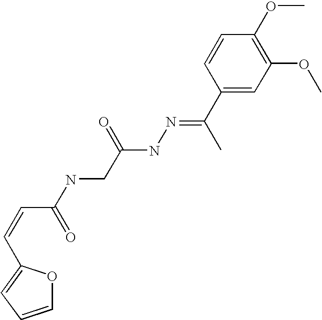 | 19.9 |
| A4.054 | 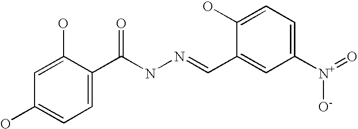 | 20.2 |
| A4.055 | 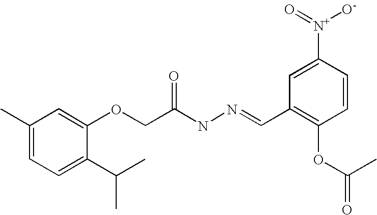 | 20.3 |
| A4.056 | 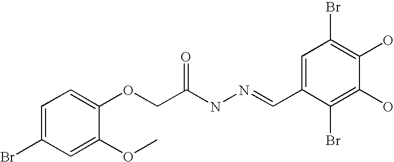 | 20.5 |
| A4.057 | 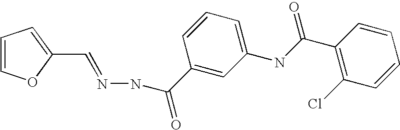 | 20.6 |
| A4.058 | 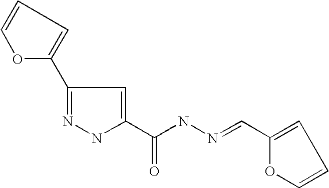 | 20.9 |
| A4.059 | 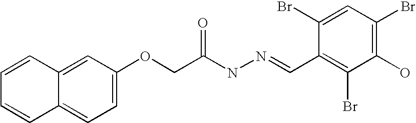 | 21.0 |
| A4.060 | 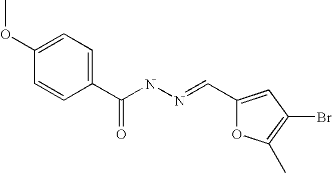 | 21.8 |
| A4.061 | 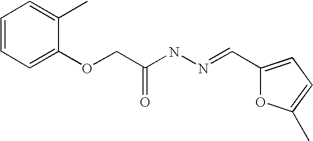 | 22.2 |
| A4.063 | 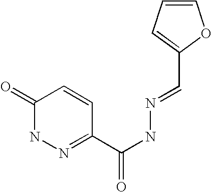 | 22.4 |
| A4.064 | 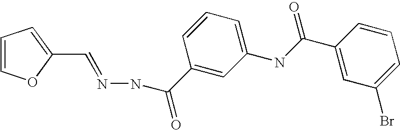 | 22.4 |
| A4.065 | 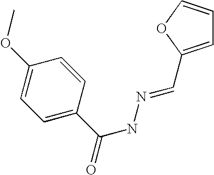 | 22.6 |
| A4.066 | 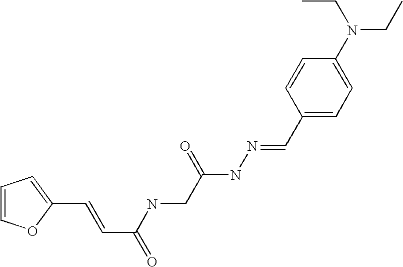 | 22.8 |
| A4.067 | 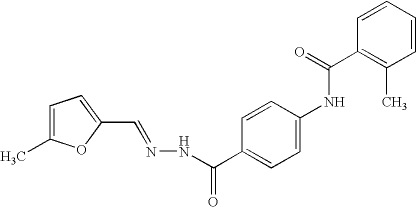 | 22.8 |
| A4.068 | 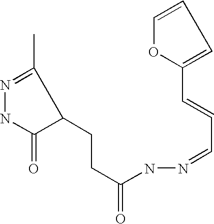 | 23.0 |
| A4.069 | 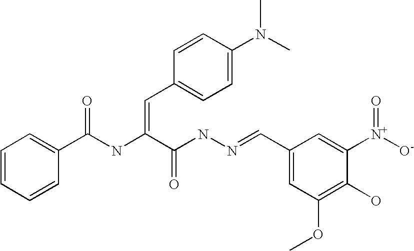 | 23.0 |
| A4.070 | 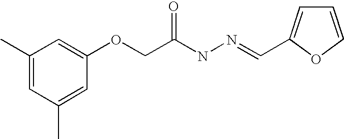 | 23.4 |
| A4.071 | 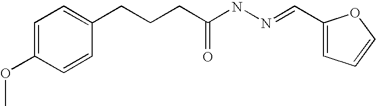 | 23.4 |
| A4.072 | 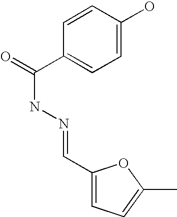 | 23.4 |
| A4.073 | 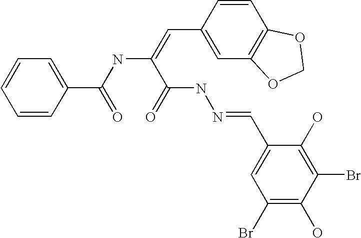 | 23.6 |
| A4.074 | 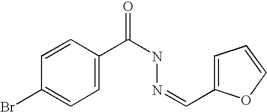 | 24.5 |
| A4.075 | 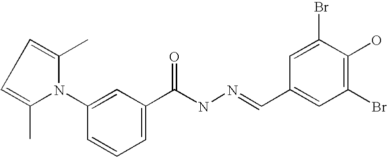 | 24.9 |
| A4.076 | 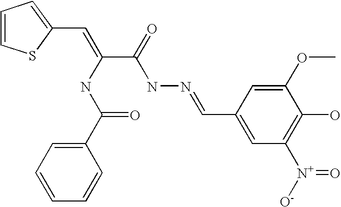 | 25.1 |
| A4.077 | 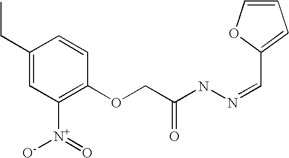 | 25.1 |
| A4.078 | 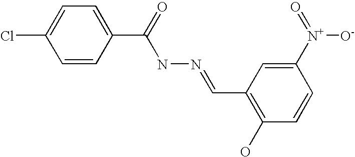 | 25.7 |
| A4.079 | 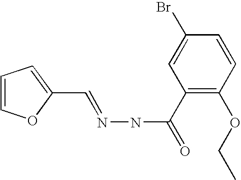 | 25.8 |
| A4.080 | 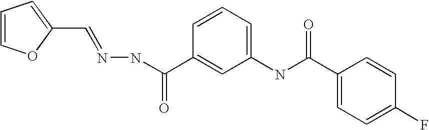 | 25.9 |
| A4.081 | 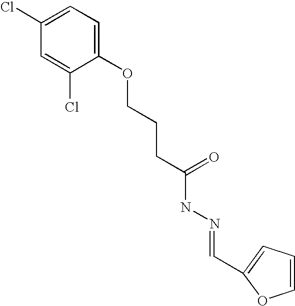 | 26.3 |
| A4.082 | 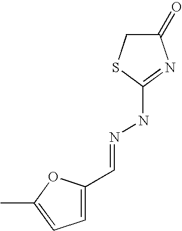 | 26.4 |
| A4.083 | 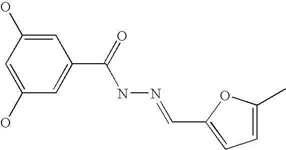 | 26.4 |
| A4.084 | 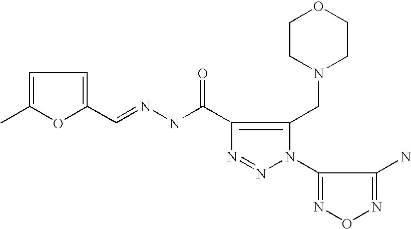 | 26.7 |
| A4.085 | 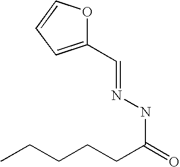 | 26.7 |
| A4.086 | 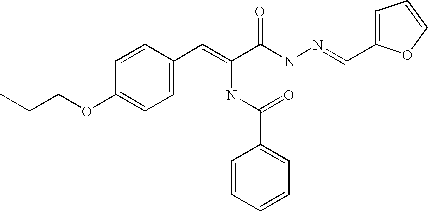 | 27.3 |
| A4.087 | 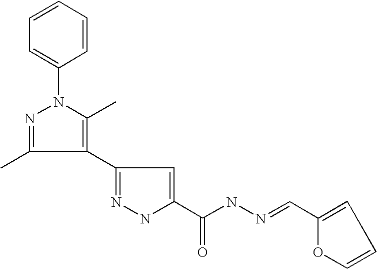 | 27.4 |
| A4.088 | 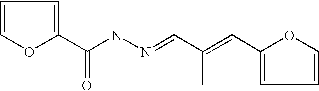 | 27.5 |
| A4.089 | 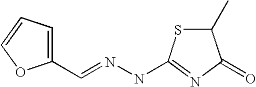 | 27.7 |
| A4.090 | 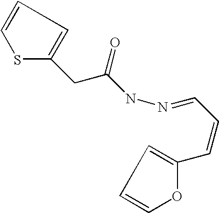 | 28.0 |
| A4.091 | 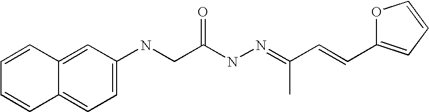 | 28.5 |
| A4.092 | 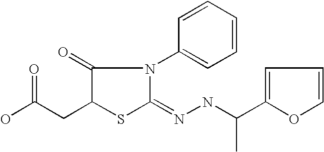 | 28.6 |
| A4.093 | 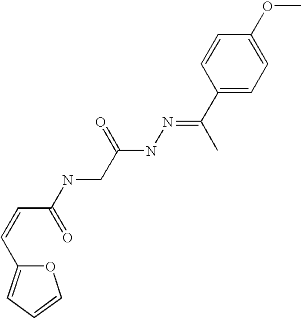 | 29.4 |
| A4.094 | 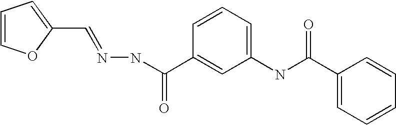 | 29.5 |
| A4.095 | 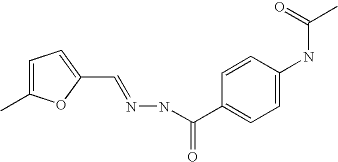 | 29.7 |
| A4.096 | 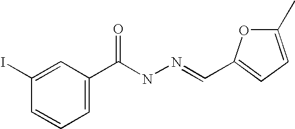 | 30.0 |
| A4.097 | 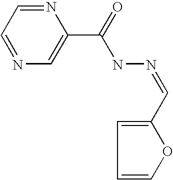 | 30.1 |
| A4.098 | 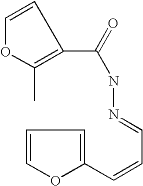 | 31.0 |
| A4.099 | 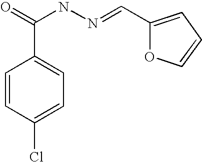 | 30.8 |
| A4.100 | 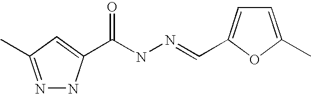 | 31.8 |
| A4.101 | 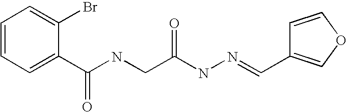 | 31.9 |
| A4.102 | 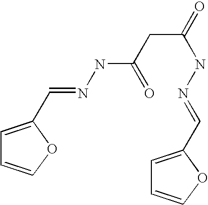 | 32.4 |
| A4.103 | 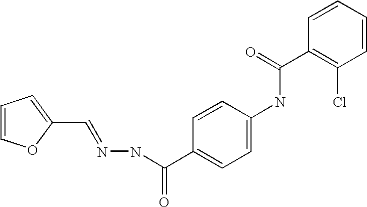 | 32.5 |
| A4.104 | 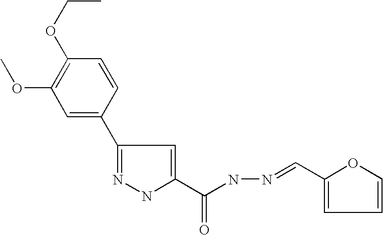 | 33.4 |
| A4.105 | 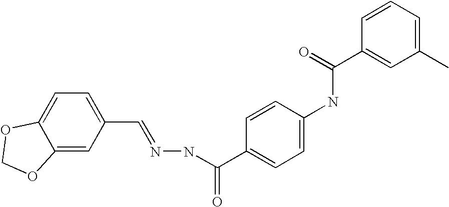 | 33.8* |
| A4.106 | 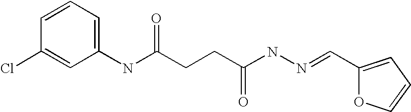 | 34.0 |
| A4.107 | 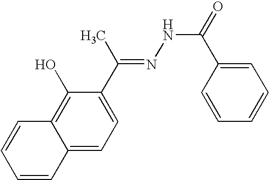 | 34.2 |
| A4.108 | 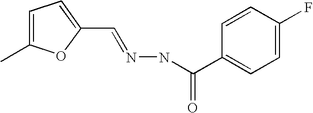 | 34.4 |
| A4.109 | 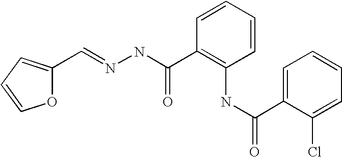 | 34.7 |
| A4.110 | 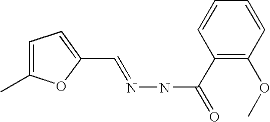 | 34.7 |
| A4.111 | 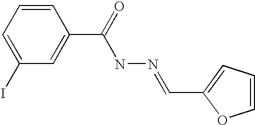 | 34.7 |
| A4.112 | 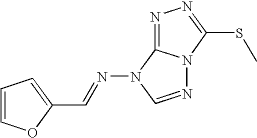 | 34.7 |
| A4.113 | 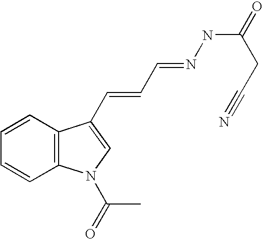 | 35.5 |
| A4.114 | 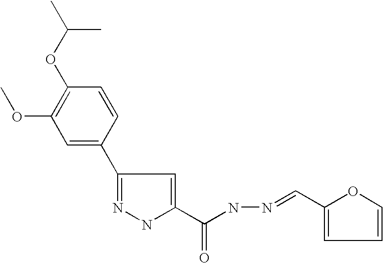 | 36.1 |
| A4.115 | 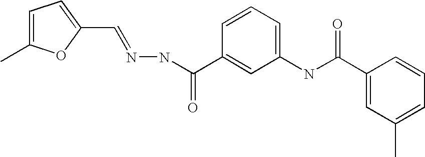 | 36.2 |
| A4.116 | 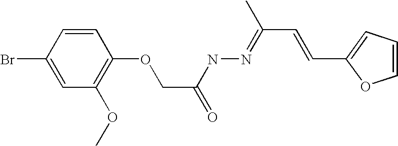 | 36.3 |
| A4.117 | 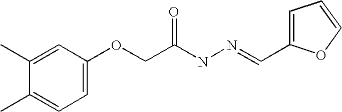 | 36.3 |
| A4.118 | 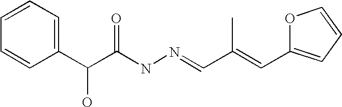 | 36.3 |
| A4.119 | 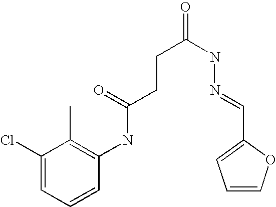 | 36.6 |
| A4.120 | 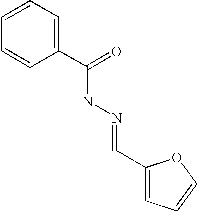 | 37.7 |
| A4.121 | 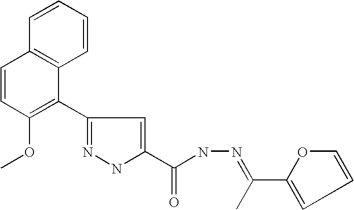 | 38.2 |
| A4.122 | 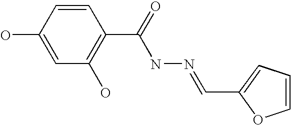 | 38.3 |
| A4.123 | 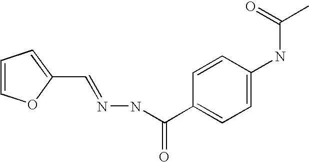 | 38.4 |
| A4.124 | 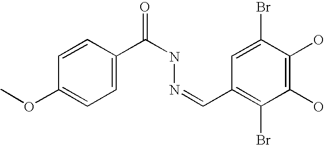 | 39.7* |
| A4.125 | 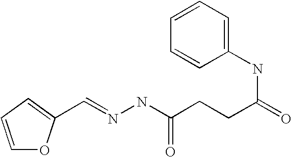 | 41.1 |
| A4.126 | 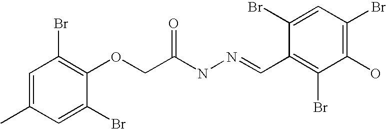 | 42.2 |
| A4.127 | 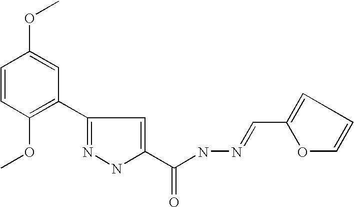 | 43.2 |
| A4.128 | 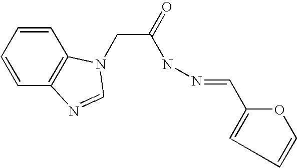 | 43.3 |
| A4.129 | 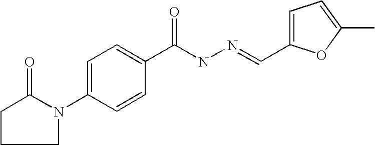 | 44.2 |
| A4.130 | 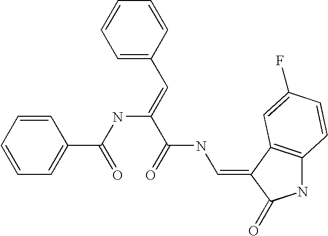 | 44.2 |
| A4.131 | 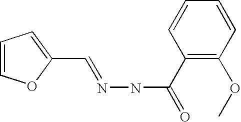 | 44.7 |
| A4.132 | 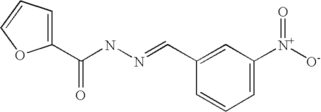 | 45.4 |
| A4.133 | 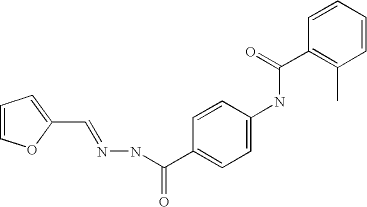 | 45.4 |
| A4.134 | 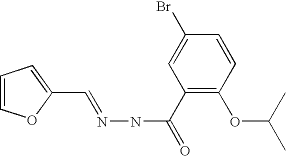 | 46.6 |
| A4.135 | 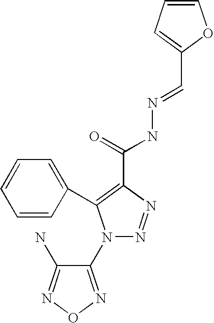 | 46.6 |
| A4.136 | 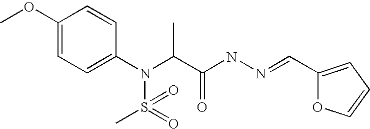 | 47.0 |
| A4.137 | 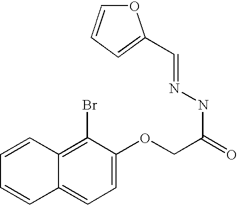 | 47.3 |
| A4.138 | 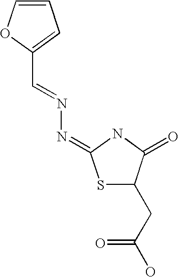 | 48.1* |
| A4.139 | 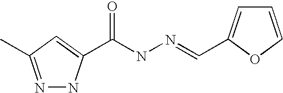 | 48.2 |
| A4.140 | 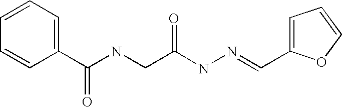 | 49.4* |
| A4.141 | 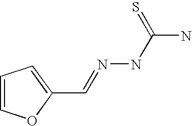 | 50.0 |
| A4.142 | 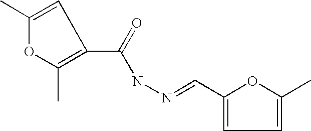 | 50.2 |
| A4.143 | 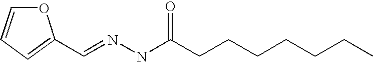 | 50.3 |
| A4.144 | 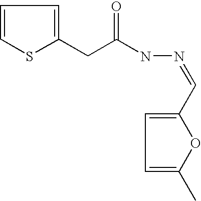 | 51.1 |
| A4.145 | 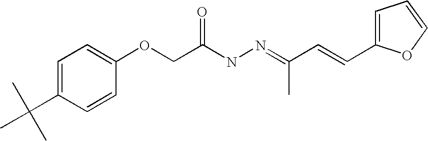 | 53.4 |
| A4.146 | 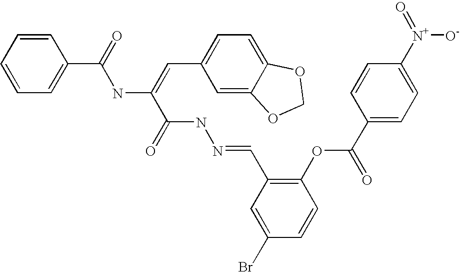 | 54.2 |
| A4.147 | 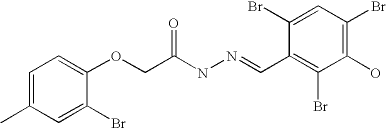 | 54.2* |
| A4.148 | 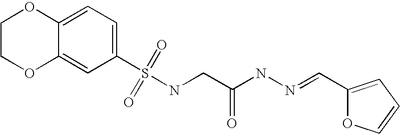 | 55.6 |
| A4.149 | 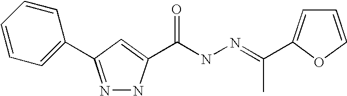 | 58.3 |
| A4.150 | 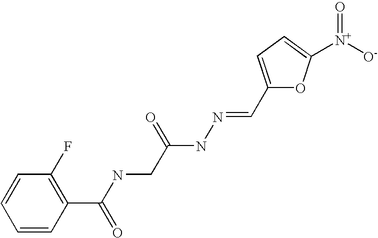 | 60.0 |
| A4.151 | 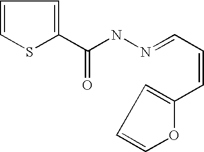 | 62.7 |
| A4.152 | 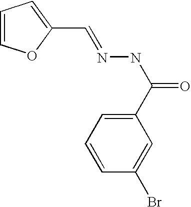 | 62.7 |
| A4.153 | 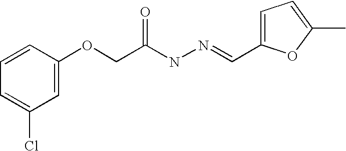 | 62.7 |
| A4.154 | 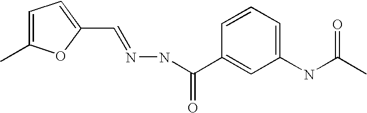 | 63.1* |
| A4.155 | 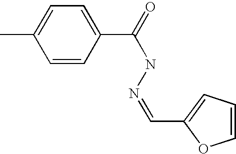 | 64.2 |
| A4.156 | 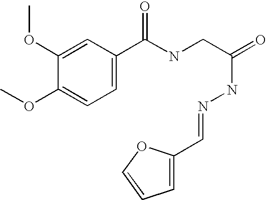 | 64.9* |
| A4.157 | 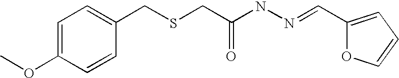 | 66.0* |
| A4.158 | 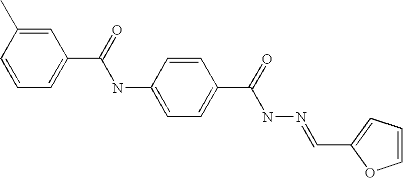 | 67.1* |
| A4.159 | 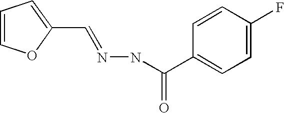 | 68.9 |
| A4.160 | 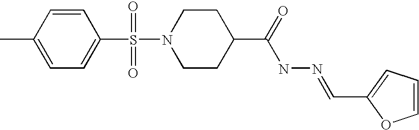 | 69.3 |
| A4.161 | 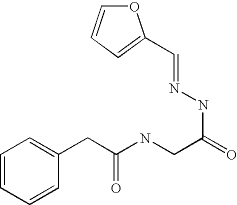 | 73.1* |
| A4.162 | 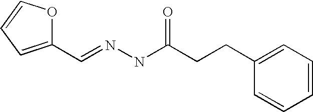 | 74.7 |
| A4.163 | 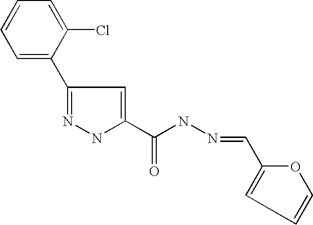 | 75.8* |
| A4.164 | 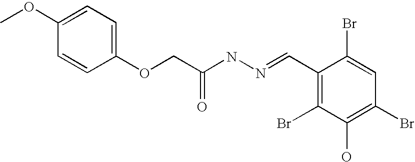 | 76.4* |
| A4.166 | 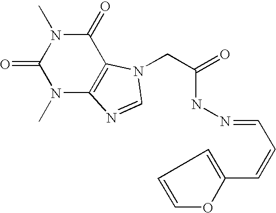 | 79.8* |
| A4.167 | 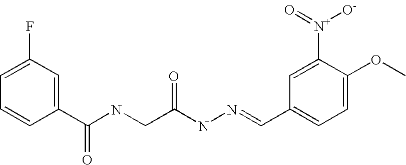 | 80.4* |
| A4.168 | 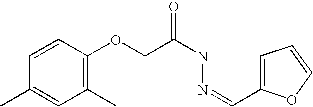 | 81.8* |
| A4.169 | 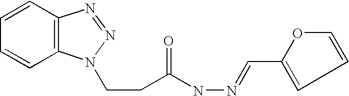 | 82.9 |
| A4.170 | 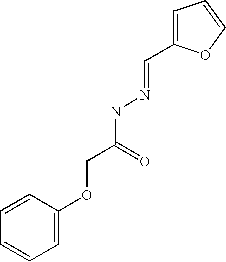 | 87.5 |
| A4.171 | 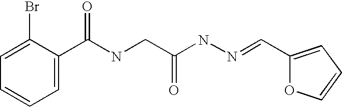 | 91.2* |
| A4.172 | 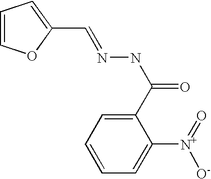 | 91.2 |
| A4.173 | 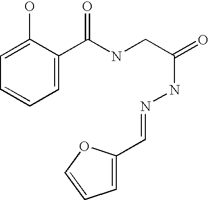 | 98.8* |
| A4.174 | 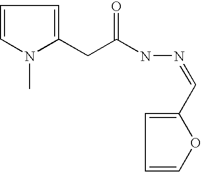 | 112.8* |
| A4.175 | 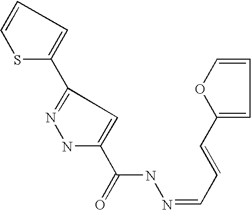 | 131.9* |
| A4.176 | 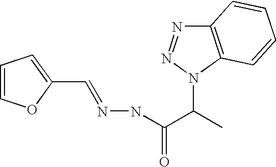 | 146.5* |
| A4.177 | 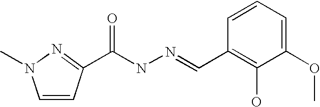 | 142.2 |
| A4.178 | 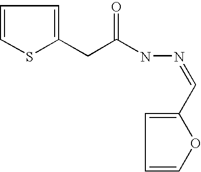 | 143.1 |
| A4.179 | 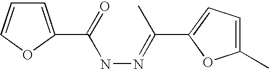 | 152.4 |
| A4.180 | 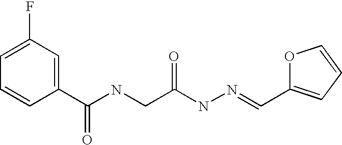 | 155.0* |
| A4.181 | 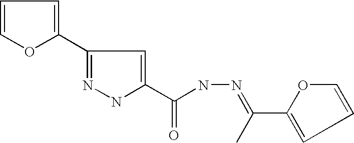 | 160.7* |
| A4.182 | 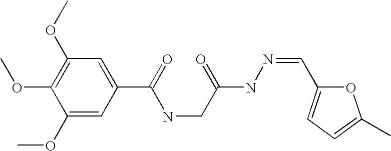 | 161.2* |
| A4.183 | 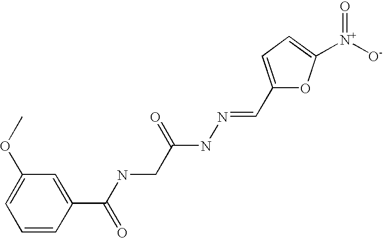 | 221.8* |
| A4.184 | 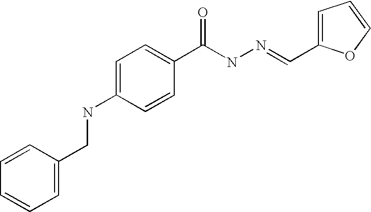 | 293.8* |
| A4.185 | 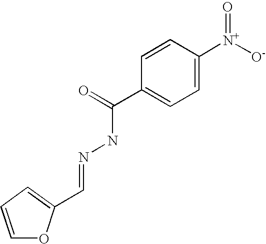 | 305.2* |
| A4.186 | 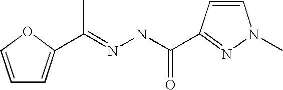 | 322.8* |
| A4.187 | 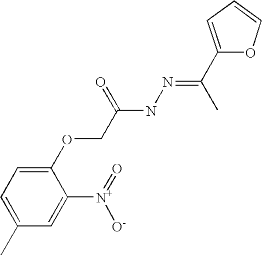 | 422.4* |

TABLE 5

| Compound ID. | Structure | IC50_{APN} [μM] |
|---|---|---|
| A5.001 | 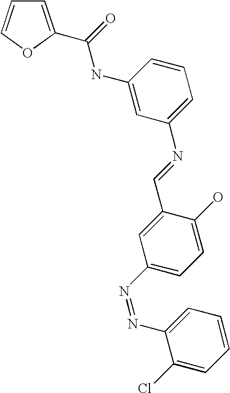 | 3.6 |
| A5.003 | 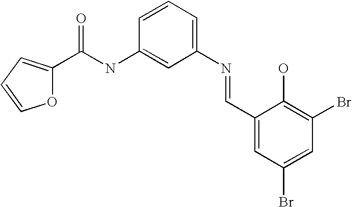 | 8.2 |
| A5.004 | 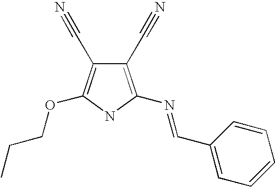 | 20.0 |
| A5.005 | 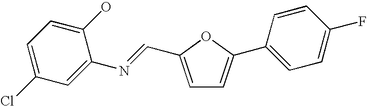 | 77.3 |
| A5.006 | 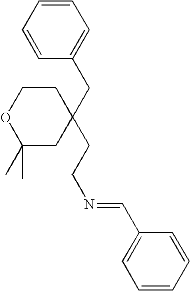 | 93.6 |
| A5.007 | 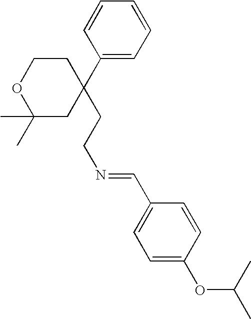 | 197.4* |

TABLE 6

| Compound ID. | Structure | IC50_{APN} [μM] |
|---|---|---|
| A6.001 | 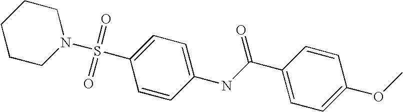 | 8.4 |
| A6.002 | 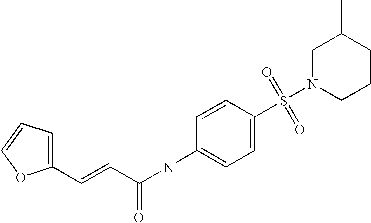 | 8.4 |
| A6.003 | 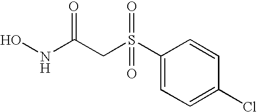 | 11.5 |
| A6.004 | 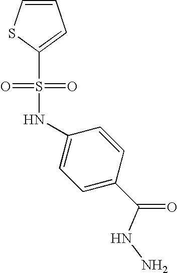 | 11.8 |
| A6.005 | 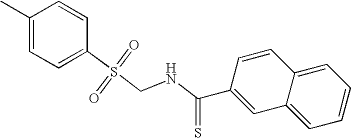 | 11.8 |
| A6.006 | 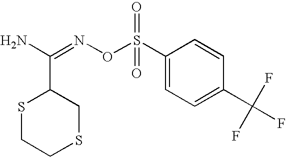 | 13.4 |
| A6.007 | 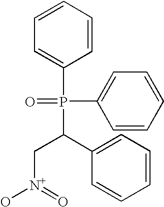 | 16.3 |
| A6.008 | 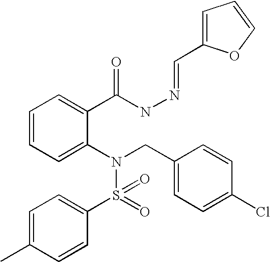 | 16.4 |
| A6.009 | 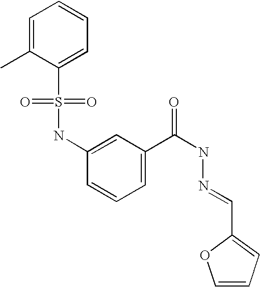 | 19.2 |
| A6.010 | 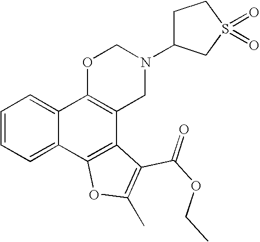 | 22.3 |
| A6.011 | 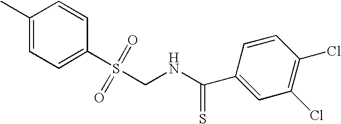 | 18.0 |
| A6.012 | 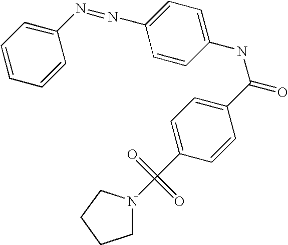 | 21.6 |
| A6.013 | 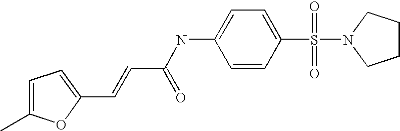 | 24.8 |
| A6.015 | 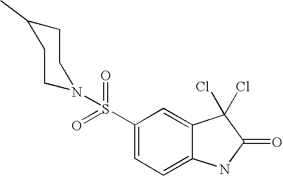 | 37.2 |
| A6.016 | 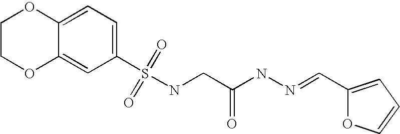 | 55.6 |
| A6.017 | 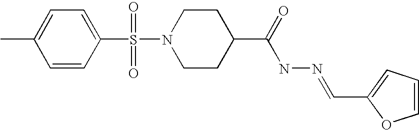 | 69.3 |

TABLE 7

| Compound ID. | Structure | IC50_{APN} [μM] |
|---|---|---|
| A7.001 | 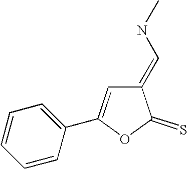 | 6.0 |
| A7.002 | 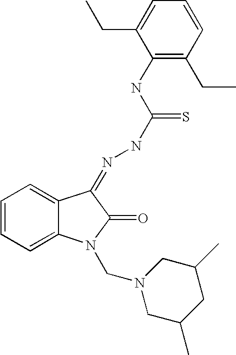 | 6.7 |
| A7.004 | 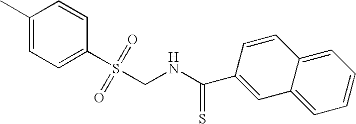 | 11.8 |
| A7.005 | 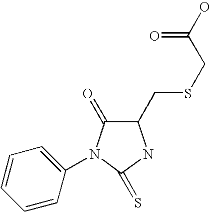 | 12.0 |
| A7.006 | 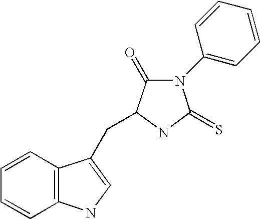 | 12.4 |
| A7.007 | 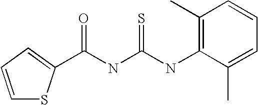 | 12.9 |
| A7.008 | 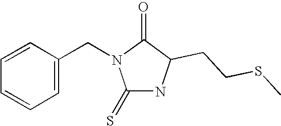 | 14.1 |
| A7.010 | 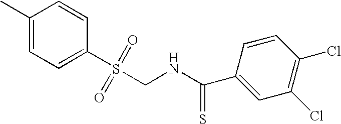 | 18.0 |
| A7.011 | 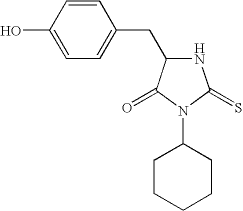 | 21.3 |
| A7.012 | 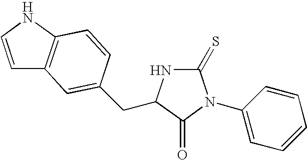 | 36.9 |

TABLE 8

| Compound ID. | Structure | IC50_{APN} [μM] |
|---|---|---|
| A8.001 | 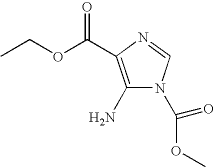 | 0.9* |
| A8.002 | 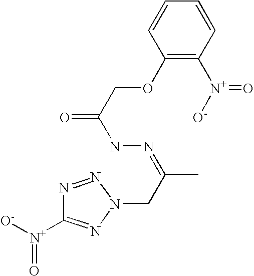 | 3.4 |
| A8.003 | 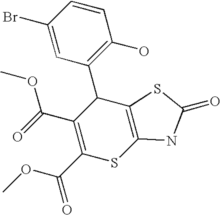 | 4.7 |
| A8.004 | 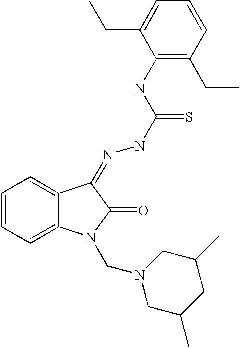 | 6.7 |
| A8.005 | 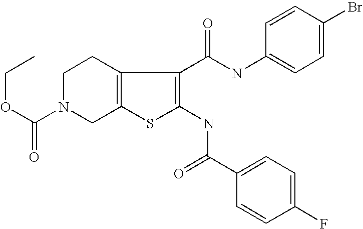 | 7.3 |
| A8.006 | 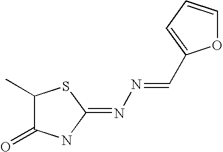 | 8.0 |
| A8.007 | 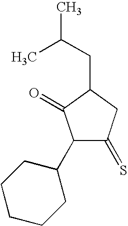 | 8.0 |
| A8.008 | 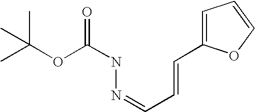 | 10.8 |
| A8.009 | 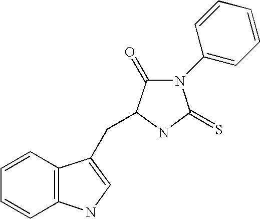 | 12.4 |
| A8.010 | 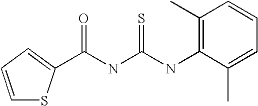 | 12.9 |
| A8.011 | 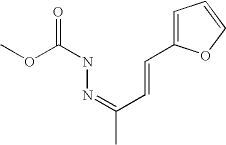 | 13.5 |
| A8.012 | 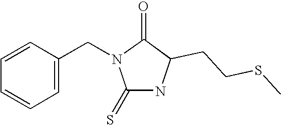 | 14.1 |
| A8.013 | 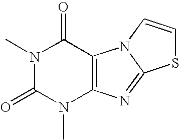 | 14.3 |
| A8.014 | 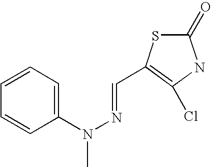 | 14.4 |
| A8.015 | 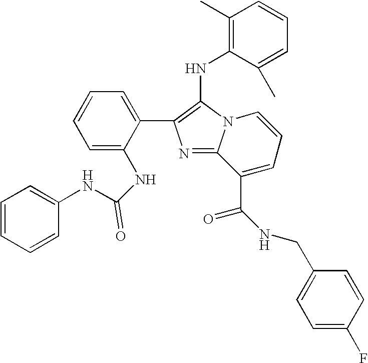 | 14.9 |
| A8.016 | 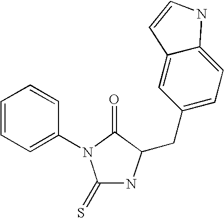 | 14.9 |
| A8.017 | 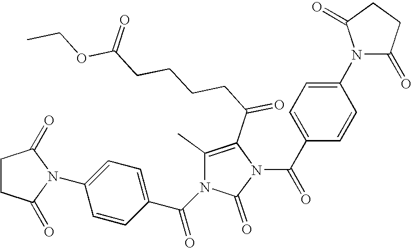 | 15.6 |
| A8.018 | 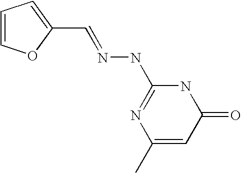 | 18.2 |
| A8.019 | 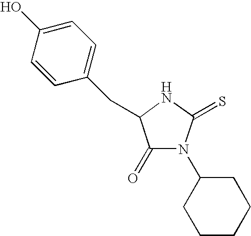 | 21.3 |
| A8.020 | 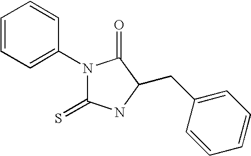 | 26.1 |
| A8.021 | 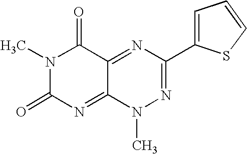 | 25.0 |
| A8.022 | 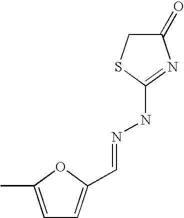 | 26.4 |
| A8.023 | 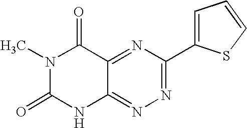 | 22.6 |
| A8.024 | 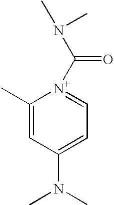 | 27.6 |
| A8.025 | 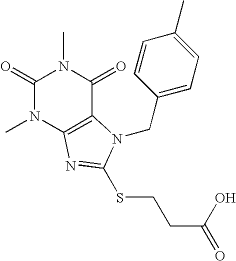 | 28.0 |
| A8.026 | 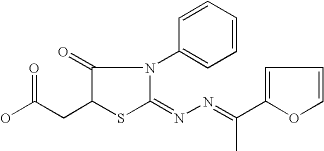 | 28.6 |
| A8.027 | 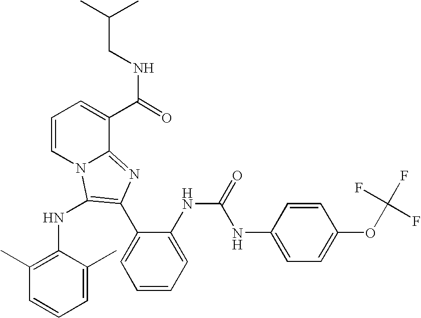 | 31.4 |
| A8.028 | 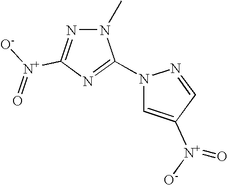 | 34.4 |
| A8.029 | 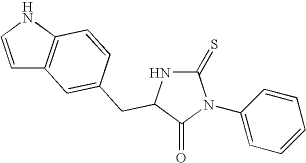 | 36.9 |
| A8.030 | 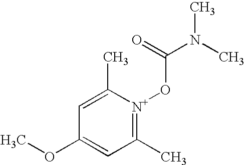 | 42.4 |
| A8.031 | 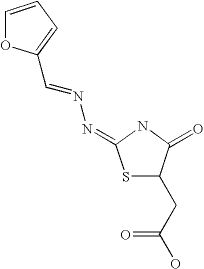 | 48.1* |
| A8.032 | 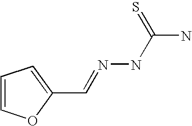 | 50.0 |
| A8.033 | 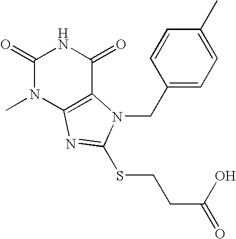 | 53.2* |
| A8.034 | 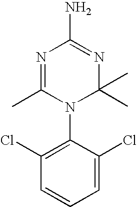 | 59.8 |
| A8.035 | 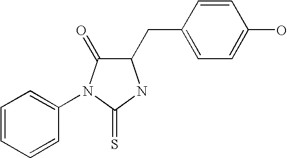 | 66.6 |
| A8.036 | 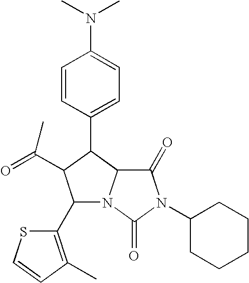 | 68.0 |
| A8.037 | 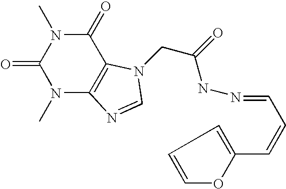 | 79.8* |
| A8.038 | 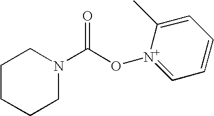 | 96.7* |
| A8.040 | 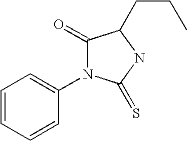 | 154.4* |
| A8.041 | 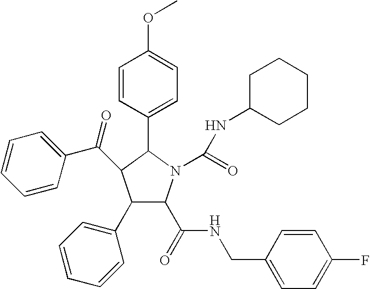 | 231.1* |

TABLE 9

| | Structure | IC50_{APN} [μM] |
|---|---|---|
| A9.001 | 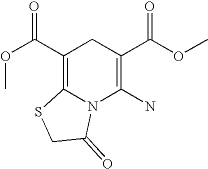 | ‘very high ac- tive’, beyond measure |
| A9.002 | 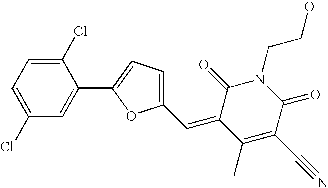 | 1.2* |
| A9.003 | 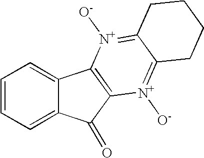 | 2.0* |
| A9.005 | 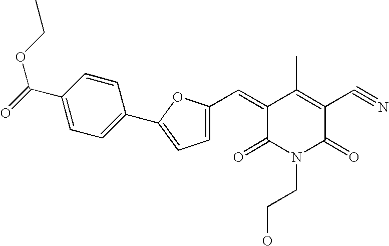 | 2.7* |
| A9.006 | 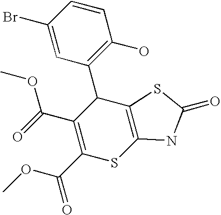 | 4.7 |
| A9.007 | 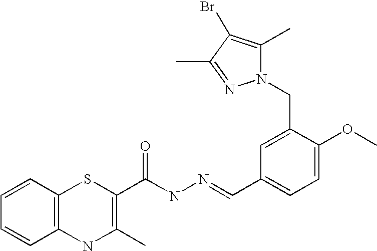 | 4.8 |
| A9.008 | 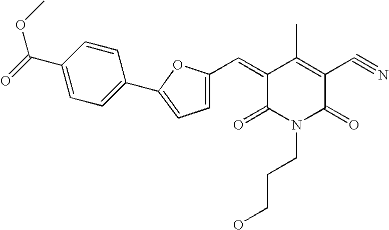 | 5.0 |
| A9.009 | 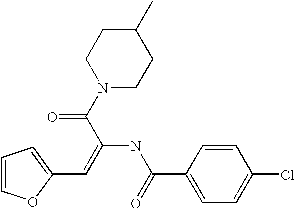 | 6.2 |
| A9.010 | 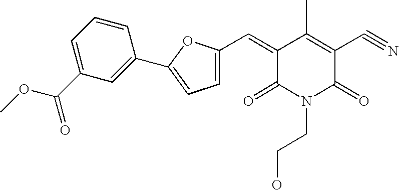 | 6.3 |
| A9.011 | 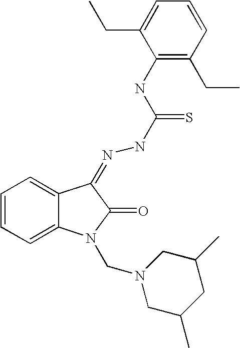 | 6.7 |
| A9.012 | 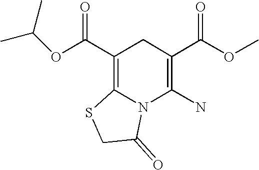 | 5.9 |
| A9.013 | 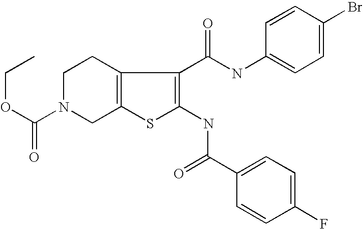 | 7.3 |
| A9.014 | 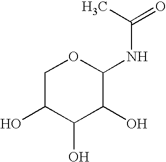 | 7.3 |
| A9.015 | 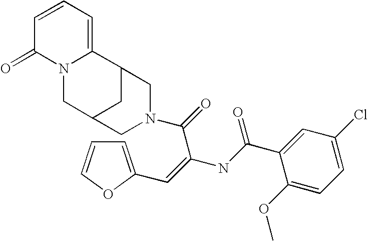 | 8.1 |
| A9.016 | 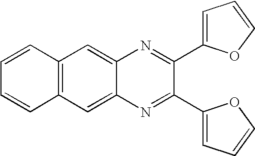 | 8.9 |
| A9.017 | 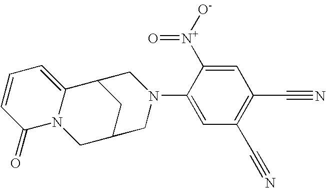 | 8.9 |
| A9.018 | 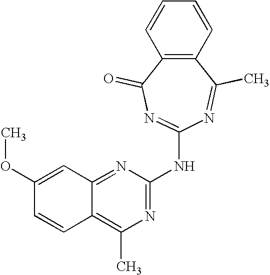 | 8.9 |
| A9.019 | 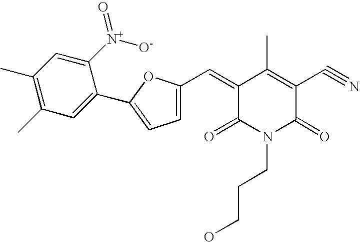 | 9.7 |
| A9.020 | 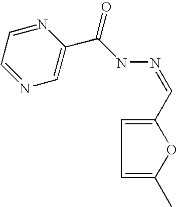 | 9.8 |
| A9.021 | 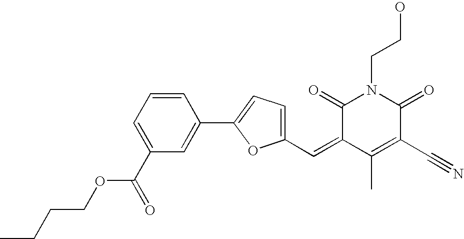 | 10.2* |
| A9.022 | 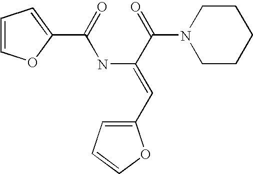 | 10.6 |
| A9.023 | 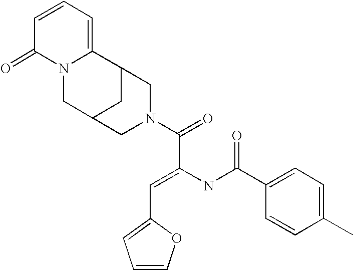 | 11.0 |
| A9.024 | 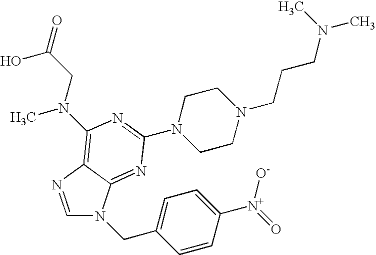 | 11.8 |
| A9.025 | 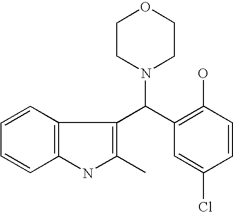 | 11.7 |
| A9.026 | 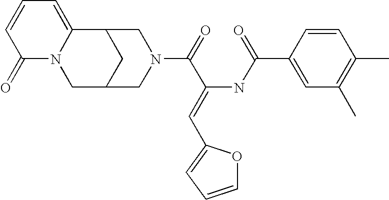 | 11.8 |
| A9.027 | 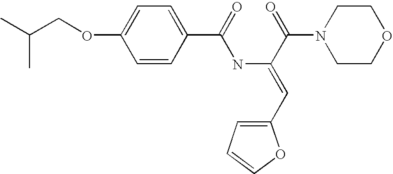 | 13.1 |
| A9.028 | 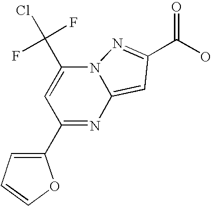 | 13.2 |
| A9.029 | 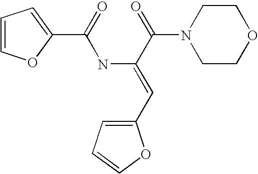 | 13.5 |
| A9.030 | 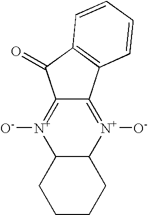 | 13.7 |
| A9.031 | 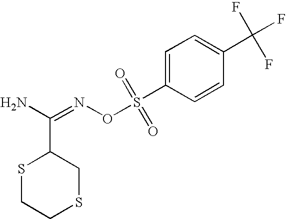 | 13.4 |
| A9.032 | 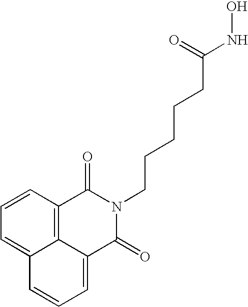 | 14.1 |
| A9.033 | 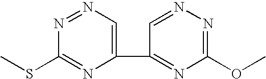 | 14.3 |
| A9.034 | 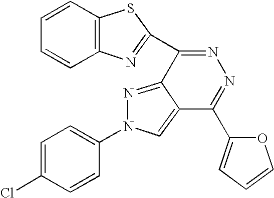 | 14.3 |
| A9.035 | 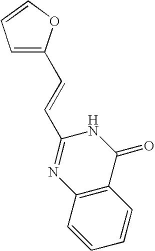 | 14.6 |
| A9.036 | 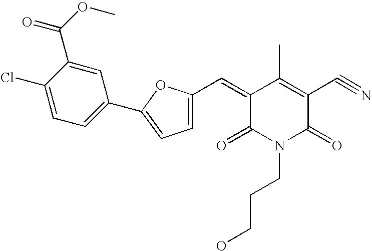 | 15.2 |
| A9.037 | 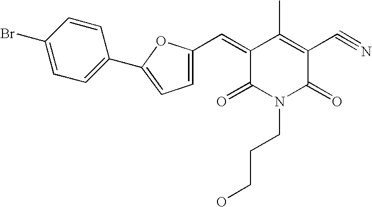 | 16.0 |
| A9.038 | 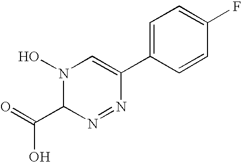 | 17.1 |
| A9.039 | 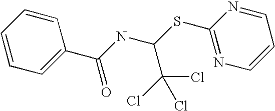 | 17.9 |
| A9.040 | 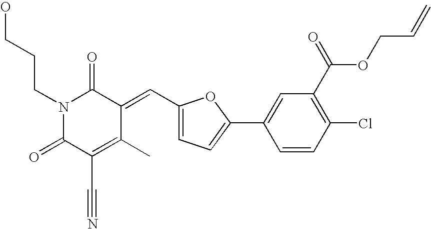 | 18.1 |
| A9.041 | 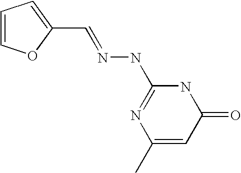 | 18.2 |
| A9.042 | 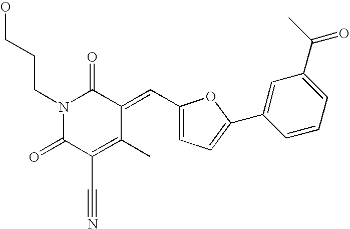 | 18.2 |
| A9.043 | 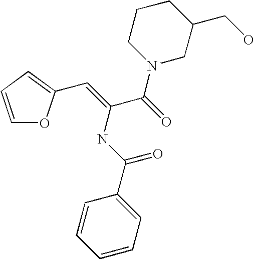 | 19.0 |
| A9.044 | 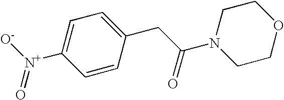 | 19.1 |
| A9.045 | 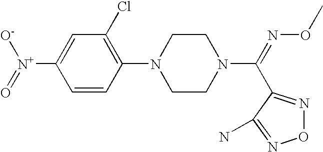 | 19.2 |
| A9.046 | 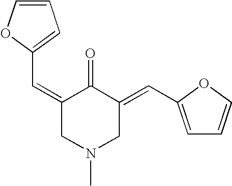 | 19.2 |
| A9.047 | 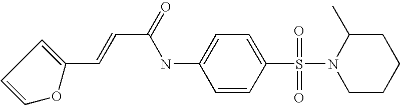 | 19.9 |
| A9.048 | 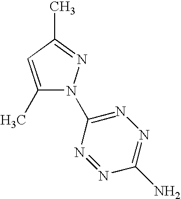 | 20.3 |
| A9.049 | 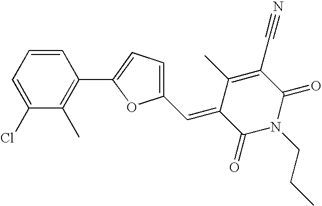 | 20.8 |
| A9.050 | 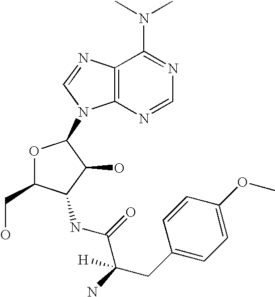 | 20.9 |
| A9.051 | 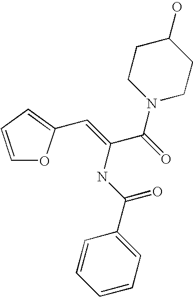 | 21.1 |
| A9.052 | 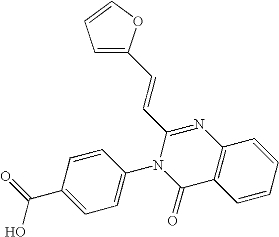 | 21.2 |
| A9.053 | 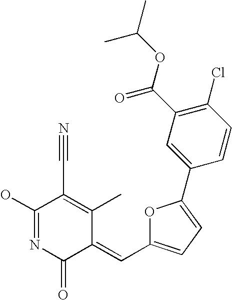 | 21.3 |
| A9.054 | 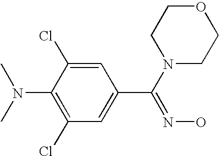 | 21.4 |
| A9.055 | 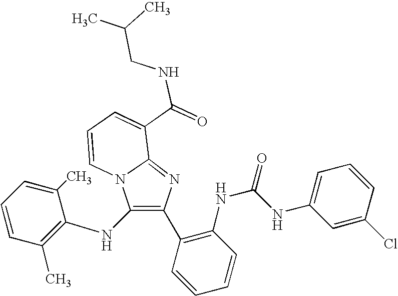 | 21.6 |
| A9.056 | 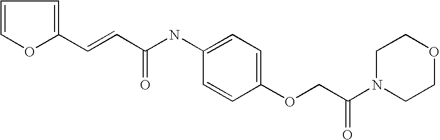 | 21.9 |
| A9.057 | 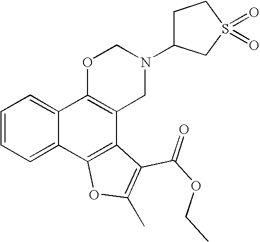 | 22.3 |
| A9.058 | 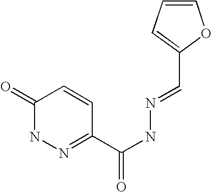 | 22.4 |
| A9.059 | 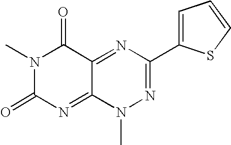 | 25.0 |
| A9.060 | 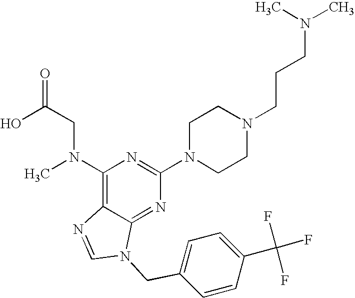 | 23.0 |
| A9.061 | 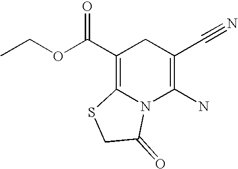 | 23.1 |
| A9.062 | 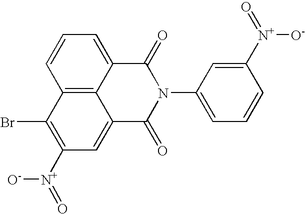 | 23.3 |
| A9.063 | 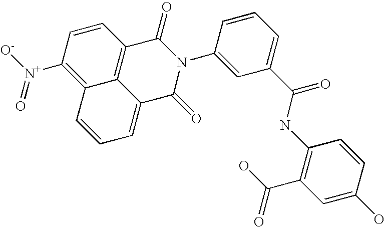 | 23.6 |
| A9.064 | 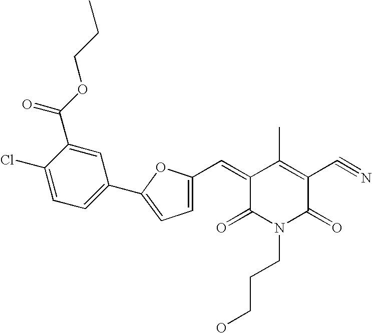 | 23.8 |
| A9.065 | 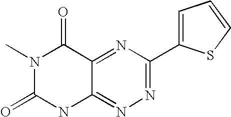 | 22.6 |
| A9.066 | 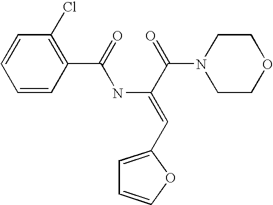 | 25.5 |
| A9.067 | 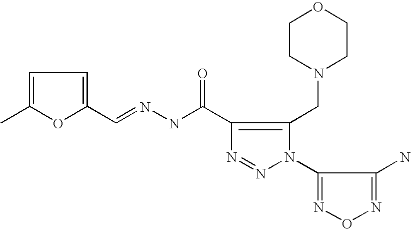 | 26.7 |
| A9.068 | 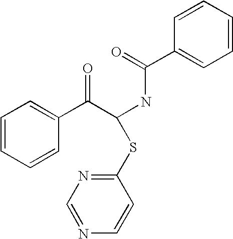 | 26.9 |
| A9.069 | 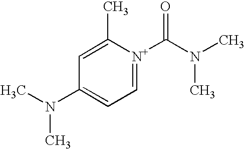 | 27.6 |
| A9.070 | 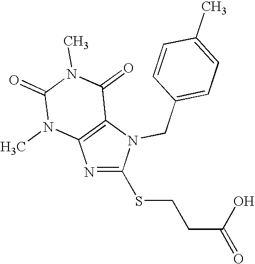 | 28.0 |
| A9.071 | 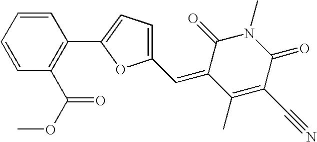 | 28.1 |
| A9.072 | 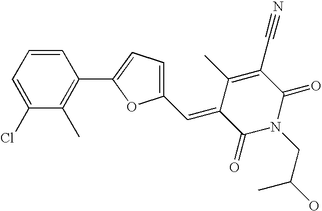 | 28.6 |
| A9.073 | 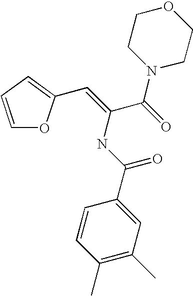 | 28.8 |
| A9.074 | 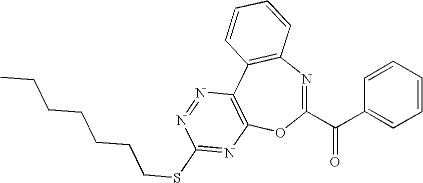 | 28.9 |
| A9.075 | 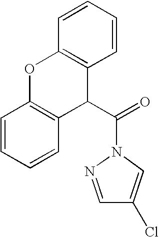 | 29.1 |
| A9.076 | 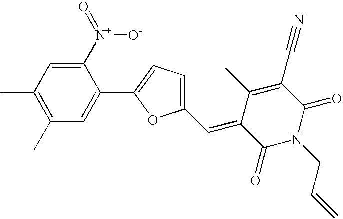 | 29.3 |
| A9.077 | 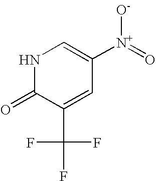 | 29.9 |
| A9.078 | 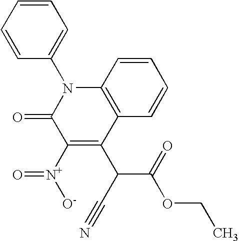 | 30.0 |
| A9.079 | 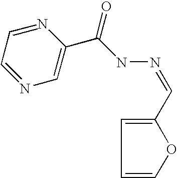 | 30.1 |
| A9.080 | 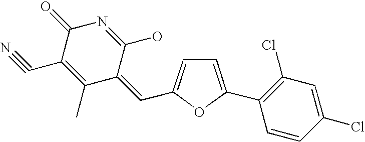 | 30.3 |
| A9.081 | 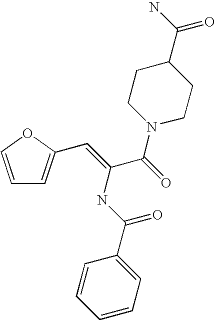 | 30.5 |
| A9.082 | 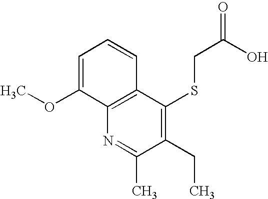 | 31.1 |
| A9.083 | 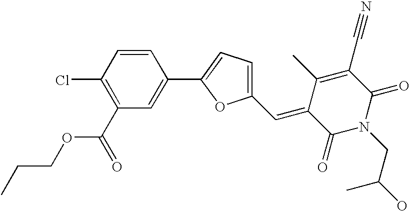 | 32.0 |
| A9.084 | 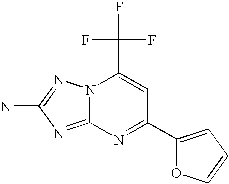 | 32.1 |
| A9.085 | 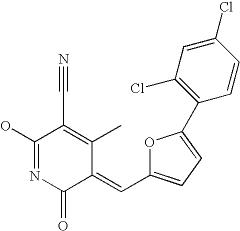 | 32.4 |
| A9.086 | 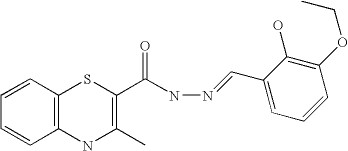 | 33.2 |
| A9.087 | 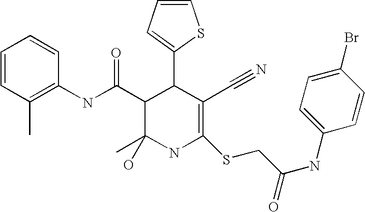 | 33.9 |
| A9.088 | 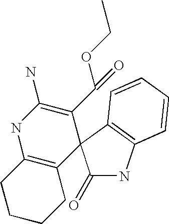 | 33.6 |
| A9.089 | 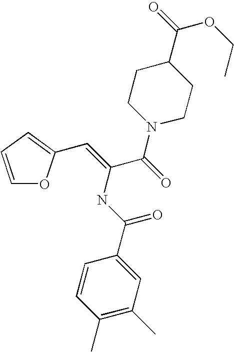 | 34.5 |
| A9.090 | 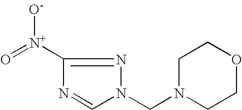 | 36.8 |
| A9.091 | 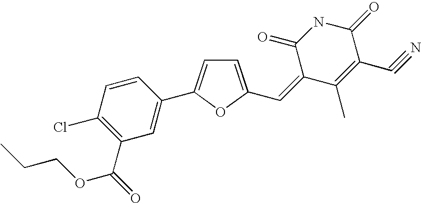 | 36.8 |
| A9.092 | 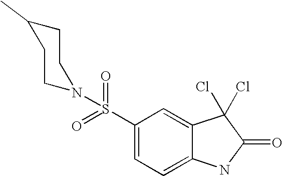 | 37.2 |
| A9.093 | 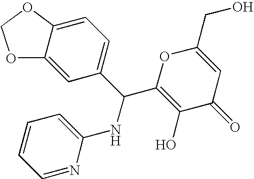 | 37.3 |
| A9.094 | 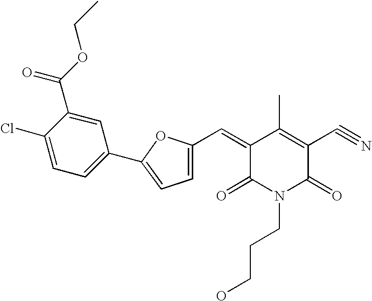 | 41.0 |
| A9.095 | 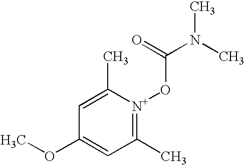 | 42.4 |
| A9.096 | 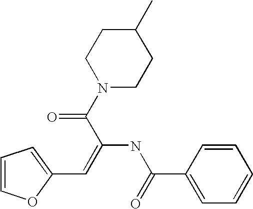 | 45.0 |
| A9.097 | 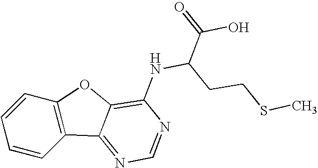 | 48.4 |
| A9.098 | 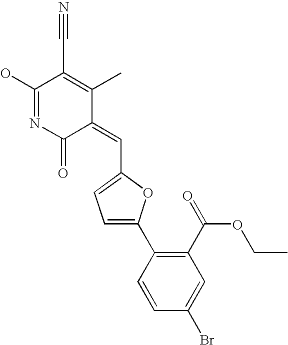 | 49.9 |
| A9.099 | 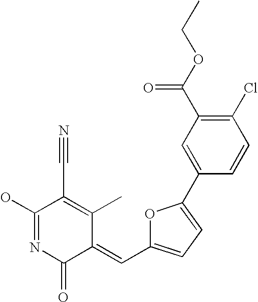 | 52.1 |
| A9.100 | 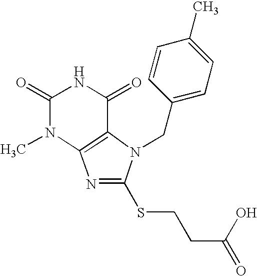 | 53.2* |
| A9.101 | 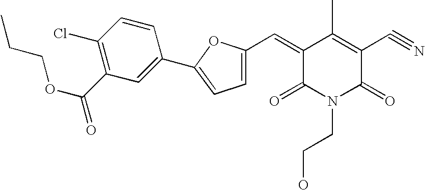 | 54.0 |
| A9.102 | 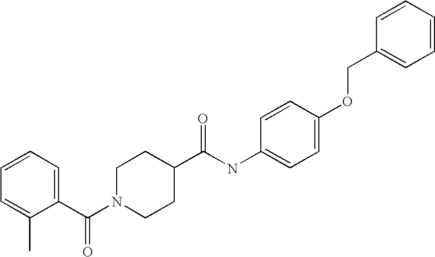 | 54.0 |
| A9.103 | 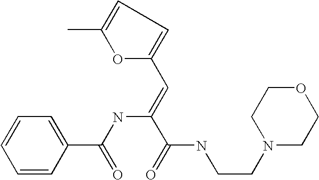 | 55.0* |
| A9.104 | 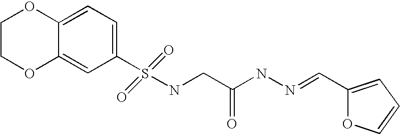 | 55.6 |
| A9.105 | 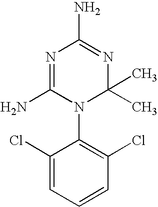 | 59.8 |
| A9.106 | 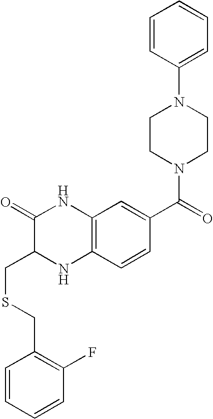 | 61.5 |
| A9.107 | 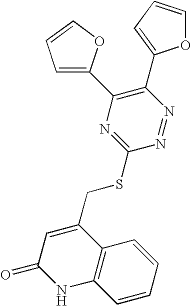 | 65.1* |
| A9.108 | 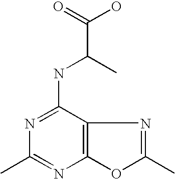 | 65.8 |
| A9.109 | 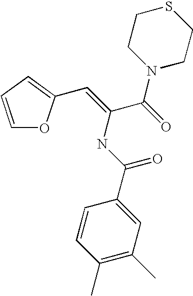 | 68.9 |
| A9.110 | 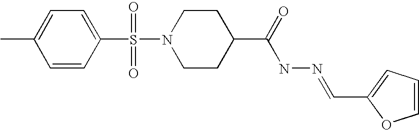 | 69.3 |
| A9.111 | 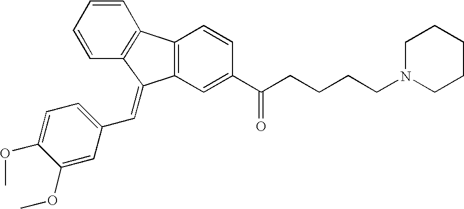 | 74.6 |
| A9.112 | 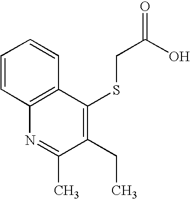 | 77.4 |
| A9.113 | 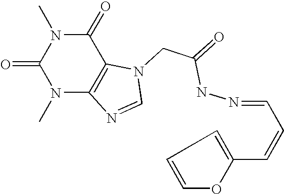 | 79.8* |
| A9.114 | 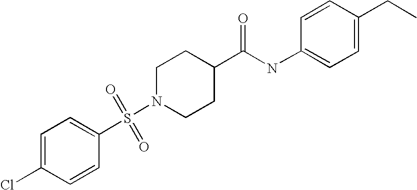 | 80.4* |
| A9.115 | 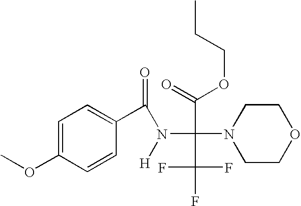 | 90.2 |
| A9.116 | 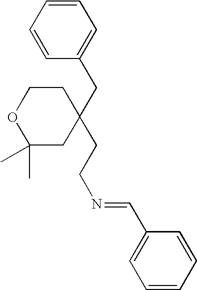 | 93.6 |
| A9.117 | 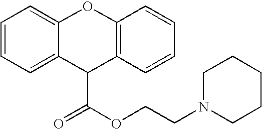 | 94.8* |
| A9.118 | 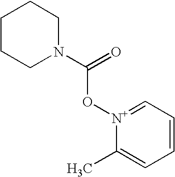 | 96.7* |
| A9.119 | 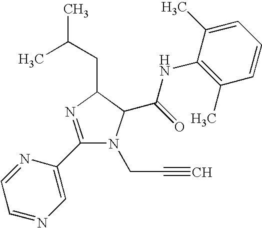 | 102.6* |
| A9.120 | 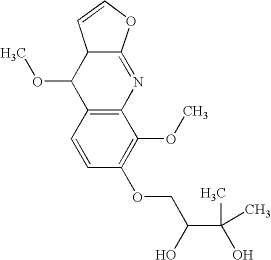 | 117.3* |
| A9.121 | 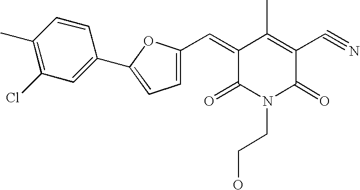 | 125.4 |
| A9.122 | 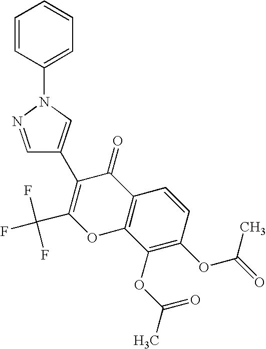 | 133.7* |
| A9.123 | 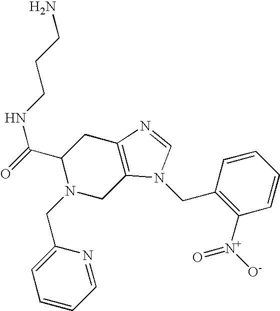 | 138.4 |
| A9.124 | 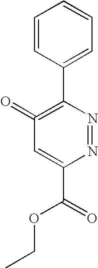 | 142.9 |
| A9.125 | 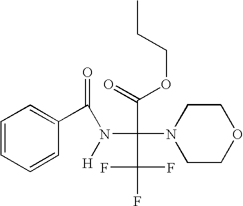 | 186.0* |
| A9.126 | 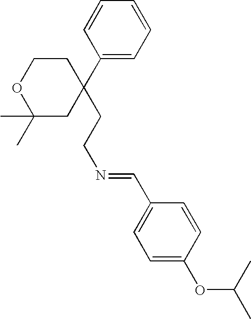 | 197.4* |
| A9.127 | 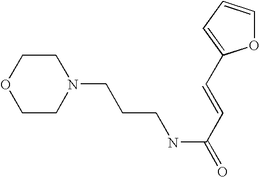 | 208.5* |
| A9.128 | 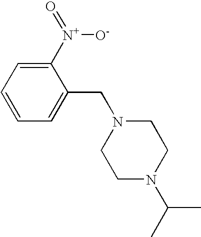 | 252.2* |

TABLE 10

| Compound | | |
|---|---|---|
| ID. | Structure | IC50_{APN} [μM] |
| A10.001 | 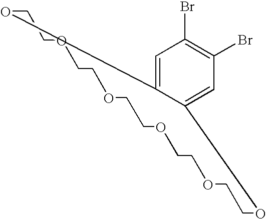 | 0.7 |
| A10.002 | 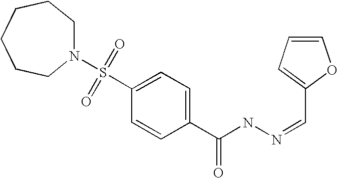 | 8.0 |
| A10.003 | 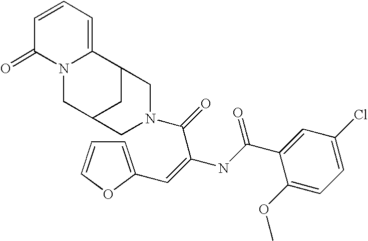 | 8.1 |
| A10.004 | 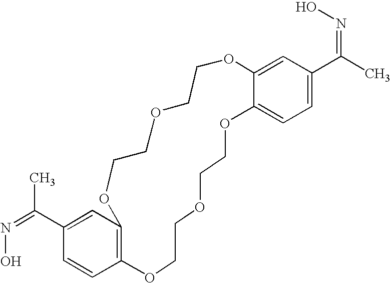 | 8.6 |
| A10.005 | 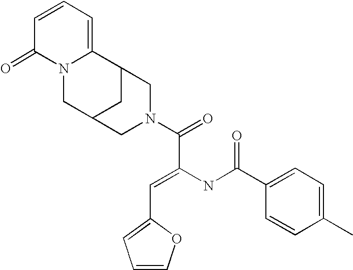 | 11.0 |
| A10.006 | 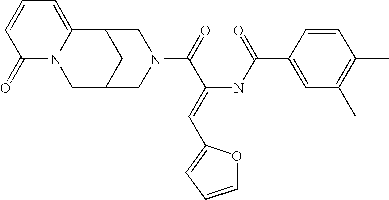 | 11.8 |
| A10.007 | 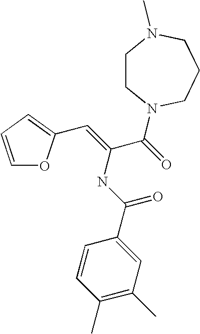 | 32.1 |
| A10.008 | 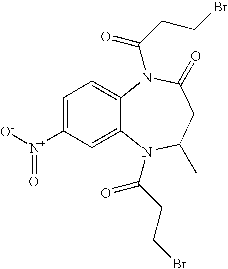 | 99.8* |

TABLE 11

| Compound | | |
|---|---|---|
| ID. | Structure | IC50_{APN} [μM] |
| A11.001 | 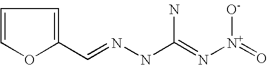 | 7.6 |
| A11.002 | 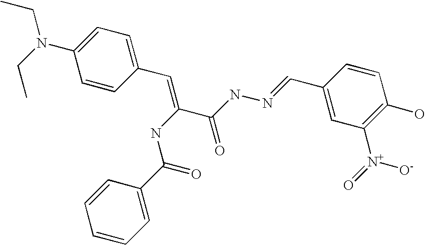 | 7.6 |
| A11.003 | 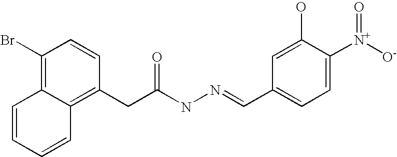 | 7.0 |
| A11.004 | 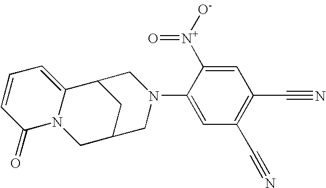 | 8.9 |
| A11.005 | 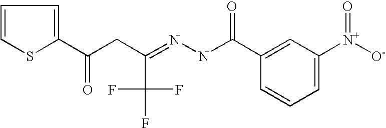 | 10.7 |
| A11.006 | 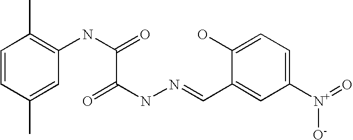 | 10.8 |
| A11.007 | 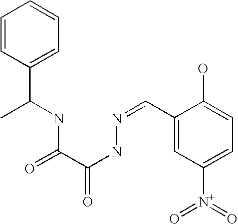 | 11.4 |
| A11.008 | 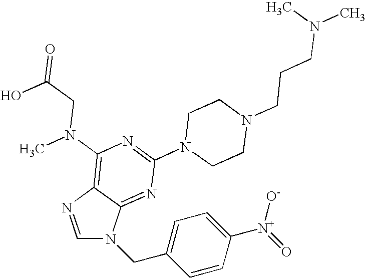 | 11.8 |
| A11.009 | 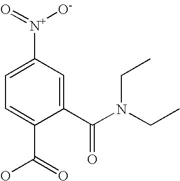 | 12.1 |
| A11.010 | 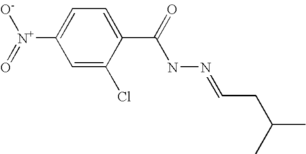 | 15.6 |
| A11.011 | 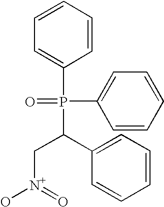 | 16.3 |
| A11.012 | 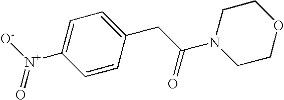 | 19.1 |
| A11.013 | 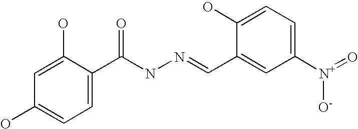 | 20.2 |
| A11.014 | 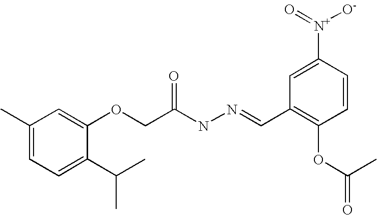 | 20.3 |
| A11.015 | 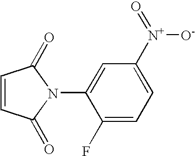 | 20.8 |
| A11.016 | 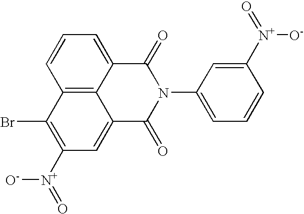 | 23.3 |
| A11.017 | 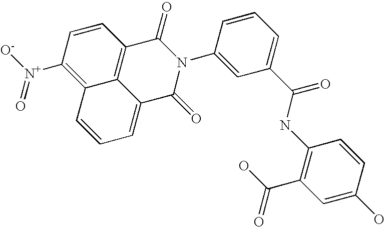 | 23.6 |
| A11.018 | 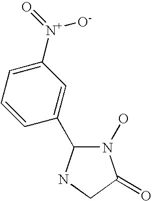 | 26.4 |
| A11.019 | 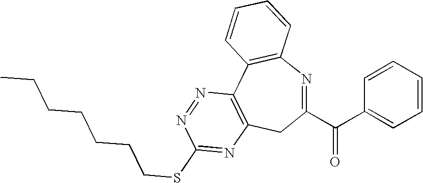 | 28.9 |
| A11.020 | 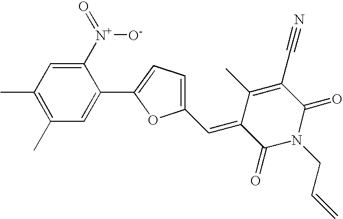 | 29.3 |
| A11.021 | 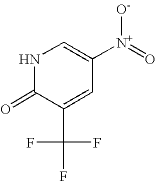 | 29.9 |
| A11.022 | 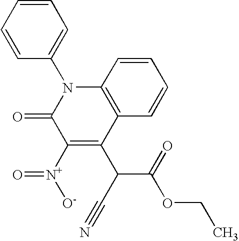 | 30.0 |
| A11.023 | 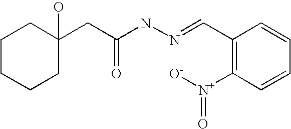 | 31.1 |
| A11.024 | 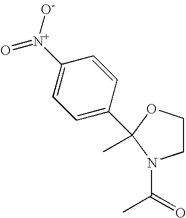 | 32.8 |
| A11.025 | 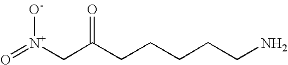 | 33.2 |
| A11.026 | 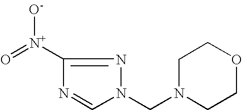 | 36.8 |
| A11.027 | 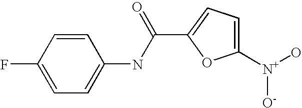 | 37.4 |
| A11.028 | 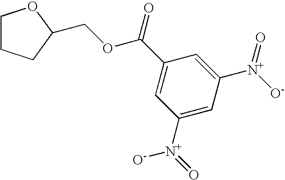 | 39.3 |
| A11.029 | 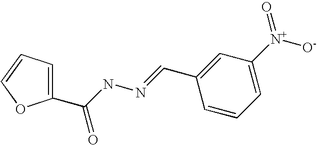 | 45.4 |
| A11.030 | 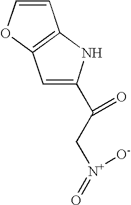 | 49.5 |
| A11.031 | 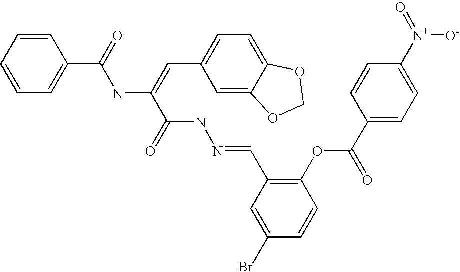 | 54.2 |
| A11.032 | 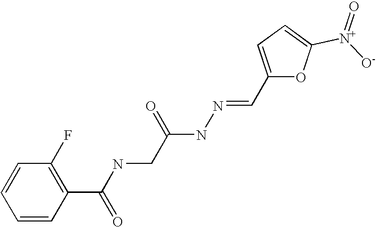 | 60.0 |
| A11.033 | 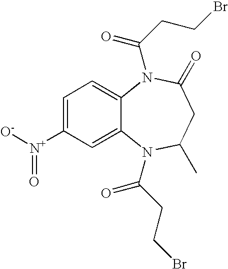 | 99.8* |
| A11.034 | 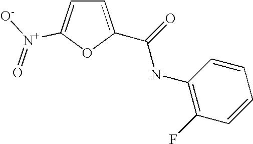 | 62.4 |
| A11.035 | 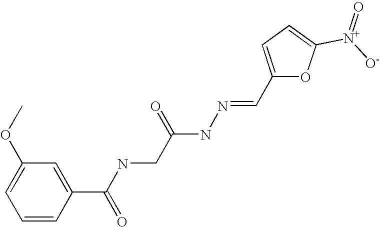 | 221.8* |
| A11.036 | 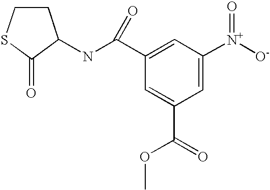 | 238.4* |
| A11.037 | 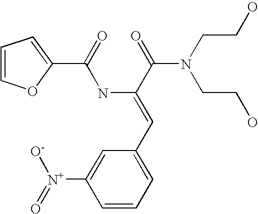 | 243.8* |
| A11.038 | 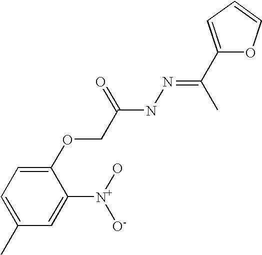 | 422.4* |
| A11.039 | 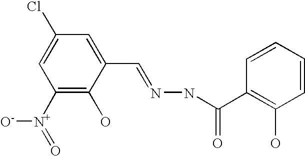 | 5.8 |

TABLE 12

| Compound | | |
|---|---|---|
| ID. | Structure | IC50_{APN} [μM] |
| A12.002 | 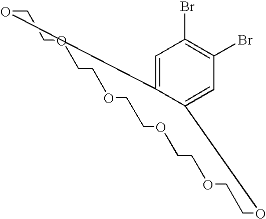 | 0.7* |
| A12.003 | 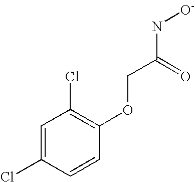 | 0.8* |
| A12.004 | 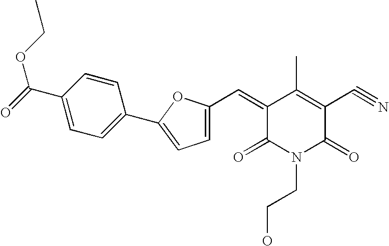 | 2.7* |
| A12.005 | 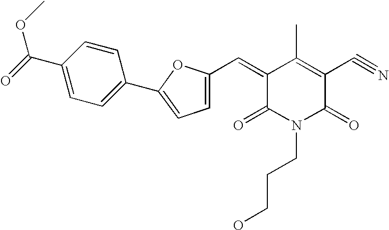 | 5.0 |
| A12.006 | 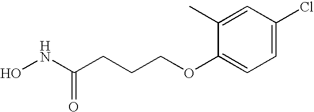 | 5.6 |
| A12.007 | 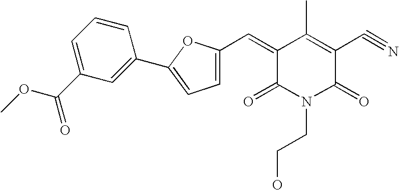 | 6.3 |
| A12.008 | 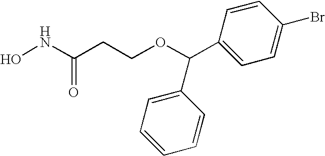 | 6.3 |
| A12.009 | 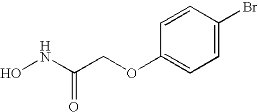 | 6.4 |
| A12.010 | 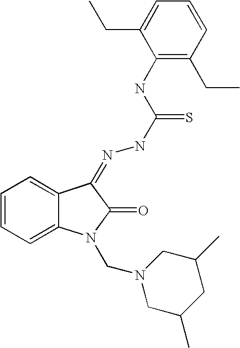 | 6.7 |
| A12.011 | 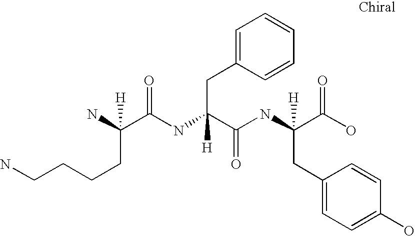 | 7.3 |
| A12.013 | 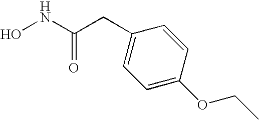 | 7.5 |
| A12.015 | 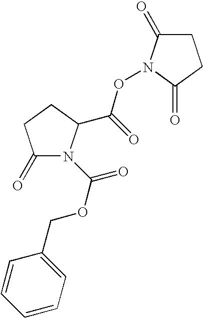 | 7.5 |
| A12.016 | 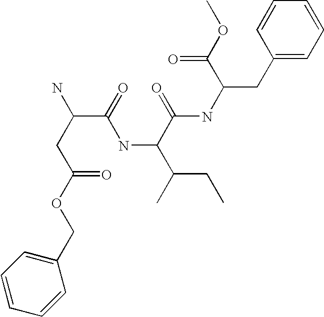 | 8.6 |
| A12.017 | 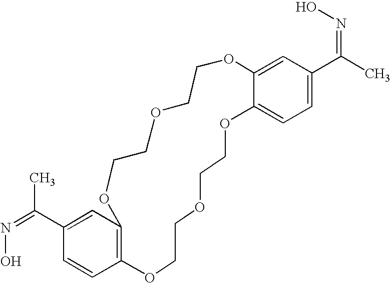 | 8.6 |
| A12.018 | 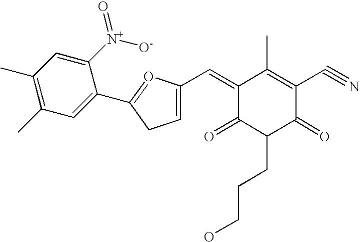 | 9.7 |
| A12.019 | 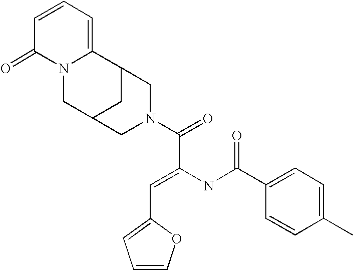 | 11.0 |
| A12.021 | 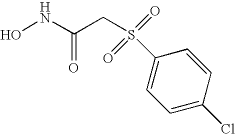 | 11.5 |
| A12.022 | 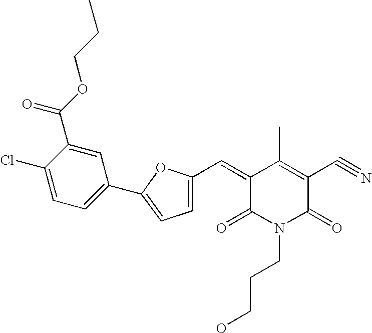 | 23.8 |
| A12.023 | 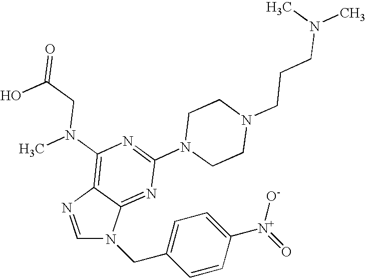 | 11.8 |
| A12.024 | 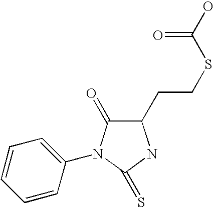 | 12.0 |
| A12.025 | 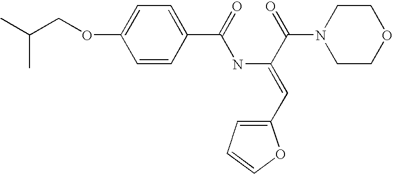 | 13.1 |
| A12.026 | 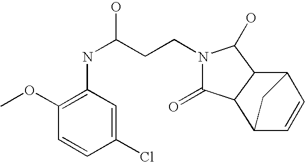 | 13.4 |
| A12.027 | 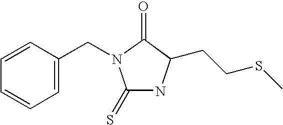 | 14.1 |
| A12.028 | 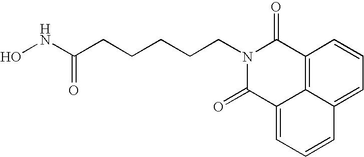 | 14.1 |
| A12.029 | 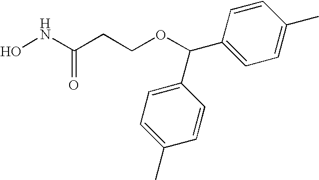 | 14.2 |
| A12.030 | 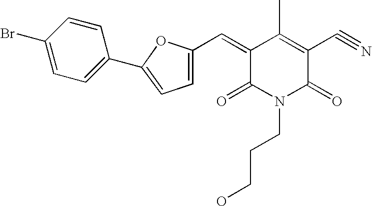 | 16.0 |
| A12.031 | 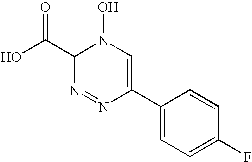 | 17.1 |
| A12.032 | 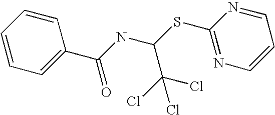 | 17.9 |
| A12.033 | 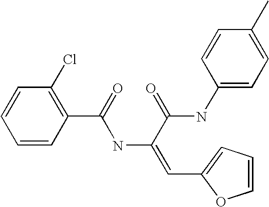 | 17.9 |
| A12.034 | 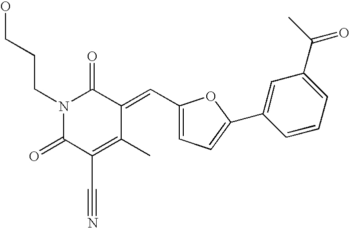 | 18.2 |
| A12.035 | 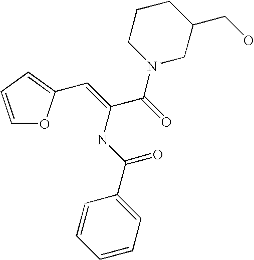 | 19.0 |
| A12.036 | 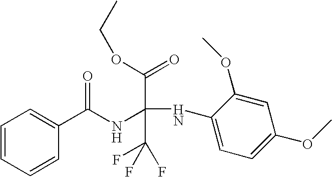 | 19.0 |
| A12.037 | 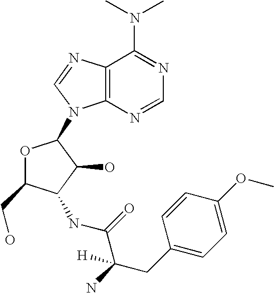 | 20.9 |
| A12.038 | 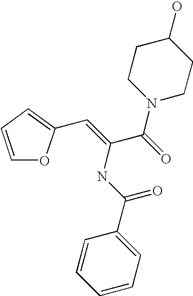 | 21.1 |
| A12.039 | 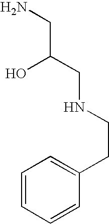 | 21.2 |
| A12.040 | 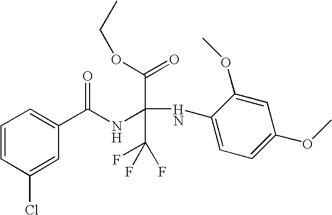 | 21.7 |
| A12.041 | 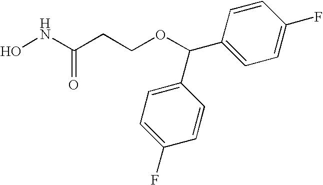 | 21.8 |
| A12.042 | 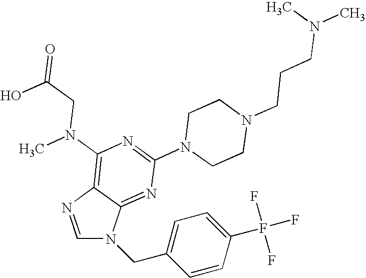 | 23.0 |
| A12.044 | 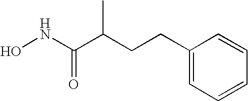 | 25.2 |
| A12.045 | 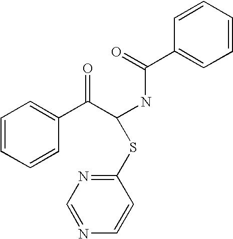 | 26.9 |
| A12.047 | 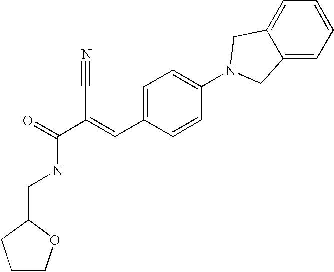 | 28.1 |
| A12.048 | 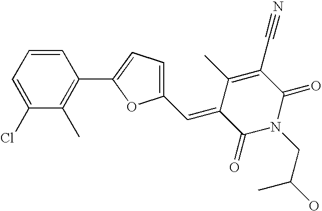 | 28.6 |
| A12.049 | 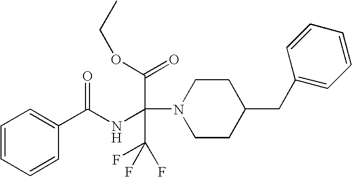 | 29.9 |
| A12.052 | 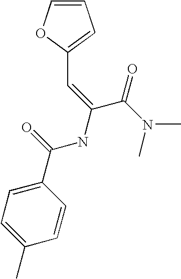 | 32.5 |
| A12.053 | 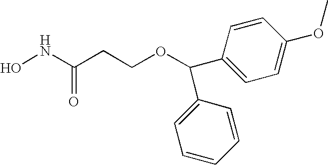 | 33.0 |
| A12.054 | 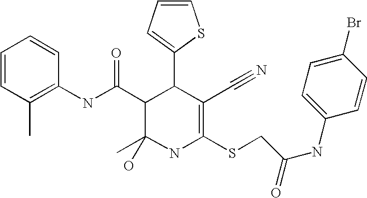 | 33.9 |
| A12.055 | 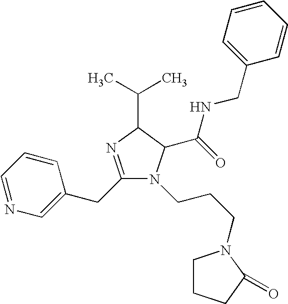 | 34.0 |
| A12.056 | 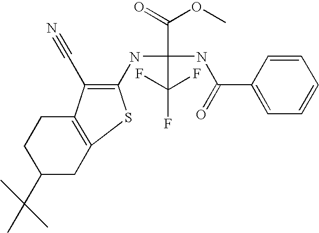 | 43.6 |
| A12.058 | 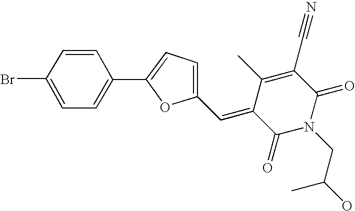 | 47.2* |
| A12.060 | 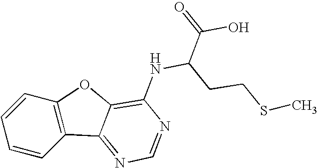 | 48.4 |
| A12.061 | 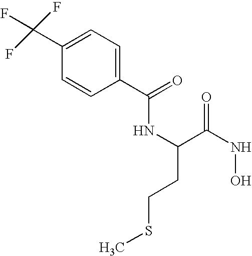 | 51.2 |
| A12.063 | 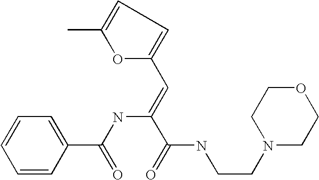 | 55.0 |
| A12.065 | 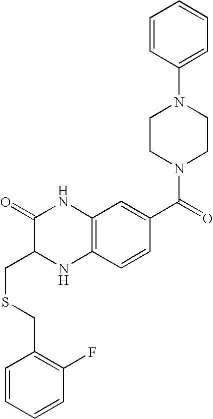 | 61.5 |
| A12.069 | 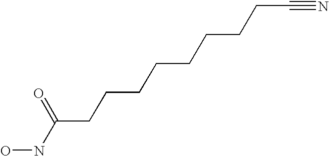 | 80.6 |
| A12.070 | 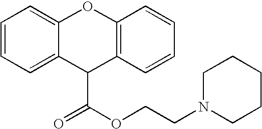 | 94.8 |
| A12.072 | 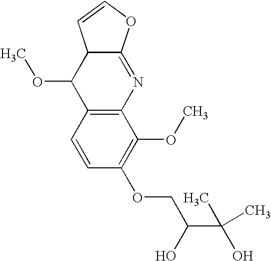 | 117.3* |

TABLE 13

| Compound ID. | Structure | IC50_{APN} [μM] |
|---|---|---|
| A13.001 | 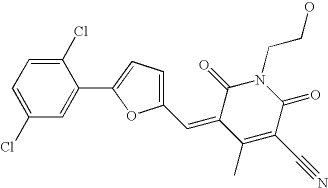 | 1.2* |
| A13.002 | 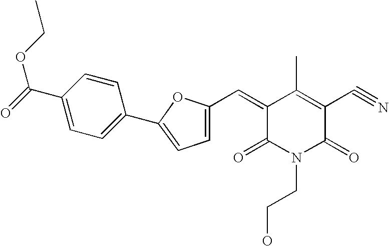 | 2.7* |
| A13.003 | 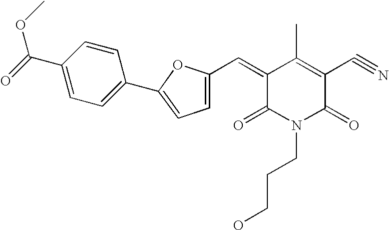 | 5.0 |
| A13.004 | 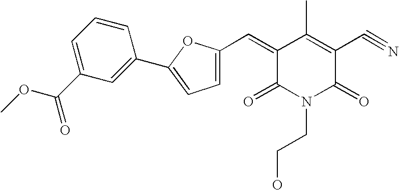 | 6.3 |
| A13.005 | 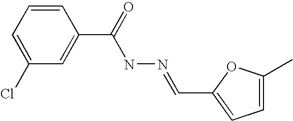 | 10.5 |
| A13.006 | 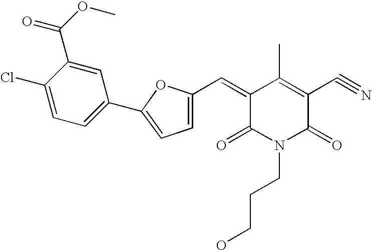 | 15.2 |
| A13.007 | 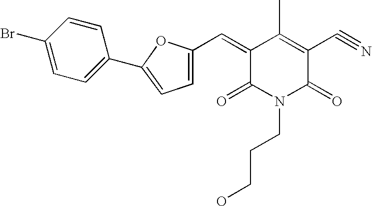 | 16.0 |
| A13.008 | 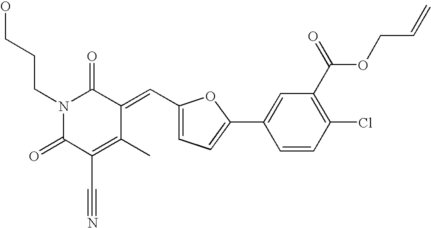 | 18.1 |
| A13.009 | 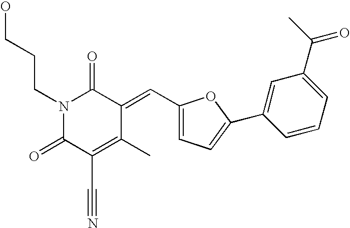 | 18.2 |
| A13.010 | 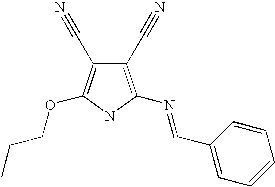 | 20.0 |
| A13.011 | 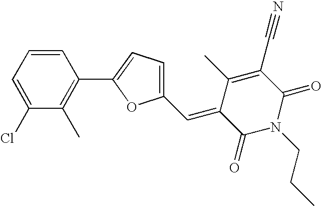 | 20.8 |
| A13.012 | 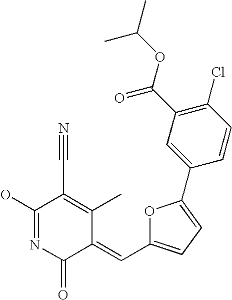 | 21.4 |
| A13.013 | 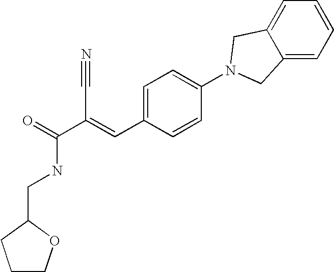 | 28.1 |
| A13.014 | 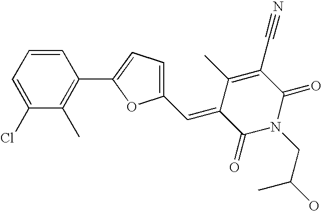 | 28.6 |
| A13.015 | 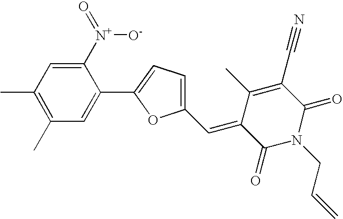 | 29.3 |
| A13.016 | 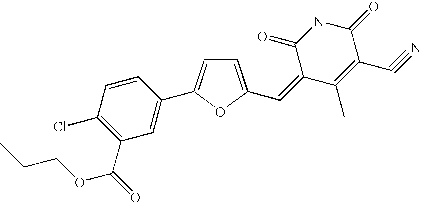 | 36.8 |
| A13.017 | 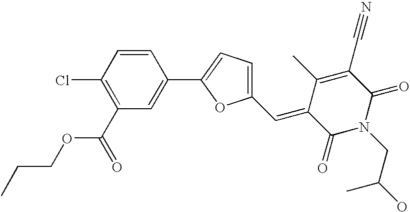 | 32.0 |
| A13.018 | 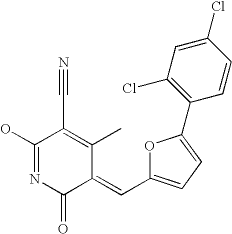 | 32.4 |
| A13.019 | 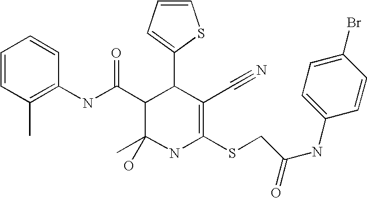 | 33.9 |
| A13.020 | 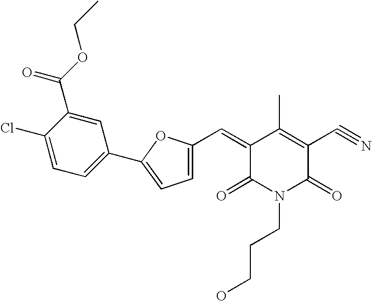 | 41.0 |
| A13.021 | 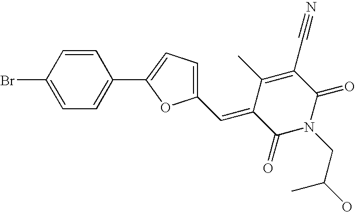 | 47.2* |
| A13.022 | 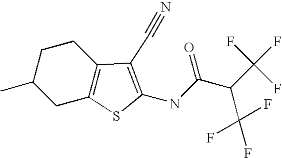 | 51.3 |
| A13.023 | 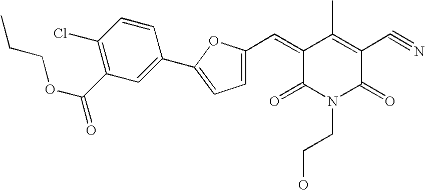 | 54.0 |
| A13.024 | 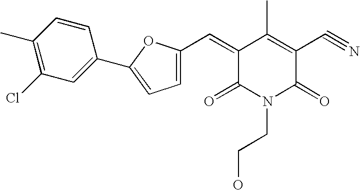 | 125.4 |

TABLE 14

| Compound | | |
|---|---|---|
| ID. | Structure | IC50_{APN} [μM] |
| A14.001 | 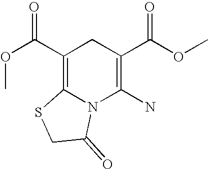 | “positively very highly active”, means beyond measurement- scale” |
| A14.002 | 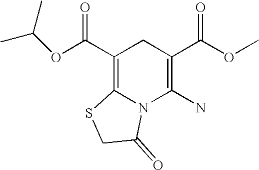 | 5.9 |
| A14.003 | 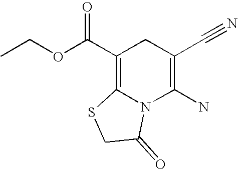 | 23.1 |

### Example 2

Therapeutic Effect of the Combined Inhibition of the Alanyl Aminopeptidases and of Enzymes Having an Analogous Effect as Well as of the Dipeptidyl Peptidase IV and of Enzymes Having an Analogous Effect on the Experimental Autoimmune Encephalomyelitis (EAE) of Mice (Animal Model of Multiple Sclerosis)

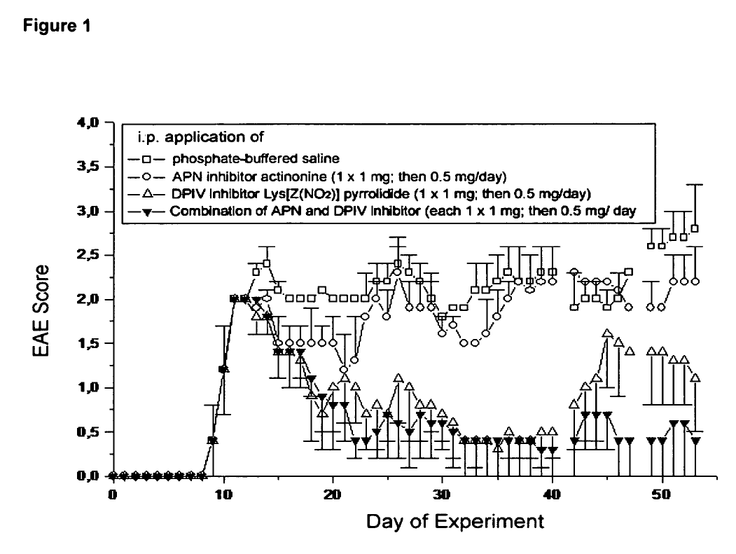

The disease EAE was induced by a daily injection of PLP139-151 (myelin antigen proteolipide protein peptide 139-151) to SJL/J mice (n=10). After the outbreak of the disease, there was, on the 11^{th} day after the immunization, a therapeutic intervention by an intraperitoneal injection of 1 mg of each of the peptidase inhibitors on the first day and further injections of 0.5 mg of each of the inhibitors every second day. The disease scores [vD1] are defined by differently distinct degrees of paralysis. Healthy animals have the disease score 0. Actinonine was used as the alanyl aminopeptidase inhibitor, Lys[Z(NO₂)]pyrrolidide was used as the dipeptidyl peptidase IV inhibitor. The treatment was effected for the time of 46 days after the immunization. The results are shown in FIG. 1. The course of the curves demonstrate unequivocally a most strong and long-lasting [vD2] therapeutic effect after a combined inhibition of both peptidases.

### Example 3

Therapeutic Effect of the Combined Inhibition of the Alanyl Aminopeptidases and of Enzymes Having an Analogous Effect as Well as of Dipeptidyl Peptidase IV and of Enzymes Having an Analogous Effect on the Dextran Sulfate-Induced Colitis in Mice (Animal Model of Chronic Inflammatory Intestinal Diseases)

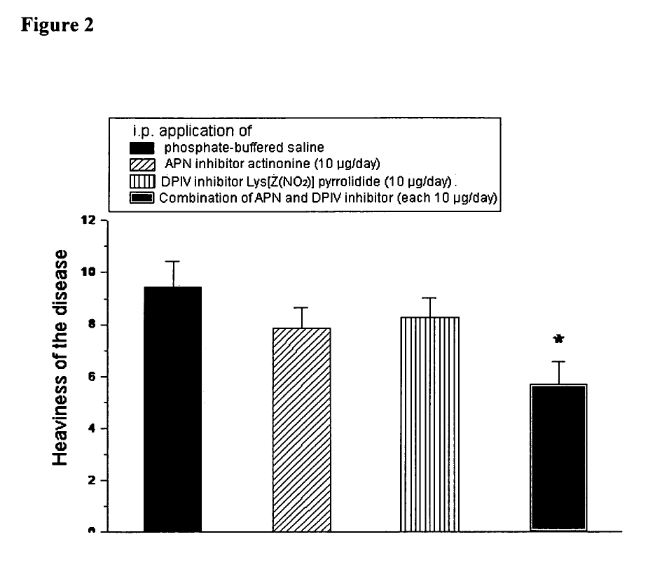

An inflammation relating predominantly to the colon (equivalent to the disease of human Colitis ulcerosa) was induced by an administration of 3% sodium dextran sulfate dissolved in the drinking water of female Balb/c mice having an age of 8 weeks. After three days, all animals showed clear symptoms typical for the disease. The peptidase inhibitors (or phosphate-buffered saline as a placebo) were administered intraperitoneally from day 5 on for three successive days. The degree of the disease was determined in accordance with an acknowledged evaluation system (score). The following parameters were considered when determining the score: Consistency of the excrements (solid=0 points (pts.); pasty=2 pts.; liquid/like diarrhea=4 pts.); detection of blood in the excrements (no blood=0 pts.; occult blood=2 pts.; evident=4 pts.); loss of weight (0-5%=0 pts.; 5 to 10%=1 pts.; 10-15%=2 pts.; 15-20%=3 pts.; >20%=4 pts.). Healthy animals have a score value of 0 pts.; the maximum value are 12 pts. From 10 pts. on, the disease is lethal. In the course of the disease, the score value increased due to the change of the excrement parameters. Later-on (starting from day 5), the loss of weight increased the score. FIG. 2 shows the disease intensity for untreated and treated animals on the day 7 after three days of therapy.

The application of 10 μg of the respective single prior art inhibitors (n=14 per group; see explanation) achieved a slight, but insignificant reduction of the heaviness of the disease (−16.5% by a treatment with actinonine; −12.3% by a treatment with Lys[Z(NO₂)]pyrrolidide). An i.p. application of a combination of the two peptidase inhibitors resulted into a statistically significant (p=0.00189) improvement of the disease by 40%.

### Example 4

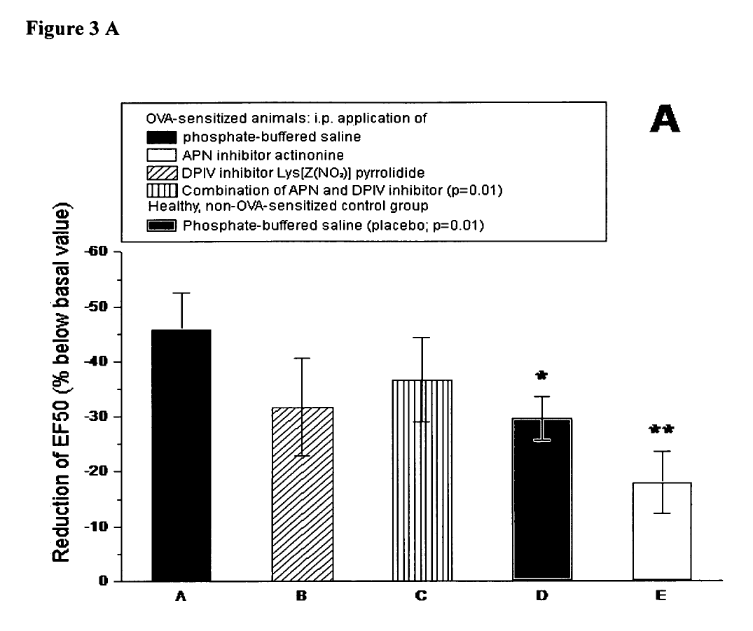

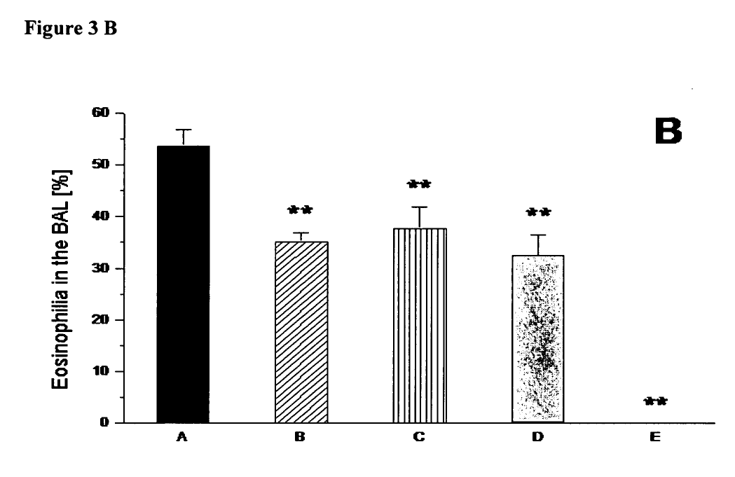

Therapeutic effect of the combined inhibition of alanyl aminopeptidases and of enzymes having an analogous effect as well as of dipeptidyl peptidase IV and of enzymes having an analogous effect on the ovalbumine-induced asthma bronchiale ib mice (animal model of human asthma bronchiale). FIG. 3 shows the influence of the combined peptidase inhibition on the reduction of the average expiratory flux (EF 50) as a measure of the pulmonal function (FIG. 3 A) as well as on the eosinophilia as a characteristic feature of the asthma bronchiale pulmonal inflammation (FIG. 3 B).

Female Balb/c mice were sensitized for the antigen ovalbumine capable of inducing asthma bronchiale by an intreperitoneal administration of 10 μg ovalbumine on the days 0, 14 and 21. On day 27/28, the animals received a boostering dose of ovalbumine by inhalation [vD3]. After an intreperitoneal administration of the peptidase inhibitors on the days 28-35, there was effected an intranasal ovalbumine challenge on day 35, as well as a check of the allergic premature reaction via the pulmonal function. There were measured: the average expiratory flux (EF50), the tidal volume, the respiration rate and the minute volume as well as the number of eosinophilic granulocytes in the bronchoalveolar lavage.

8 to 10 animals were used per experimental group. By way of example, in FIG. 3 A, there is summarized the effect of the peptidase inhibitors on the reduction of the EF50 value. The alanyl aminopeptidase inhibitor actinonine (group B; 0.1 mg), and the dipeptidyl peptidase IV inhibitor Lys[Z(NO₂)]pyrrolidide as well (group C, 0.1 mg), showed a therapeutic effect. Significant therapeutic effects, however, were obtained only when using combinations of both inhibitors (group D; 0.1 mg of each of the inhibitors). Group E represents animals which were not sensitized by OVA, but which were subjected—beyond that—all procedures to which the animal groups A to D were subjected. Hence, this group is a group of healthy, non-allergic animals allowing to calculate stress-induced effects on the pulmonal function.

1.-76. (canceled)

77. A pharmaceutical or cosmetic composition comprising at least one of a pharmaceutically or cosmetically acceptable carrier and a pharmaceutically or cosmetically acceptable adjuvant and at least one active ingredient selected from compounds of formulae A1 to A14, including tautomers, stereoisomers thereof, pharmaceutically acceptable salts, salt derivatives, tautomers and stereoisomers thereof: 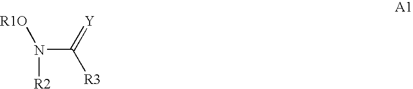
wherein
Y represents O, S or NR4;
R1, R2, R3 and R4 are selected from hydrogen, unsubstituted or substituted, straight chain or branched C₁- to C₁₂ alkyl, C₂- to C₁₂ alkenyl and C₂- to C₁₂ alkynyl, hydroxy, thiol, C₁- to C₁₂ alkoxy, C₁- to C₁₂ alkylthio, unsubstituted or substituted, uncondensed or condensed aryl and cycloalkyl optionally containing one or several heteroatoms selected from N, O, P and S, unsubstituted or substituted amino, unsubstituted or substituted carbonyl, unsubstituted or substituted thiocarbonyl and unsubstituted or substituted imino; and
heteroaromatic or heterocyclic radicals are bound to a basic structure of formula A1 via a C atom or a heteroatom; 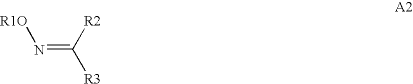
wherein
R1, R2 and R3 are selected from hydrogen, unsubstituted or substituted, straight chain or branched C₁- to C₁₂ alkyl, C₂- to C₁₂ alkenyl and C₂- to C₁₂ alkynyl, hydroxy, thiol, C₁- to C₁₂ alkoxy, C₁- to C₁₂ alkylthio, unsubstituted or substituted, uncondensed or condensed aryl and cycloalkyl optionally containing one or several heteroatoms selected from N, O, P and S, unsubstituted or substituted amino, unsubstituted or substituted carbonyl, unsubstituted or substituted thiocarbonyl and unsubstituted or substituted imino; and
heteroaromatic or heterocyclic residues are bound to a basic structure of formula A2 via a C atom or a heteroatom; 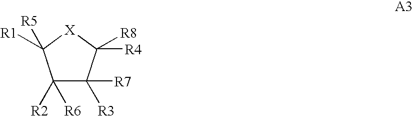
wherein
X represents O, S, NH or NR9;
a basic five-membered ring structure may additionally contain up to three further heteroatoms as defined for X, which may be identical or different;
a basic five-membered ring structure may contain zero to two double bonds;
R1 to R9 are selected from hydrogen, unsubstituted or substituted, straight chain or branched C₁- to C₁₂ alkyl, C₂- to C₁₂ alkenyl and C₂- to C₁₂ alkynyl, hydroxy, thiol, C₁- to C₁₂ alkoxy, C₁- to C₁₂ alkylthio, unsubstituted or substituted, uncondensed or condensed aryl and cycloalkyl optionally containing one or several heteroatoms selected from N, O, P and S, unsubstituted or substituted amino, unsubstituted or substituted carbonyl, unsubstituted or substituted thiocarbonyl and unsubstituted or substituted imino; and
heteroaromatic or heterocyclic radicals are bound to a basic structure of formula A3 via a C atom or a heteroatom; 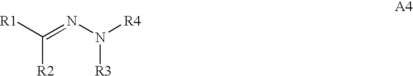
wherein
R1, R2, R3 and R4 are selected from hydrogen, unsubstituted or substituted, straight chain or branched C₁- to C₁₂ alkyl, C₂- to C₁₂ alkenyl and C₂- to C₁₂ alkynyl, hydroxy, thiol, C₁- to C₁₂ alkoxy, C₁- to C₁₂ alkylthio, unsubstituted or substituted, uncondensed or condensed aryl and cycloalkyl optionally containing one or several heteroatoms selected from N, O, P and S, unsubstituted or substituted amino, unsubstituted or substituted carbonyl, unsubstituted or substituted thiocarbonyl and unsubstituted or substituted imino; and
heteroaromatic or heterocyclic radicals are bound to a basic structure of formula A4 via a C atom or a heteroatom; 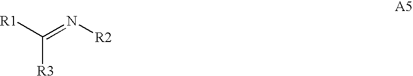
wherein
R1, R2 and R3 are selected from hydrogen, unsubstituted or substituted, straight chain or branched C₁- to C₁₂ alkyl, C₂- to C₁₂ alkenyl and C₂- to C₁₂ alkynyl, hydroxy, thiol, C₁- to C₁₂ alkoxy, C₁- to C₁₂ alkylthio, unsubstituted or substituted, uncondensed or condensed aryl and cycloalkyl optionally containing one or several heteroatoms selected from N, O, P and S, unsubstituted or substituted amino, unsubstituted or substituted carbonyl, unsubstituted or substituted thiocarbonyl and unsubstituted or substituted imino; and
heteroaromatic or heterocyclic radicals are bound to a basic structure of formula A5 via a C atom or a heteroatom; 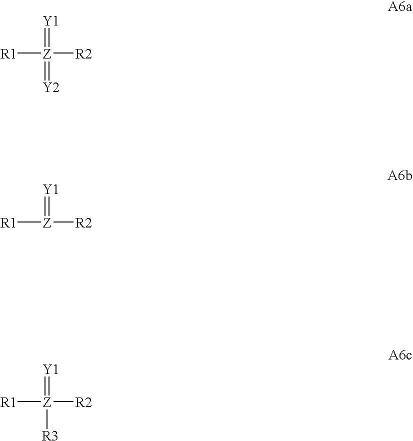
wherein
Y1 and Y2 represent O, S, NH, NR4 or NR5;
Z represents S or P;
R1 to R5 are selected from hydrogen, unsubstituted or substituted, straight chain or branched C₁- to C₁₂ alkyl, C₂- to C₁₂ alkenyl and C₂- to C₁₂ alkynyl, hydroxy, thiol, C₁- to C₁₂ alkoxy, C₁- to C₁₂ alkylthio, unsubstituted or substituted, uncondensed or condensed aryl and cycloalkyl optionally containing one or several heteroatoms selected from N, O, P and S, unsubstituted or substituted amino, unsubstituted or substituted carbonyl, unsubstituted or substituted thiocarbonyl and unsubstituted or substituted imino; and
heteroaromatic or heterocyclic radicals are bound to a basic structure of formula A6 via a C atom or a heteroatom; 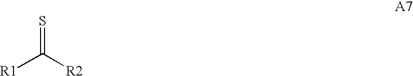
wherein
R1 and R2 are selected from hydrogen, unsubstituted or substituted, straight chain or branched C₁- to C₁₂ alkyl, C₂- to C₁₂ alkenyl and C₂- to C₁₂ alkynyl, hydroxy, thiol, C₁- to C₁₂ alkoxy, C₁- to C₁₂ alkylthio, unsubstituted or substituted, uncondensed or condensed aryl and cycloalkyl optionally containing one or several heteroatoms selected from N, O, P and S, unsubstituted or substituted amino, unsubstituted or substituted carbonyl, unsubstituted or substituted thiocarbonyl and unsubstituted or substituted imino; and
heteroaromatic or heterocyclic radicals are bound to a basic structure of formula A7 via a C atom or a heteroatom; 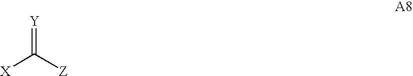
wherein
X and Z are identical or different and are independently selected from hydroxy, thiol, C₁- to C₁₂ alkoxy, C₁- to C₁₂ alkylthio, unsubstituted or substituted, uncondensed or condensed aryl and cycloalkyl optionally containing one or several heteroatoms selected from N, O, P and S, and amino (NH₂, NHR1, NR1R2);
Y represents O, S or NR3;
R1, R2 and R3 are identical or different and are selected from hydrogen, unsubstituted or substituted, straight chain or branched C₁- to C₁₂ alkyl, C₂- to C₁₂ alkenyl and C₂- to C₁₂ alkynyl, hydroxy, thiol, C₁- to C₁₂-alkoxy, C₁- to C₁₂ alkylthio, unsubstituted or substituted, uncondensed or condensed aryl and cycloalkyl optionally containing one or several heteroatoms selected from N, O, P and S, unsubstituted or substituted amino, unsubstituted or substituted carbonyl, unsubstituted or substituted thiocarbonyl and unsubstituted or substituted imino; and
heteroaromatic or heterocyclic radicals are bound to a basic structure of formula A8 via a C atom or a heteroatom; 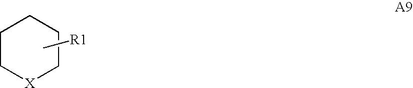
wherein
radicals R1 symbolize a substitution of a basic six-membered ring structure;
X represents O, S, NH or NR2;
a basic heterocyclic structure may contain zero to three double bonds and up to three further heteroatoms as defined for X;
heteroatoms of the group X are identical or different and represent O, S, NH and NR2;
R1 and R2 are selected from hydrogen, unsubstituted or substituted, straight chain or branched C₁- to C₁₂ alkyl, C₂- to C₁₂ alkenyl and C₂- to C₁₂ alkynyl, hydroxy, thiol, C₁- to C₁₂ alkoxy, C₁- to C₁₂ alkylthio, unsubstituted or substituted, uncondensed or condensed aryl and cycloalkyl optionally containing one or several heteroatoms selected from N, O, P and S, unsubstituted or substituted amino, unsubstituted or substituted carbonyl, unsubstituted or substituted thiocarbonyl and unsubstituted or substituted imino; and
heteroaromatic or heterocyclic radicals are bound to a basic structure of formula A9 via a C atom or a heteroatom; 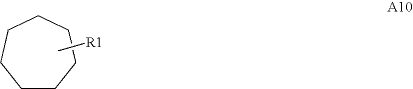
wherein
radicals R1 symbolize a substitution of a basic ring structure;
radicals R1 are selected from hydrogen, unsubstituted or substituted, straight chain or branched C₁- to C₁₂ alkyl, C₂- to C₁₂ alkenyl and C₂- to C₁₂ alkynyl, hydroxy, thiol, C₁- to C₁₂ alkoxy, C₁- to C₁₂ alkylthio, unsubstituted or substituted, uncondensed or condensed aryl and cycloalkyl optionally containing one or several heteroatoms selected from N, O, P and S, unsubstituted or substituted amino, unsubstituted or substituted carbonyl, unsubstituted or substituted thiocarbonyl and unsubstituted or substituted imino; and
heteroaromatic or heterocyclic radicals are bound to a basic structure of formula A10 via a C atom or a heteroatom; 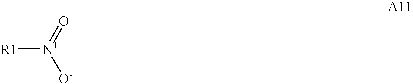
wherein
R1 is selected from hydrogen, unsubstituted or substituted, straight chain or branched C₁- to C₁₂ alkyl, C₂- to C₁₂ alkenyl and C₂- to C₁₂ alkynyl, hydroxy, thiol, C₁- to C₁₂ alkoxy, C₁- to C₁₂ alkylthio, unsubstituted or substituted, uncondensed or condensed aryl and cycloalkyl optionally containing one or several heteroatoms selected from N, O, P and S, unsubstituted or substituted amino, unsubstituted or substituted carbonyl, unsubstituted or substituted thiocarbonyl and unsubstituted or substituted imino; and
heteroaromatic or heterocyclic radicals are bound to a basic structure of formula A11 via a C atom or a heteroatom; 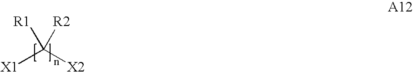
wherein
R1 and R2 are selected from hydrogen, unsubstituted or substituted, straight chain or branched C₁- to C₁₂ alkyl, C₂- to C₁₂ alkenyl and C₂- to C₁₂ alkynyl, hydroxy, thiol, C₁- to C₁₂ alkoxy, C₁- to C₁₂ alkylthio, unsubstituted or substituted, uncondensed or condensed aryl and cycloalkyl optionally containing one or several heteroatoms selected from N, O, P and S, unsubstituted or substituted amino, unsubstituted or substituted carbonyl, unsubstituted or substituted thiocarbonyl and unsubstituted or substituted imino;
X1 and X2 are identical or different and are independently selected from hydroxy, thiol, C₁- to C₁₂ alkoxy, C₁- to C₁₂ alkylthio, unsubstituted or substituted, uncondensed or condensed aryl and cycloalkyl optionally containing one or several heteroatoms selected from N, O, P and S, hydroxy, thiol and amino (NH₂, HNR1, NR1R2);
heteroaromatic or heterocyclic radicals are bound to a basic structure of formula A12 via a C atom or a heteroatom;
n is the number of C atoms between X1 and X2 and ranges from zero to four;
radicals R1 and R2 are identical or different, as far as a number of C atoms of a bridge and different C atoms of a bridge are concerned;
R1-C≡N A13
wherein
R1 is selected from hydrogen, unsubstituted or substituted, straight chain or branched C₁- to C₁₂ alkyl, C₂- to C₁₂ alkenyl and C₂- to C₁₂ alkynyl, hydroxy, thiol, C₁- to C₁₂ alkoxy, C₁- to C₁₂ alkylthio, unsubstituted or substituted, uncondensed or condensed aryl and cycloalkyl optionally containing one or several heteroatoms selected from N, O, P and S, unsubstituted or substituted amino, unsubstituted or substituted carbonyl, unsubstituted or substituted thiocarbonyl and unsubstituted or substituted imino; and
heteroaromatic or heterocyclic radicals are bound to a basic structure of formula A13 via a C atom or a heteroatom; 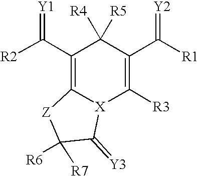
wherein
X represents N, CH, CR8, P, P═O, P(OH)₂, P(OH)(OR8) or P(OR8)(OR9), and Z represents NH, NR10, O or S;
Y1, Y2 and Y3 independently represent O, S, NH, NR11, NR12 or NR13;
R1 to R13 are selected from hydrogen, unsubstituted or substituted, straight chain or branched C₁- to C₁₂ alkyl, C₂- to C₁₂ alkenyl and C₂- to C₁₂ alkynyl, hydroxy, thiol, C₁- to C₁₂ alkoxy, C₁- to C₁₂ alkylthio, unsubstituted or substituted, uncondensed or condensed aryl and cycloalkyl optionally containing one or several heteroatoms selected from N, O, P and S, unsubstituted or substituted amino, unsubstituted or substituted carbonyl, unsubstituted or substituted thiocarbonyl and unsubstituted or substituted imino; and
heteroaromatic or heterocyclic radicals are bound to a basic structure of formula A14 via a C atom or a heteroatom.

78. The composition of claim 77, wherein the composition comprises at least one active ingredient selected from compounds of the following formulae, including tautomers, stereoisomers thereof, pharmaceutically acceptable salts, salt derivatives, tautomers and stereoisomers thereof:

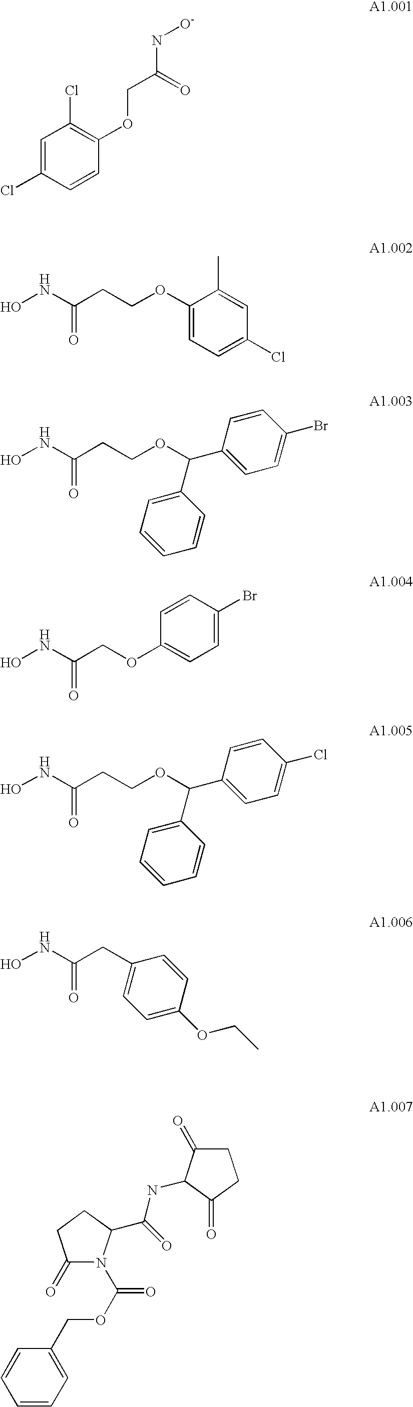

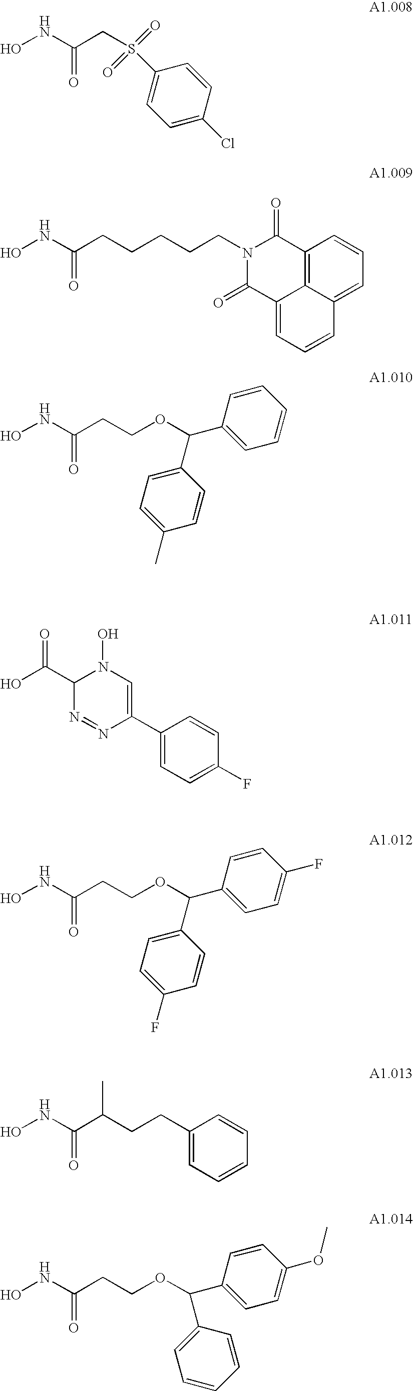

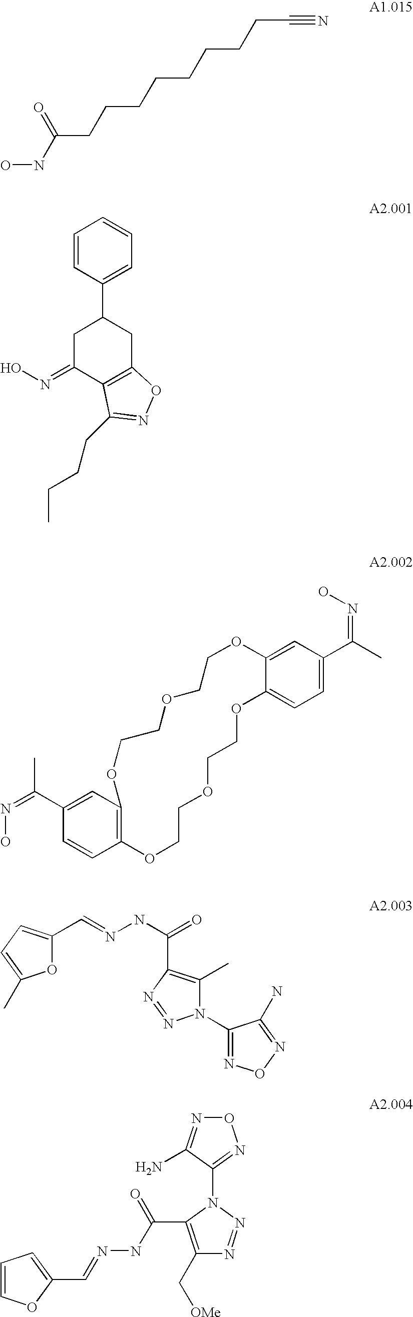

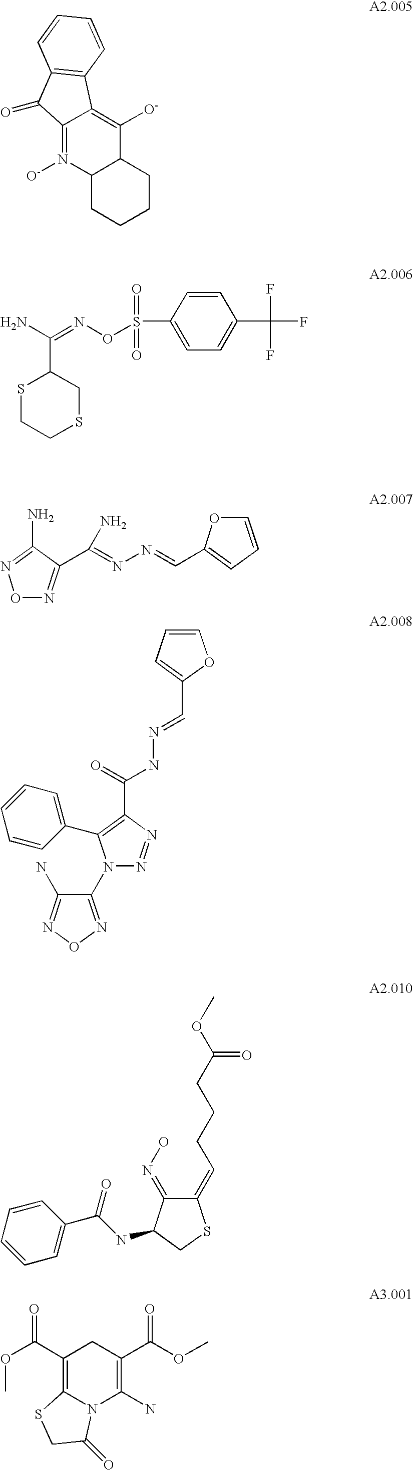

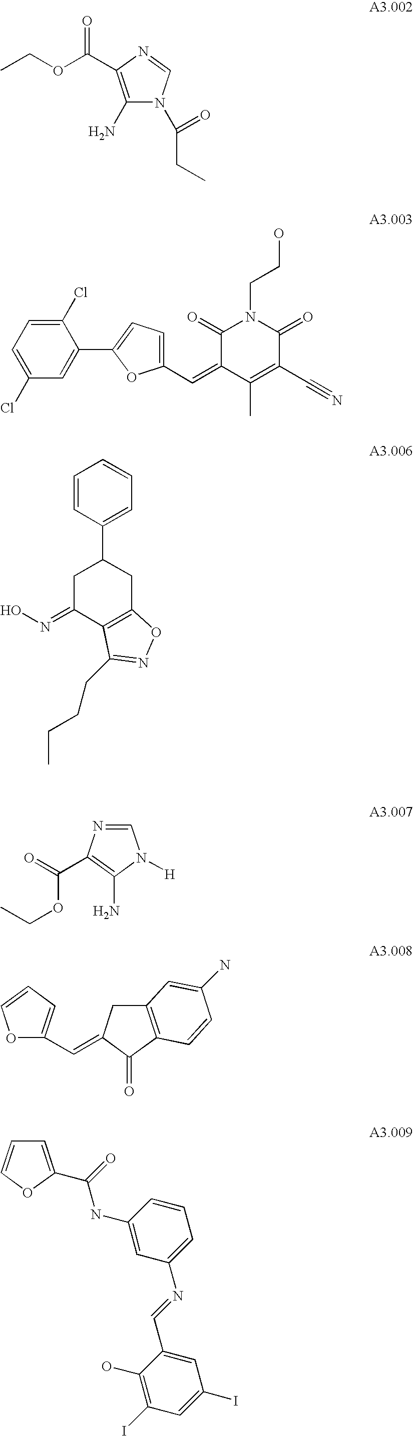

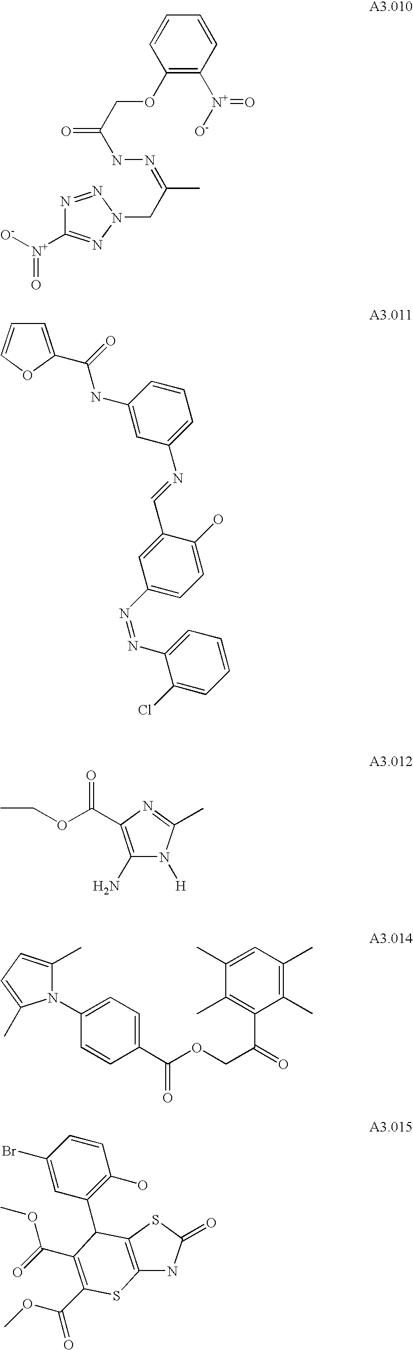

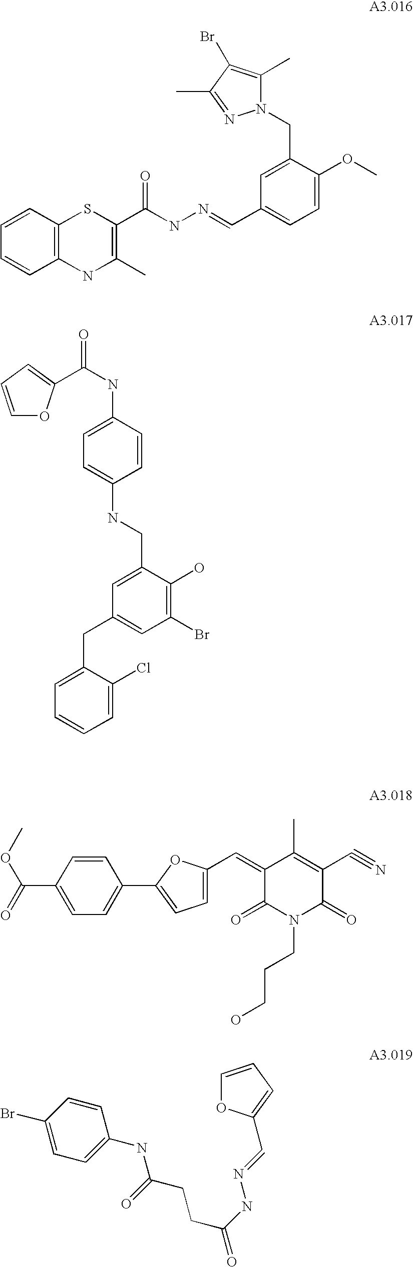

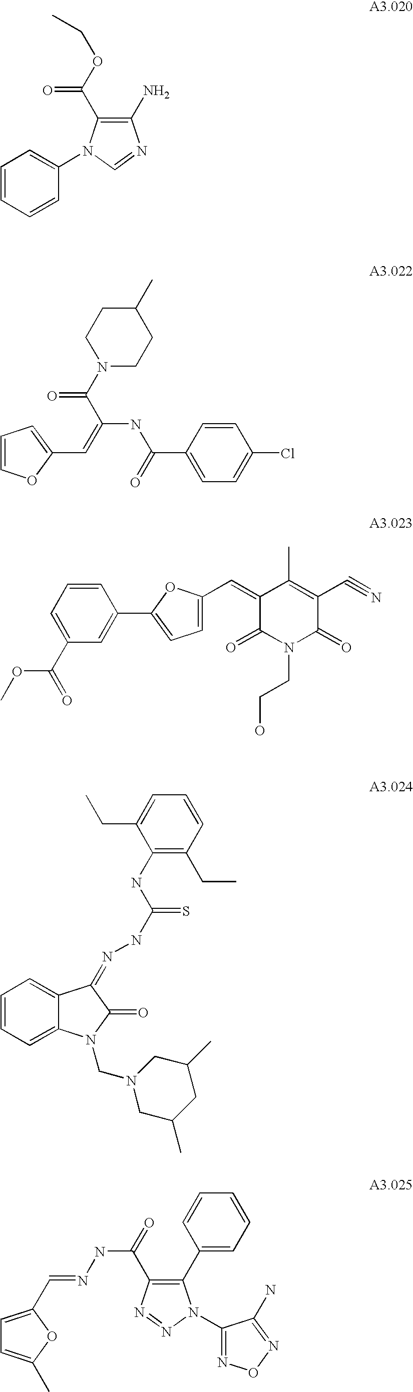

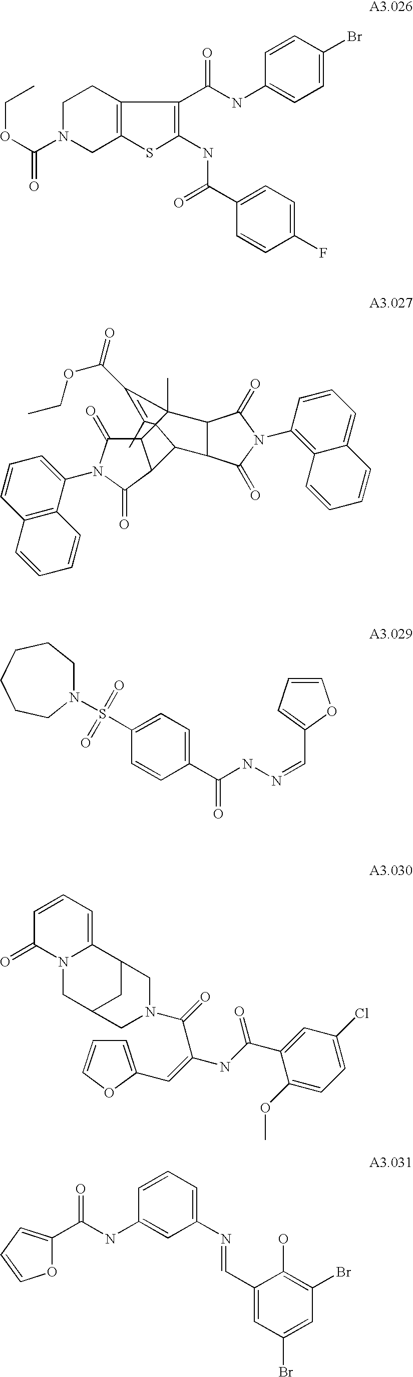

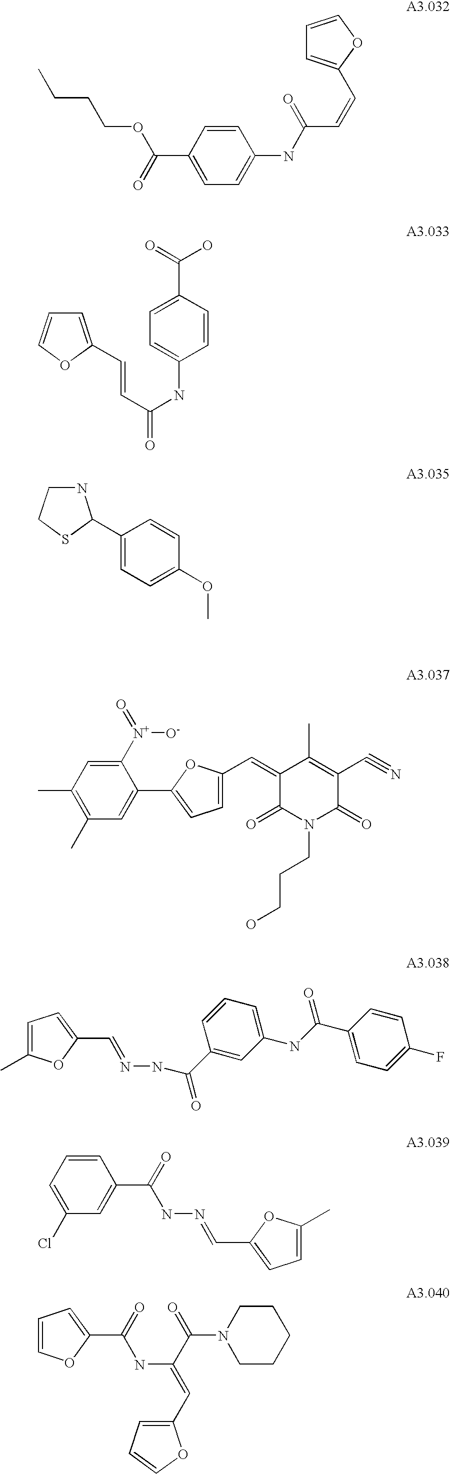

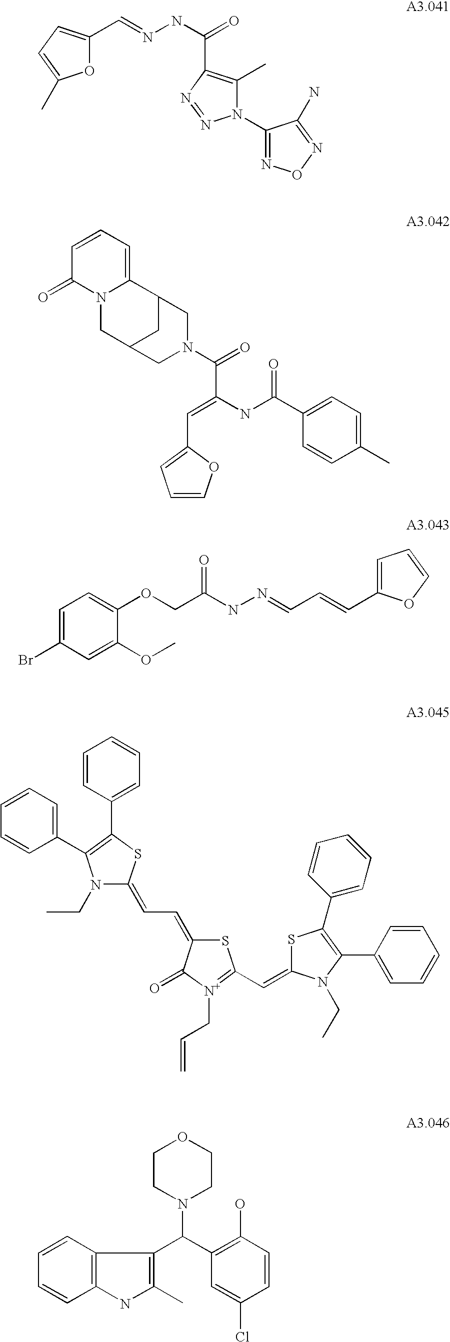

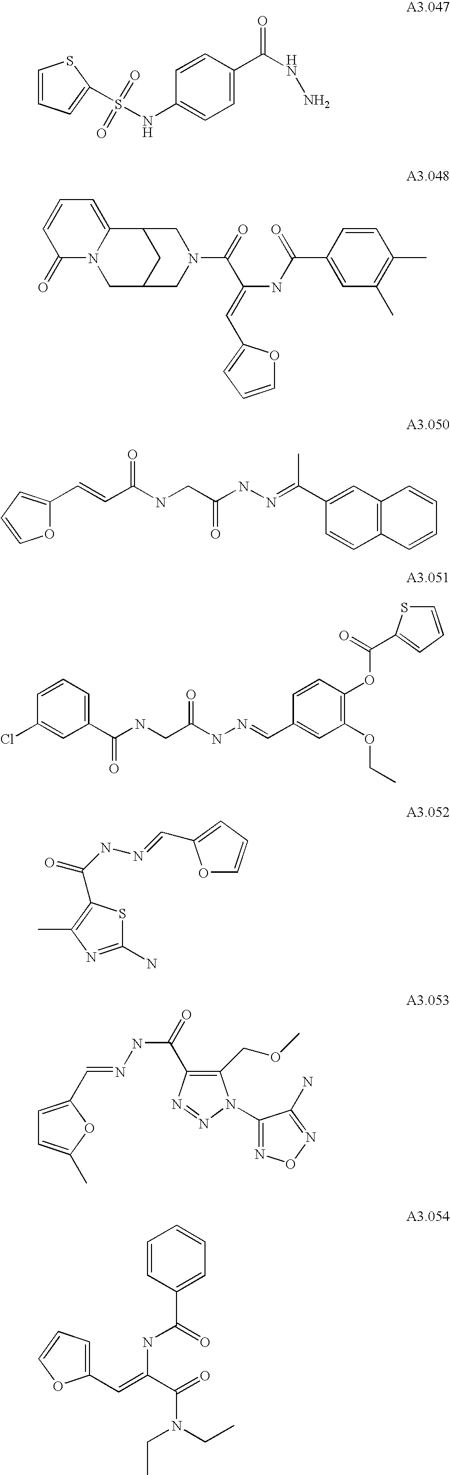

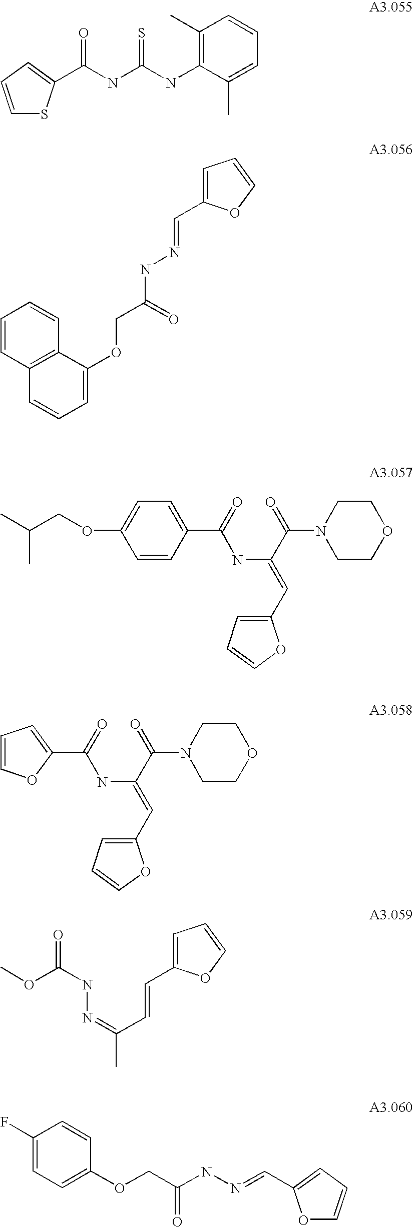

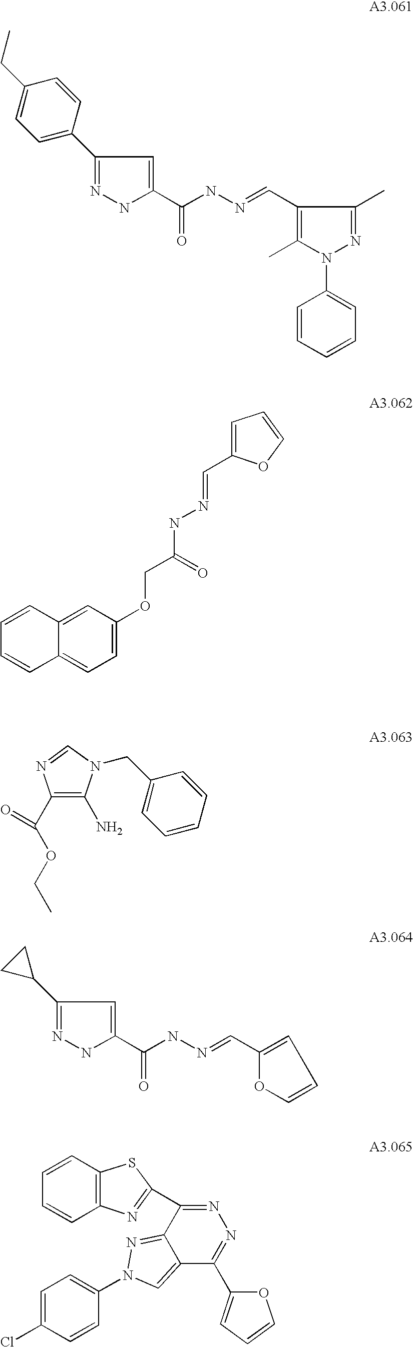

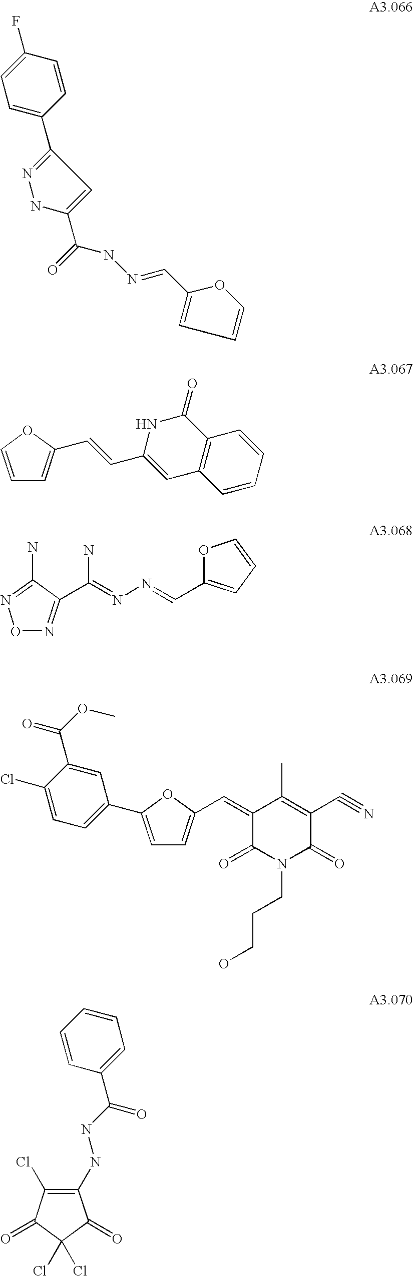

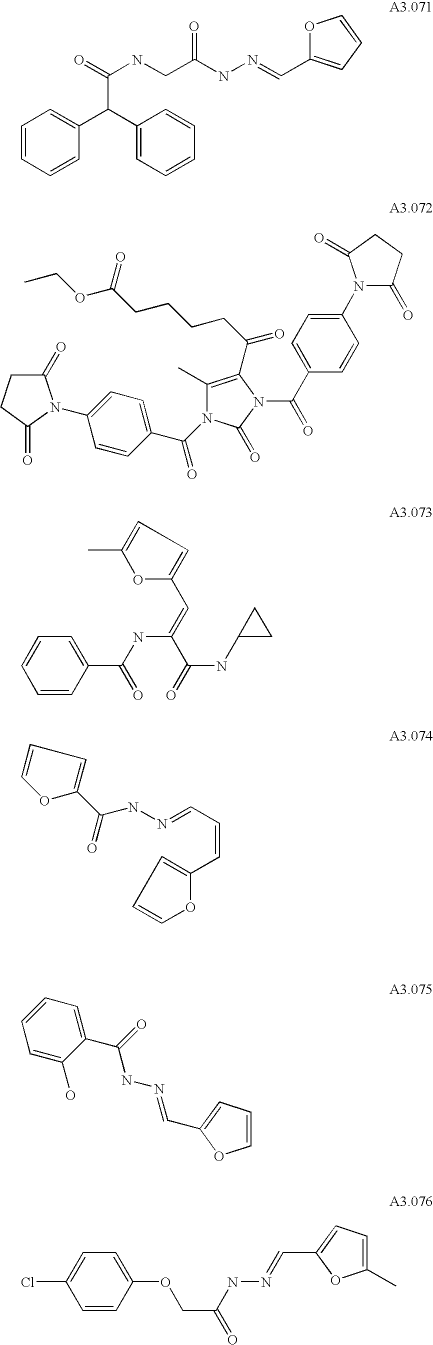

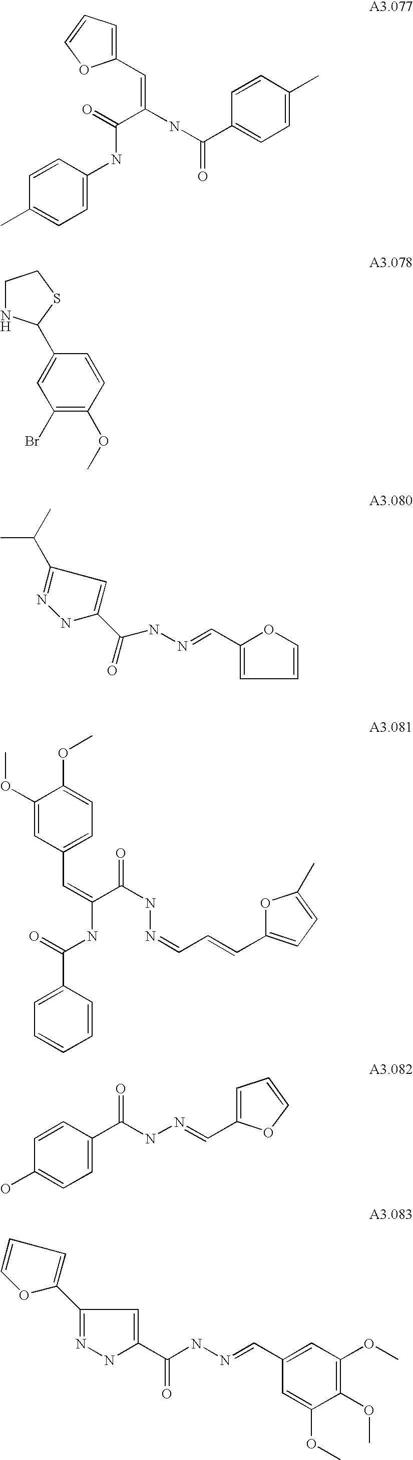

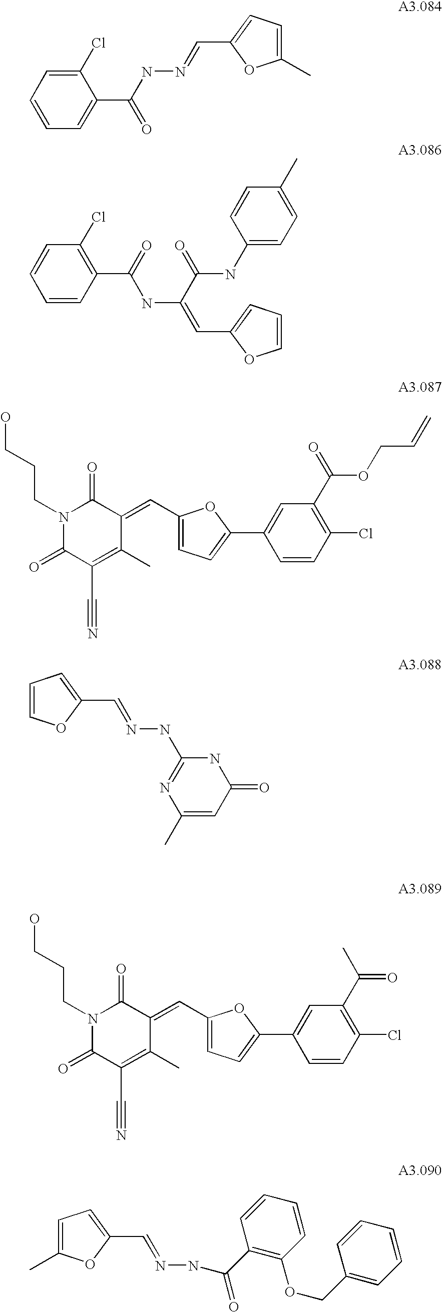

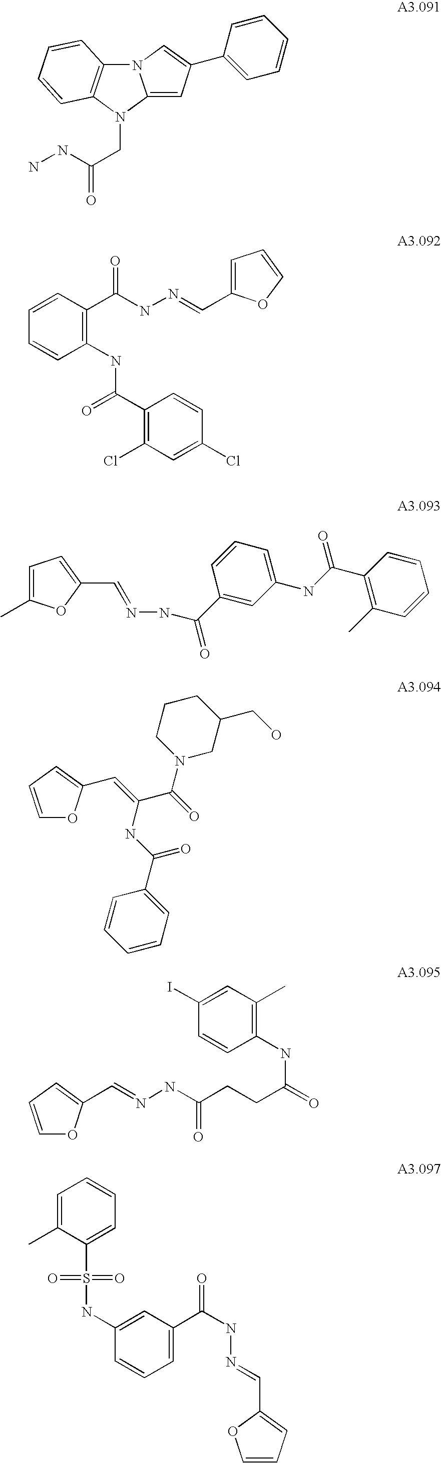

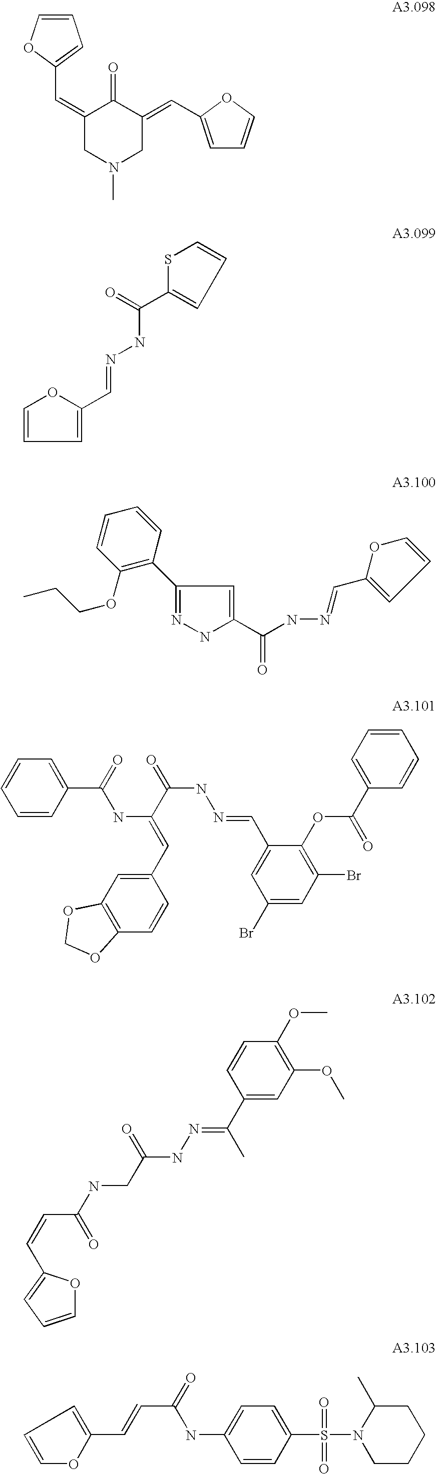

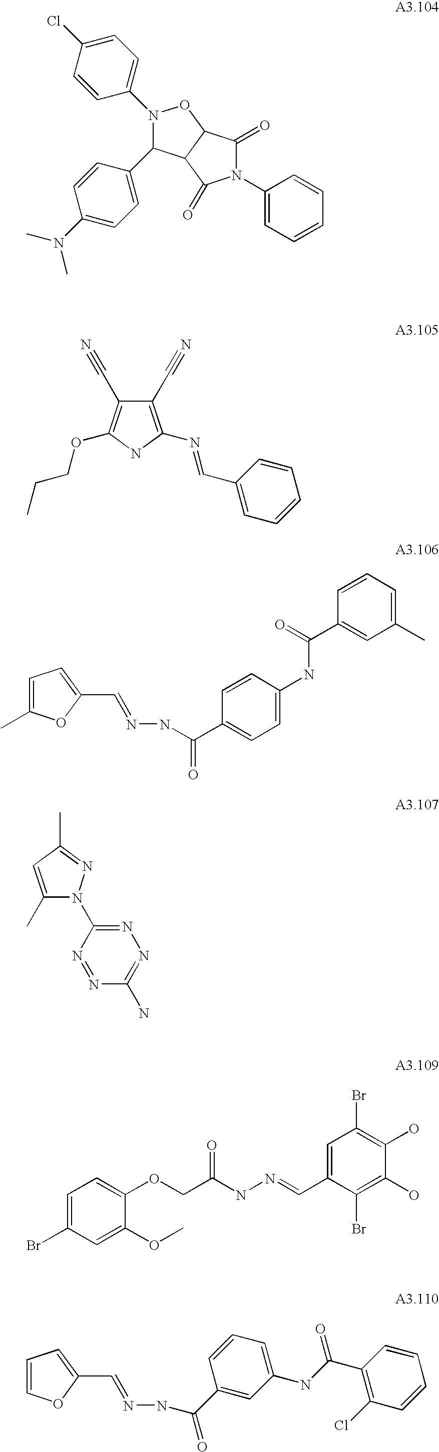

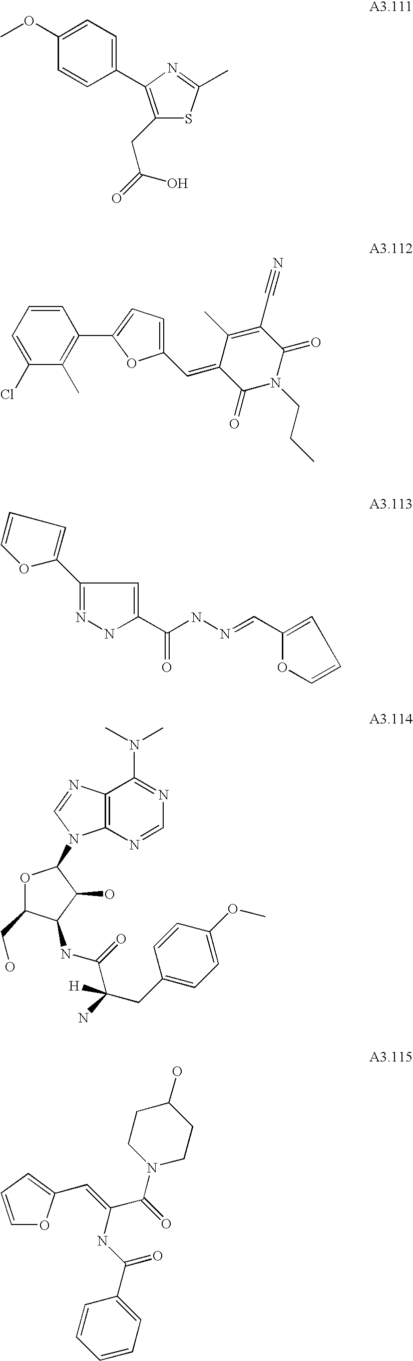

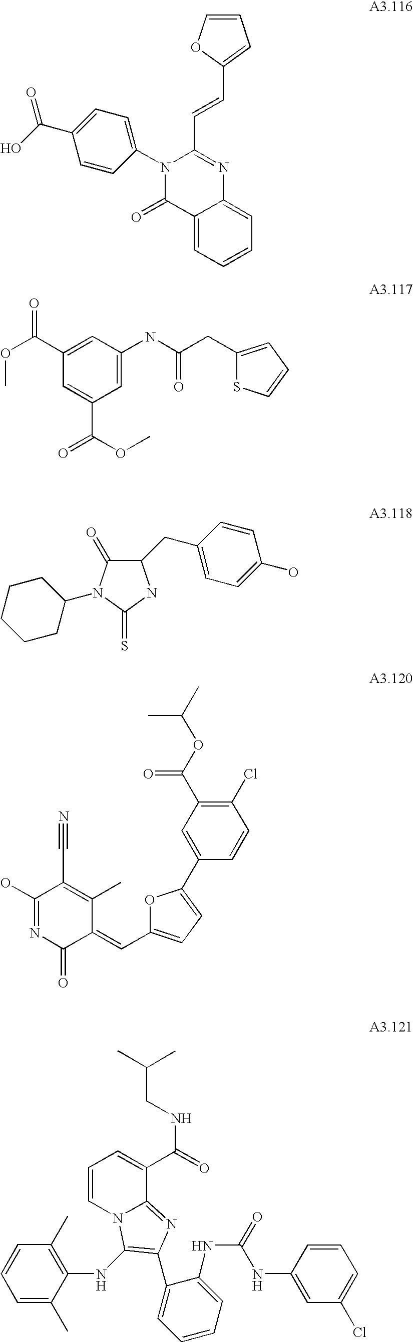

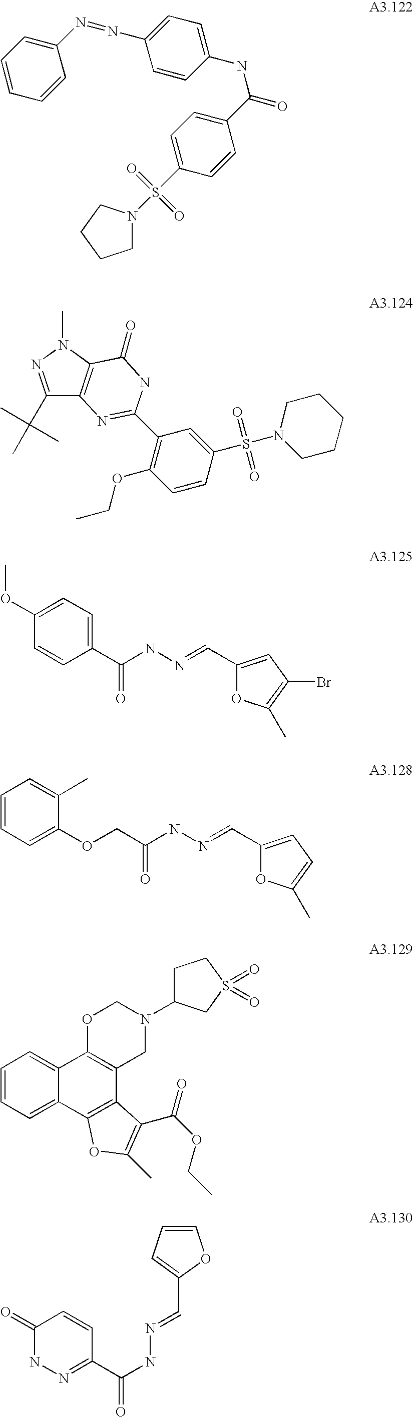

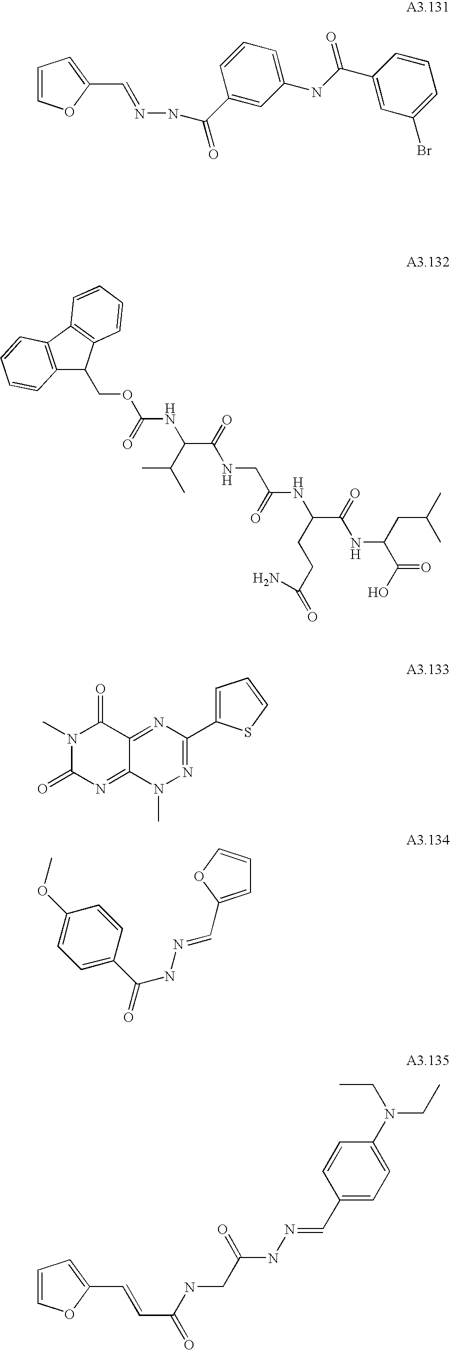

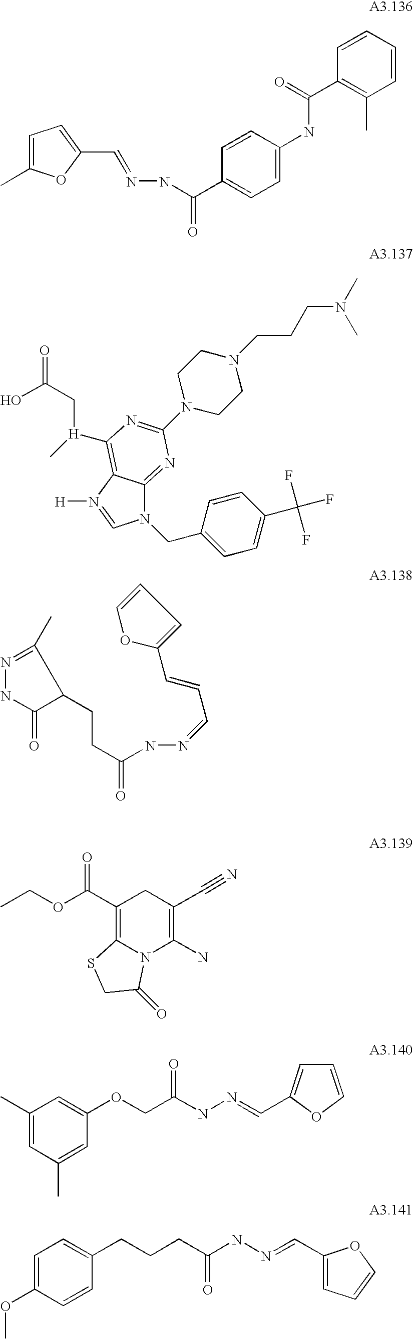

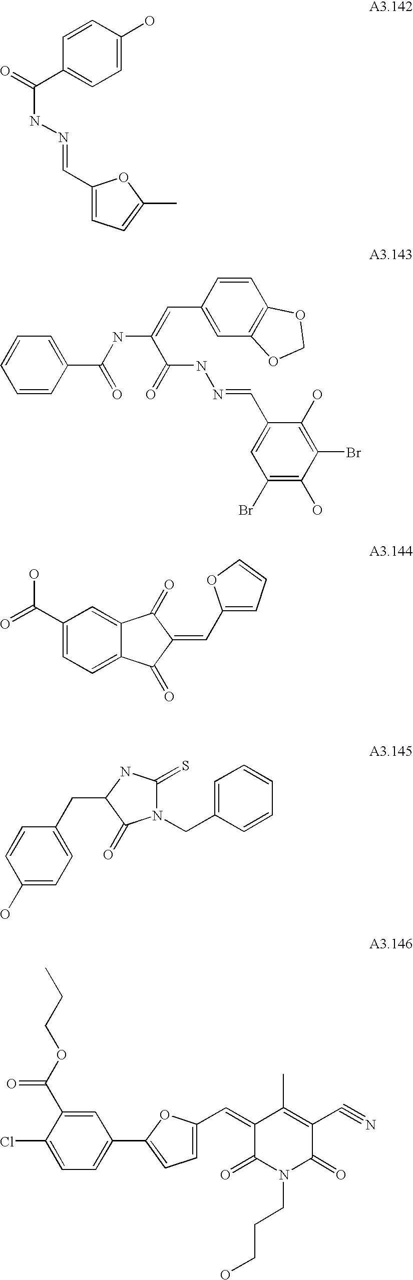

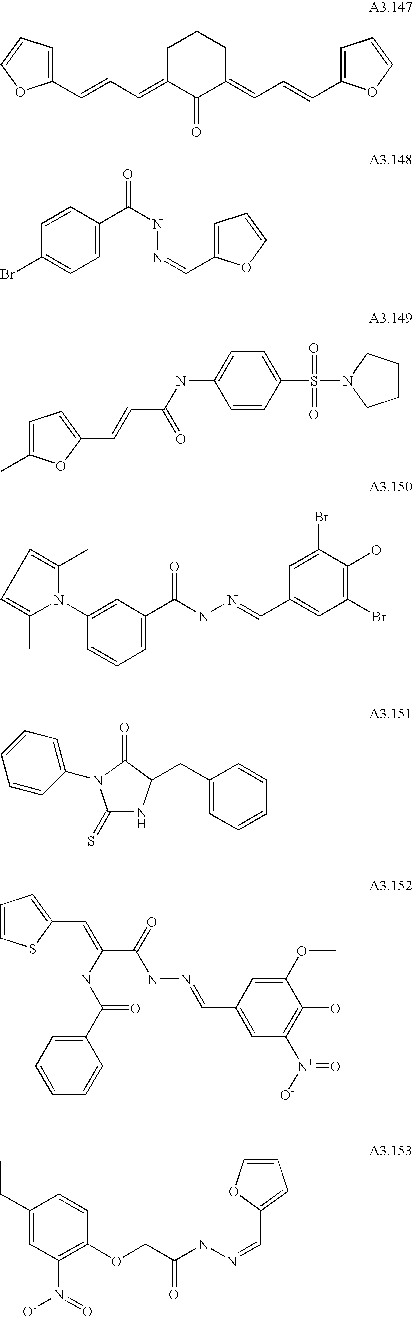

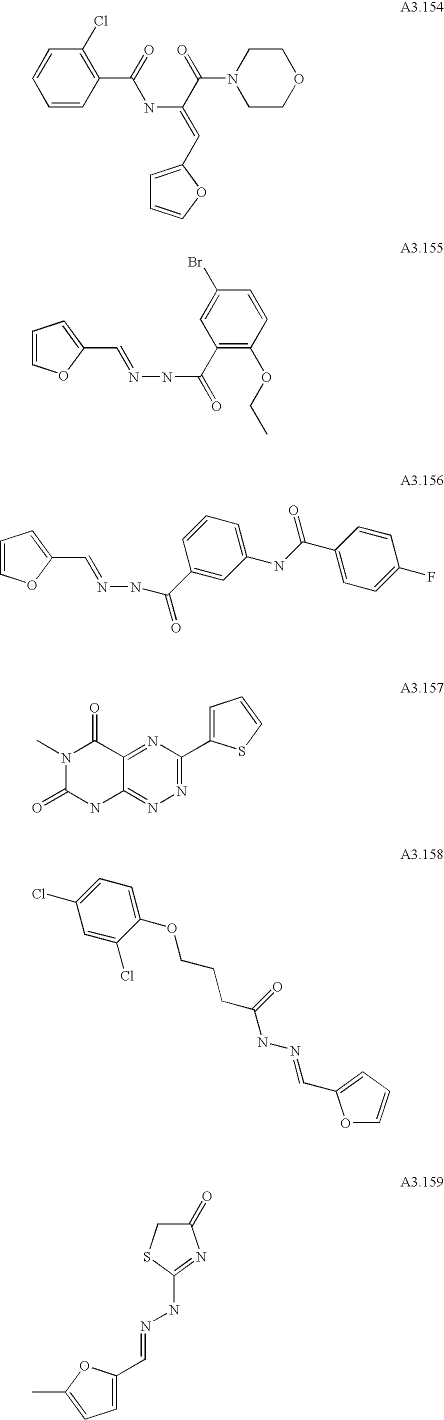

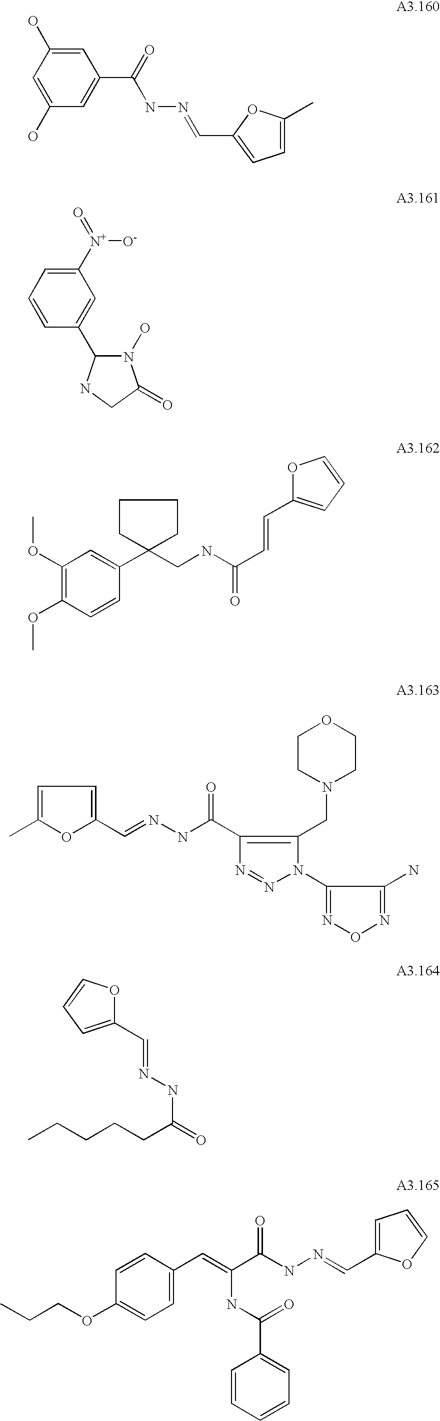

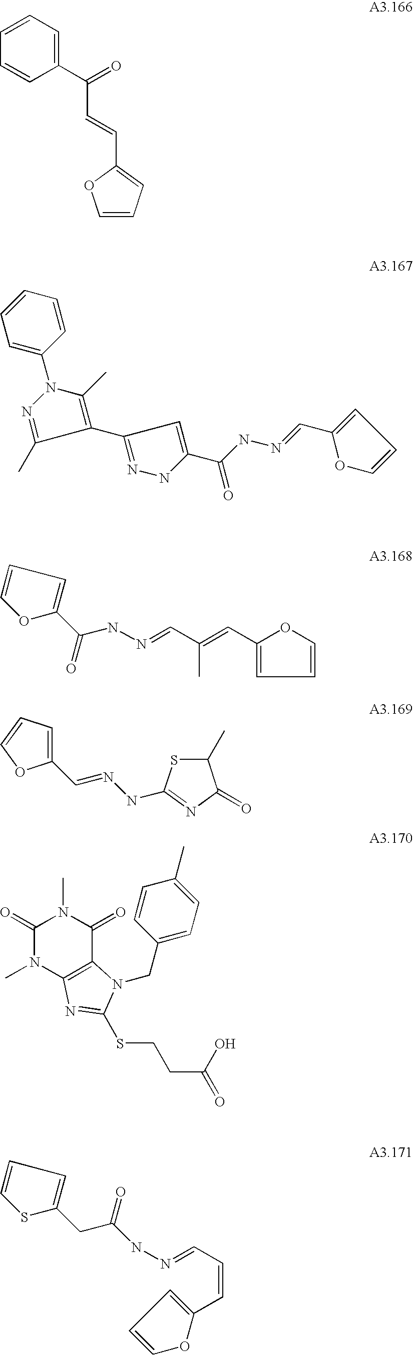

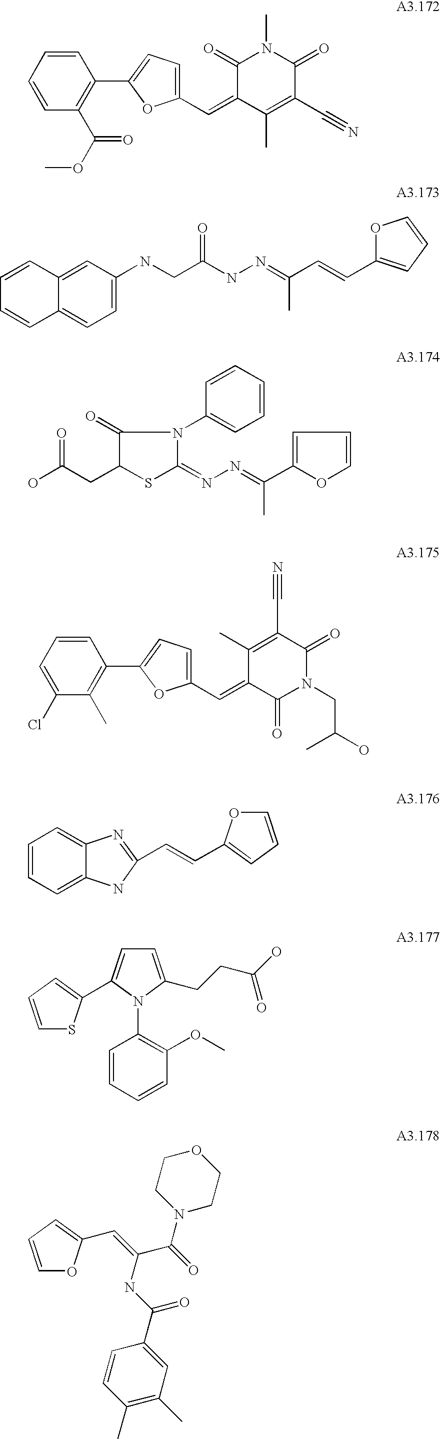

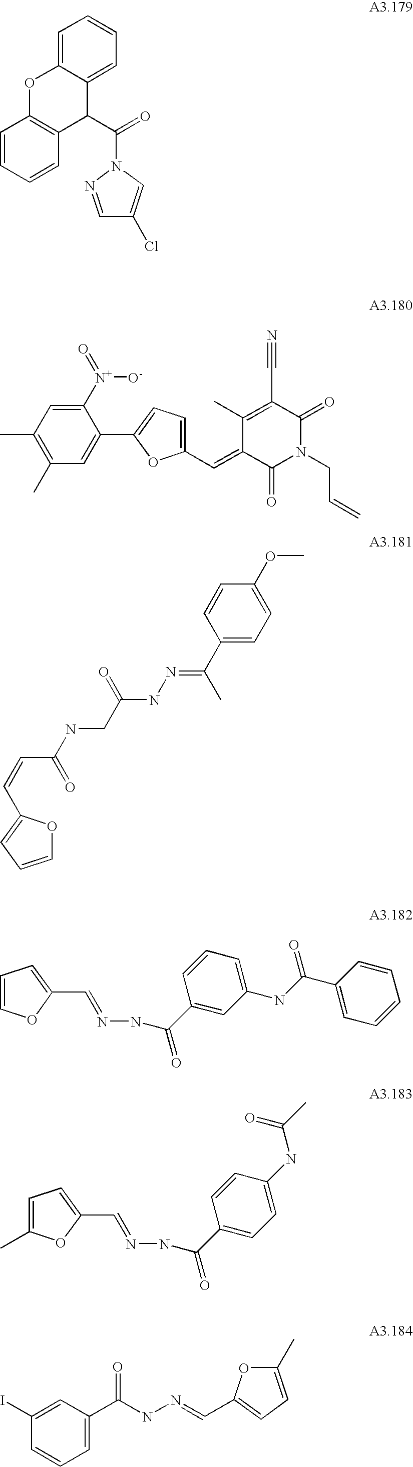

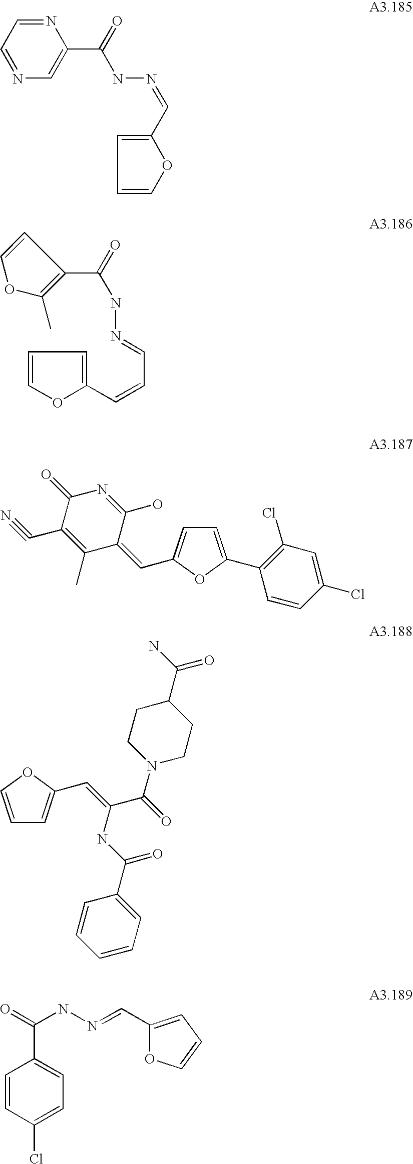

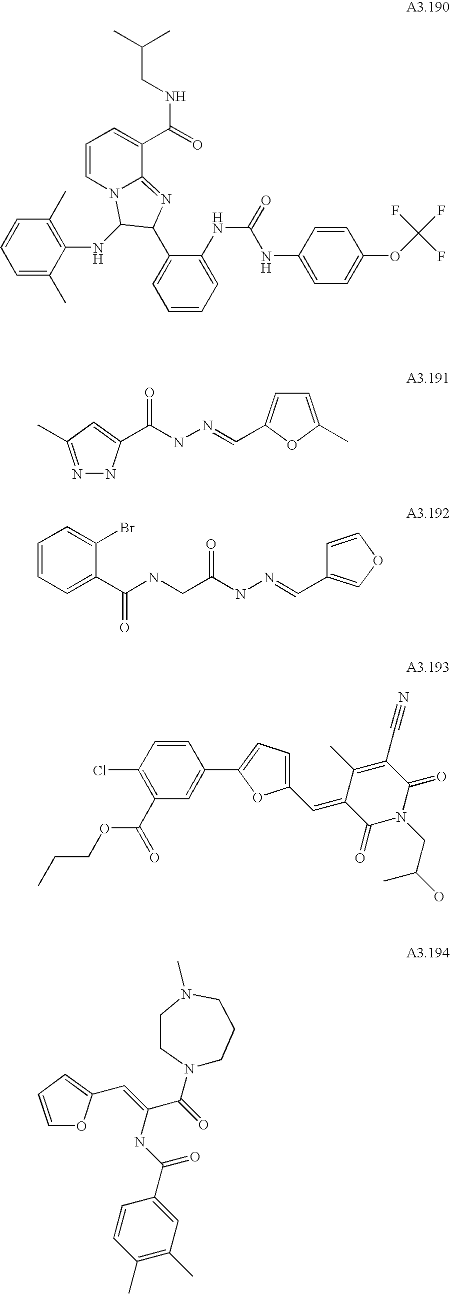

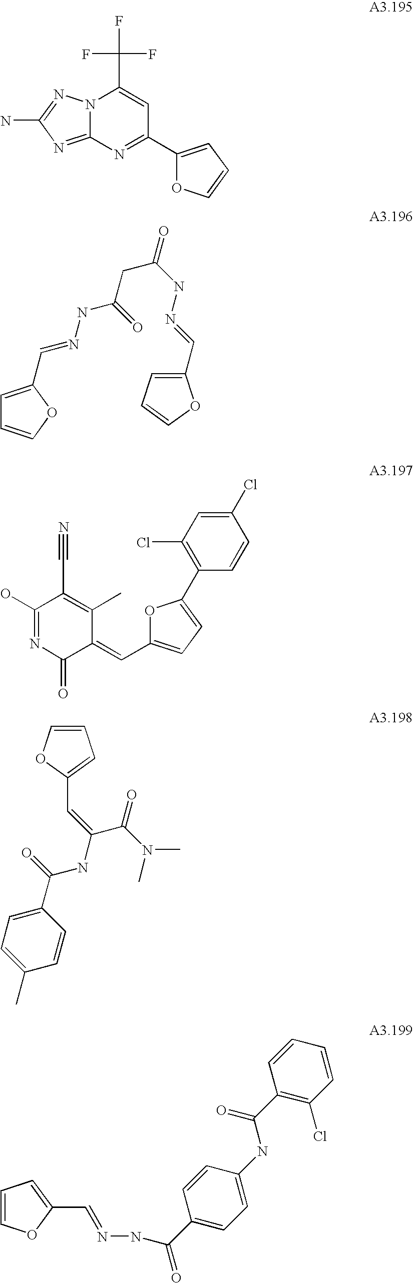

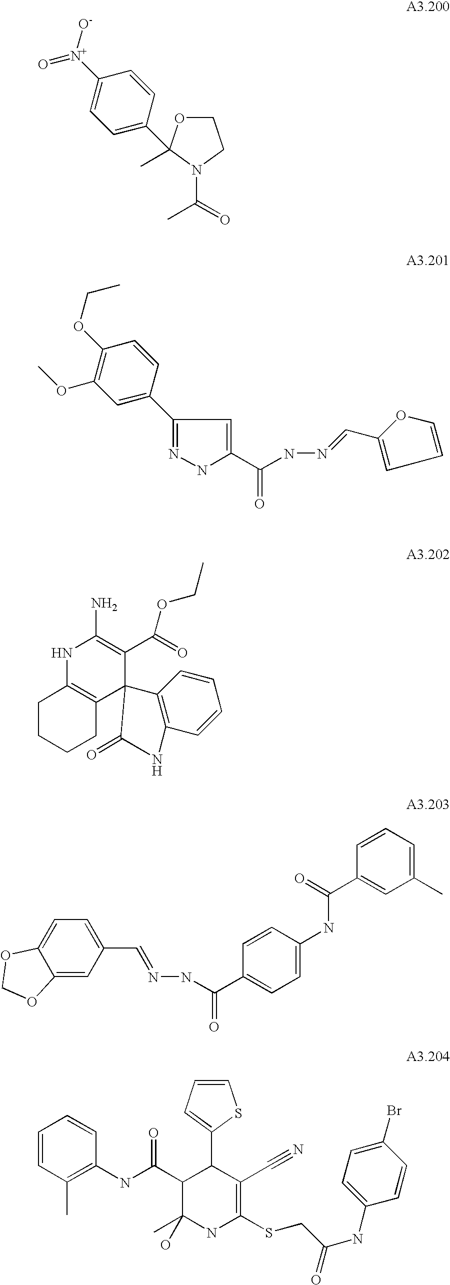

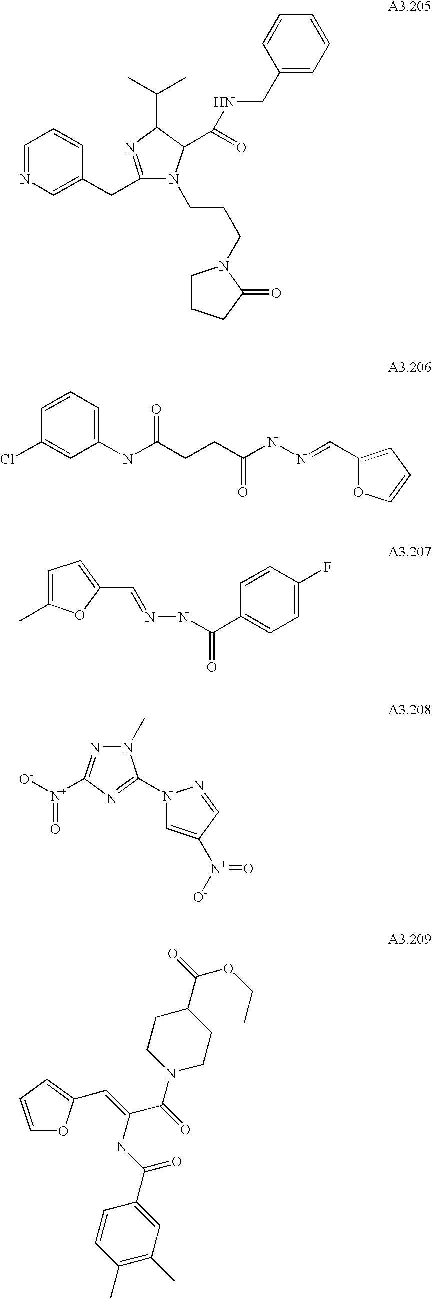

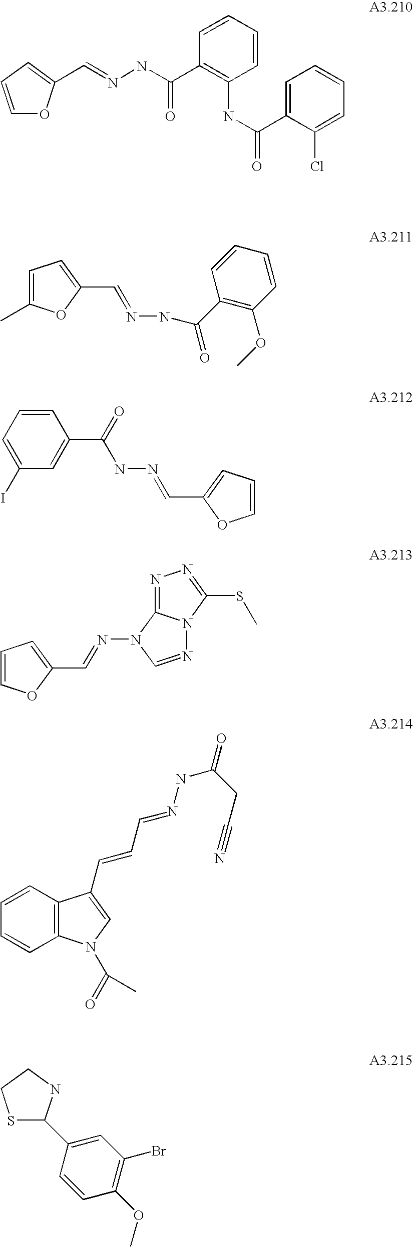

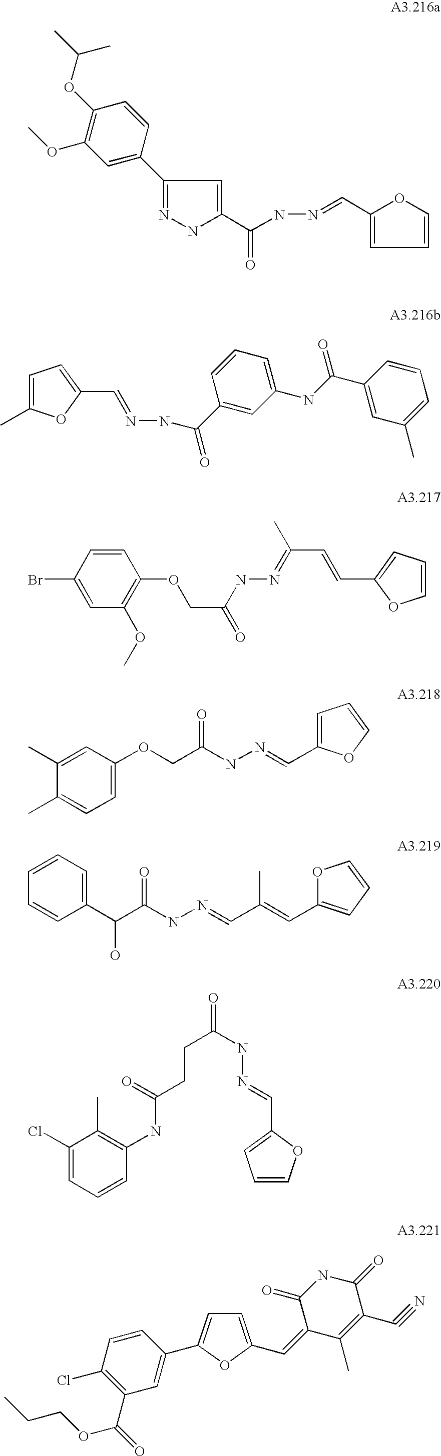

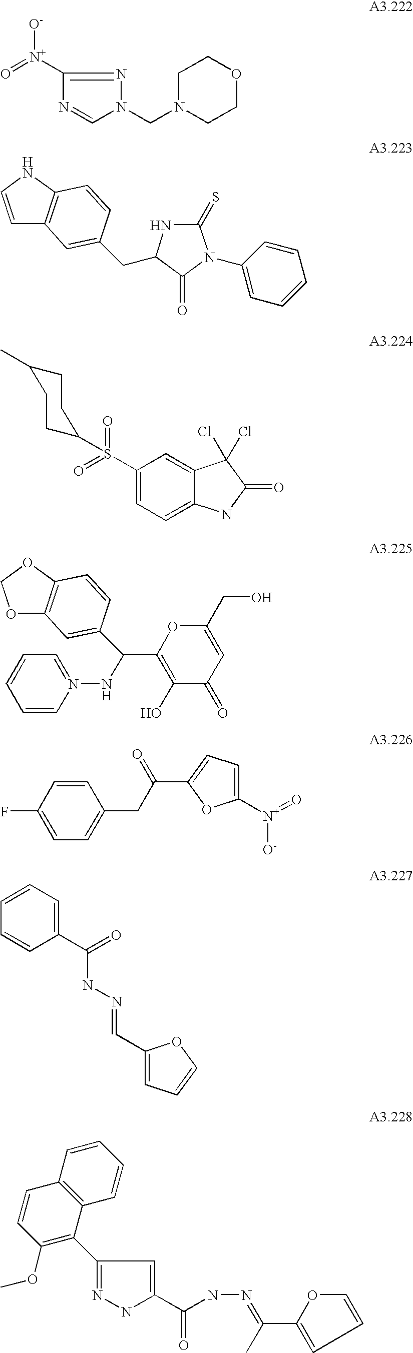

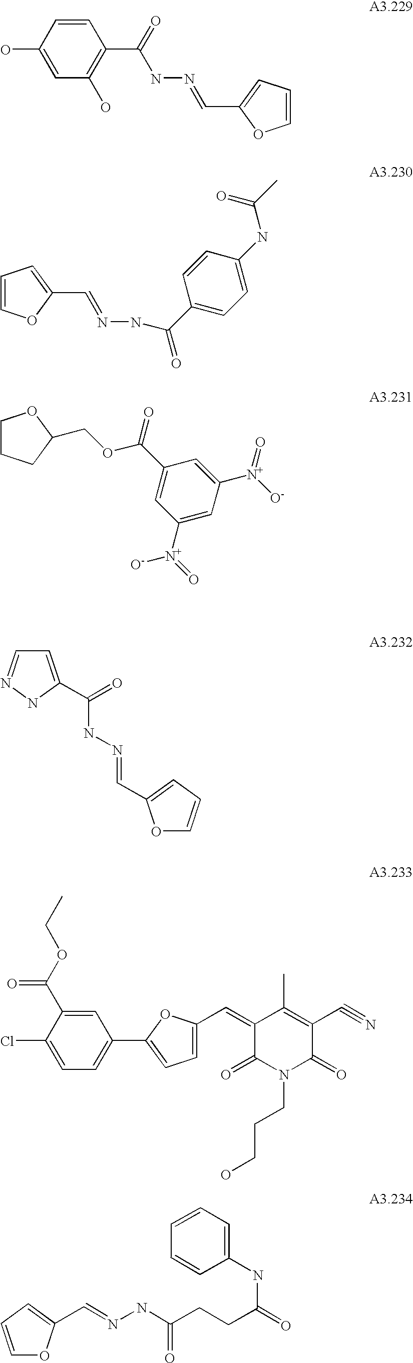

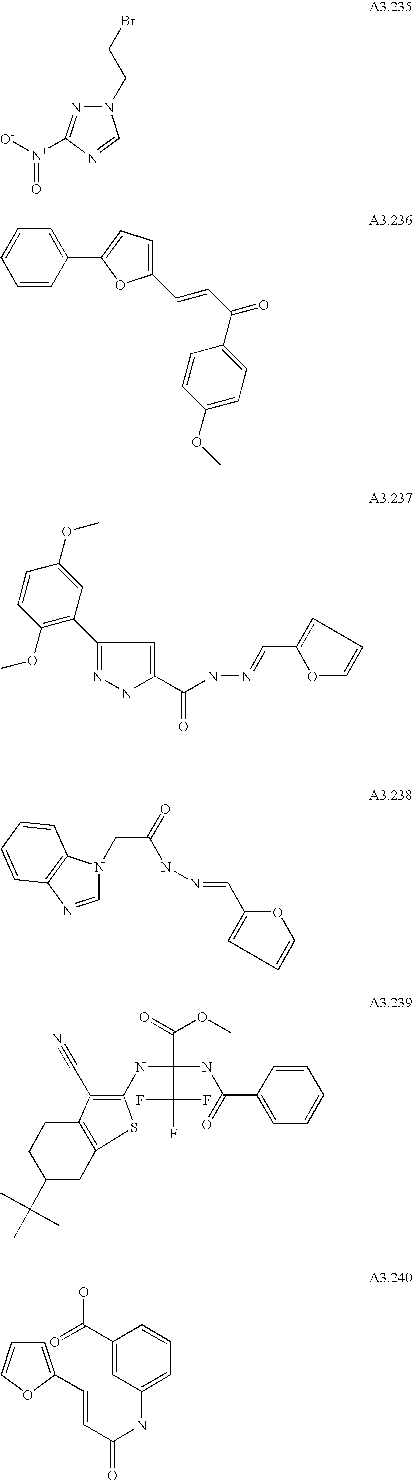

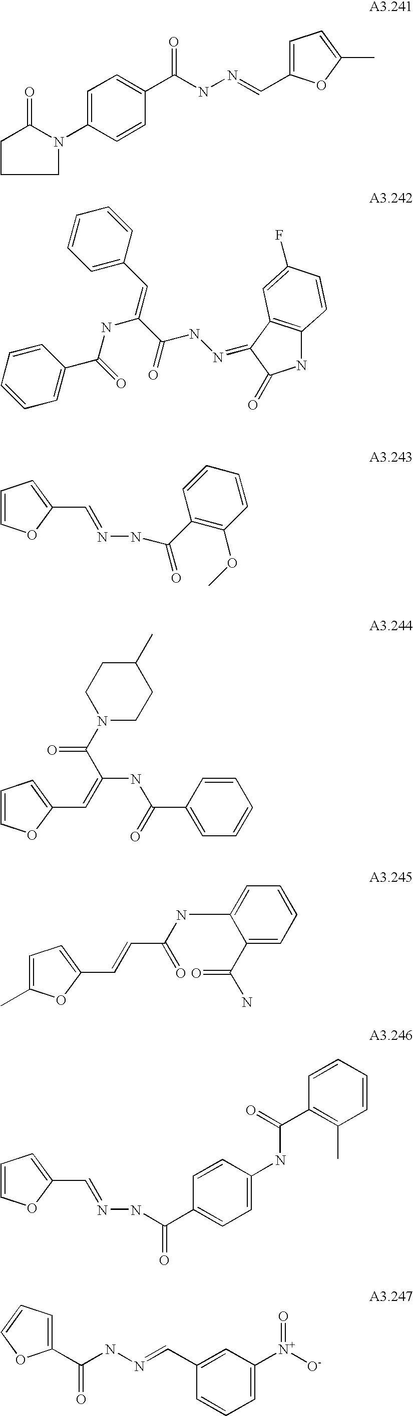

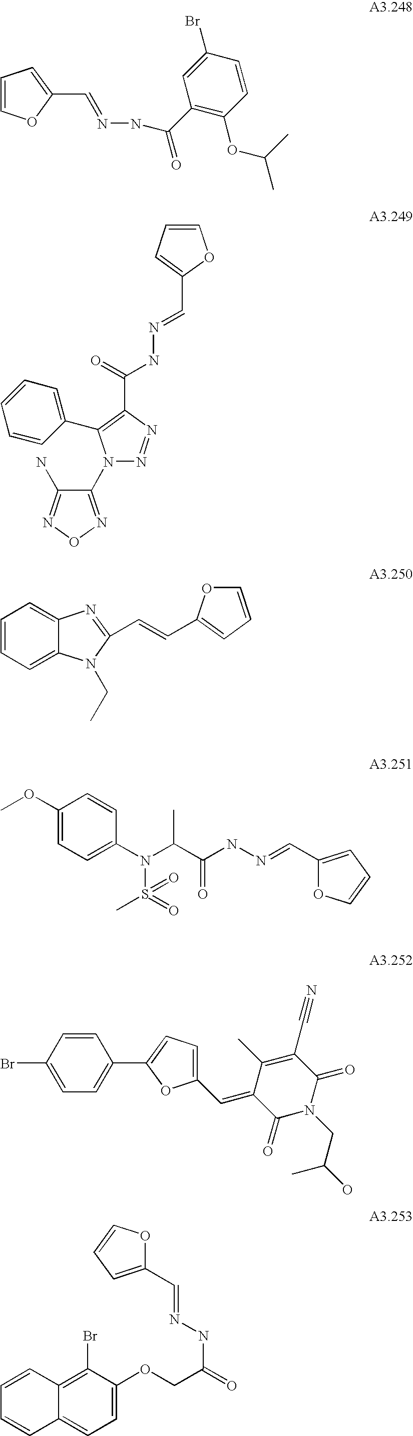

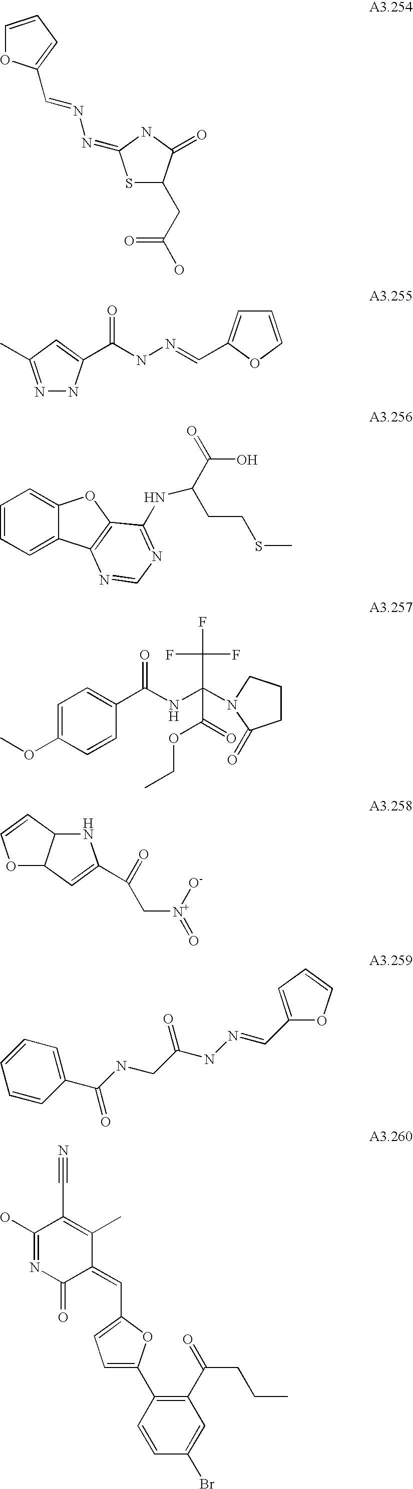

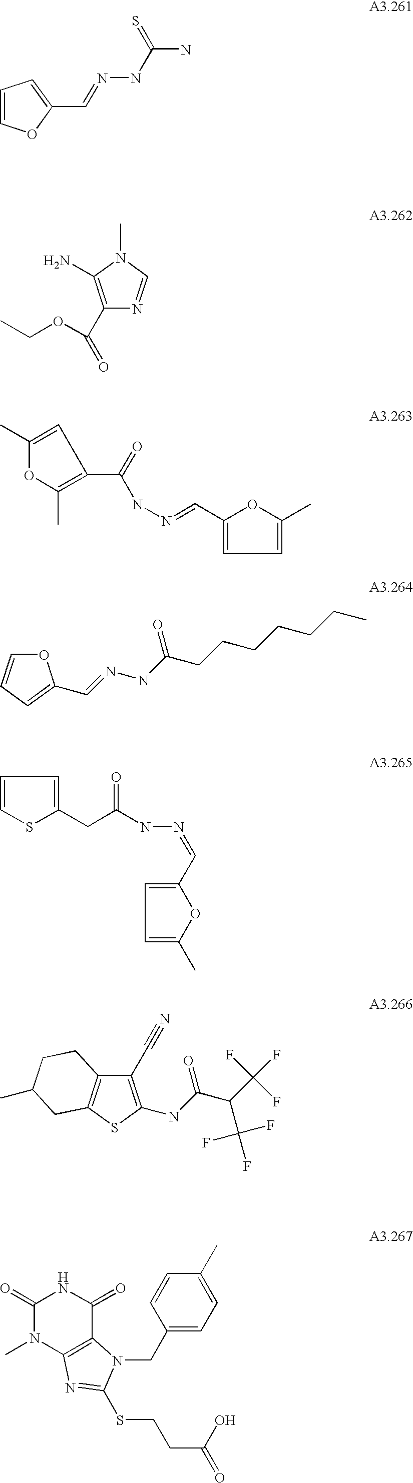

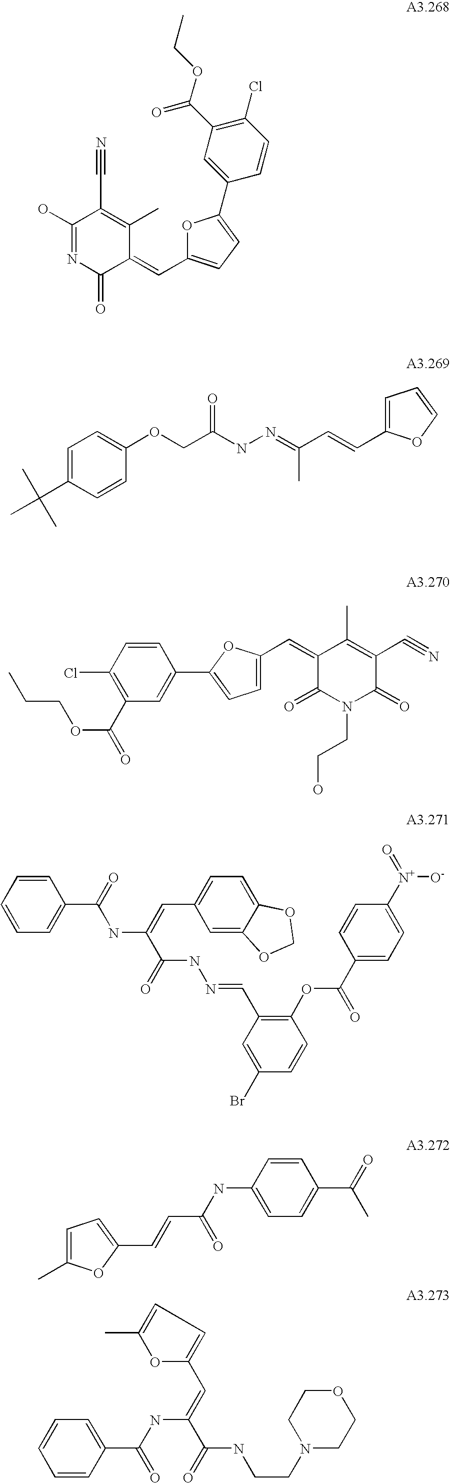

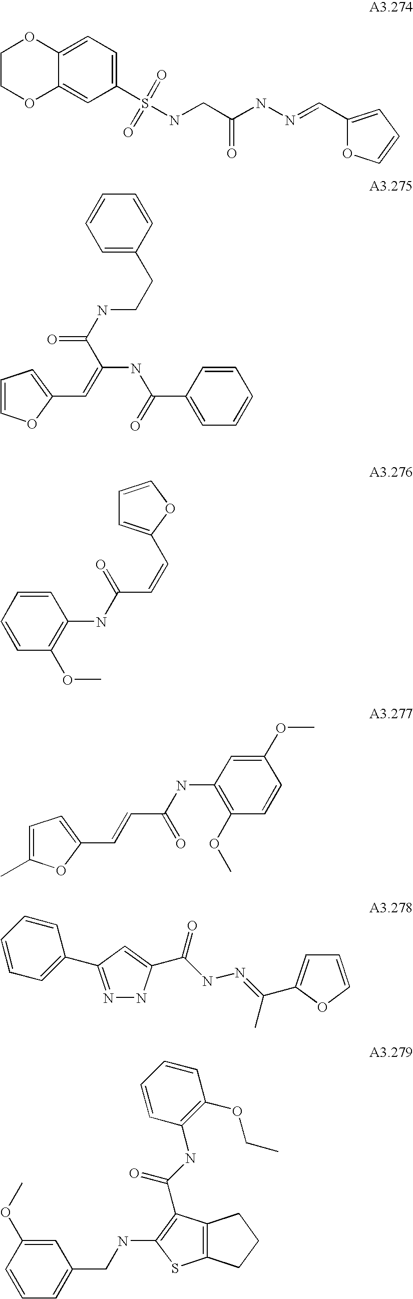

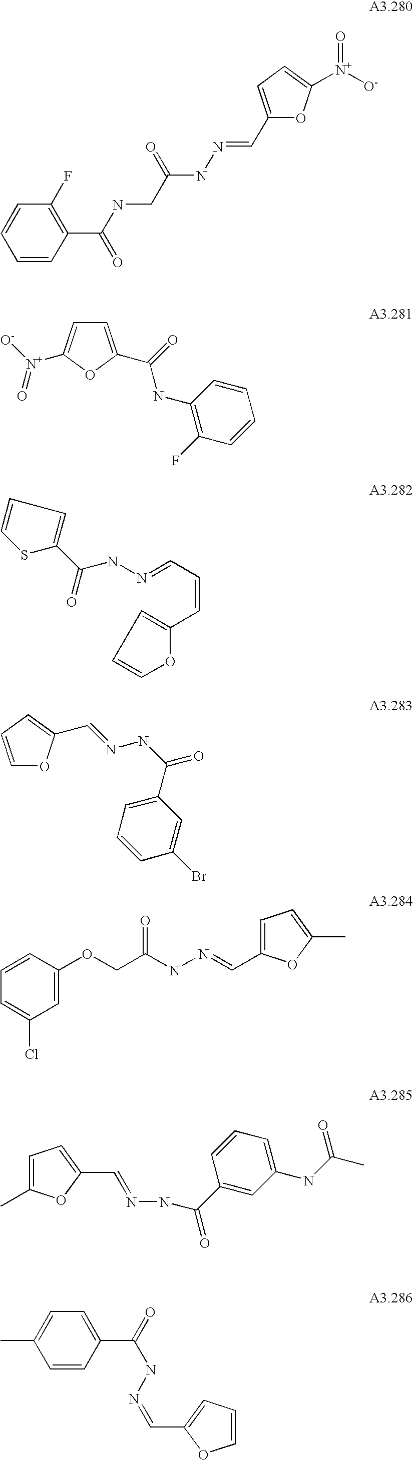

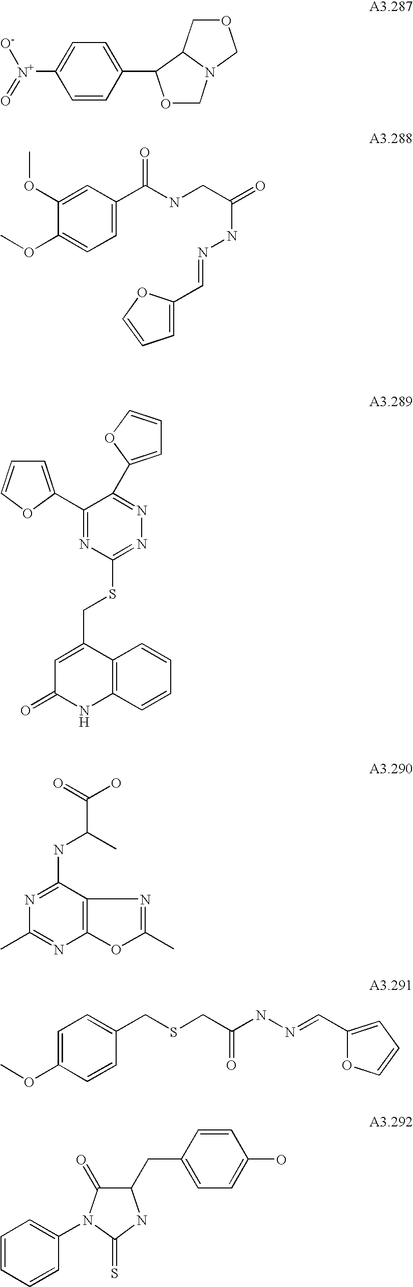

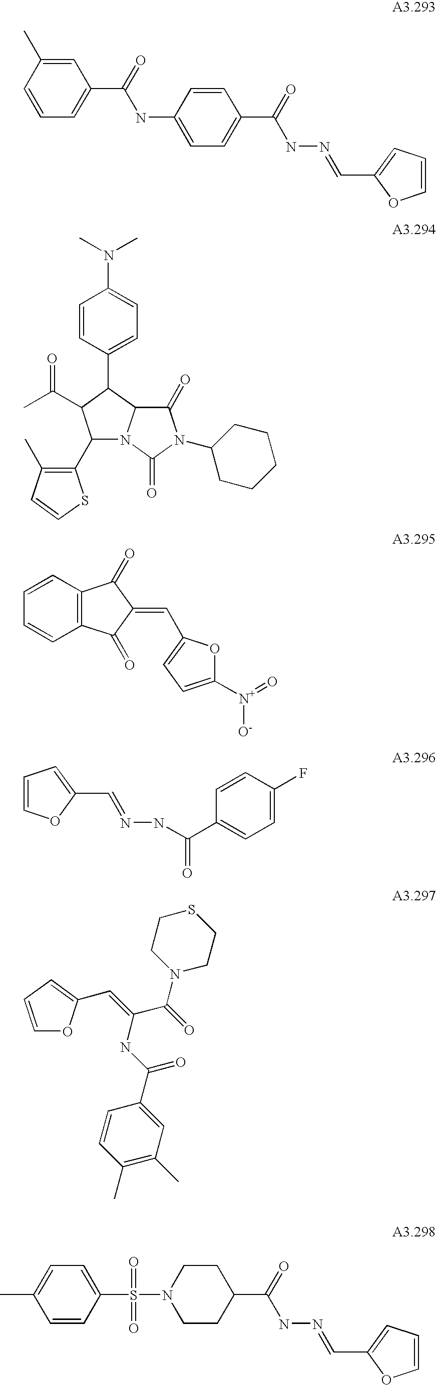

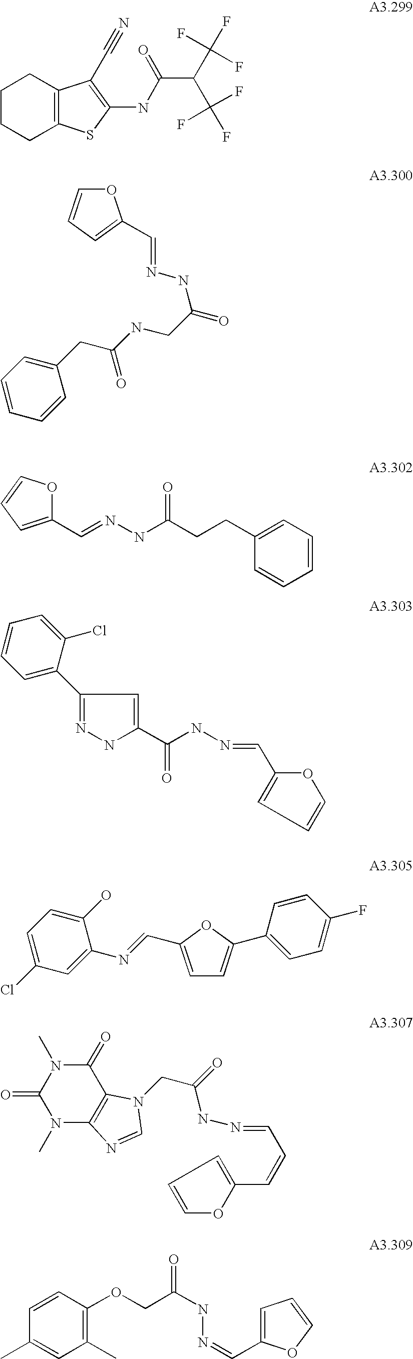

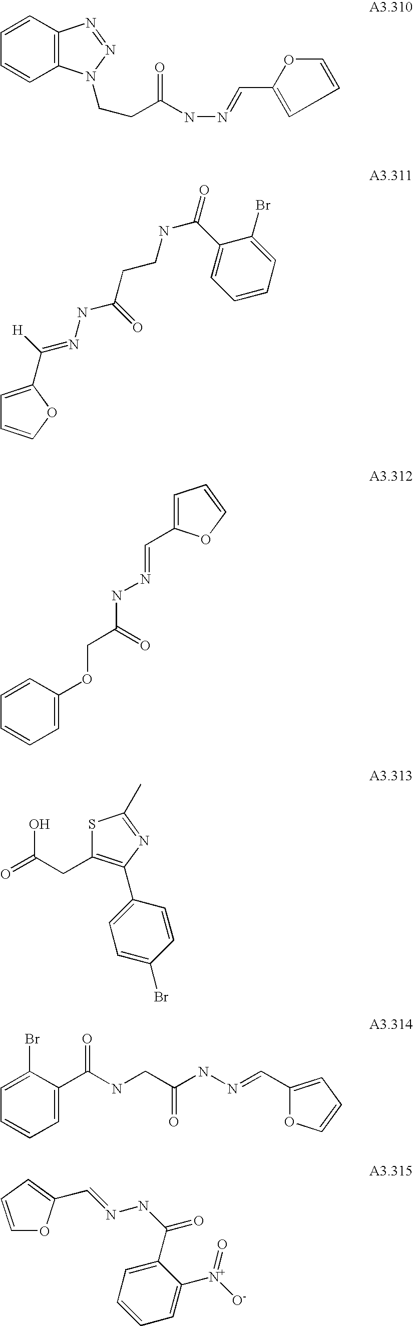

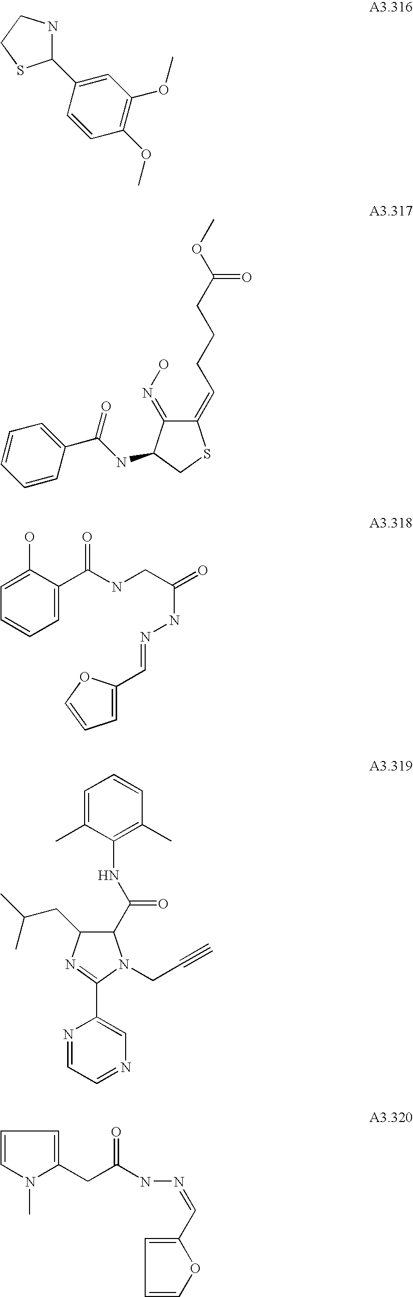

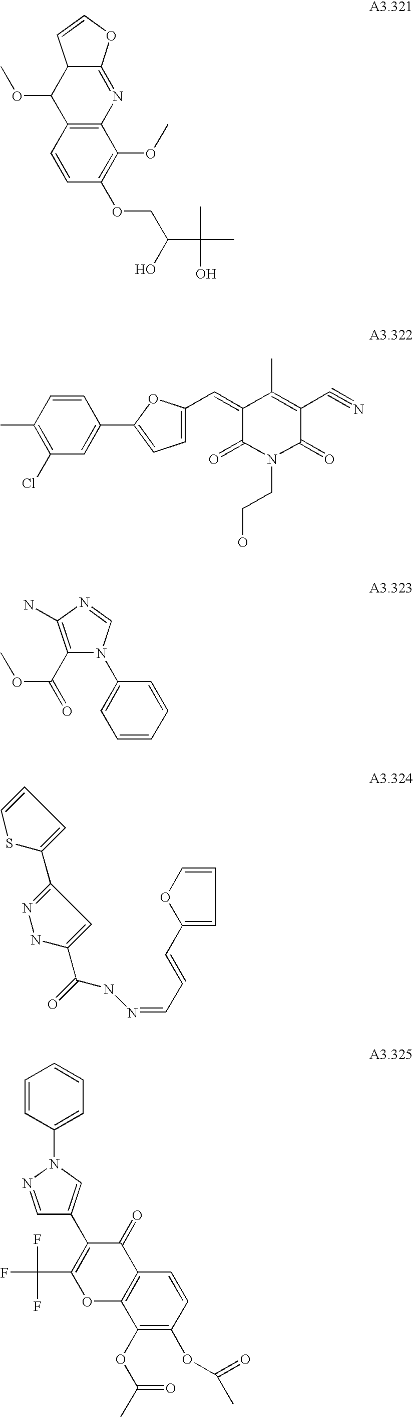

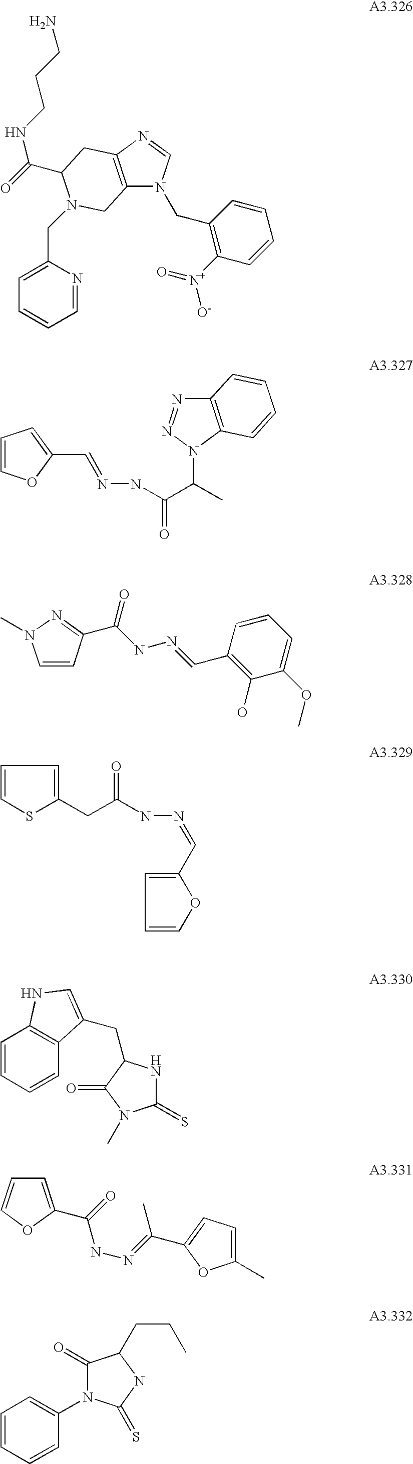

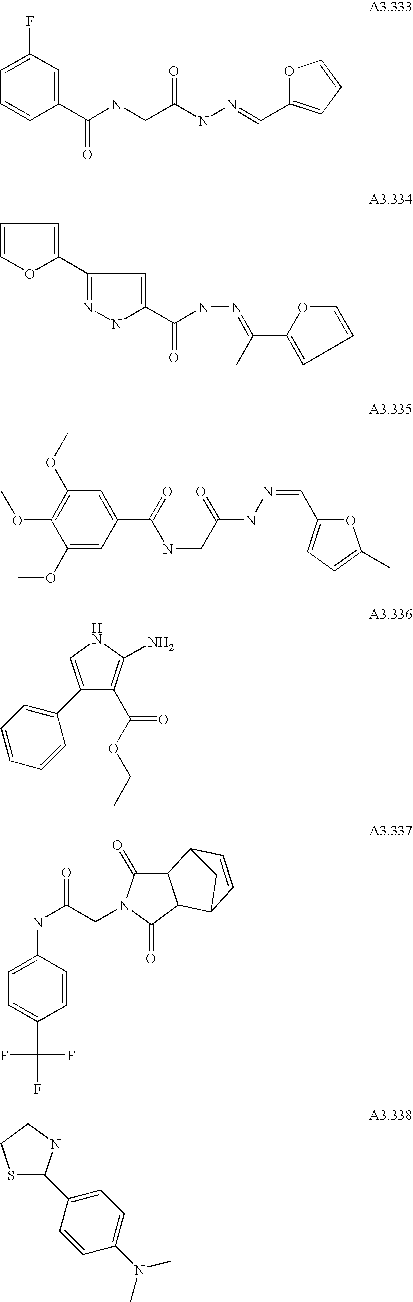

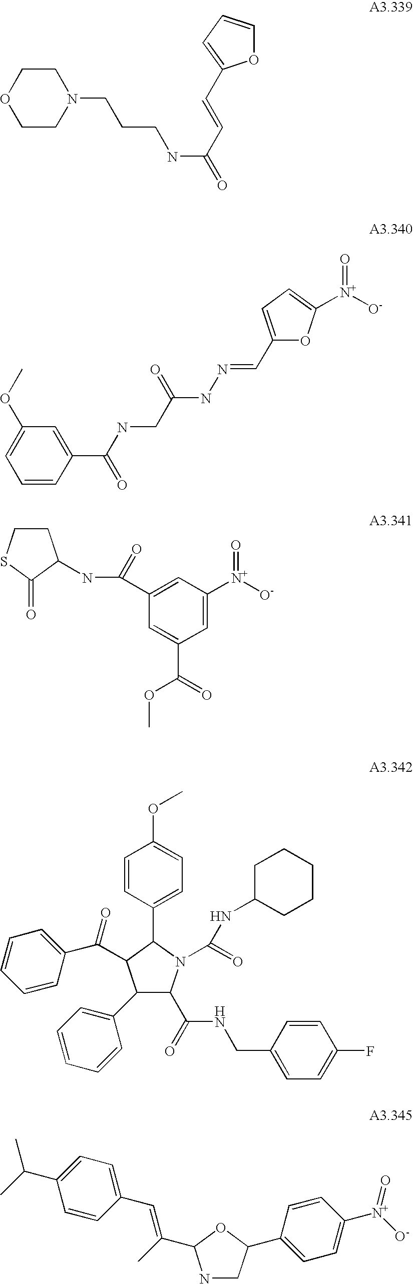

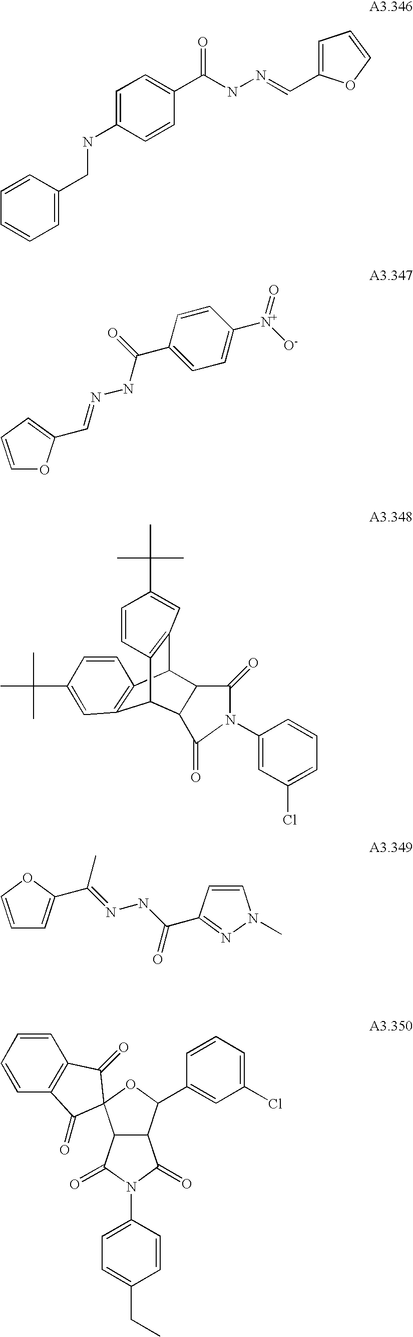

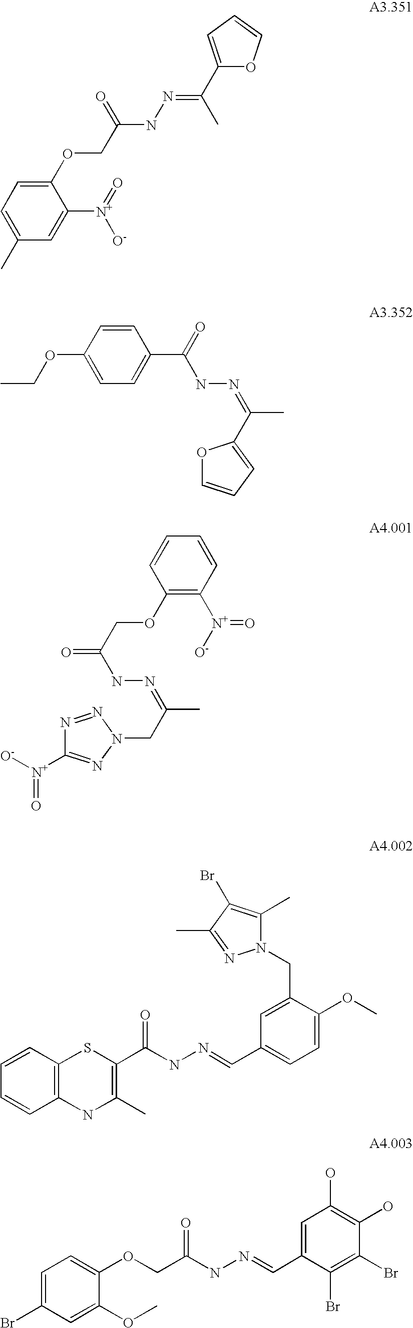

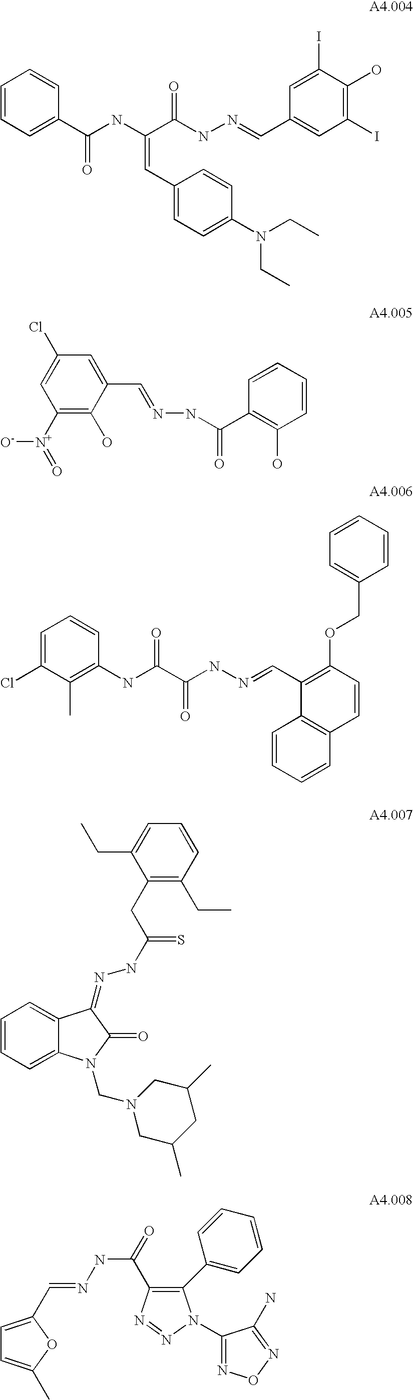

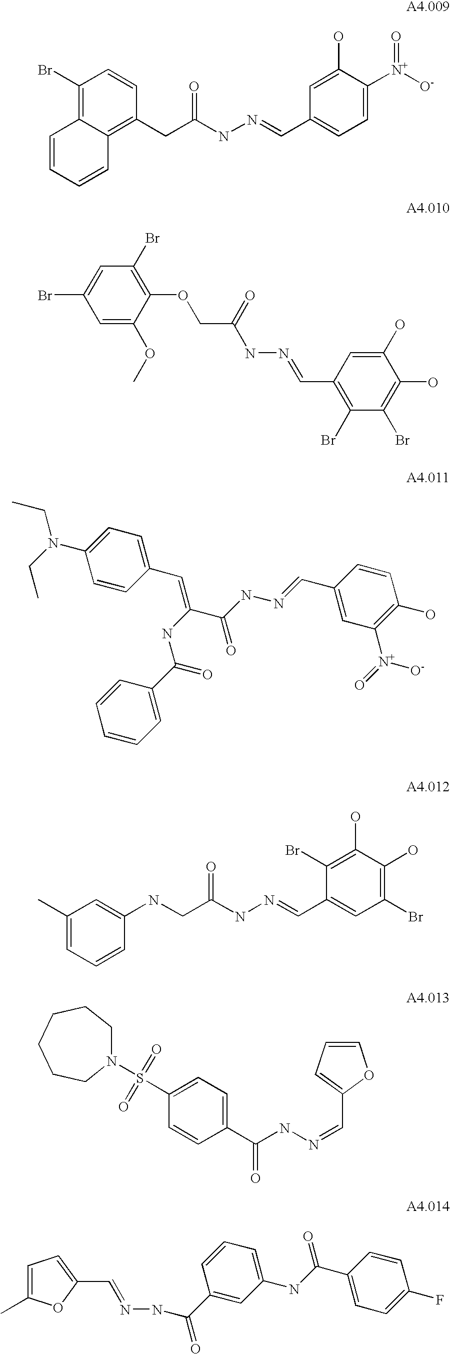

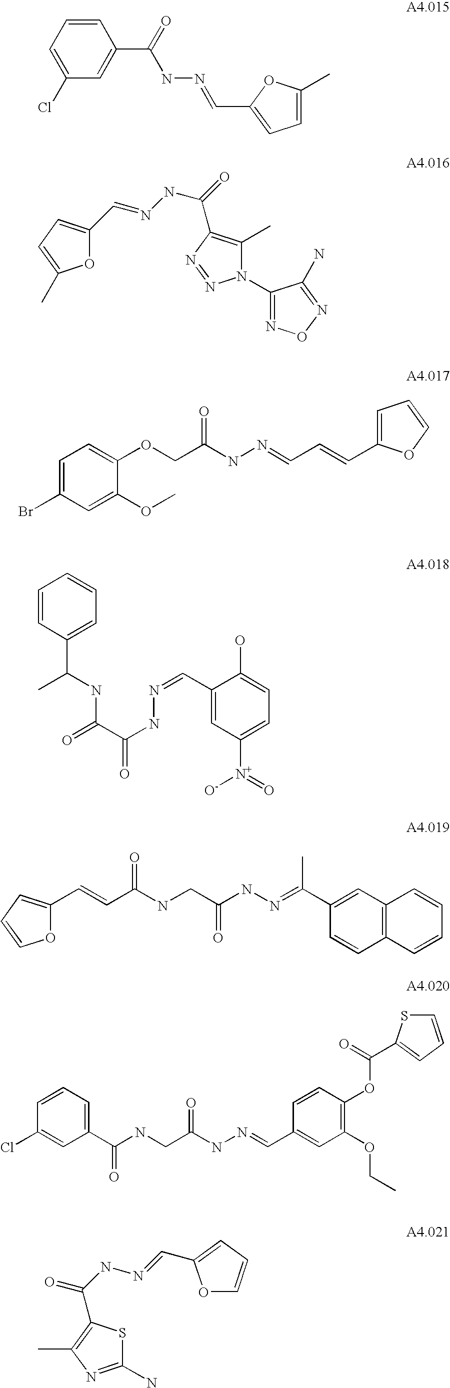

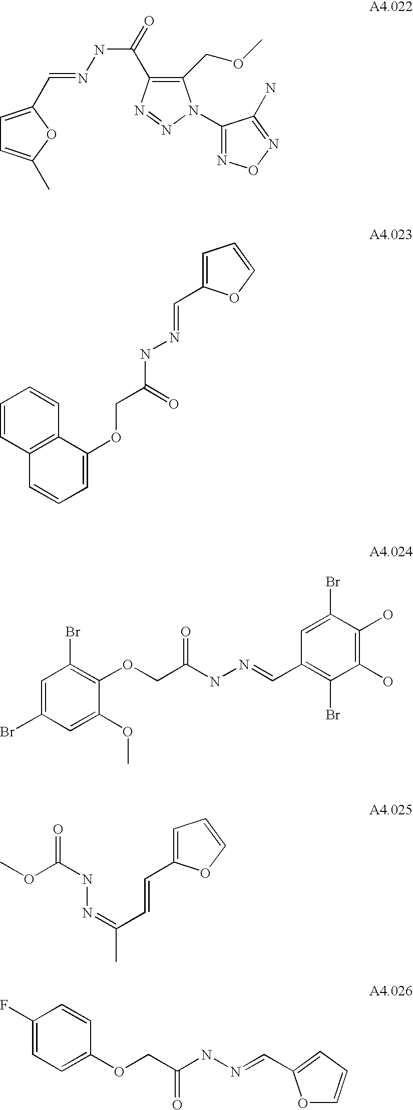

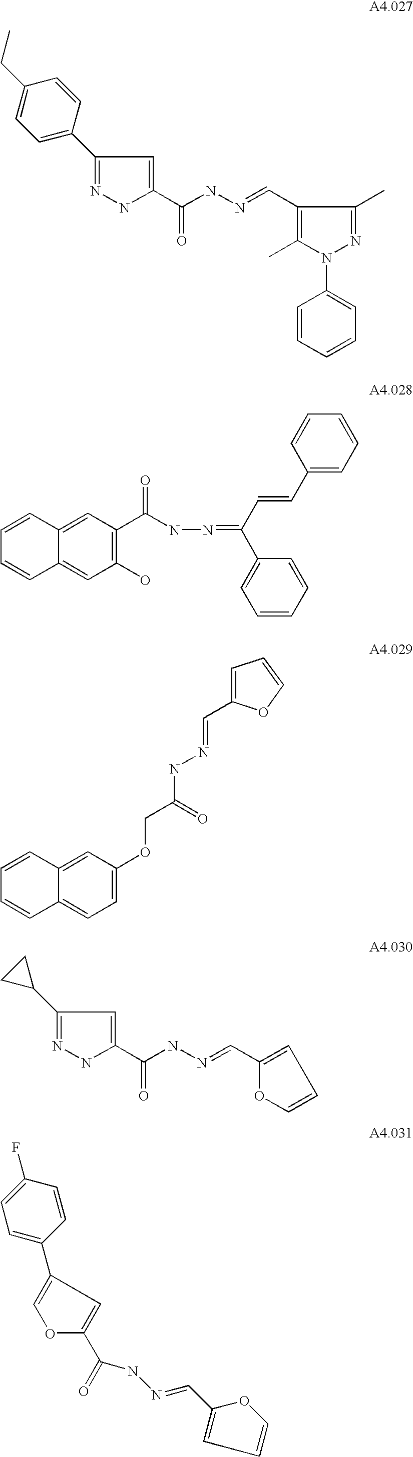

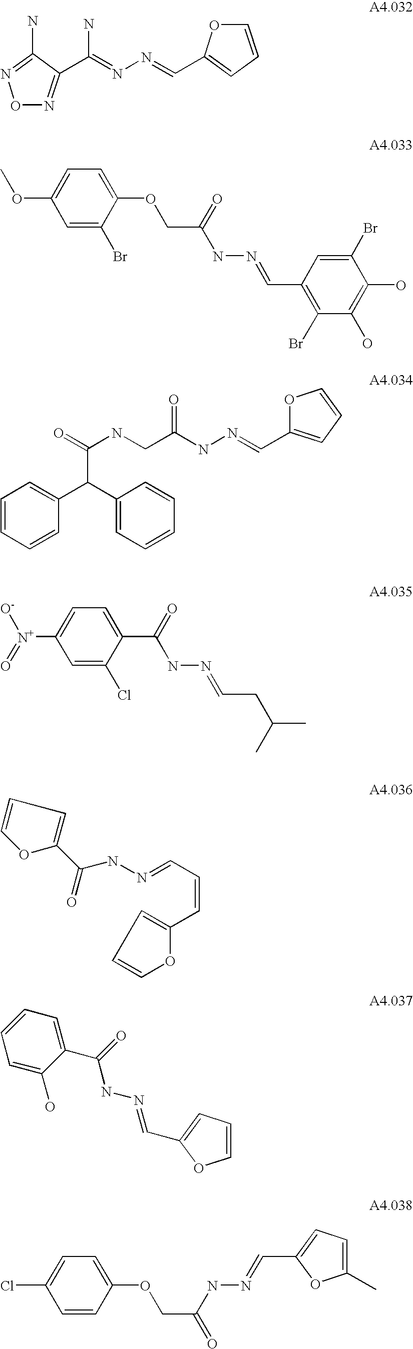

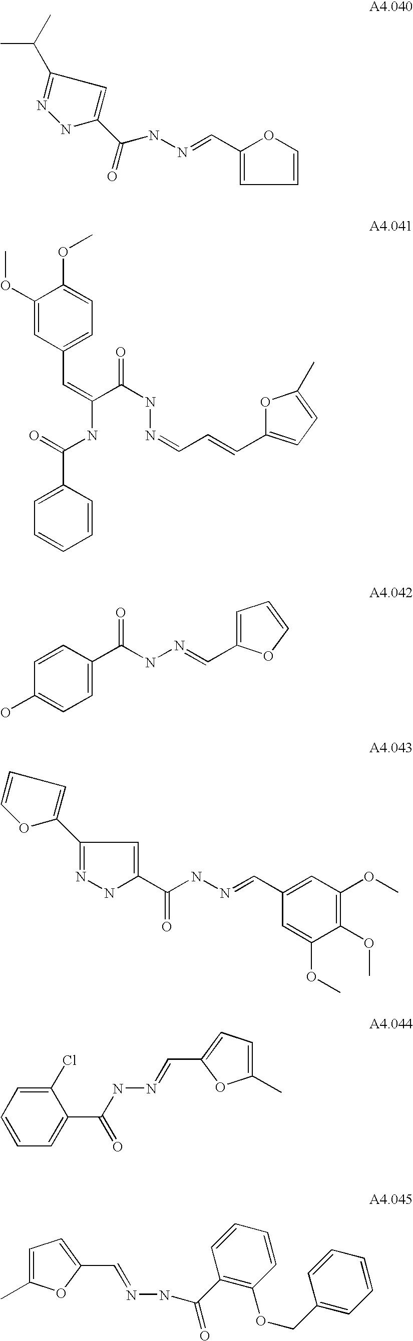

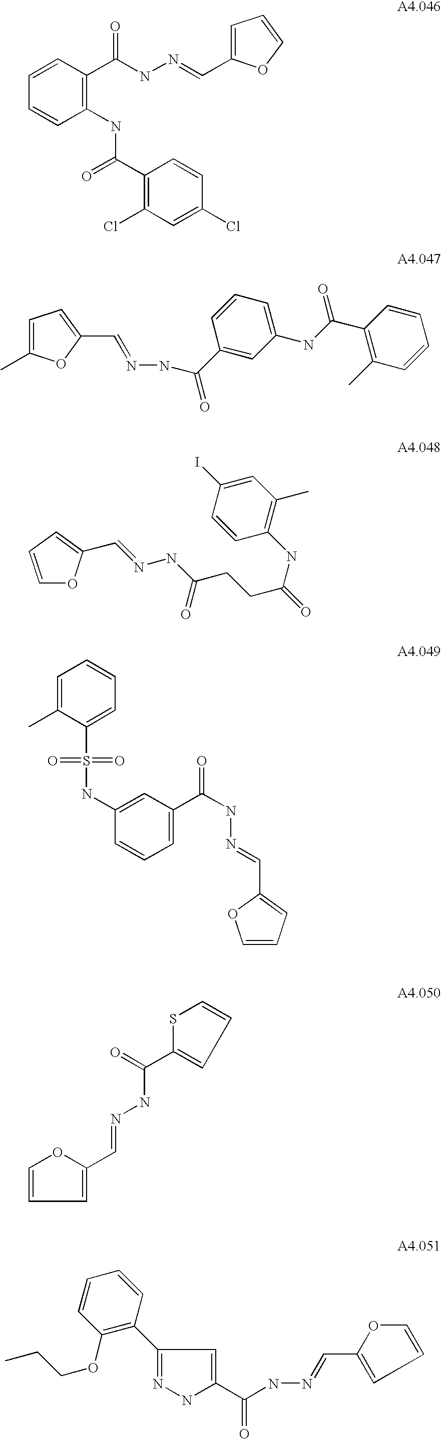

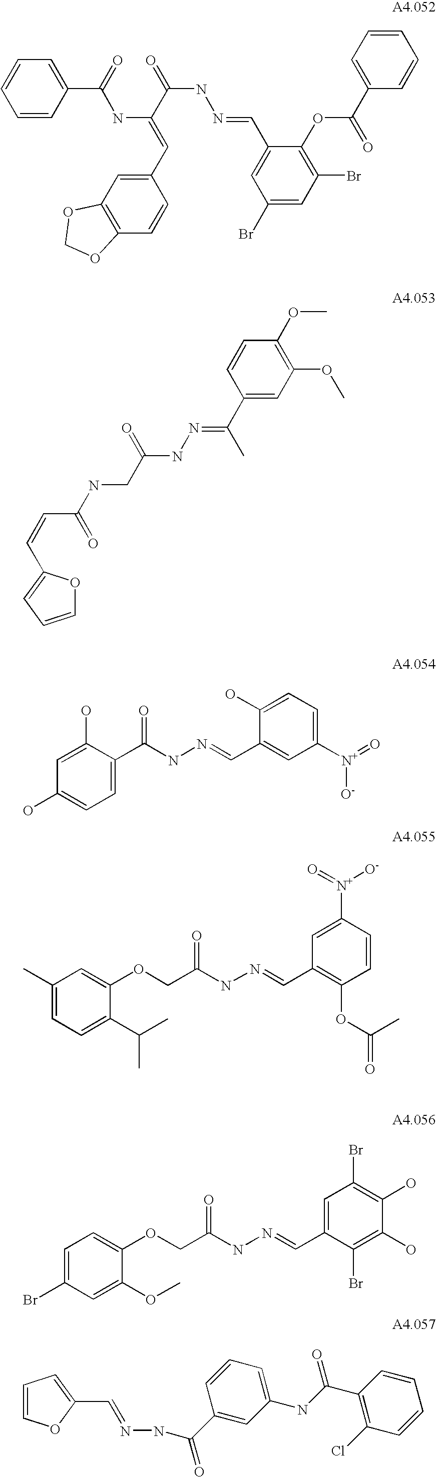

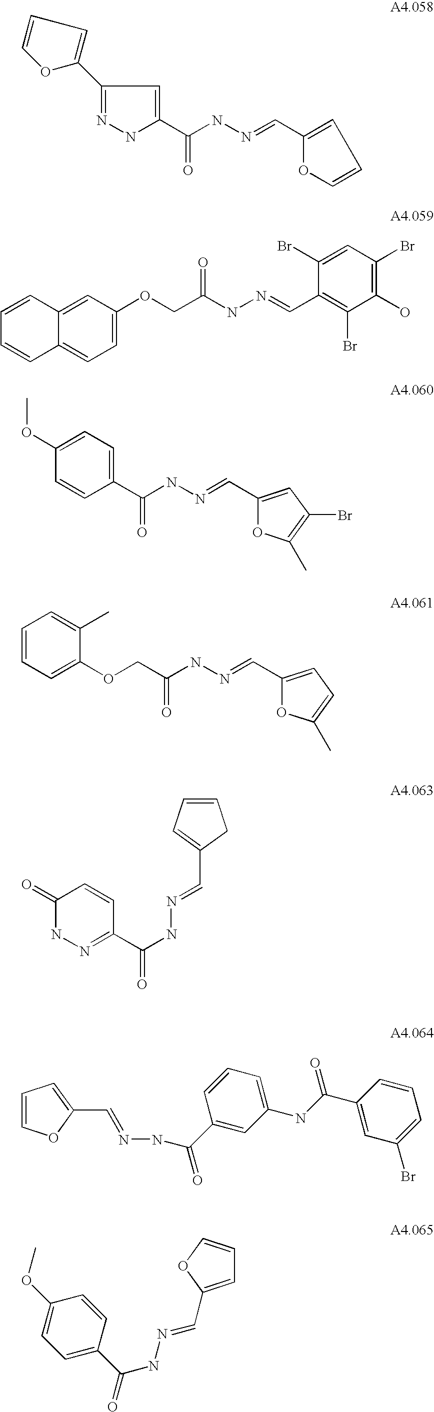

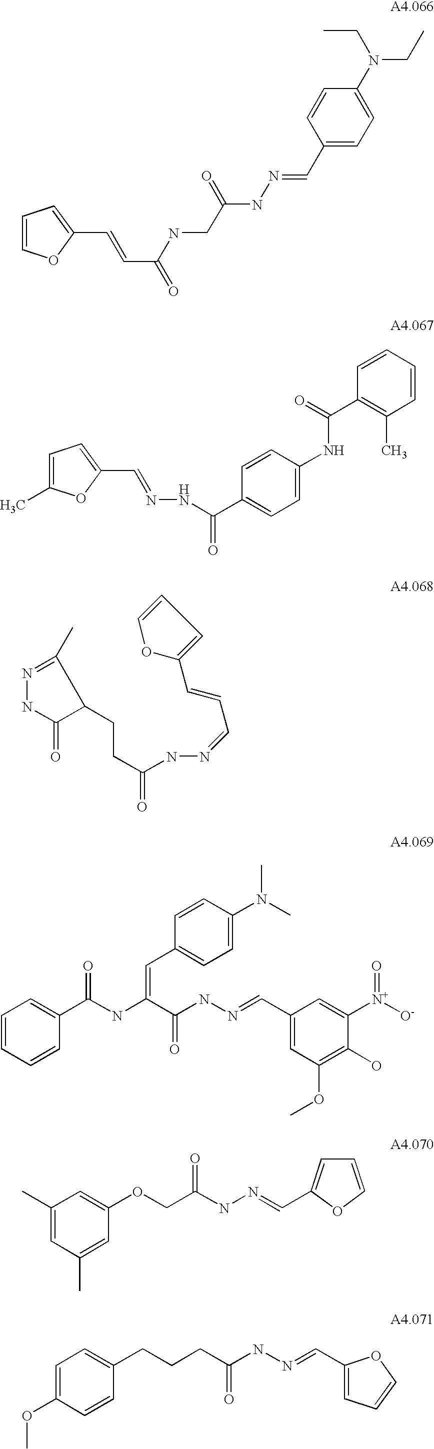

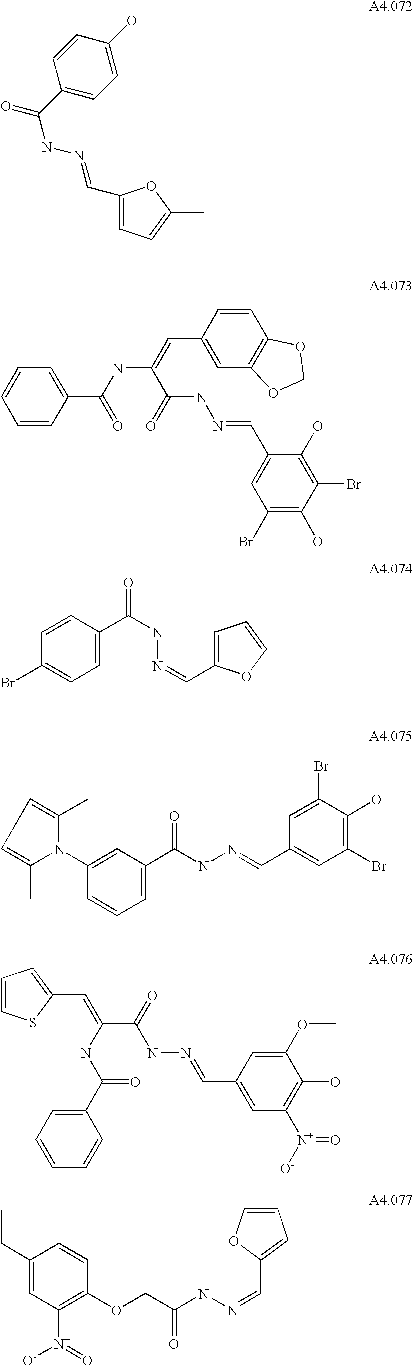

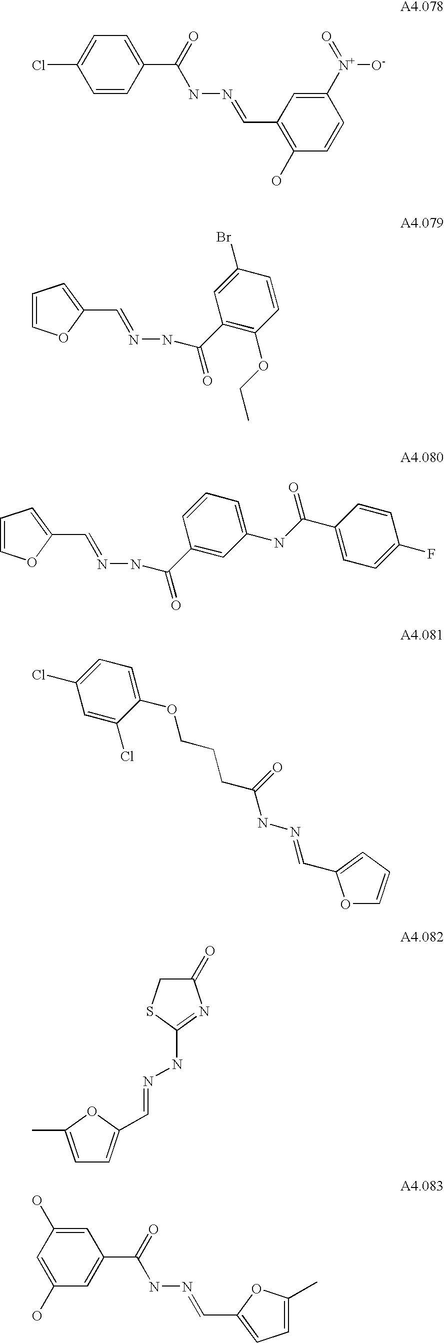

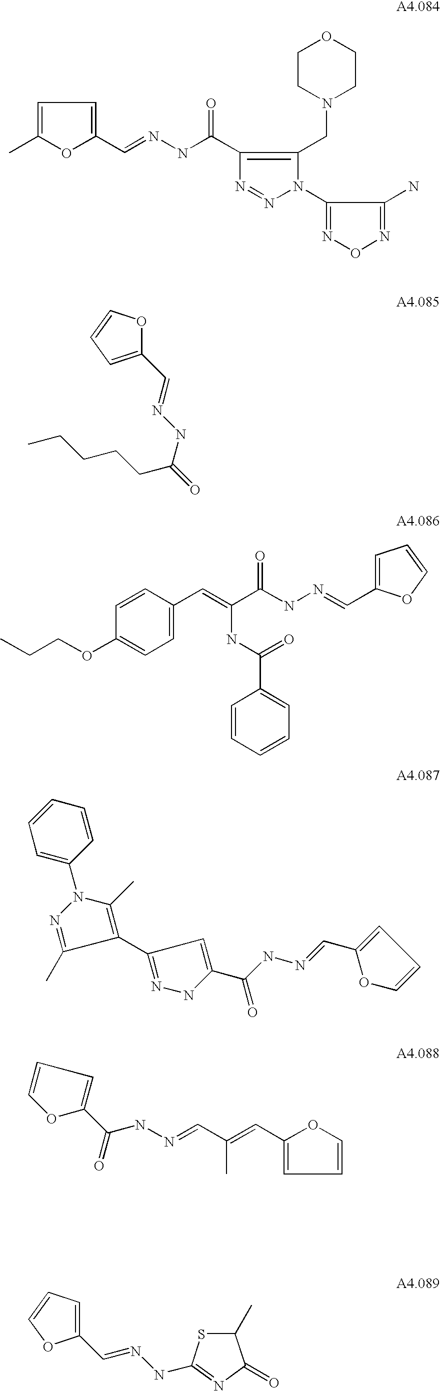

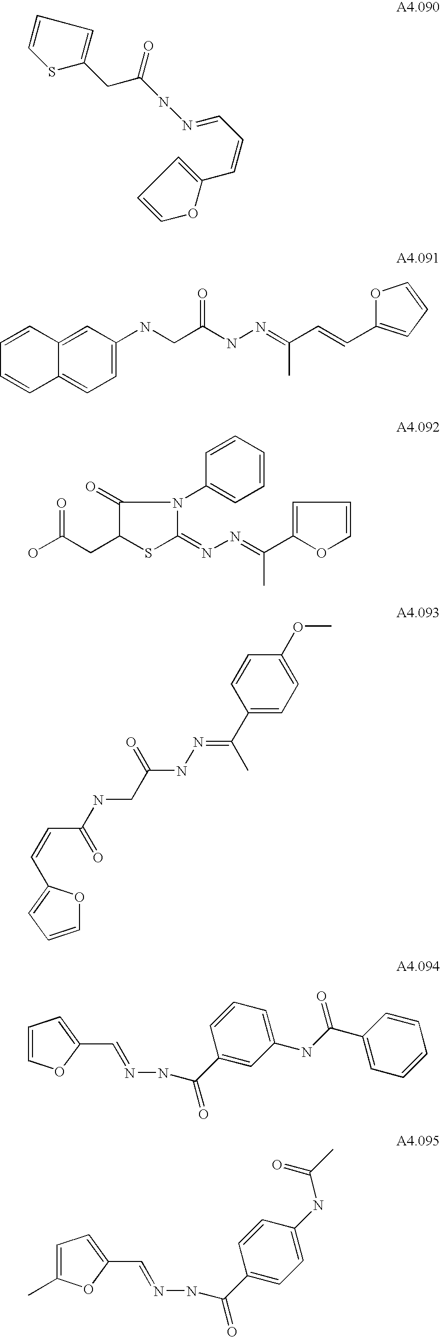

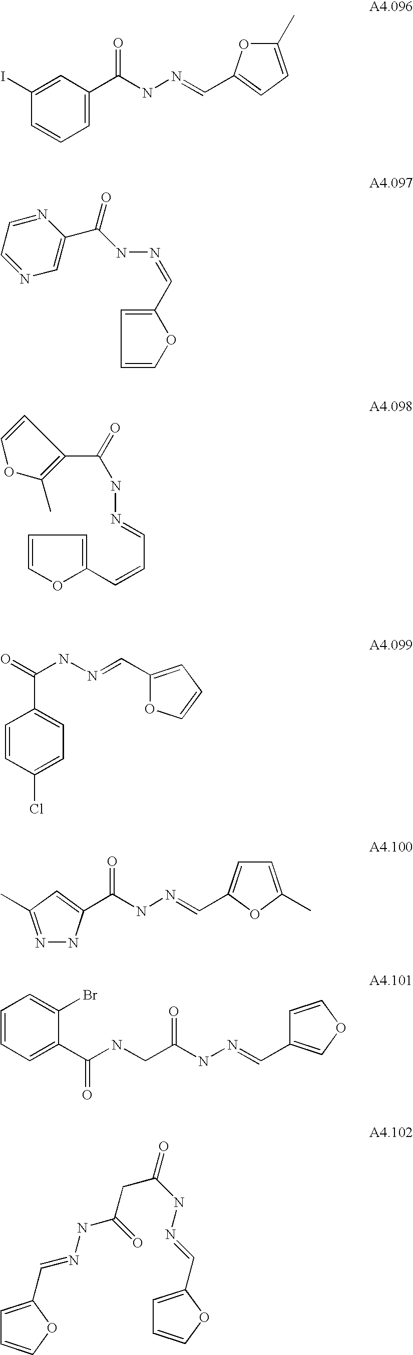

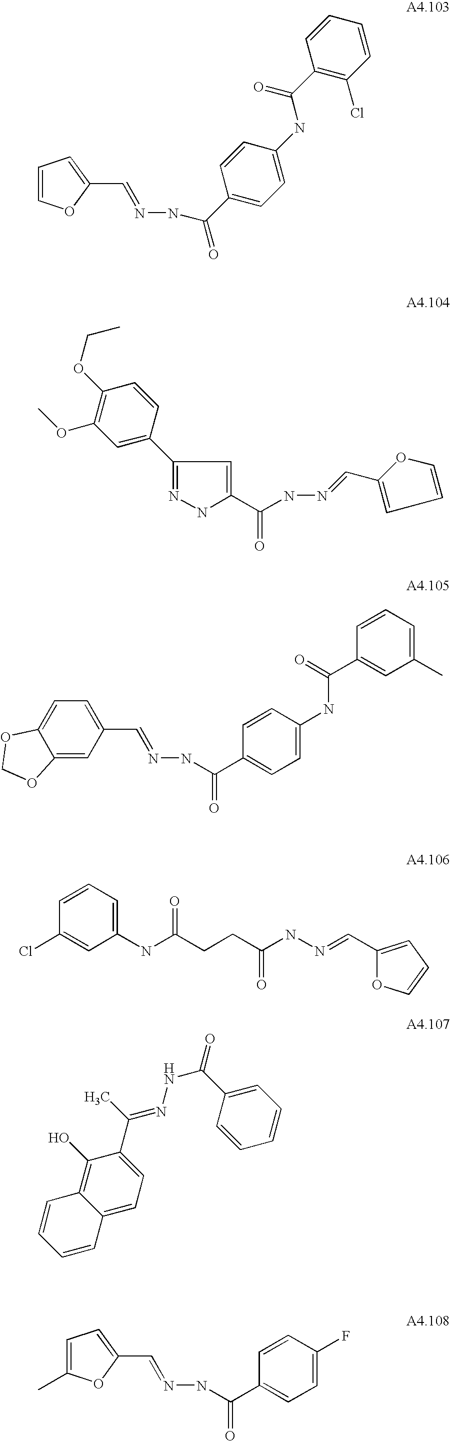

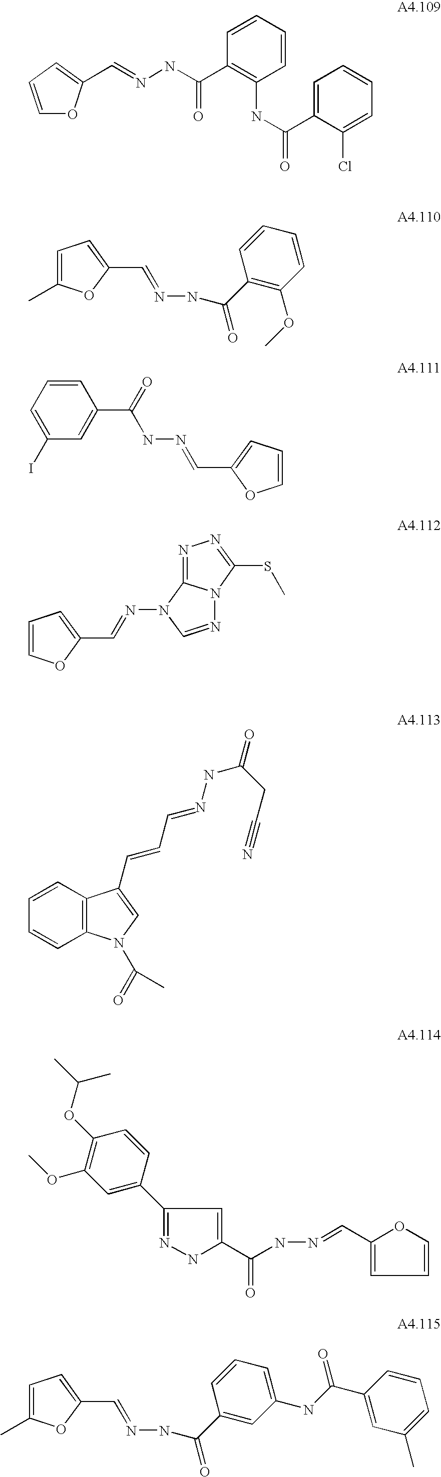

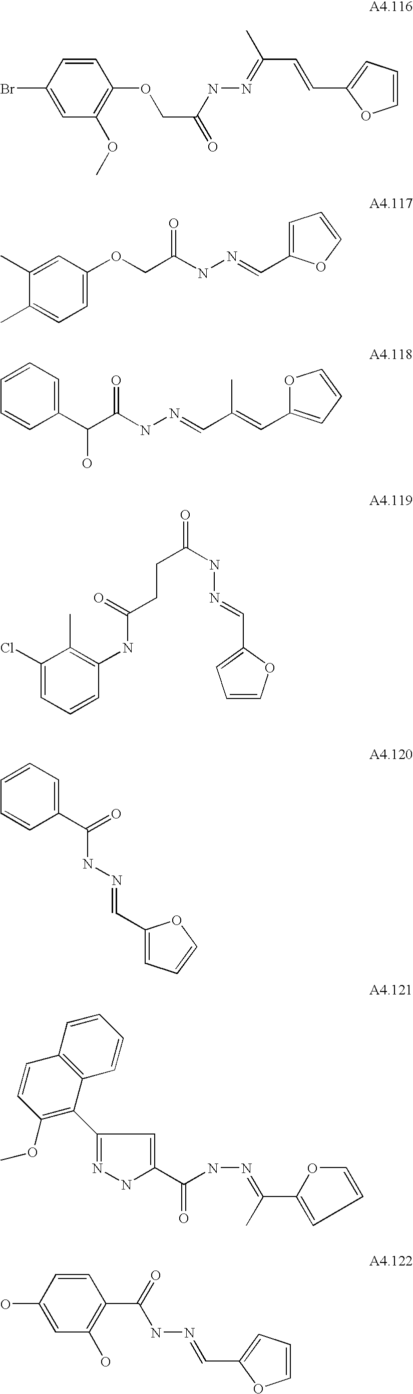

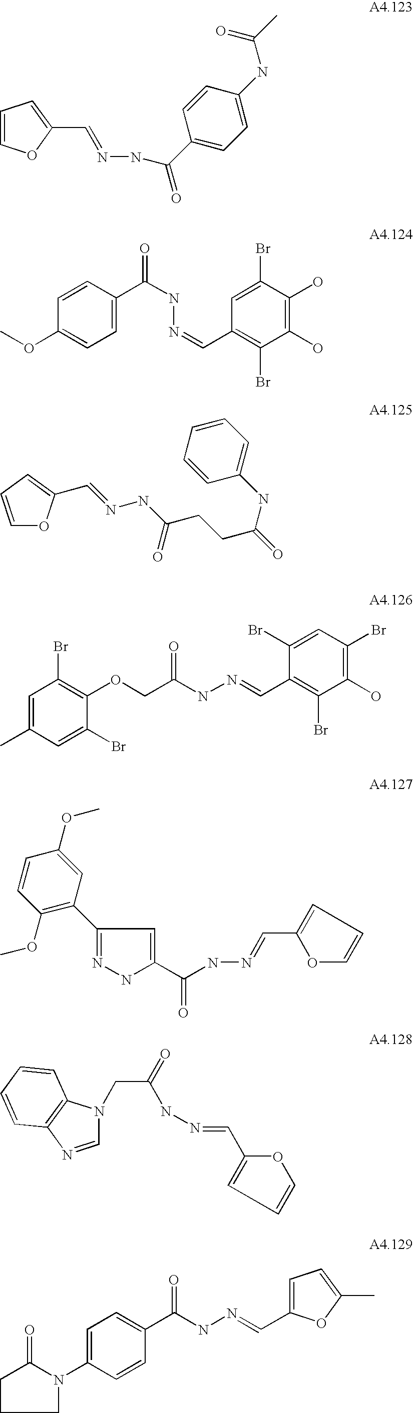

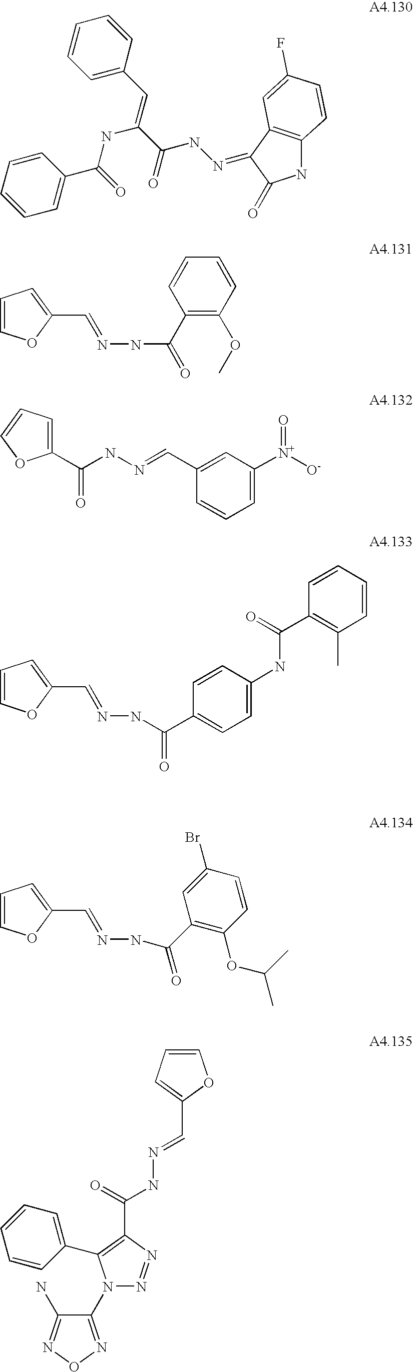

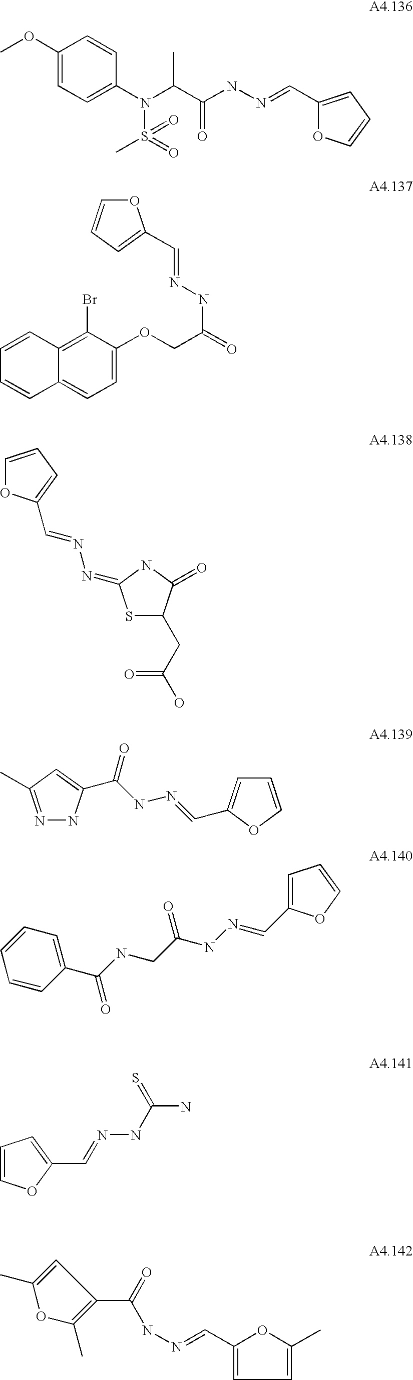

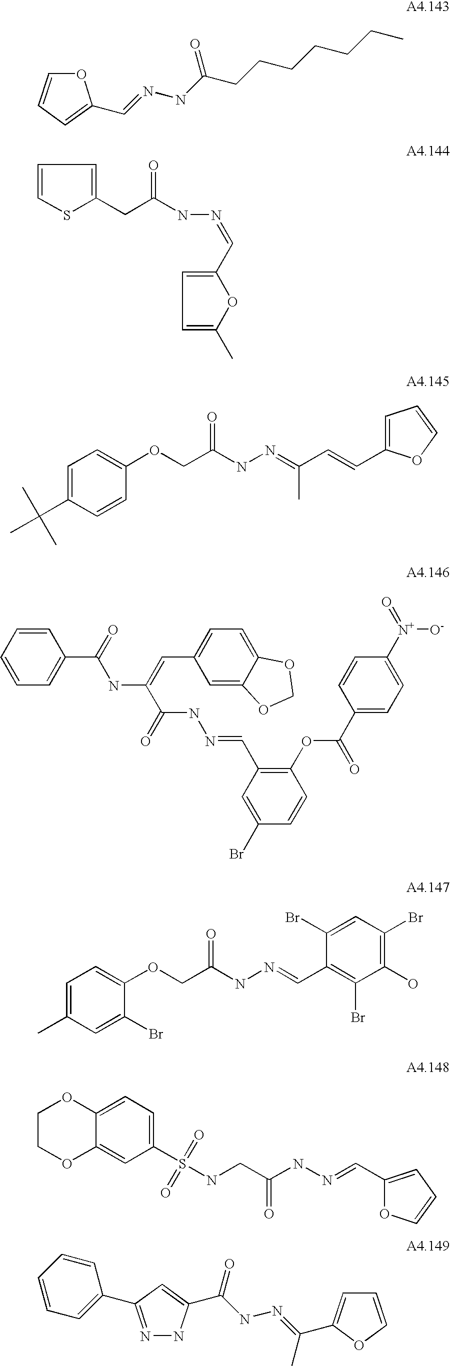

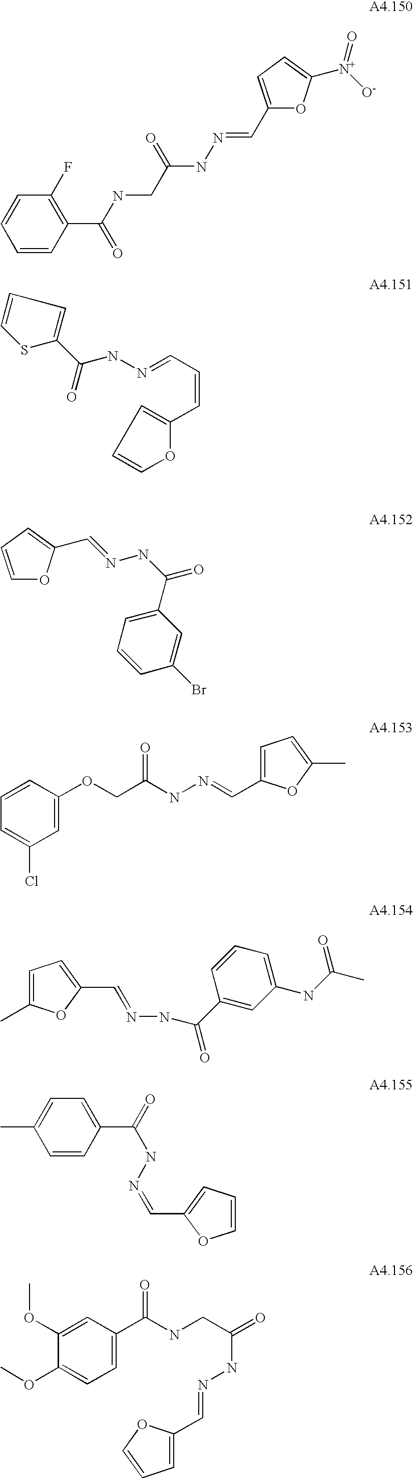

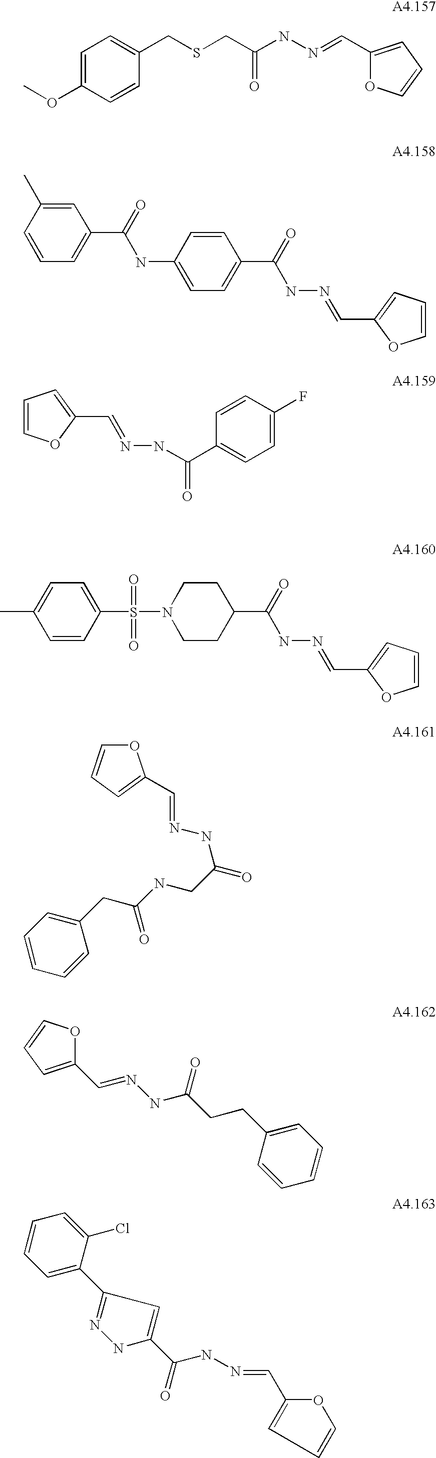

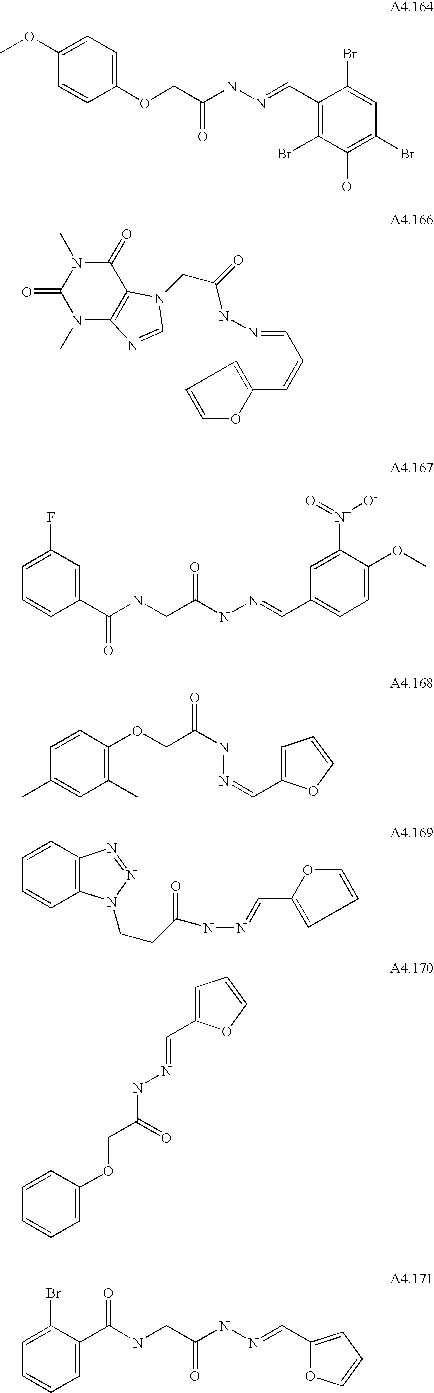

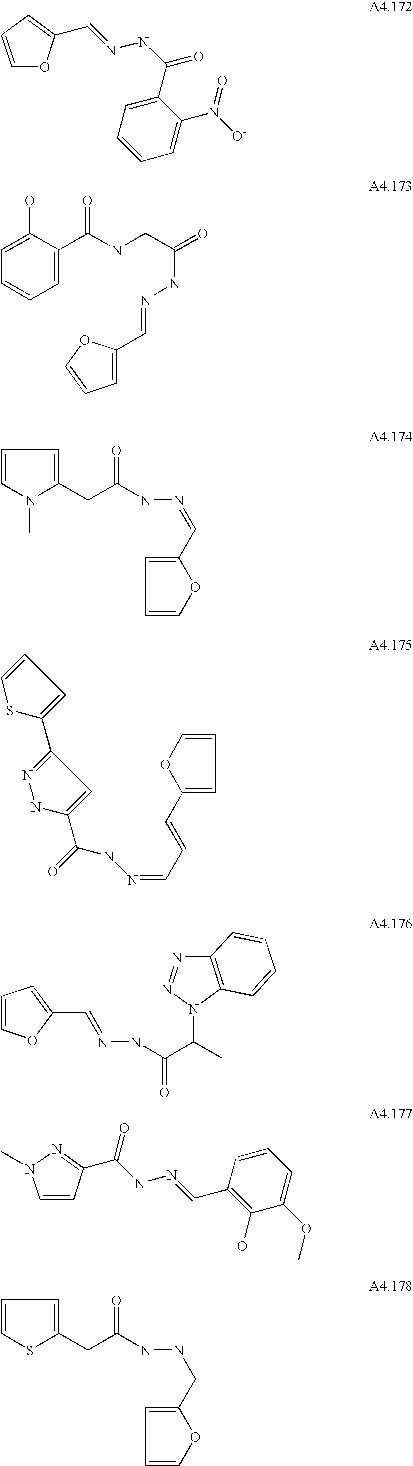

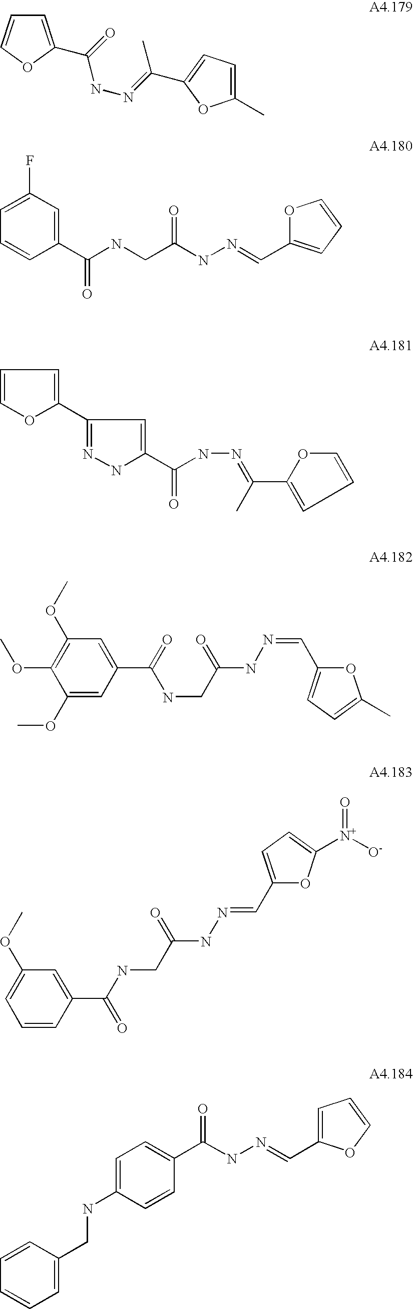

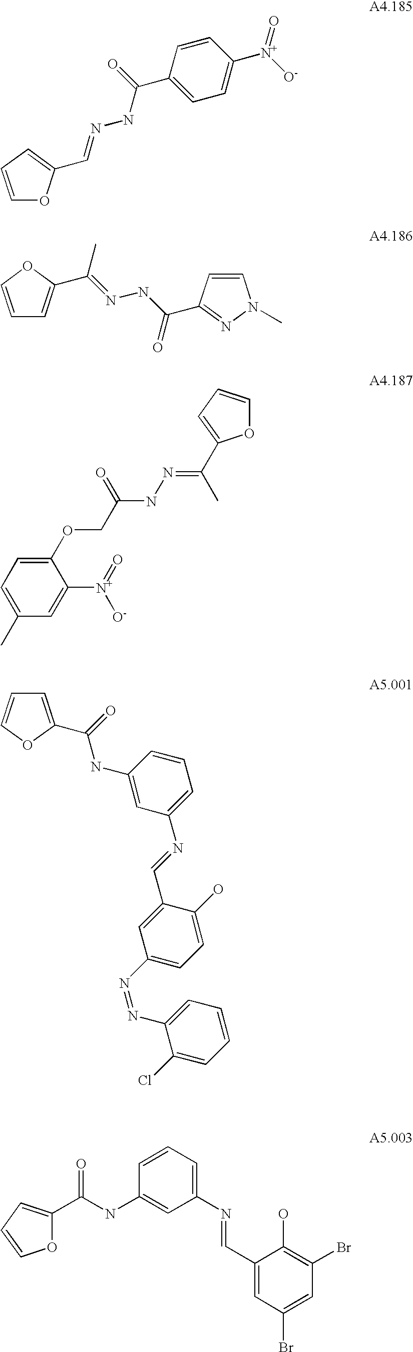

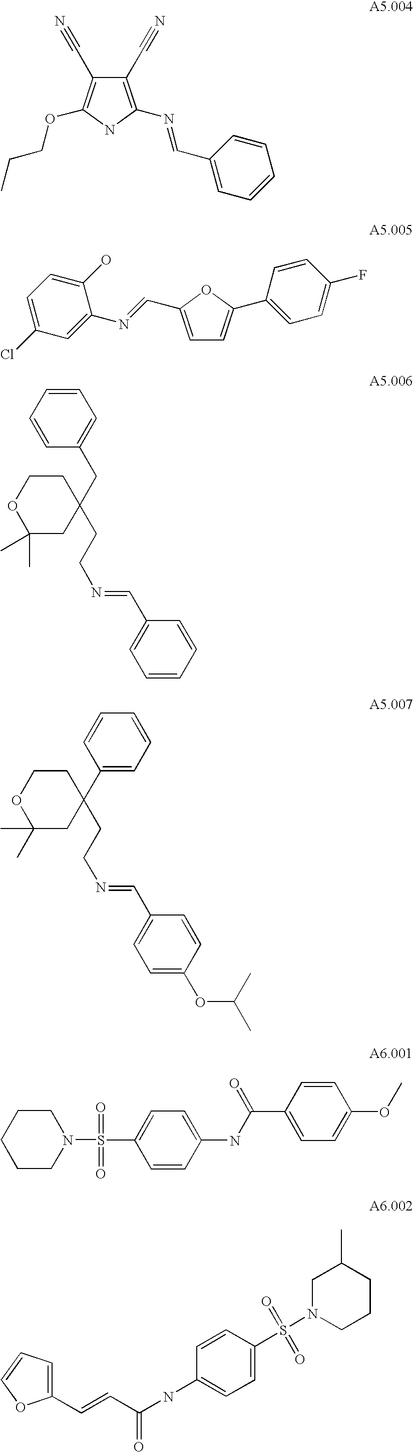

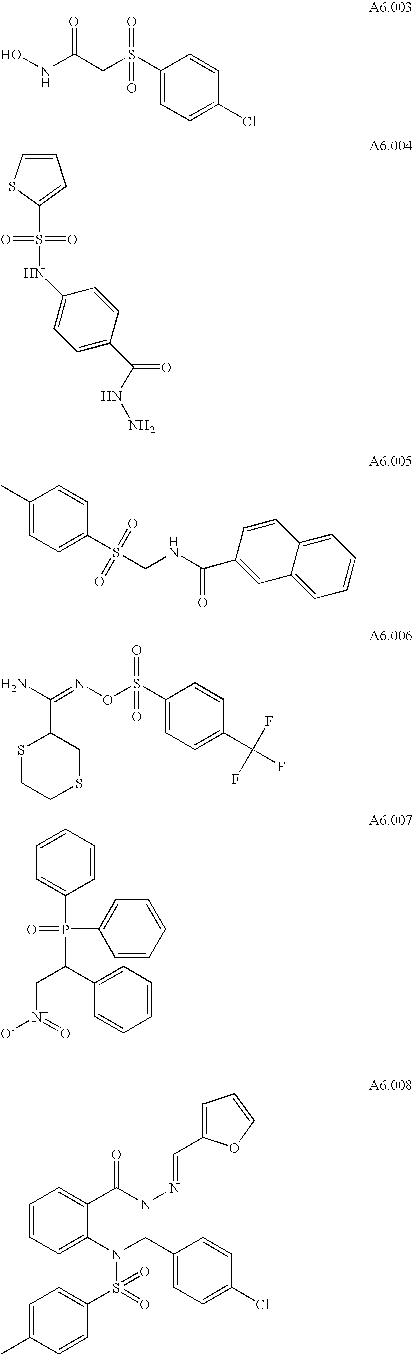

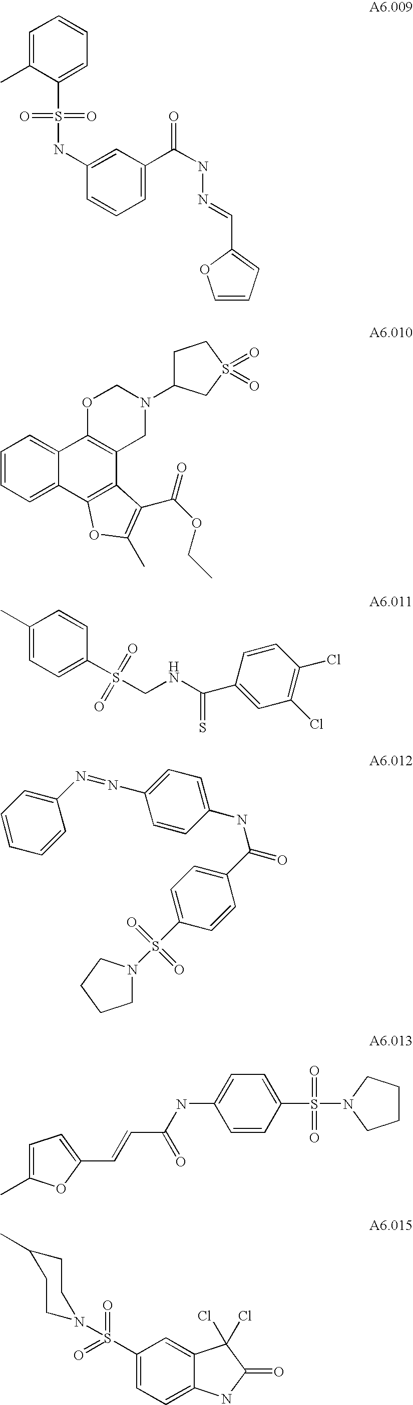

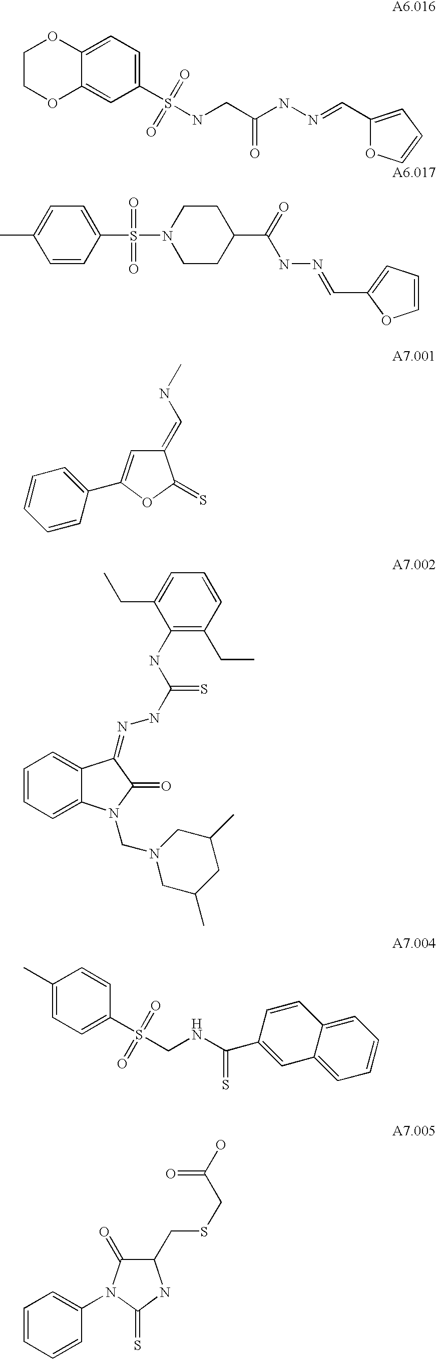

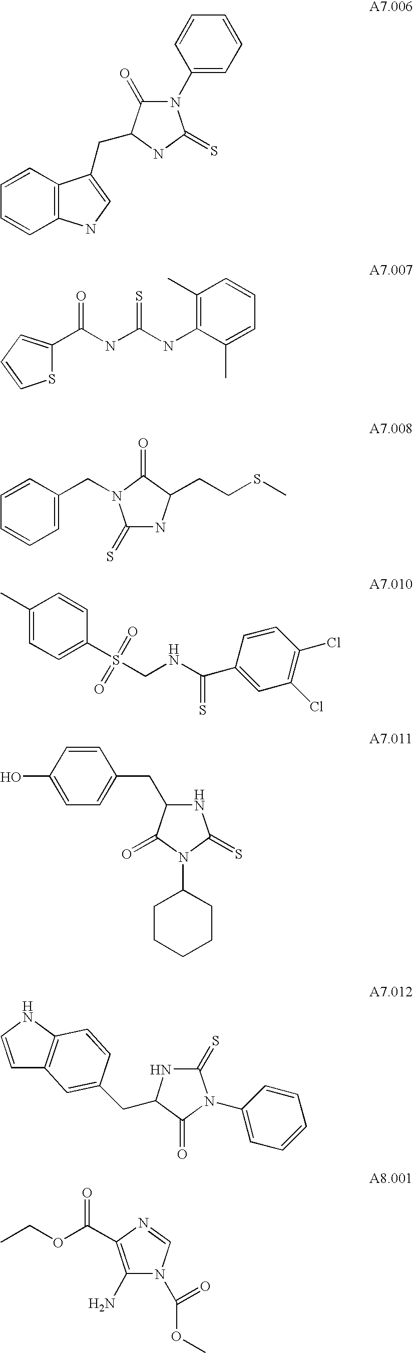

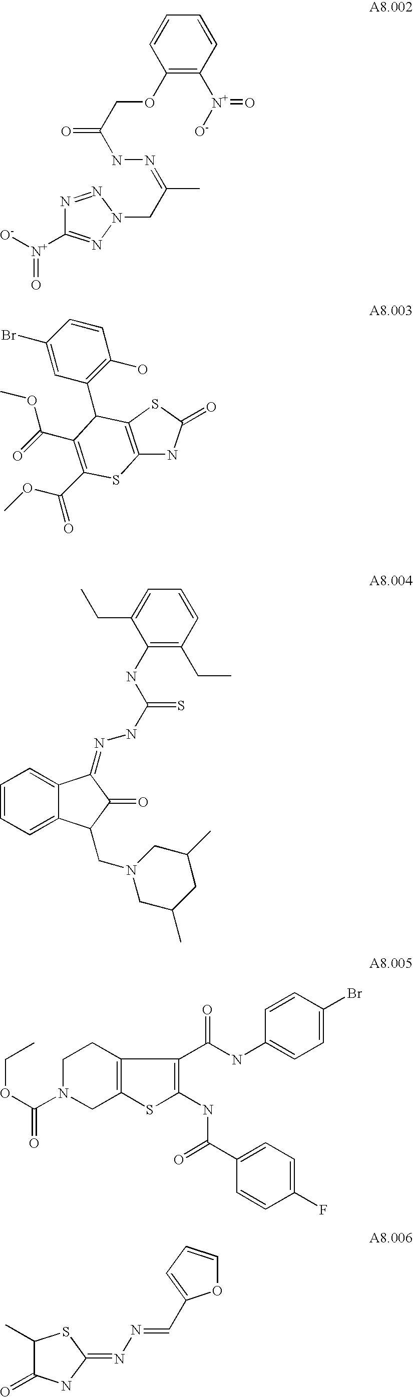

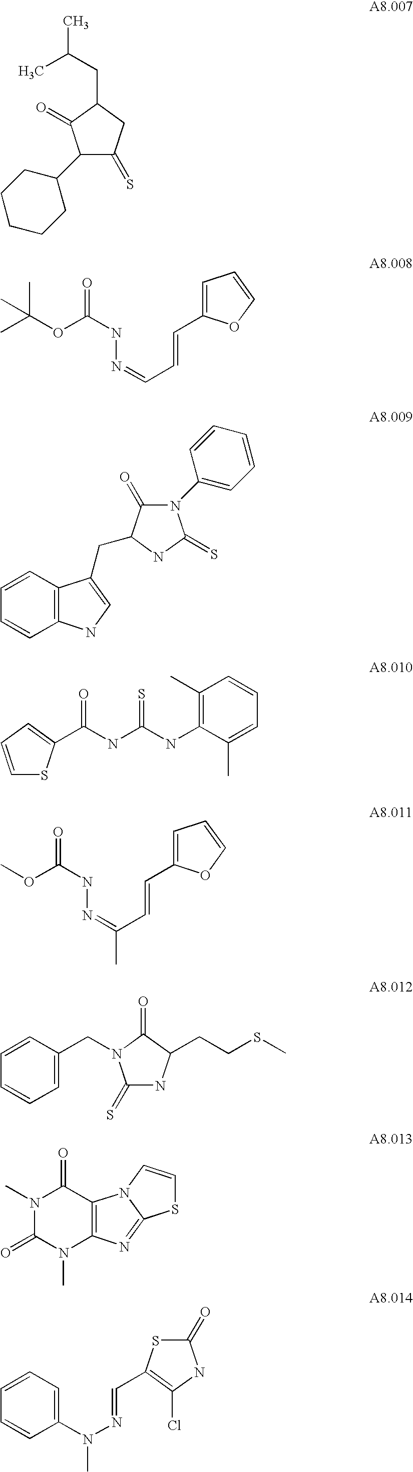

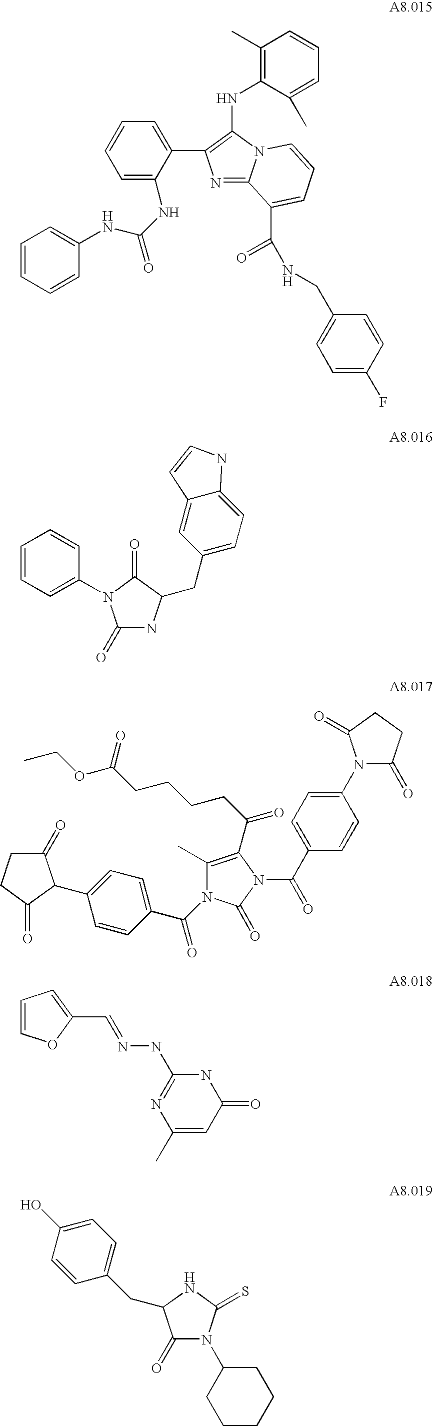

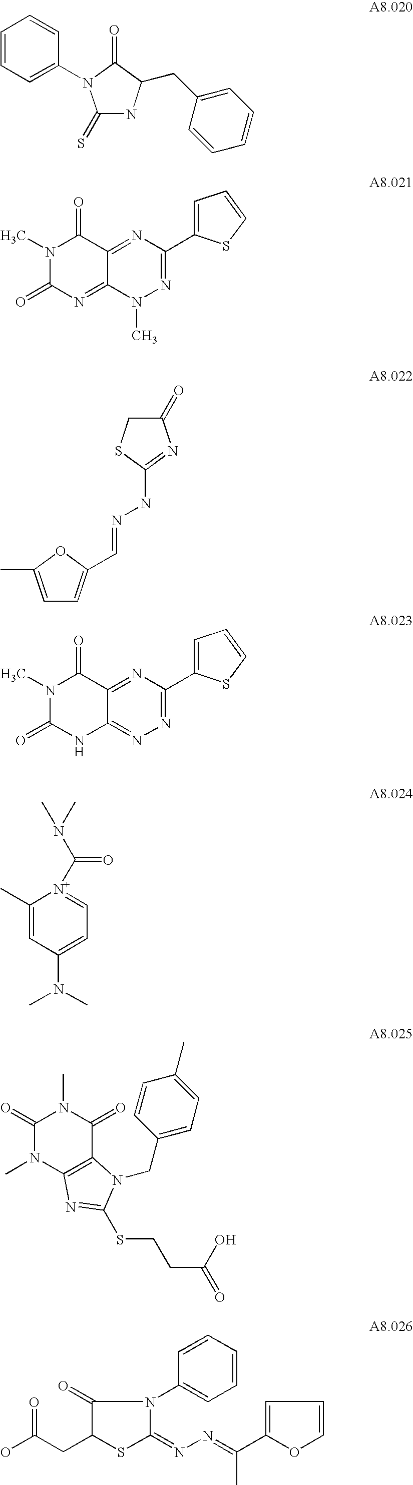

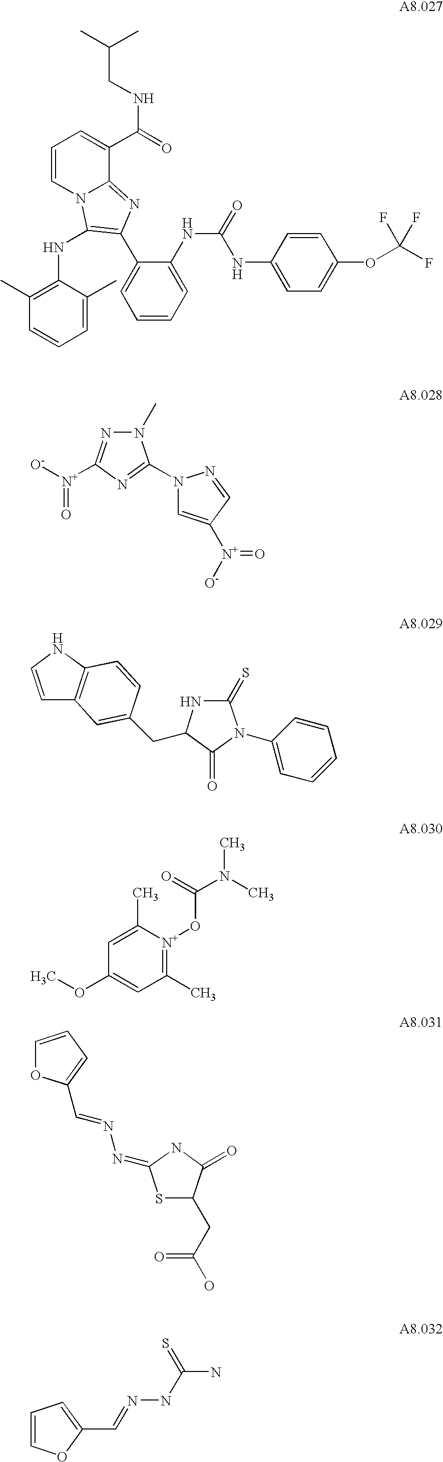

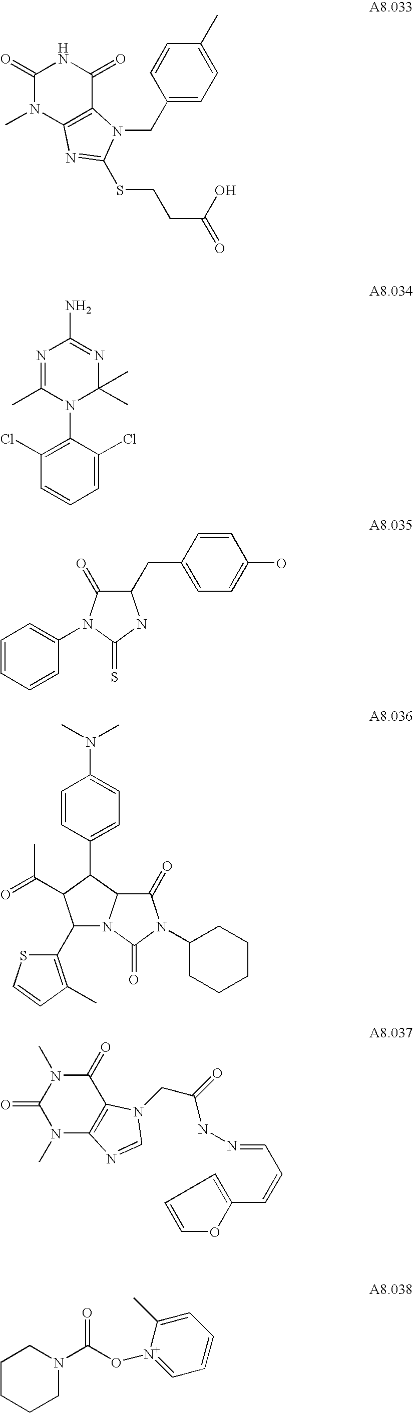

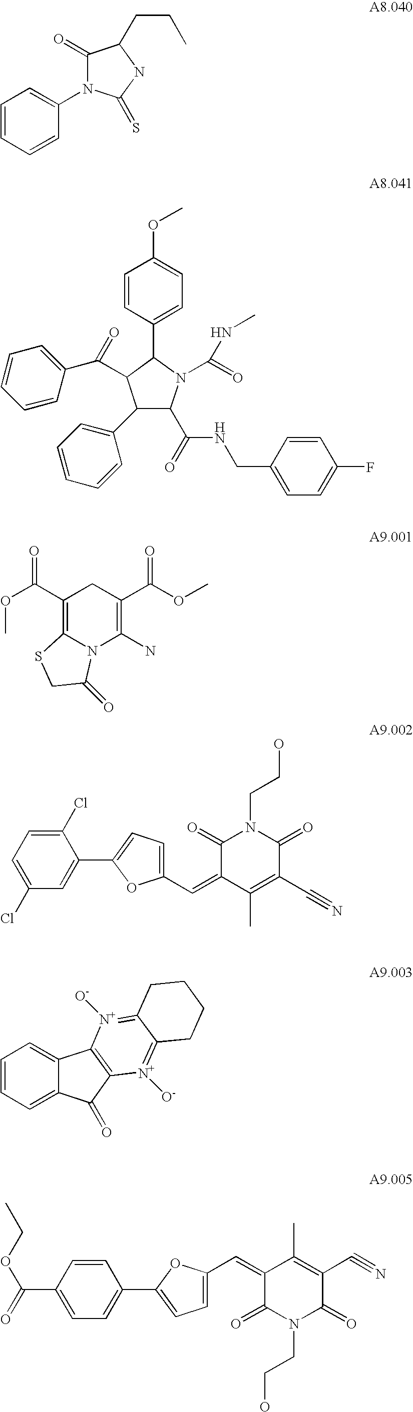

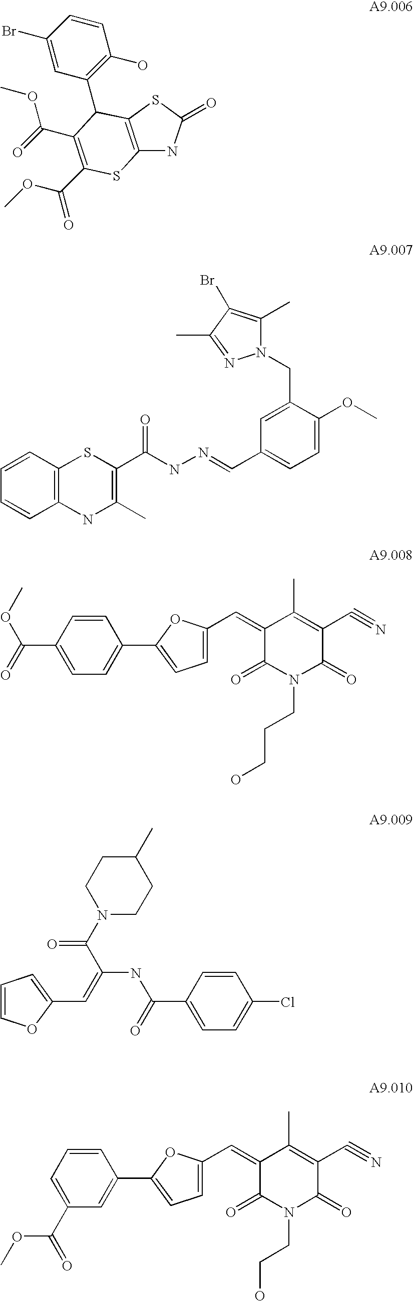

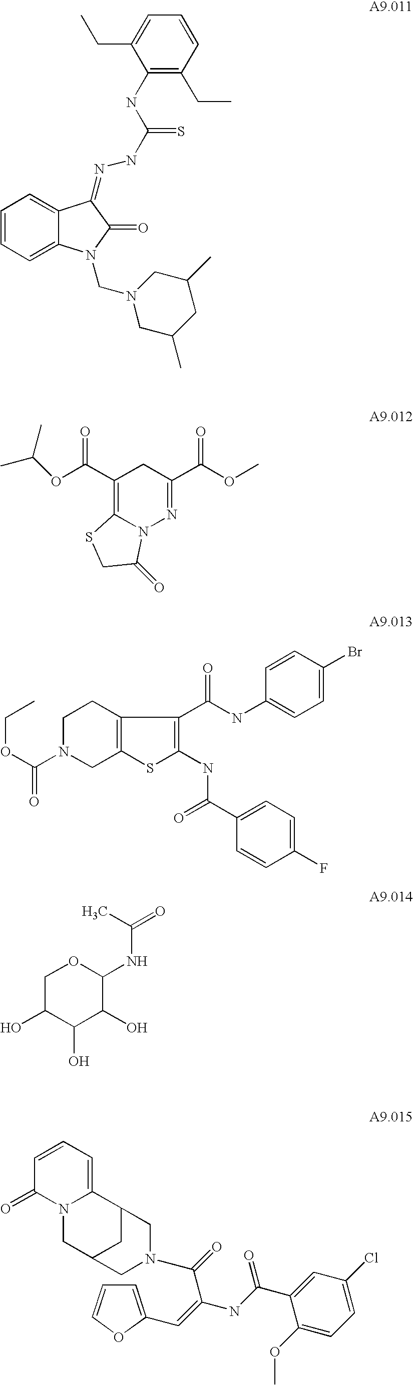

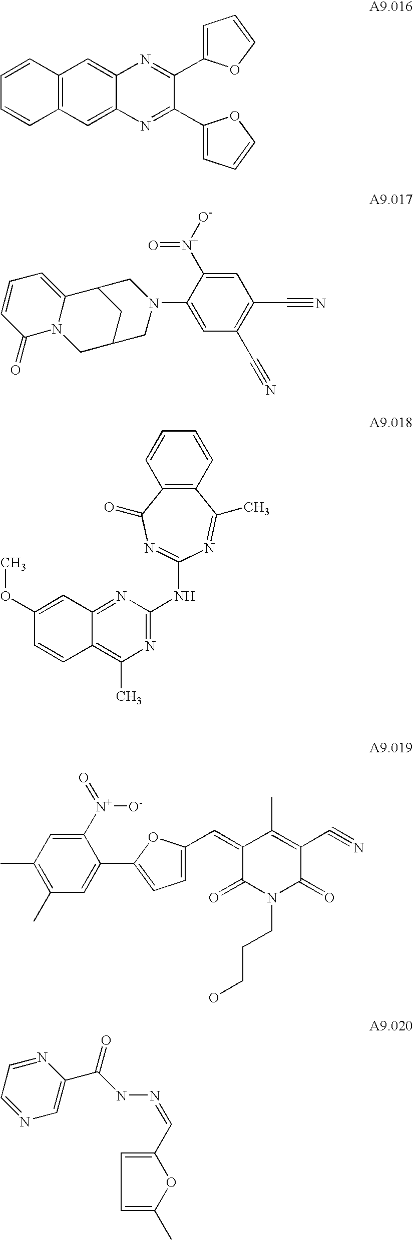

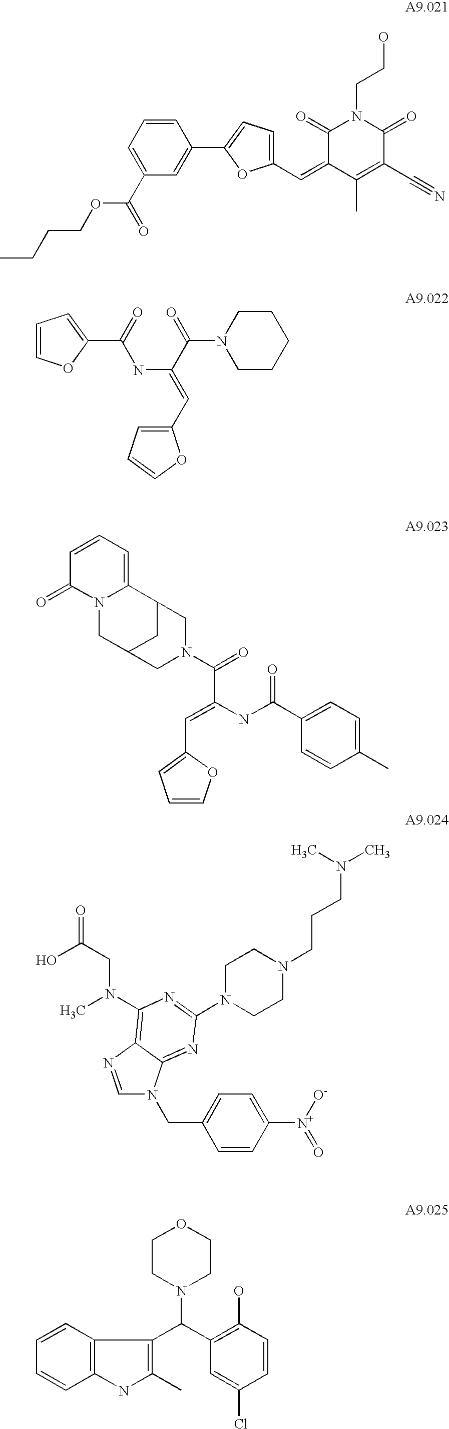

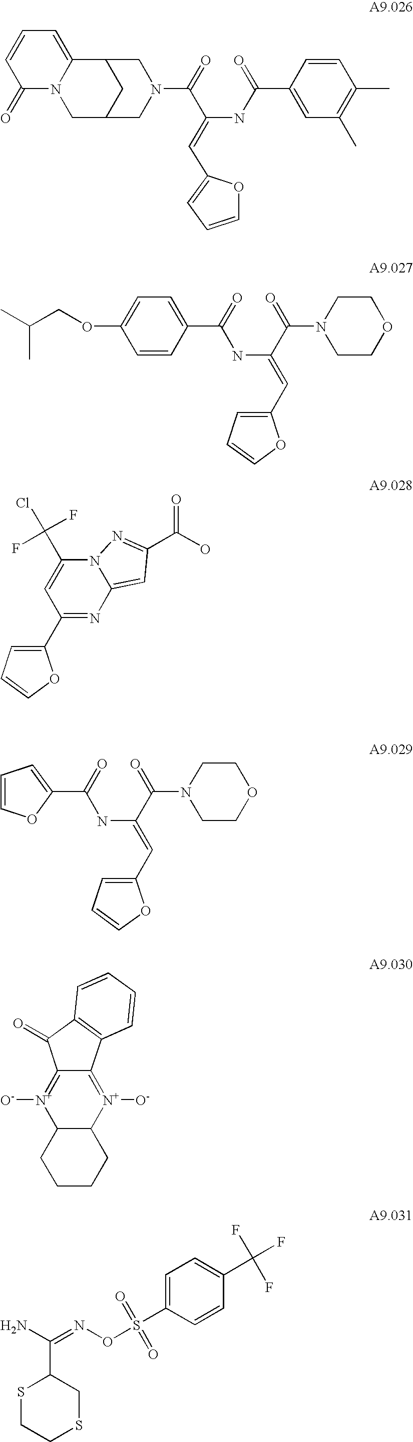

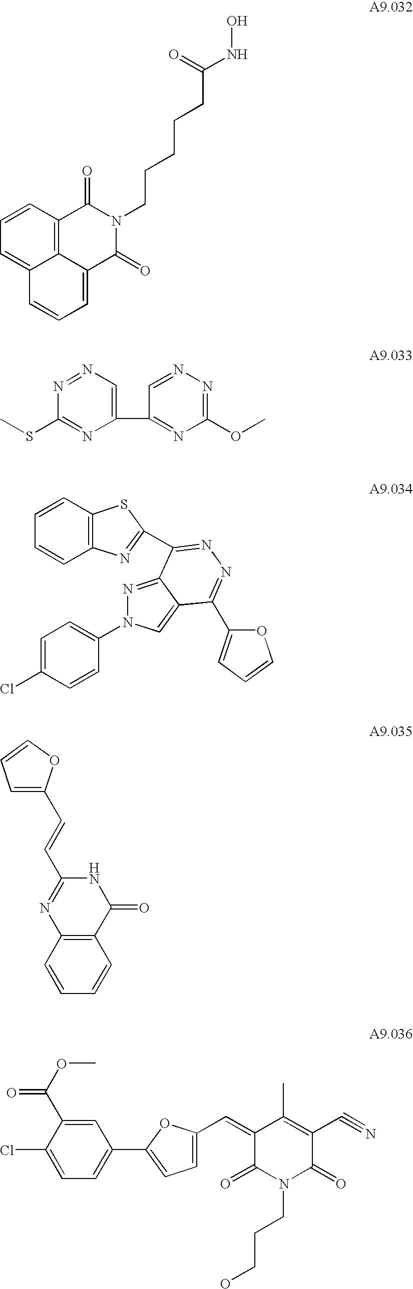

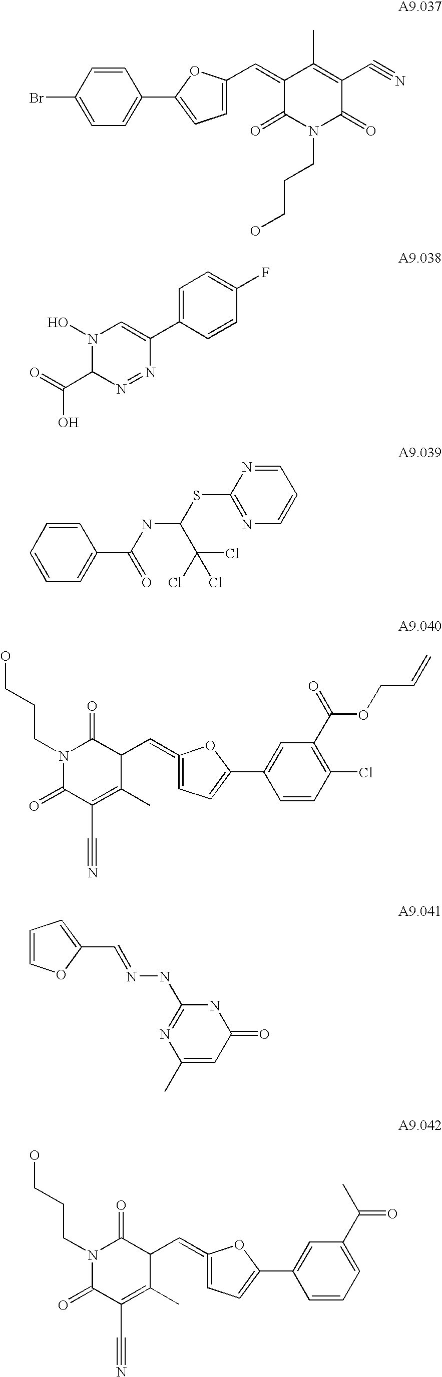

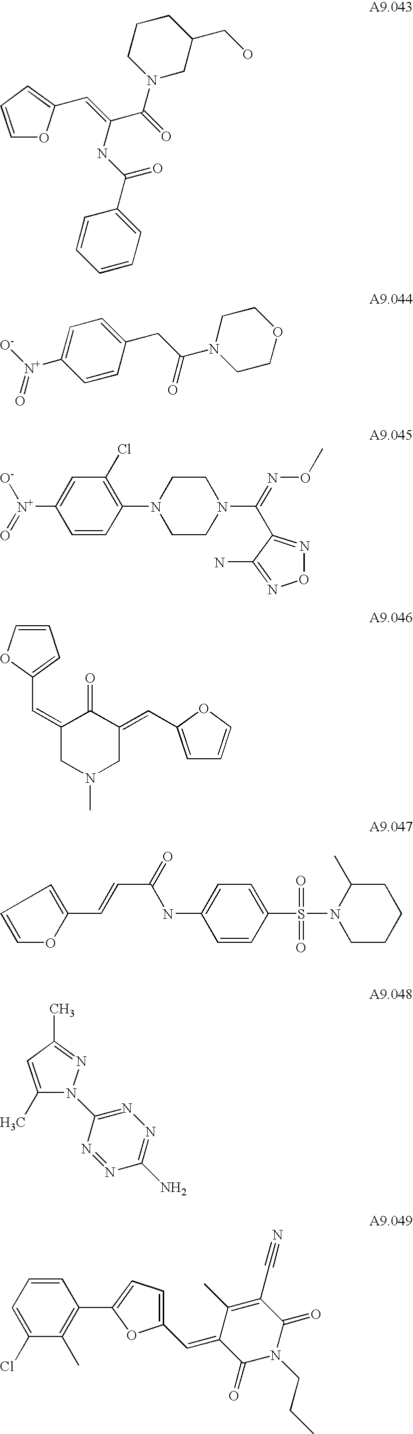

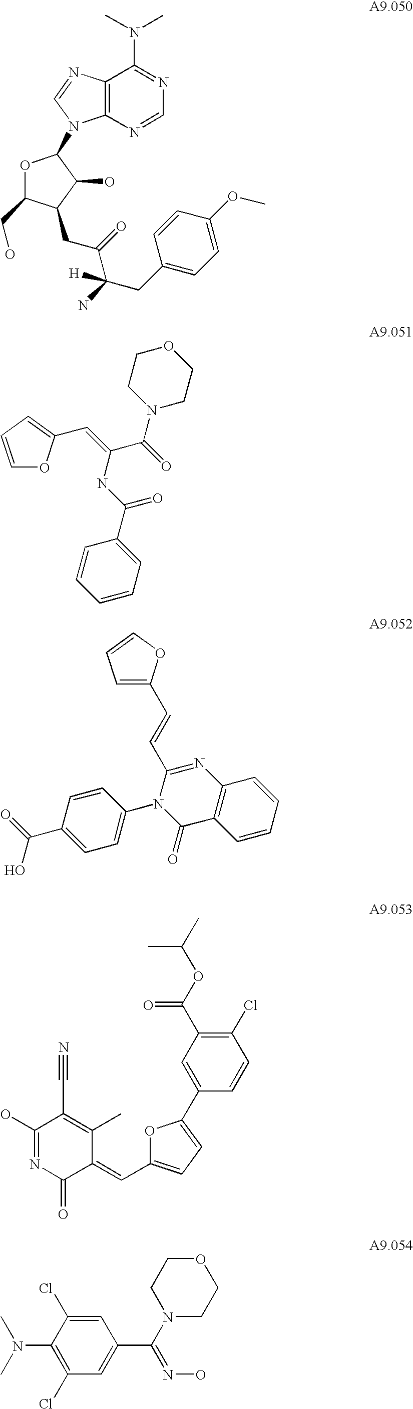

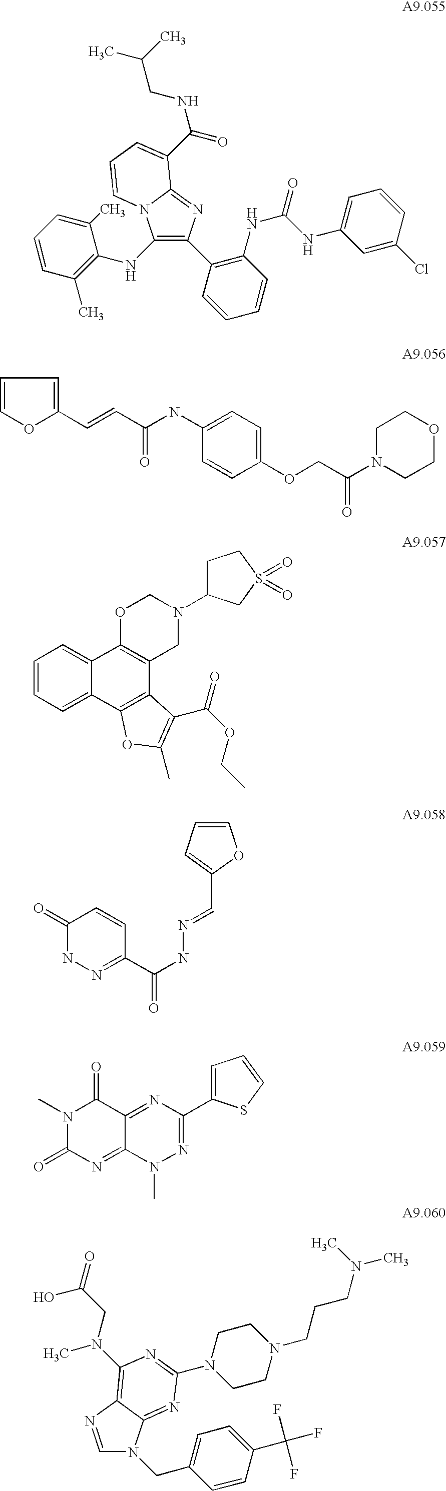

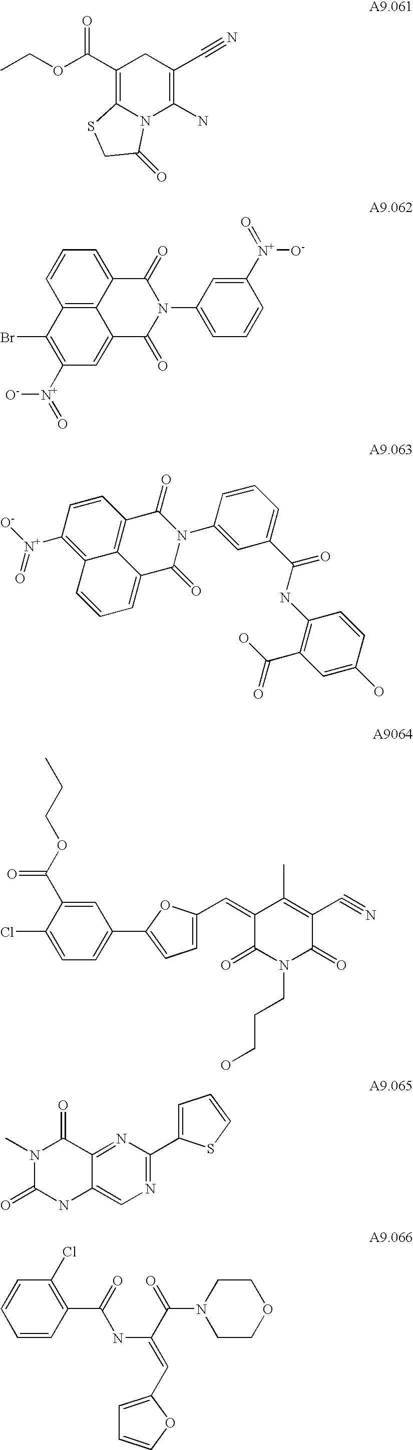

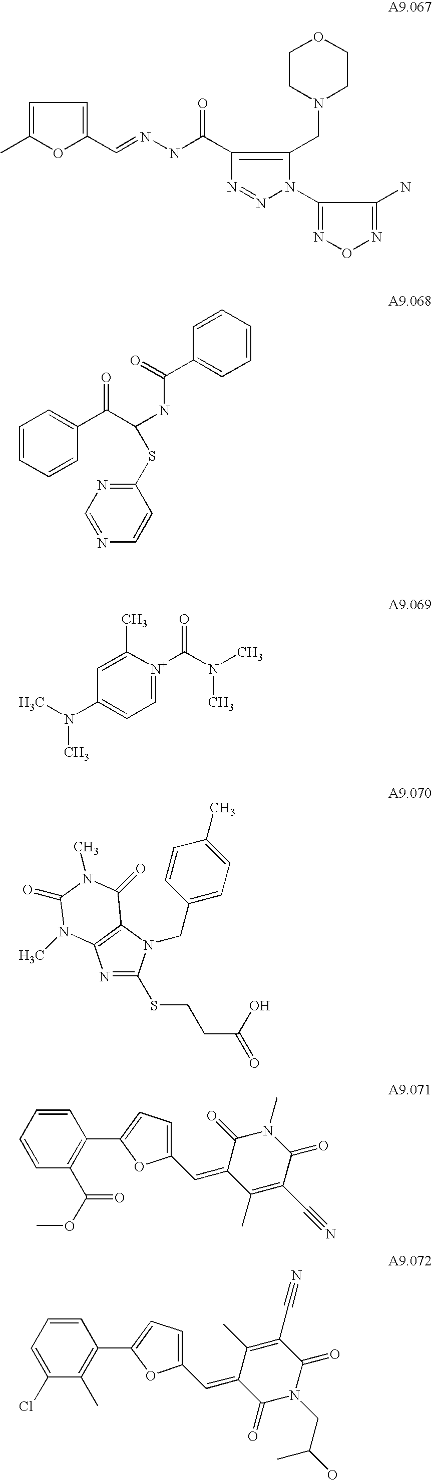

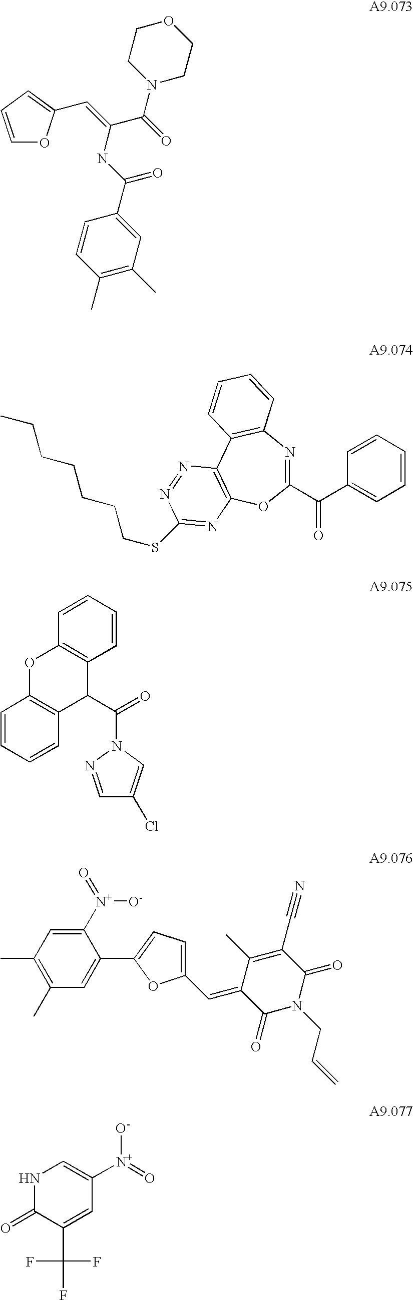

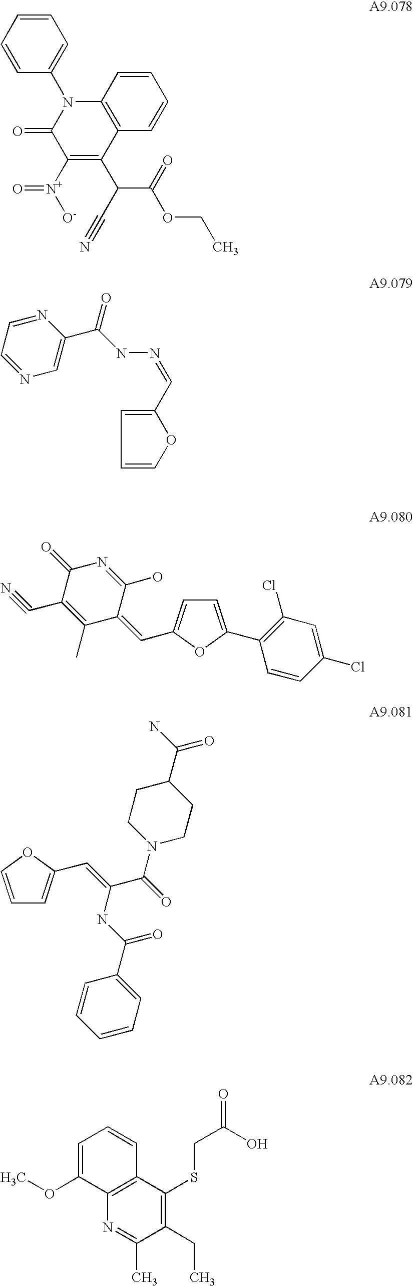

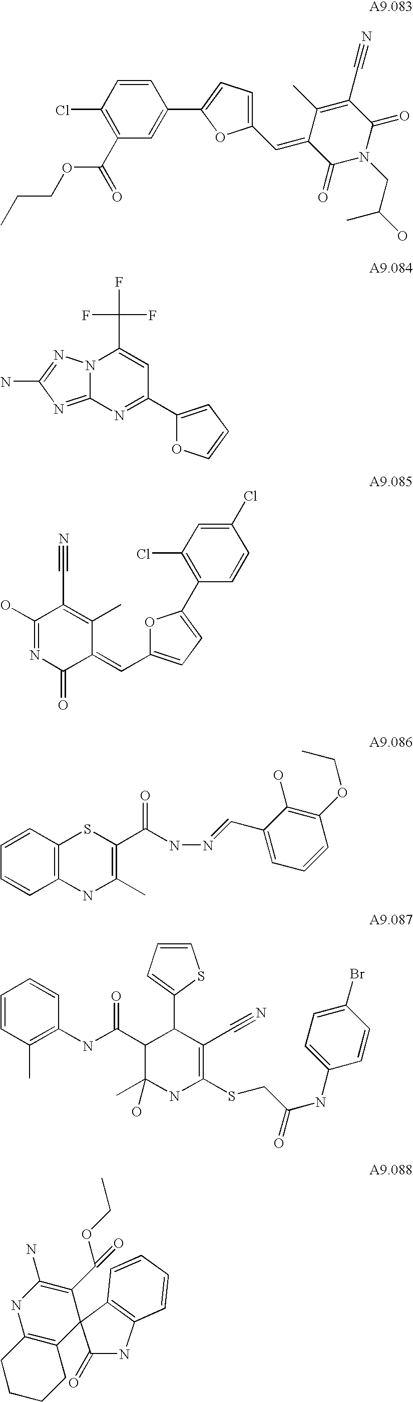

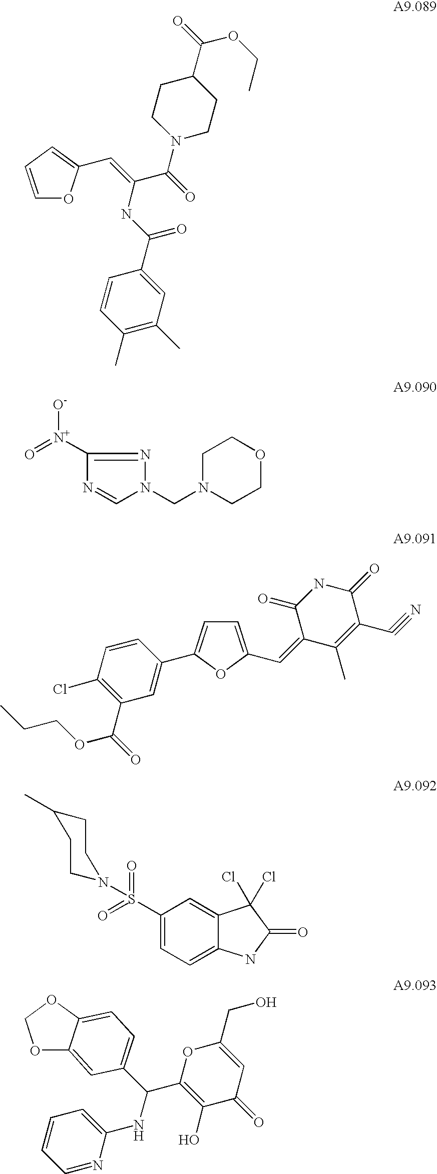

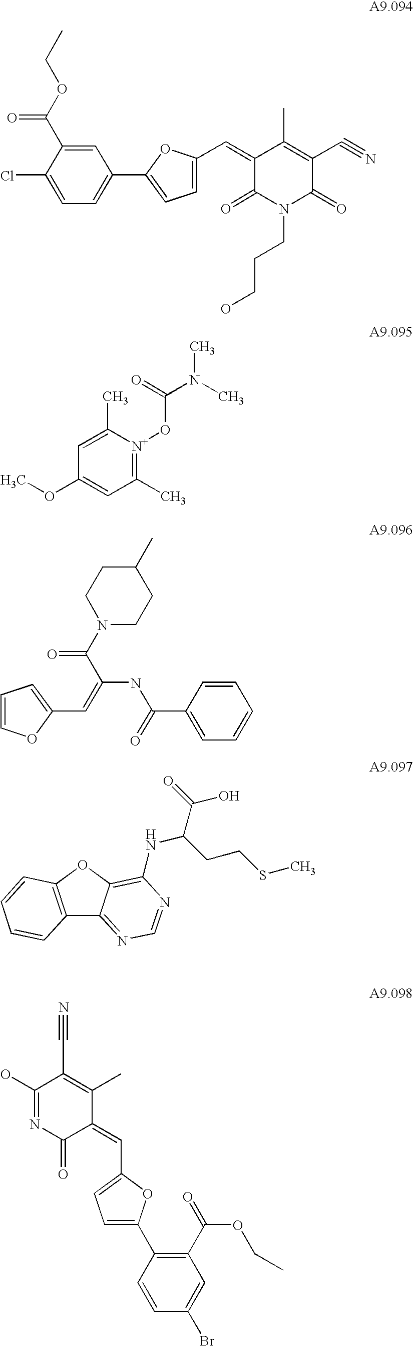

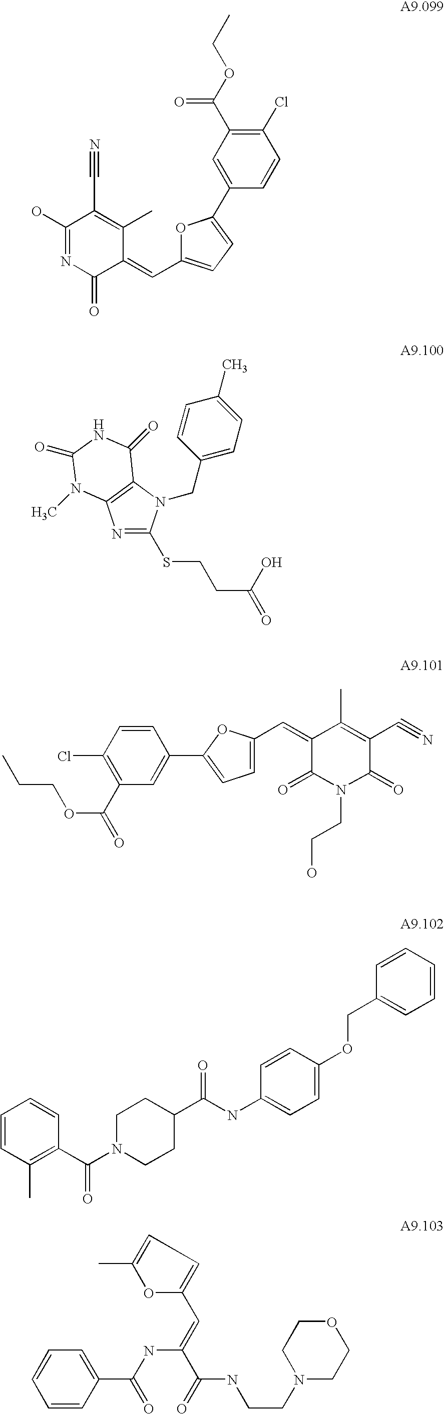

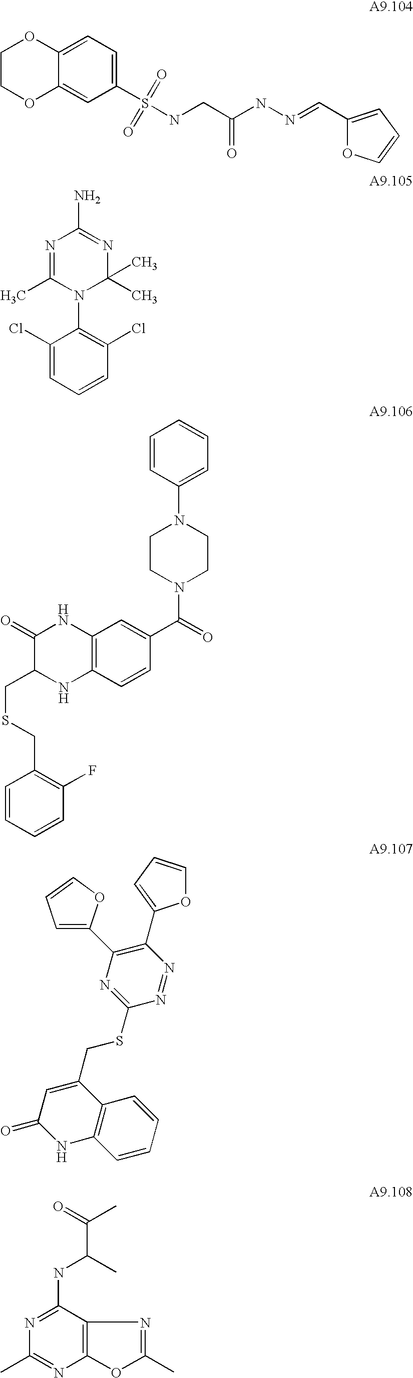

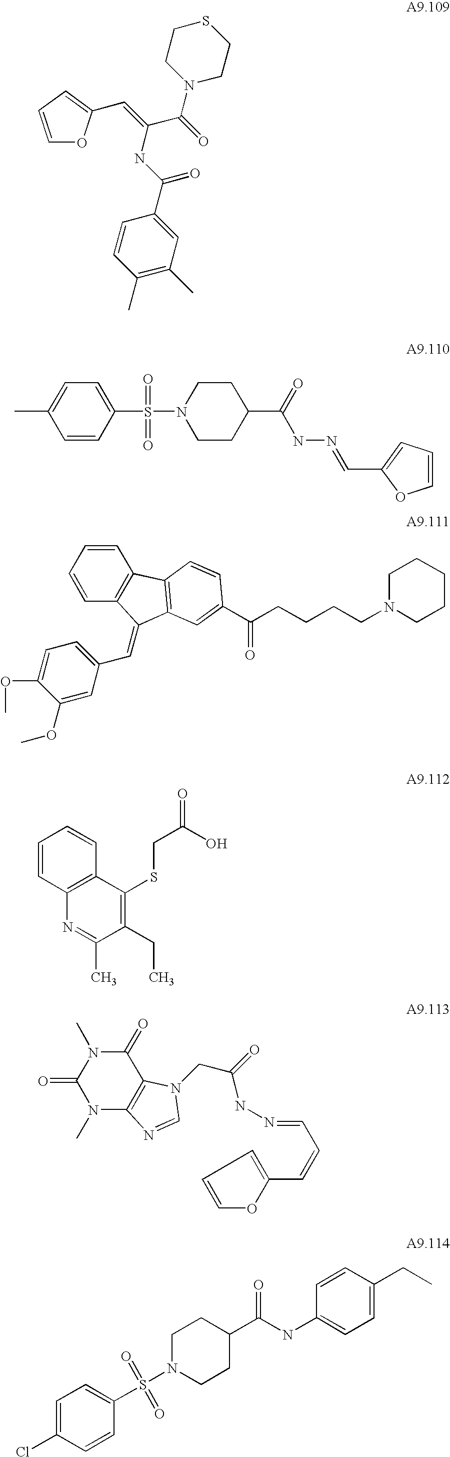

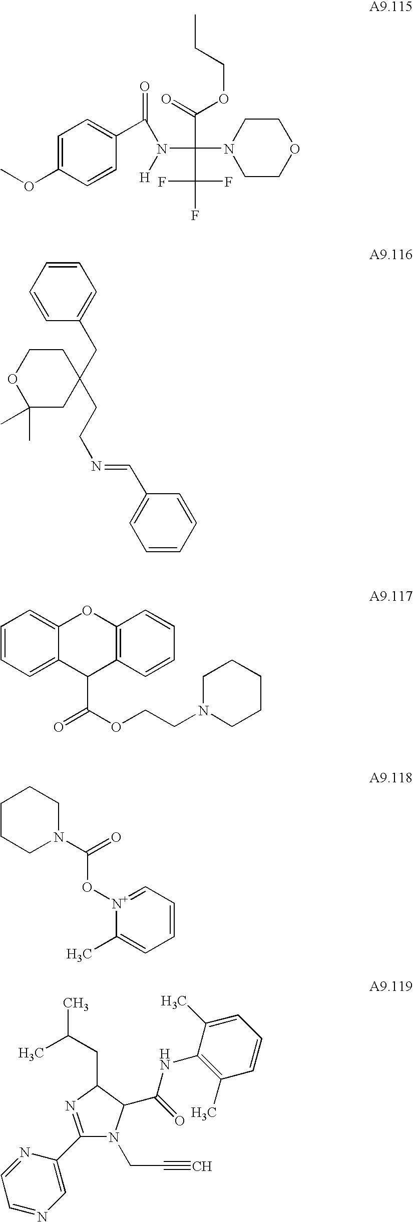

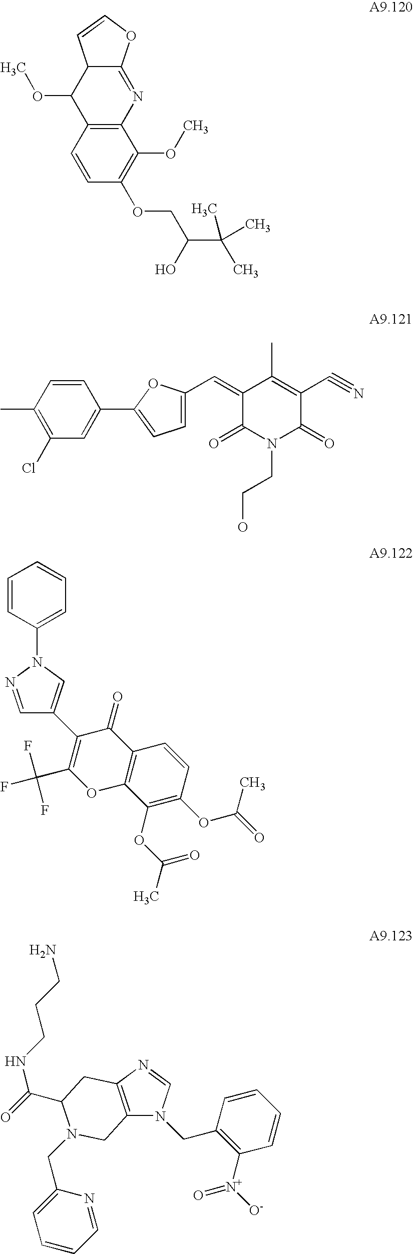

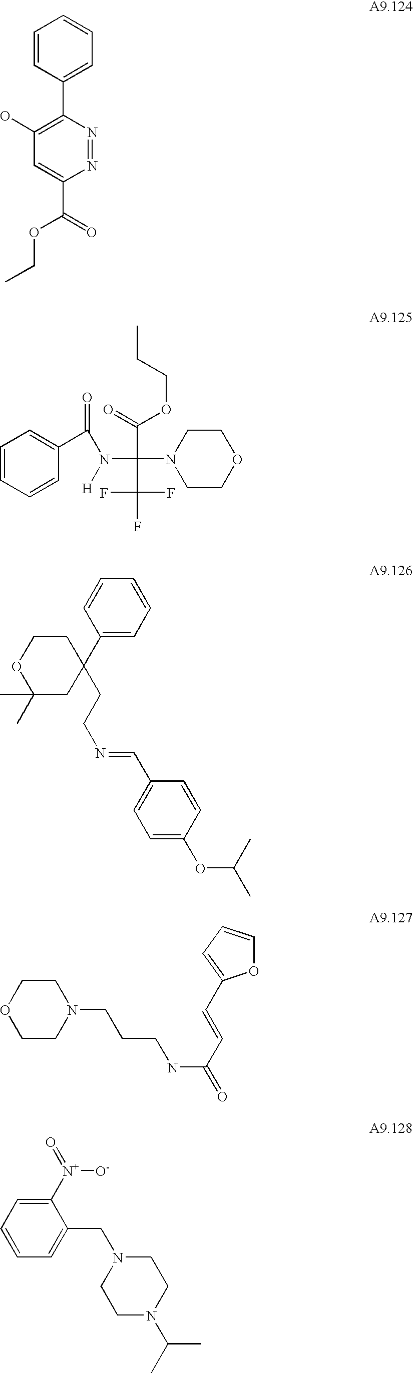

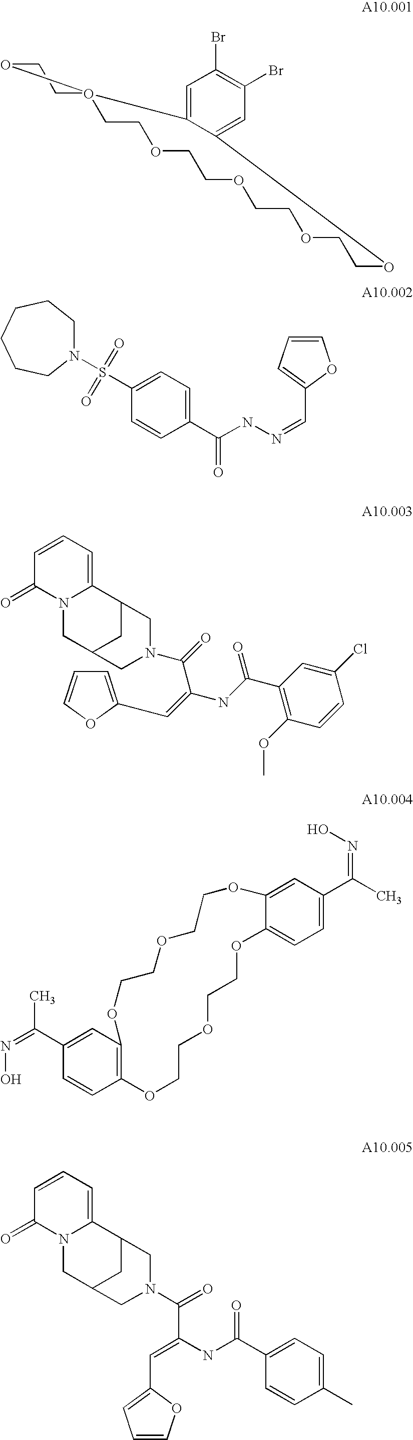

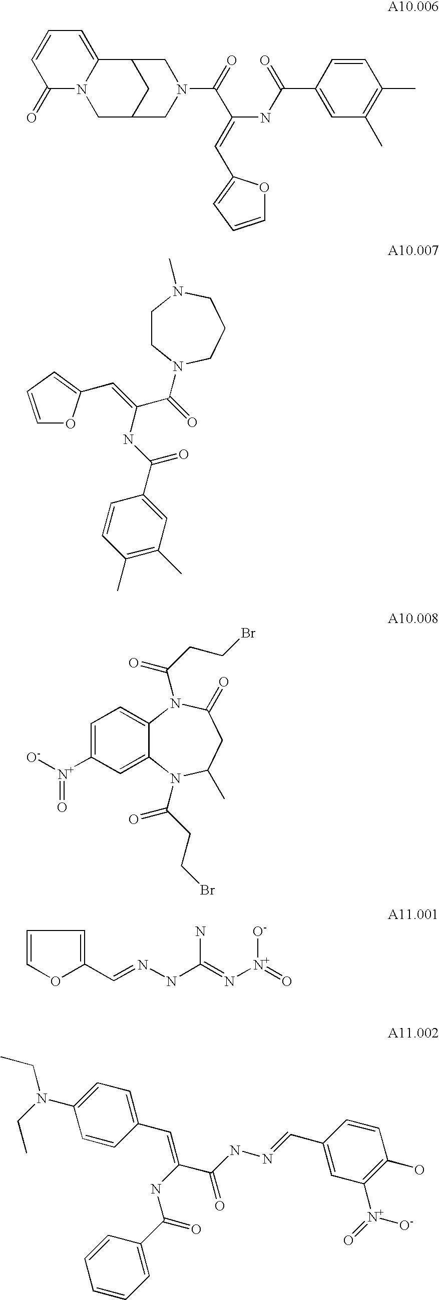

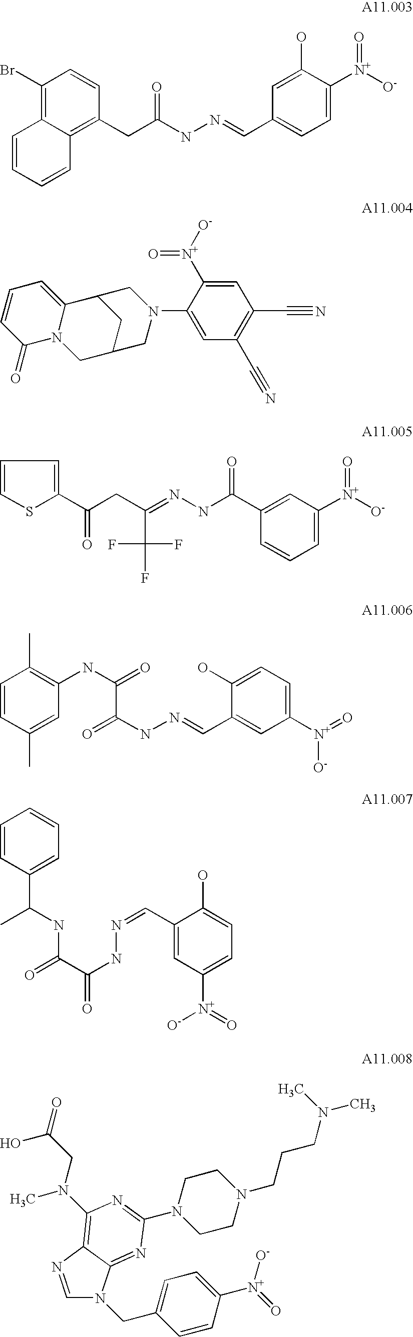

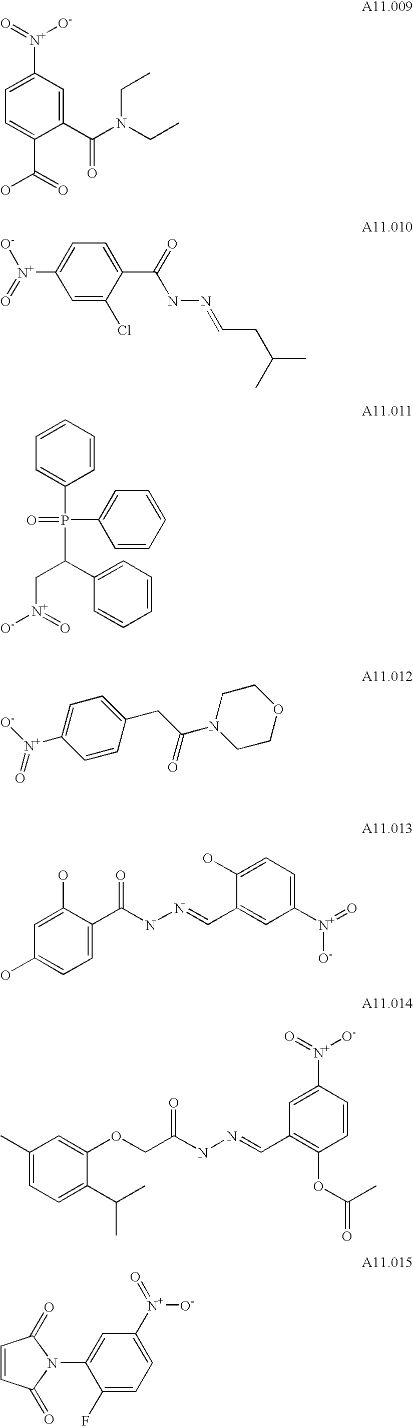

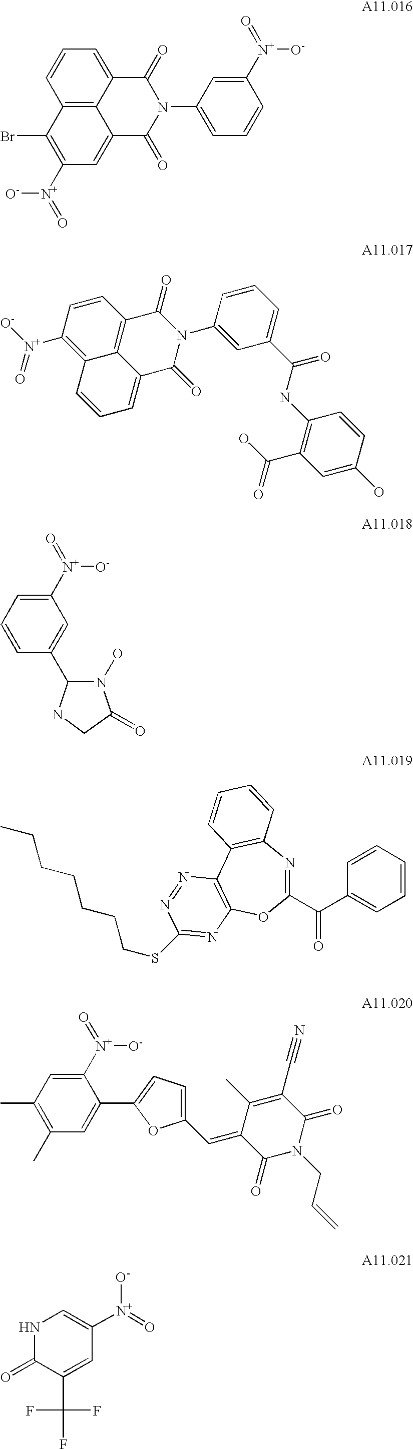

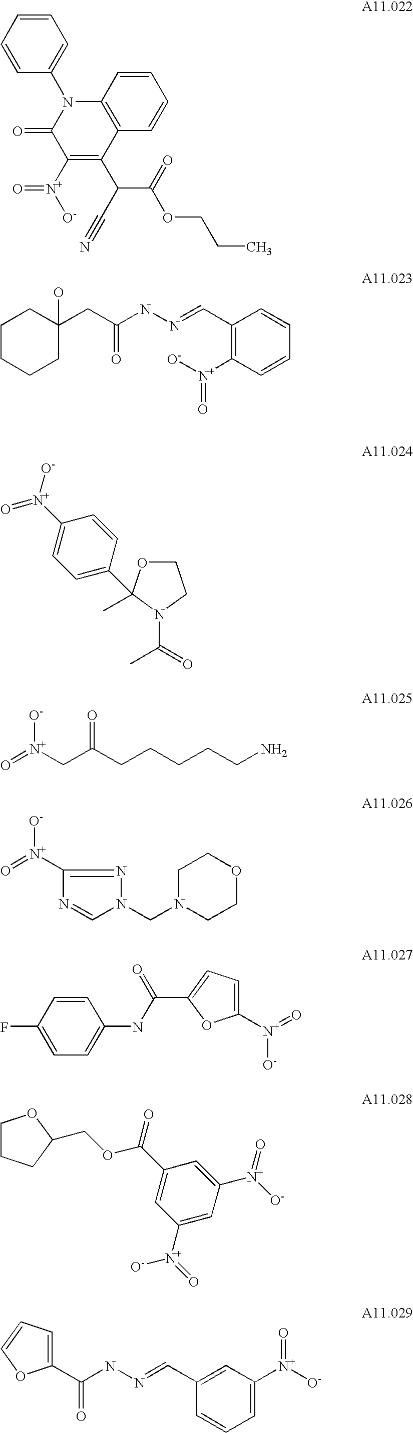

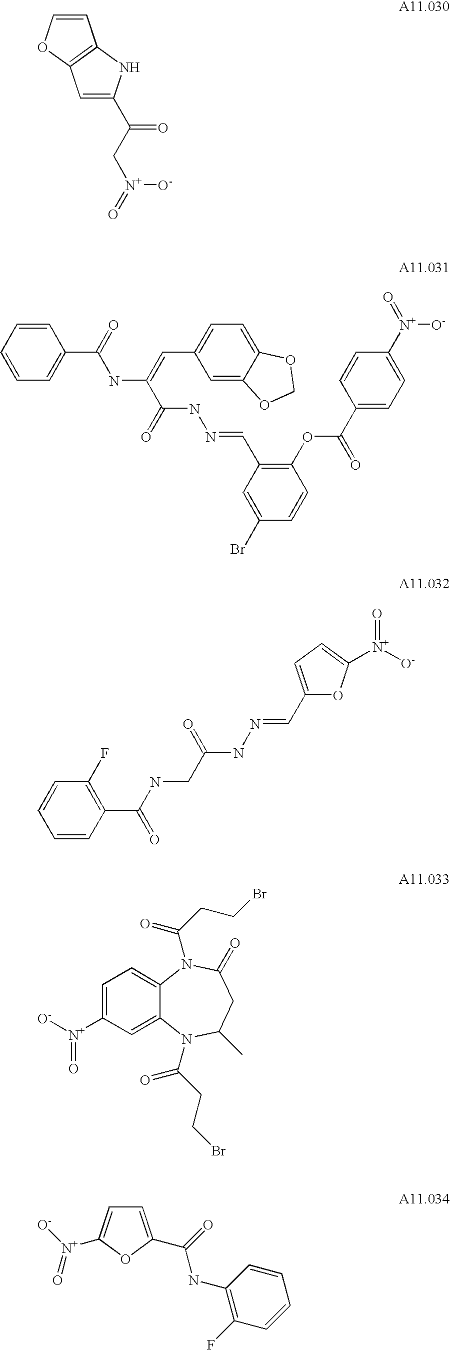

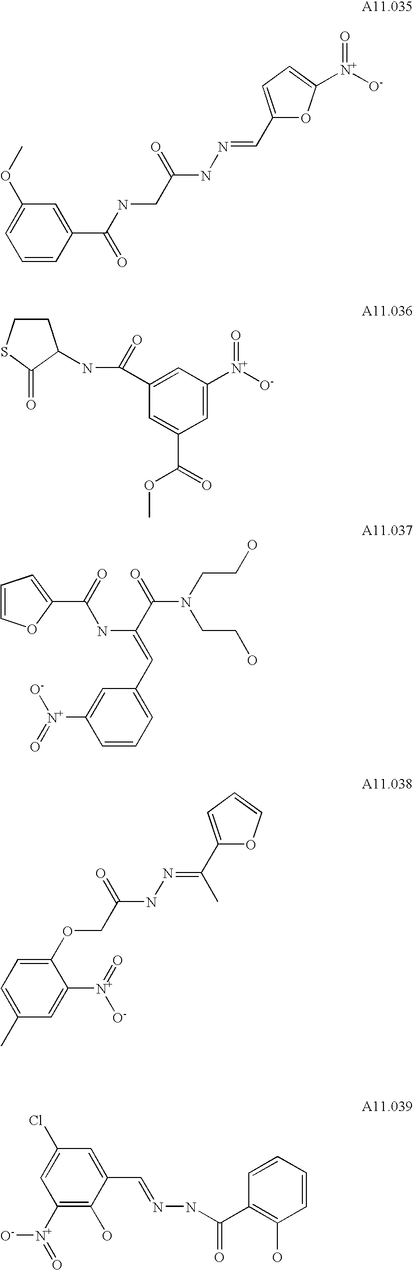

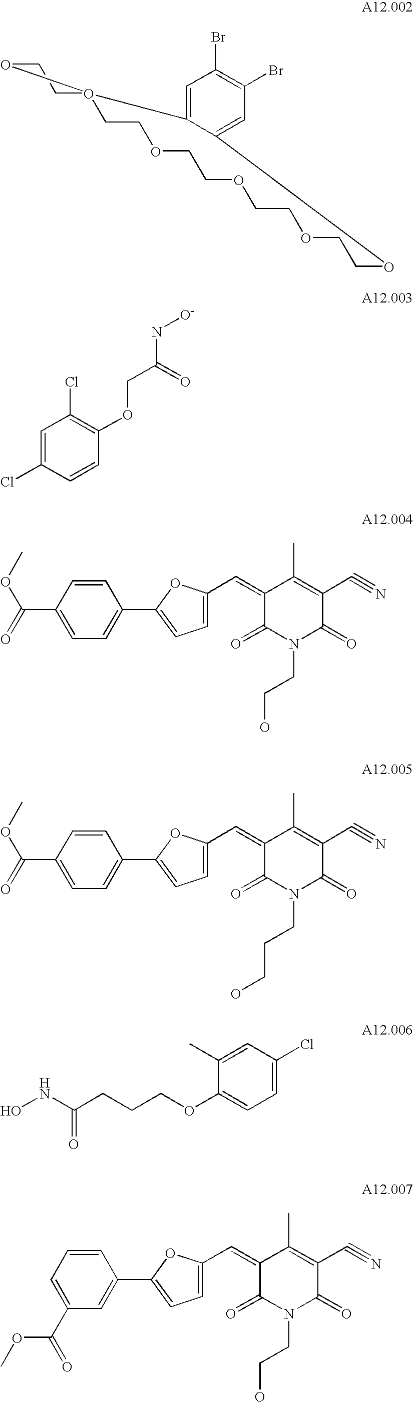

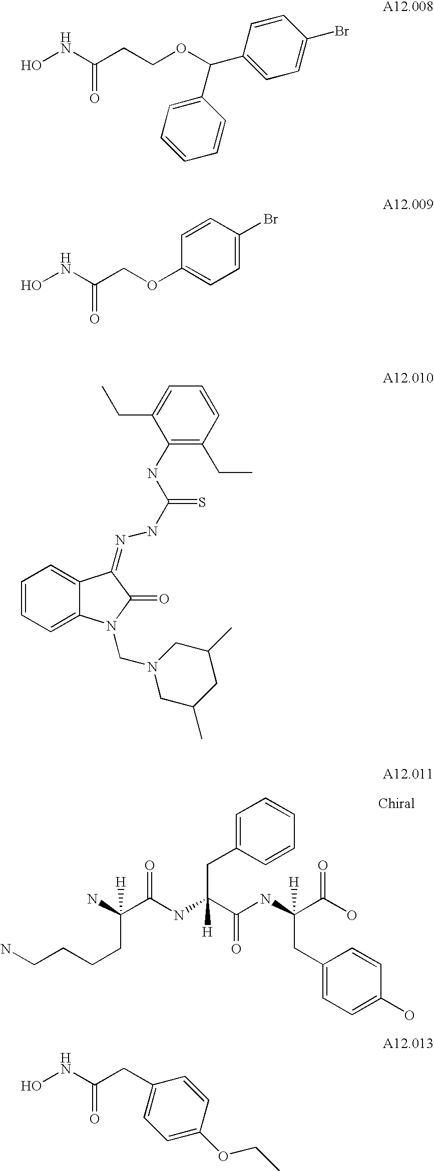

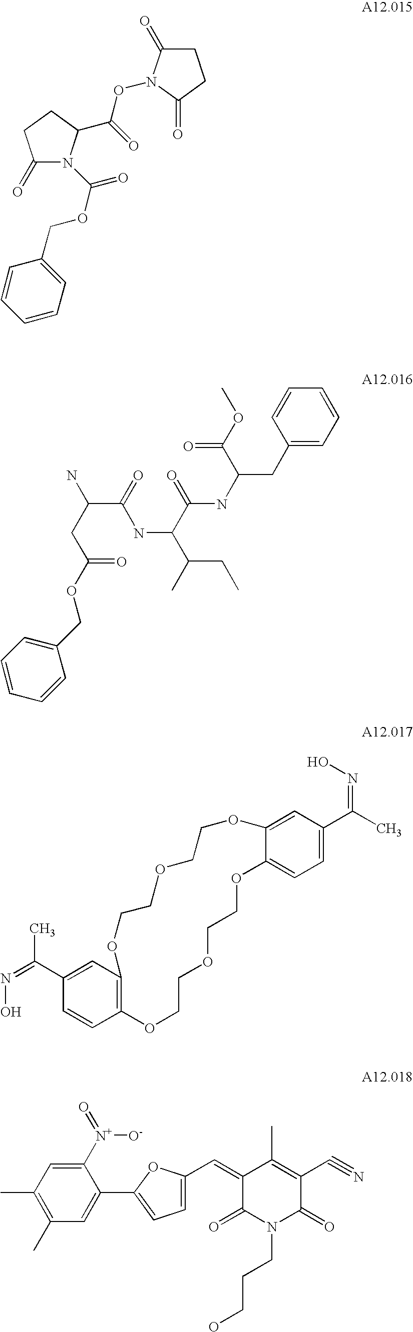

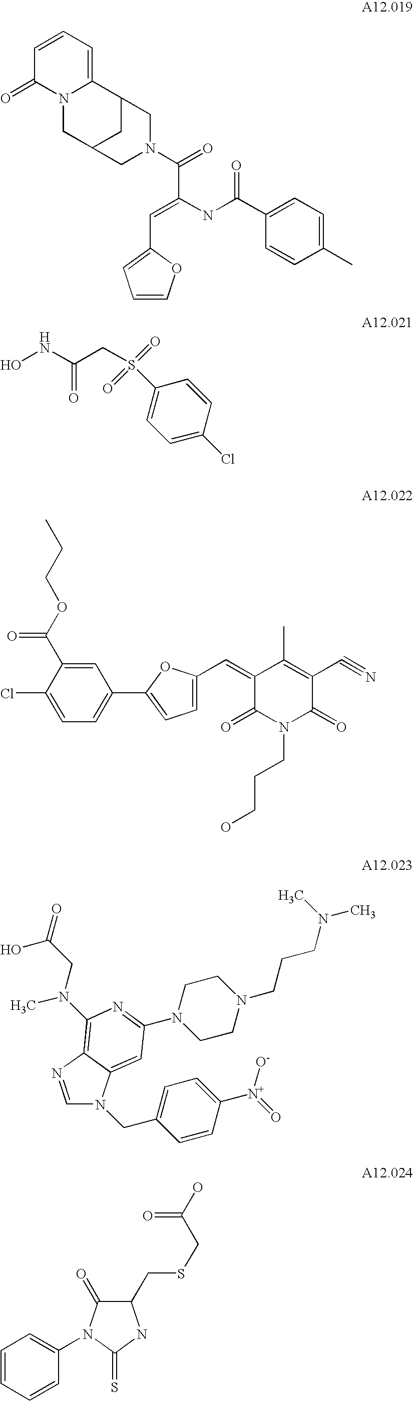

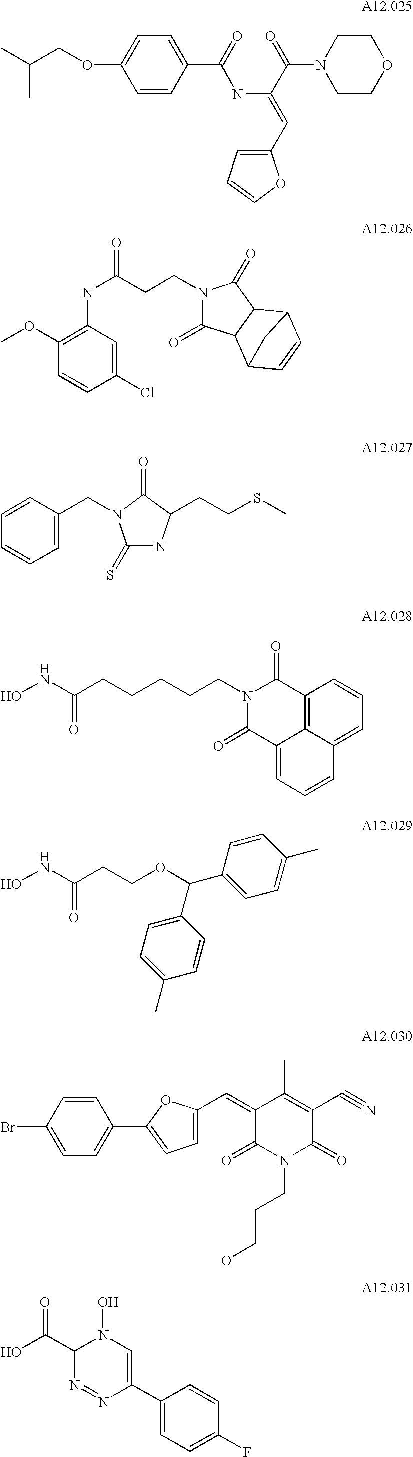

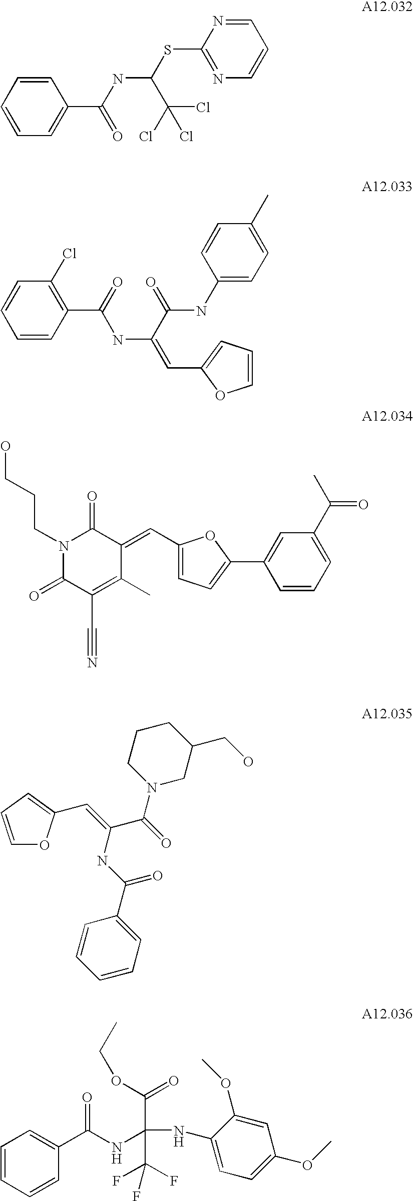

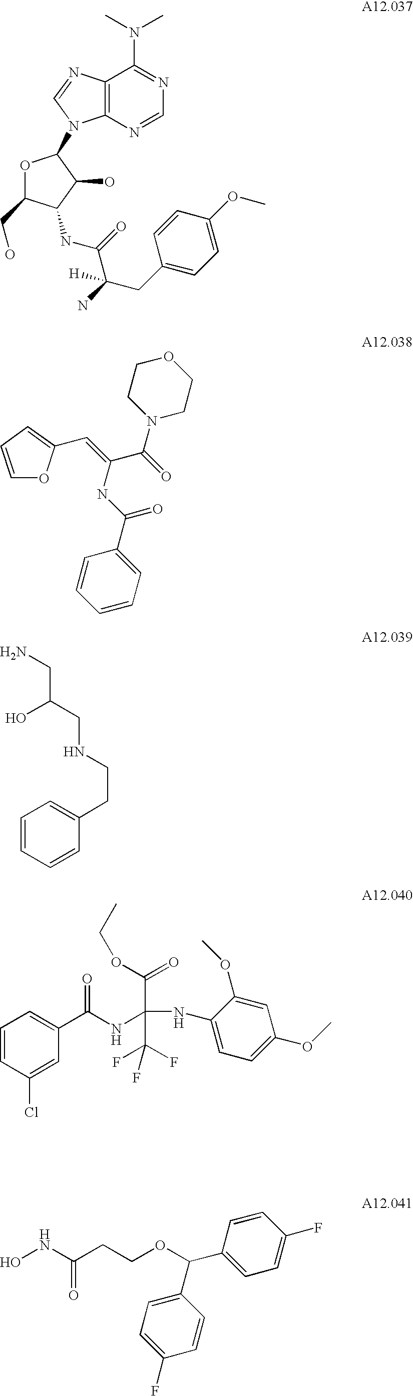

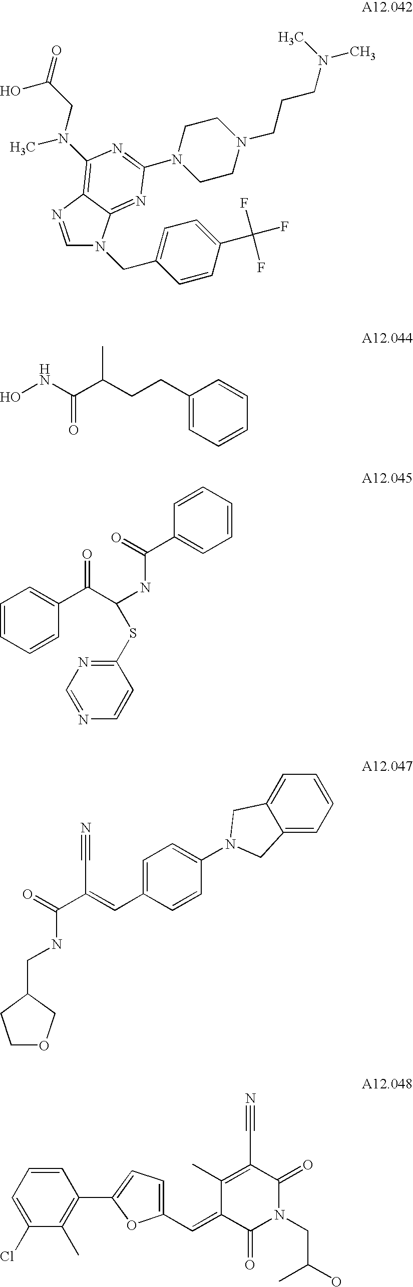

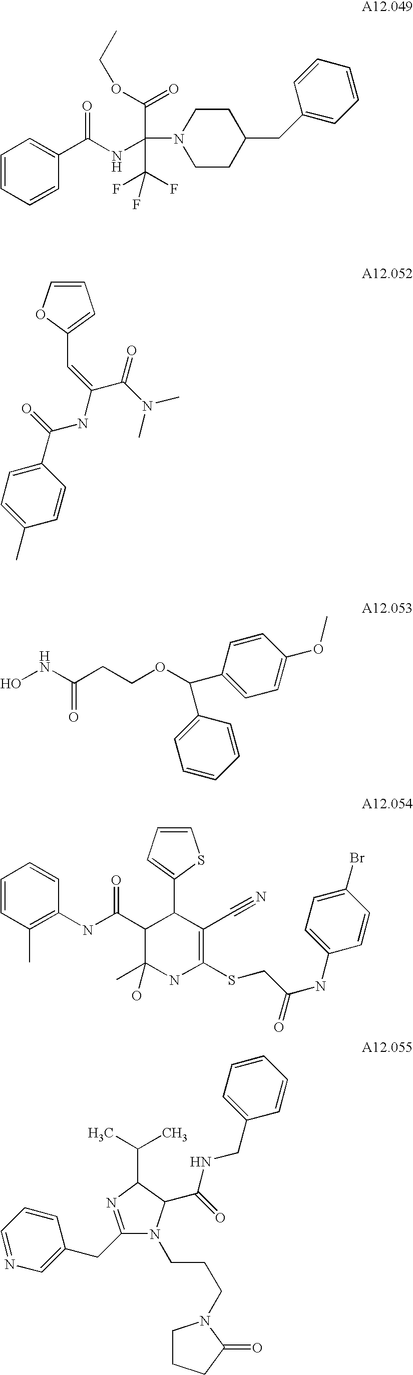

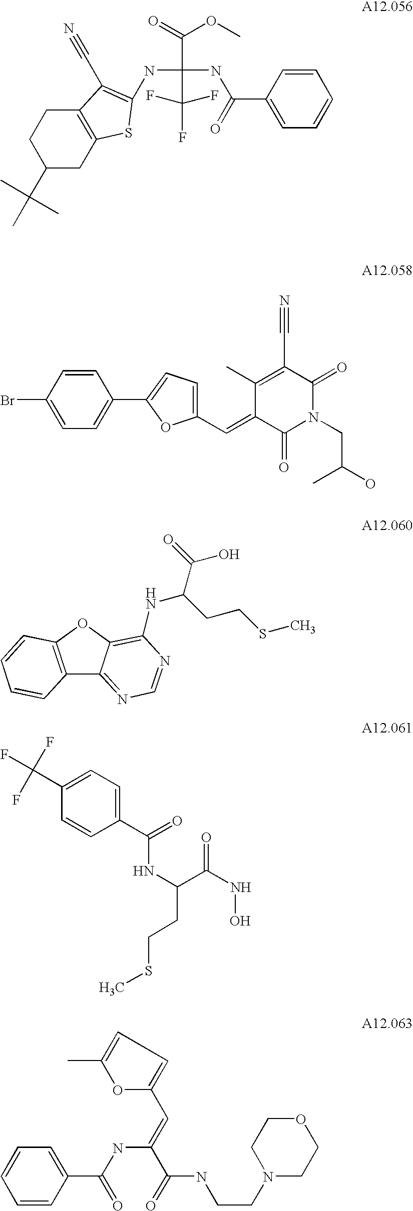

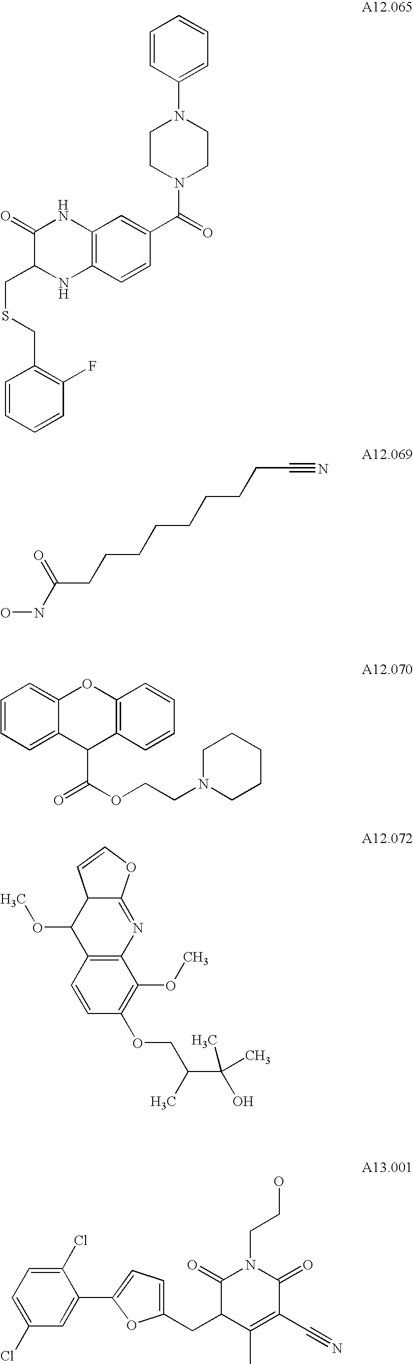

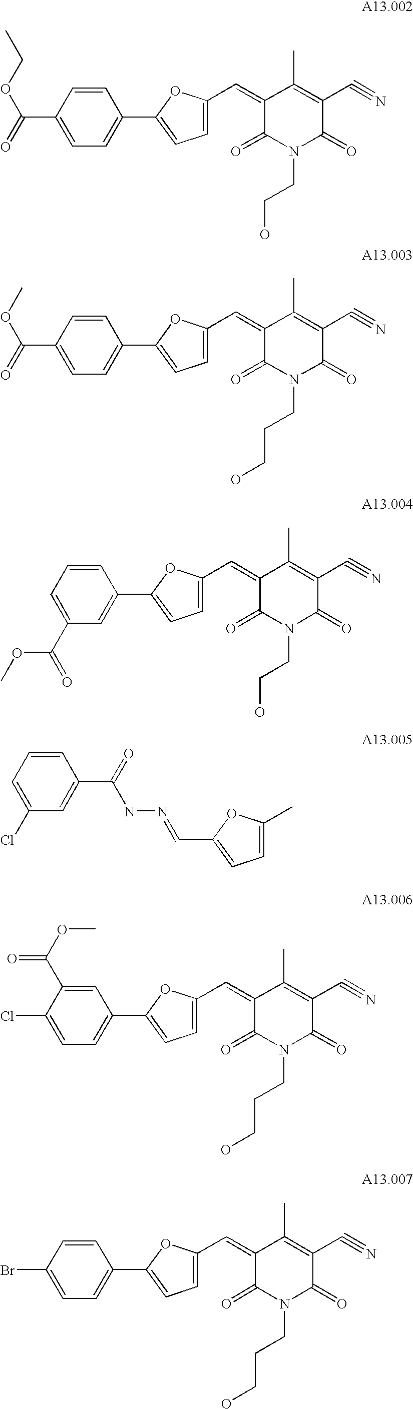

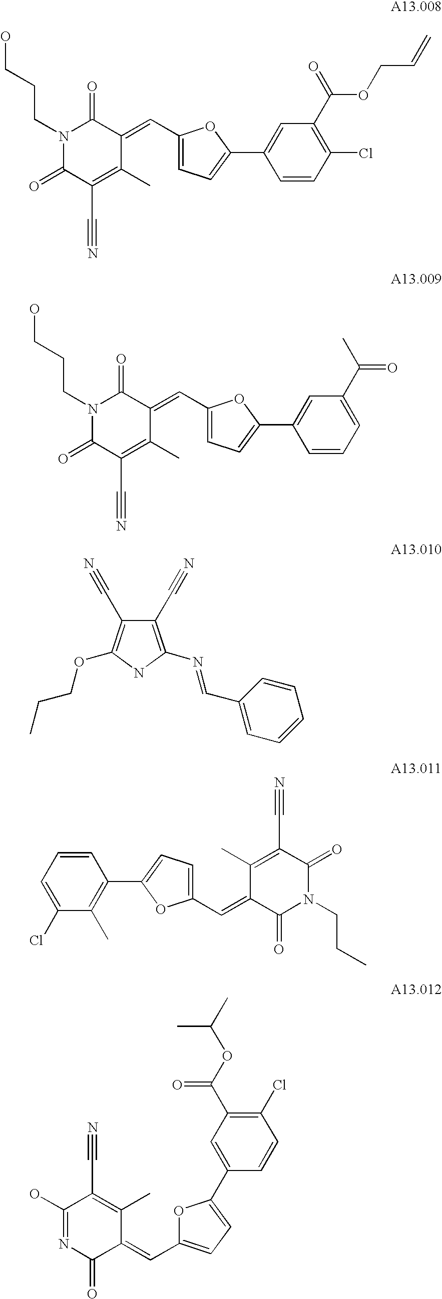

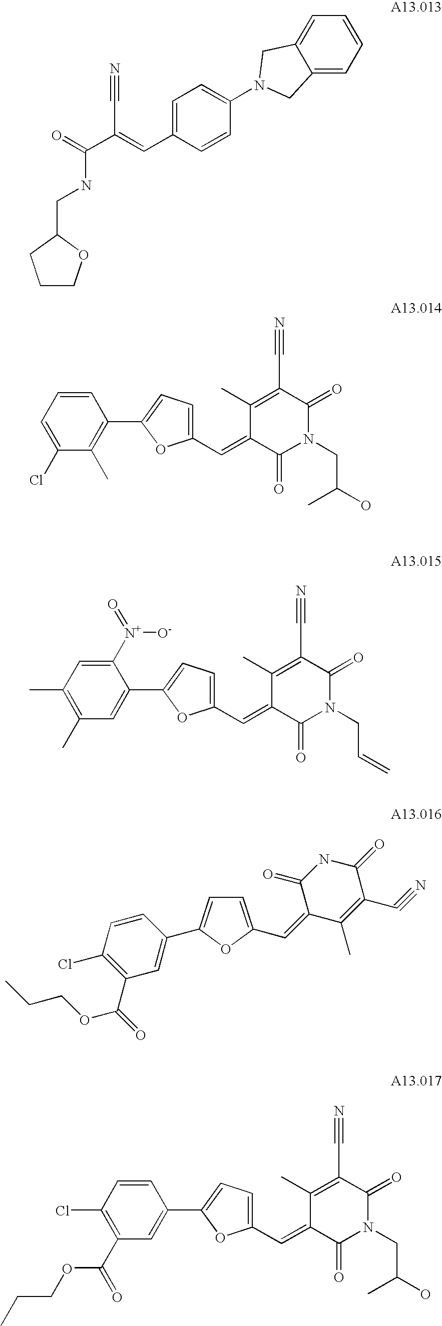

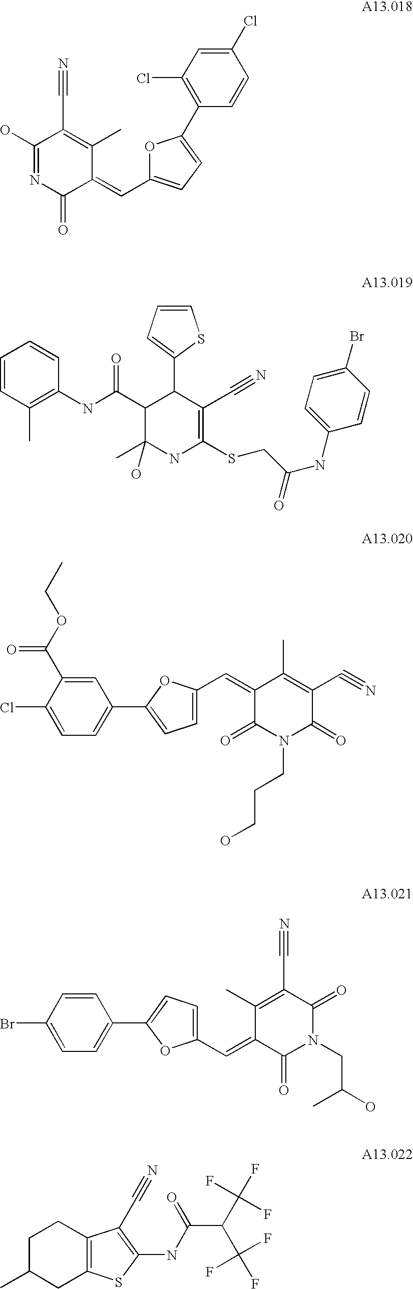

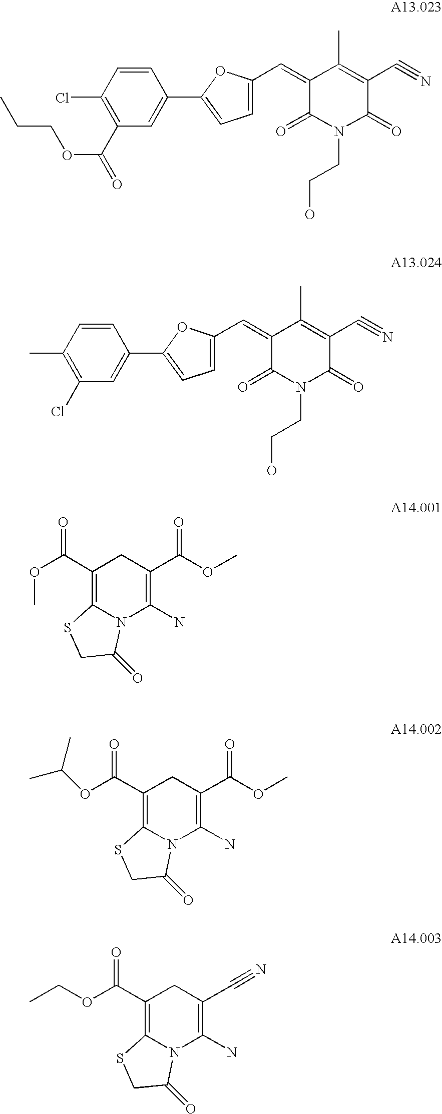

79. A method of inhibiting an activity of at least one enzyme selected from alanyl aminopeptidases and analogous enzymes in a subject in need thereof, wherein the method comprises administering to the subject at least one of a composition of claim 77 and an active ingredient thereof, alone or in combination with one or more inhibitors of dipeptidyl peptidase IV or analogous enzymes.

80. A method of inhibiting an activity of at least one enzyme selected from alanyl aminopeptidases and analogous enzymes in a subject in need thereof, wherein the method comprises administering to the subject at least one of a composition of claim 78 and an active ingredient thereof, alone or in combination with one or more inhibitors of dipeptidyl peptidase IV or analogous enzymes.

81. A method of topically influencing an activity of at least one enzyme selected from alanyl aminopeptidases and analogous enzymes in a subject in need thereof, wherein the method comprises topically administering to the subject at least one of a composition of claim 77 and an active ingredient thereof, alone or in combination with one or more inhibitors of dipeptidyl peptidase IV or analogous enzymes.

82. A method of topically influencing an activity of at least one enzyme selected from alanyl aminopeptidases and analogous enzymes in a subject in need thereof, wherein the method comprises topically administering to the subject at least one of a composition of claim 78 and an active ingredient thereof, alone or in combination with one or more inhibitors of dipeptidyl peptidase IV or analogous enzymes.

83. A method of preventing or treating at least one condition selected from multiple sclerosis, Morbus Crohn, Colitis ulcerosa and other autoimmune diseases; inflammatory diseases; allergic asthma bronchiale and other allergic diseases; rejection of transplanted tissues and cells; skin and mucosa diseases such as psoriasis and acne; dermatological diseases associated with a hyperproliferation and changed differentiation states of fibroblasts, preferably of benign fibrosing and sclerosing skin diseases and malign fibroblastar hyperproliferation states; acute neuronal diseases, in particular ischemia-caused cerebral damage after an ischemic or hemorrhagic stroke, cranio-cerebral trauma, cardiac arrest, myocardial infarction or as a consequence of heart surgery; chronic neuronal diseases, in particular Morbus Alzheimer, Pick's disease, Progressive Supranuclear Palsy, corticobasal degeneration, frontotemporal dementia, Morbus Parkinson, in particular Morbus Parkinson coupled to chromosome 17, Morbus Huntington, prion-caused diseases and amyotrophic lateral sclerosis; chronic obstructive pulmonal disease (COPD); prostata carcinoma and other tumors as well as metastases; Heavy Acute Respiratory Syndrome (SARS); and sepsis and sepsis-like conditions in a subject in need thereof, wherein the method comprises administering to the subject at least one of a composition of claim 77 and an active ingredient thereof in an amount sufficient for preventing or treating the at least one condition.

84. A method of preventing or treating at least one condition selected from multiple sclerosis, Morbus Crohn, Colitis ulcerosa and other autoimmune diseases; inflammatory diseases; allergic asthma bronchiale and other allergic diseases; rejection of transplanted tissues and cells; skin and mucosa diseases such as psoriasis and acne; dermatological diseases associated with a hyperproliferation and changed differentiation states of fibroblasts, preferably of benign fibrosing and sclerosing skin diseases and malign fibroblastar hyperproliferation states; acute neuronal diseases, in particular ischemia-caused cerebral damage after an ischemic or hemorrhagic stroke, cranio-cerebral trauma, cardiac arrest, myocardial infarction or as a consequence of heart surgery; chronic neuronal diseases, in particular Morbus Alzheimer, Pick's disease, Progressive Supranuclear Palsy, corticobasal degeneration, frontotemporal dementia, Morbus Parkinson, in particular Morbus Parkinson coupled to chromosome 17, Morbus Huntington, prion-caused diseases and amyotrophic lateral sclerosis; chronic obstructive pulmonal disease (COPD); prostata carcinoma and other tumors as well as metastases; Heavy Acute Respiratory Syndrome (SARS); and sepsis and sepsis-like conditions in a subject in need thereof, wherein the method comprises administering to the subject at least one of a composition of claim 78 and an active ingredient thereof in an amount sufficient for preventing or treating the at least one condition.

85. A method of preventing or treating at least one condition selected from atherosclerosis, arterial inflammation, reperfusion syndrome and stent restenosis, for example after a percutaneous transluminal angioplasty, in a subject in need thereof, wherein the method comprises administering to the subject at least one of a composition of claim 77 and an active ingredient thereof in an amount sufficient for preventing or treating the at least one condition.

86. The method of claim 85, wherein the method comprises administering the at least one of a composition and an active ingredient thereof by using a stent which is coated with the at least one of a composition and an active ingredient thereof.

87. A stent which is coated with at least one of a composition of claim 77 and an active ingredient thereof.

88. A method of preventing or treating at least one condition selected from atherosclerosis, arterial inflammation, reperfusion syndrome and stent restenosis, for example after a percutaneous transluminal angioplasty, in a subject in need thereof, wherein the method comprises administering to the subject at least one of a composition of claim 78 and an active ingredient thereof in an amount sufficient for preventing or treating the at least one condition.

89. The method of claim 88, wherein the method comprises administering the at least one of a composition and an active ingredient thereof by using a stent which is coated with the at least one of a composition and an active ingredient thereof.

90. A stent which is coated with at least one of a composition of claim 78 and an active ingredient thereof.

91. A method of preventing or treating an inflammation reaction at, or caused by, a medical device implanted into an organism, wherein the method comprises administering to the organism at least one of a composition of claim 77 and an active ingredient thereof in an amount sufficient for preventing or treating the inflammation reaction.

92. The method of claim 91, wherein the method comprises administering the at least one of a composition and an active ingredient thereof at least one of as a coating or layer on the medical device and incorporated in the medical device.

93. The method of claim 91, wherein the method comprises administering the at least one of a composition and an active ingredient thereof by at least one of a local and a systemic administration successively or concurrently.

94. A method of preventing or treating an inflammation reaction at, or caused by, a medical device implanted into an organism, wherein the method comprises administering to the organism at least one of a composition of claim 78 and an active ingredient thereof in an amount sufficient for preventing or treating the inflammation reaction.

95. The method of claim 94, wherein the method comprises administering the at least one of a composition and an active ingredient thereof at least one of as a coating or layer on the medical device and incorporated in the medical device.

96. The method of claim 94, wherein the method comprises administering the at least one of a composition and an active ingredient thereof by at least one of a local and a systemic administration successively or concurrently.
